(19) United States
(12) Patent Application Publication
(10) Pub. No.: US 2024/0335565 A1
(43) Pub. Date: Oct. 10, 2024

(54) JUNCTOPHILIN-2 (JPH2) GENE THERAPY USING AAV VECTOR

(71) Applicant: Spacecraft Seven, LLC, Cranbury, NJ (US)

(72) Inventors: Christopher Dean HERZOG, Cranbury, NJ (US); Chester Bittencort SACRAMENTO, Cranbury, NJ (US); David RICKS, Cranbury, NJ (US); Raj PRABHAKAR, Cranbury, NJ (US)

(21) Appl. No.: 18/718,021

(22) PCT Filed: Dec. 7, 2022
(86) PCT No.: PCT/US2022/081122

§ 371 Date: Jun. 8, 2024

Related U.S. Application Data
(60) Provisional application No. 63/287,393, filed on Dec. 8, 2021.

(51) Int. Cl. A61K 48/00; A61K 38/17; A61P 9/10; C12N 15/86

(52) U.S. Cl. CPC A61K 48/0058; A61K 38/1709; A61K 48/0083; A61P 9/10; C12N 15/86; C12N 2750/14143; C12N 2830/48; C12N 2830/50

(57) ABSTRACT

Provided herein is a gene therapy for JPH2 (Junctophilin-2), e.g., using an adeno-associated virus (AAV) vector. The promoter of the vector may be a MHCK7 promoter or a cardiac troponin T (HTNNT2) promoter. The capsid may be an AAV9 or AAVrh74 capsid or a functional variant thereof. Other promoters or capsids may be used. Further provided are methods of treatment, such as by intravenous, intracoronary, intracarotid or intracardiac administration of the rAAV vector, and other compositions and methods.

### CROSS-REFERENCE TO RELATED APPLICATIONS

This application claims benefit of priority to U.S. Provisional Patent Application No. 63/287,393, filed Dec. 8, 2021, the disclosure of which is incorporated herein by reference in its entirety for all purposes.

### STATEMENT REGARDING THE SEQUENCE LISTING

The Sequence Listing associated with this application is provided in text format in lieu of a paper copy and is hereby incorporated by reference into the specification. The name of the text file containing the Sequence Listing is ROPA_025_01WO_SeqList_ST26.xml. The text file is about 272,918 bytes, was created on Dec. 6, 2022, and is being submitted electronically via EFS-Web.

### BACKGROUND

Both hypertrophic cardiomyopathy (HCM) and dilated cardiomyopathy (DCM) are serious life-threatening diseases. Loss-of-function mutations in the gene Junctophilin-2 (JPH2) have been associated with HCM and DCM, as well as other heart conditions, such as atrial fibrillation (AF).

Junctophilin-2 (JPH2) is a structural protein that provides a bridge between transverse (T)-tubule associated cardiac L-type Ca²⁺ channels and type-2 ryanodine receptors on the sarcoplasmic reticulum within junctional membrane complexes (JMCs) in cardiomyocytes. Effective signaling between these channels ensures adequate Ca²⁺ release which is required for normal cardiac contractility. JPH2 downregulation is detected in disrupted JMC subcellular domains, a common feature of failing hearts.

Current treatment modalities including pharmacological therapies and cardiac ablation remain ineffective for JPH2-deficient cardiomyopathy patients. There remains, therefore, an unmet need in the art for treatments for JPH2-related diseases and disorders, including cardiomyopathy and other heart conditions. The compositions and methods disclosed herein address this need.

### SUMMARY

The present disclosure relates generally to gene therapy for a disease or disorder, e.g., a cardiac disease or disorder, using a vector expressing JPH2 or a functional variant thereof.

In one aspect, the disclosure provides a polynucleotide, comprising an expression cassette and optionally flanking adeno-associated virus (AAV) inverted terminal repeats (ITRs), wherein the polynucleotide comprises a polynucleotide sequence encoding a Junctophilin-2 (JPH2), or a functional variant thereof, operatively linked to a promoter.

In some embodiments, the promoter is a cardiac-specific promoter. In some embodiments, the promoter is a muscle-specific promoter. In some embodiments, the promoter is a cardiomyocyte-specific promoter.

In some embodiments, the promoter is a Myosin Heavy-chain Creatine Kinase 7 (MHCK7) promoter. In some embodiments, the MHCK7 promoter shares at least 75%, 80%, 85%, 90%, 95%, 96%, 97%, 98%, 99% or 100% identity with SEQ ID NO: 31.

In some embodiments, the promoter is a cardiac troponin T (hTNNT2) promoter. In some embodiments, the hTNNT2 promoter shares at least 75%, 80%, 85%, 90%, 95%, 96%, 97%, 98%, 99% or 100% identity with SEQ ID NO: 32. In some embodiments, the expression cassette comprises exon 1 of the cardiac troponin T (hTNNT2) gene, wherein optionally the hTNNT2 promoter and exon 1 together share at least 75%, 80%, 85%, 90%, 95%, 96%, 97%, 98%, 99% or 100% identity with SEQ ID NO: 32.

In some embodiments, the promoter is a ubiquitous promoter, optionally a CMV promoter or a CAG promoter.

In some embodiments, the expression cassette comprises a polyA signal. In some embodiments, the polyA signal is a human growth hormone (hGH) polyA.

In some embodiments, the expression cassette comprises a Woodchuck Hepatitis Virus Posttranscriptional Regulatory Element (WPRE), optionally a WPRE(x).

In some embodiments, the expression cassette comprises a Green Fluorescence Protein (GFP).

In some embodiments, the Junctophilin-2 (JPH2) or functional variant thereof is a JPH2. In some embodiments, the JPH2 is a human JPH2. In some embodiments, the polynucleotide sequence encoding JPH2 is a human JPH2 polynucleotide.

In some embodiments, the polynucleotide sequence encoding JPH2 shares at least 75%, 80%, 85%, 90%, 95%, 96%, 97%, 98%, 99% or 100% identity with SEQ ID NO: 2.

In some embodiments, the polynucleotide sequence encoding JPH2 shares at least 75%, 80%, 85%, 90%, 95%, 96%, 97%, 98%, 99% or 100% identity with SEQ ID NO: 4.

In some embodiments, the polynucleotide sequence encoding JPH2 shares at least 75%, 80%, 85%, 90%, 95%, 96%, 97%, 98%, 99% or 100% identity with SEQ ID NO: 6.

In some embodiments, the polynucleotide sequence encoding JPH2 shares at least 75%, 80%, 85%, 90%, 95%, 96%, 97%, 98%, 99% or 100% identity with SEQ ID NO: 8.

In some embodiments, the polynucleotide sequence encoding JPH2 shares at least 75%, 80%, 85%, 90%, 95%, 96%, 97%, 98%, 99% or 100% identity with SEQ ID NO: 10.

In some embodiments, the polynucleotide comprises at least about 3.0 kb, at least about 3.2 kb, at least about 3.4 kb, at least about 3.5 kb, at least about 3.7 kb, at least about 4.0 kb, at least about 4.1 kb, at least about 4.2 kb, at least about 4.3 kb, at least about 4.4 kb, at least about 4.5 kb, at least about 4.6 kb, at least about 4.7 kb, at least about 4.8 kb, or at least about 5.0 kb.

In some embodiments, the polynucleotide comprises at most about 3.1 kb, at most about 3.3 kb, at most about 3.5 kb, at most about 3.7 kb, at most about 3.9 kb, at most about 4.1 kb, at most about 4.2 kb, at most about 4.3 kb, at most about 4.4 kb, at most about 4.5 kb, at most about 4.6 kb, at most about 4.7 kb, at most about 4.8 kb, at most about 4.9 kb, or at most about 5.0 kb.

In some embodiments, the polynucleotide comprises 4.4 kb to 5.0 kb, 4.4 kb to 4.9 kb, or 4.4 kb to 4.8 kb, wherein the polynucleotide comprises 4.0 kb to 4.6 kb, 4.0 kb to 4.5 kb, or 4.0 kb to 4.4 kb, wherein the polynucleotide comprises 4.0 kb to 4.3 kb, 4.0 kb to 4.2 kb, or 4.0 kb to 4.1 kb, or wherein the polynucleotide comprises 3.0 kb to 3.9 kb, 3.0 kb to 3.8 kb, or 3.0 kb to 3.7 kb.

In some embodiments, the JPH2 or functional variant thereof comprises at least 600 or at least 630 amino acids.

In some embodiments, the JPH2 or functional variant thereof comprises at least 600 or at least 696 amino acids.

In some embodiments, the JPH2 or functional variant thereof comprises at least 100 or at least 129 amino acids.

In some embodiments, the expression cassette is flanked by 5′ and 3′ inverted terminal repeats (ITRs). In some embodiments, the ITRs are AAV2 ITRs and/or the ITRs share at least 75%, 80%, 85%, 90%, 95%, 96%, 97%, 98%, 99% or 100% identity with any one of SEQ ID NO: 15-21.

In one aspect, the disclosure provides a gene therapy vector, comprising the polynucleotide as described in the present disclosure. In some embodiments, the gene therapy vector is a recombinant adeno-associated virus (rAAV) vector. In some embodiments, the rAAV vector is an AAV9 or a functional variant thereof. In some embodiments, the rAAV vector comprises a capsid protein that shares 90%, 91%, 92%, 93%, 94%, 95%, 96%, 97%, 98%, 99% or 100% identity to any one of SEQ ID NO: 97. In some embodiments, the rAAV vector is an AAVrh10 or a functional variant thereof. In some embodiments, the rAAV vector comprises a capsid protein that shares 90%, 91%, 92%, 93%, 94%, 95%, 96%, 97%, 98%, 99% or 100% identity to any one of SEQ ID NO: 99. In some embodiments, the rAAV vector is an AAV6 or a functional variant thereof. In some embodiments, the rAAV vector comprises a capsid protein that shares 90%, 91%, 92%, 93%, 94%, 95%, 96%, 97%, 98%, 99% or 100% identity to any one of SEQ ID NO: 98.

In some embodiments, the rAAV vector is an AAVrh74 or a functional variant thereof. In some embodiments, the rAAV vector comprises a capsid protein that shares 90%, 91%, 92%, 93%, 94%, 95%, 96%, 97%, 98%, 99% or 100% identity to any one of SEQ ID NO: 100.

In one aspect, the disclosure provides a method of treating and/or preventing a disease or disorder in a subject in need thereof, comprising administering the vector of the present disclosure to the subject.

In some embodiments, the disease or disorder is a cardiac disorder. In some embodiments, the cardiac disorder is a cardiomyopathy, such as hypertrophic cardiomyopathy (HCM) or dilated cardiomyopathy (DCM). In some embodiments, the disease or disorder is arrhythmia. In some embodiments, the arrhythmia is atrial fibrillation. In some embodiments, the arrhythmia is sinus node disease. In some embodiments, the disease or disorder is familial hypertrophic cardiomyopathy 17. In some embodiments, the disease or disorder is heart failure.

In some embodiments, the subject is a mammal. In some embodiments, the subject is a primate. In some embodiments, the subject is a human.

In some embodiments, the subject has a mutation in a JPH2 gene. In some embodiments, the subject has a truncated variant of JPH2.

In some embodiments, the vector is administered by intravenous administration, intracardiac administration, intracoronary administration, intracardiac administration, and/or cardiac catheterization. In certain embodiments, any of the routes of administration may be performed by infusion or injection.

In some embodiments, the administration increases JPH2 expression by at least about 5%. In some embodiments, the administration increases JPH2 expression by at least about 30%. In some embodiments, the administration increases JPH2 expression by at least about 70%. In some embodiments, the administration increases JPH2 expression by about 5% to about 10%. In some embodiments, the administration increases JPH2 expression by about 30% to about 50%. In some embodiments, the administration increases JPH2 expression by about 50% to about 70%. In some embodiments, the administration increases JPH2 expression by about 70% to about 100%.

In one aspect, the disclosure provides a method that treats and/or prevents the disease or disorder. In some embodiments, the method comprises administering an effective amount of the vector. In some embodiments, the disease or disorder is related to or caused by truncation of JPH2 in the subject. In some embodiments, the method comprises administering a pharmaceutical composition comprising an effective amount of the vector.

In some embodiments, the method comprises administering between about 1×10¹¹ vector genomes and about 1×10¹³ vector genomes of the vector to the subject, administering between about 1×10¹² vector genomes and about 1×10¹⁴ vector genomes of the vector to the subject, administering between about 1×10¹³ vector genomes and about 1×10¹⁵ vector genomes, or administering between about 1×10¹⁵ vector genomes and about 1×10¹⁷ vector genomes, or administering between about 1×10¹⁷ vector genomes and about 1×10¹⁸ vector genomes of the vector to the subject, or any range between any two of these values.

In one aspect, the disclosure provides a pharmaceutical composition comprising the vector of the present disclosure.

In one aspect, the disclosure provides a kit comprising the vector of the present disclosure or the pharmaceutical composition of the present disclosure and optionally instructions for use.

In one aspect, the disclosure provides a use of the vector of the present disclosure in treating a disease or disorder, optionally according to the method of the present disclosure.

In one aspect, the disclosure provides a vector according to the present disclosure for use in treating a disease or disorder, optionally according to the method of the present disclosure.

In one aspect, the disclosure provides a polynucleotide, comprising a polynucleotide sequences that shares at least 90%, 91%, 92%, 93%, 94%, 95%, 96%, 97%, 98%, 99%, or 100% identity to any one of SEQ ID NOs: 26-30 or to any one of SEQ ID NOs: 76-95.

In some embodiments, the promoter is a MHCK7 promoter. In some embodiments, the MHCK7 promoter shares at least 75%, 80%, 85%, 90%, 95%, 96%, 97%, 98%, 99% or 100% identity with SEQ ID NO: 31.

Various other aspects and embodiments are disclosed in the detailed description that follows. The disclosure is limited solely by the appended claims.

### BRIEF DESCRIPTION OF FIGURES

FIG. 1 shows a diagram illustrating a non-limiting example of a vector genome. The full polynucleotide sequence of the vector genome is SEQ ID NO: 26. The MHCK7 promoter as described herein is labelled “Enhancer/MHCK7” in the diagram.

FIG. 2 shows a diagram illustrating a non-limiting example of a vector genome. The full polynucleotide sequence of the vector genome is SEQ ID NO: 27.

FIG. 3 shows a diagram illustrating a non-limiting example of a vector genome. The full polynucleotide sequence of the vector genome is SEQ ID NO: 28. The MHCK7 promoter as described herein is labelled “Enhancer/MHCK7” in the diagram.

FIG. 4 shows a diagram illustrating a non-limiting example of a vector genome. The full polynucleotide sequence of the vector genome is SEQ ID NO: 29.

FIG. 5 shows a diagram illustrating a non-limiting example of a vector genome. The full polynucleotide sequence of the vector genome is SEQ ID NO: 30.

FIG. 6 shows a diagram illustrating a non-limiting example of a vector genome. The full polynucleotide sequence of the vector genome is SEQ ID NO: 76. The MHCK7 promoter as described herein is labelled “Enhancer/MHCK7” in the diagram.

FIG. 7 shows a diagram illustrating a non-limiting example of a vector genome. The full polynucleotide sequence of the vector genome is SEQ ID NO: 77.

FIG. 8 shows a diagram illustrating a non-limiting example of a vector genome. The full polynucleotide sequence of the vector genome is SEQ ID NO: 78. The MHCK7 promoter as described herein is labelled “Enhancer/MHCK7” in the diagram.

FIG. 9 shows a diagram illustrating a non-limiting example of a vector genome. The full polynucleotide sequence of the vector genome is SEQ ID NO: 79.

FIG. 10 shows a diagram illustrating a non-limiting example of a vector genome. The full polynucleotide sequence of the vector genome is SEQ ID NO: 80.

FIG. 11 shows a diagram illustrating a non-limiting example of a vector genome. The full polynucleotide sequence of the vector genome is SEQ ID NO: 81. The MHCK7 promoter as described herein is labelled “Enhancer/MHCK7” in the diagram.

FIG. 12 shows a diagram illustrating a non-limiting example of a vector genome. The full polynucleotide sequence of the vector genome is SEQ ID NO: 82.

FIG. 13 shows a diagram illustrating a non-limiting example of a vector genome. The full polynucleotide sequence of the vector genome is SEQ ID NO: 83. The MHCK7 promoter as described herein is labelled “Enhancer/MHCK7” in the diagram.

FIG. 14 shows a diagram illustrating a non-limiting example of a vector genome. The full polynucleotide sequence of the vector genome is SEQ ID NO: 84.

FIG. 15 shows a diagram illustrating a non-limiting example of a vector genome. The full polynucleotide sequence of the vector genome is SEQ ID NO: 85.

FIG. 16 shows a diagram illustrating a non-limiting example of a vector genome. The full polynucleotide sequence of the vector genome is SEQ ID NO: 86. The MHCK7 promoter as described herein is labelled “Enhancer/MHCK7” in the diagram.

FIG. 17 shows a diagram illustrating a non-limiting example of a vector genome. The full polynucleotide sequence of the vector genome is SEQ ID NO: 87.

FIG. 18 shows a diagram illustrating a non-limiting example of a vector genome. The full polynucleotide sequence of the vector genome is SEQ ID NO: 88. The MHCK7 promoter as described herein is labelled “Enhancer/MHCK7” in the diagram.

FIG. 19 shows a diagram illustrating a non-limiting example of a vector genome. The full polynucleotide sequence of the vector genome is SEQ ID NO: 89.

FIG. 20 shows a diagram illustrating a non-limiting example of a vector genome. The full polynucleotide sequence of the vector genome is SEQ ID NO: 90.

FIG. 21 shows a diagram illustrating a non-limiting example of a vector genome. The full polynucleotide sequence of the vector genome is SEQ ID NO: 91. The MHCK7 promoter as described herein is labelled “Enhancer/MHCK7” in the diagram.

FIG. 22 shows a diagram illustrating a non-limiting example of a vector genome. The full polynucleotide sequence of the vector genome is SEQ ID NO: 92.

FIG. 23 shows a diagram illustrating a non-limiting example of a vector genome. The full polynucleotide sequence of the vector genome is SEQ ID NO: 93. The MHCK7 promoter as described herein is labelled “Enhancer/MHCK7” in the diagram.

FIG. 24 shows a diagram illustrating a non-limiting example of a vector genome. The full polynucleotide sequence of the vector genome is SEQ ID NO: 94.

FIG. 25 shows a diagram illustrating a non-limiting example of a vector genome. The full polynucleotide sequence of the vector genome is SEQ ID NO: 95.

FIG. 26 shows JPH2 protein expression in the heart of C57BL/6J mice. The figure shows Western Blots (WB) of JPH2 (top panel) or loading control, GAPDH (bottom panel).

FIG. 27 illustrates the experimental timeline.

FIGS. 28A-28B illustrate left ventricle ejection fraction percentage (EF %) across time (FIG. 28A) and at 9 weeks post-TAC (6 weeks following AAV injection; FIG. 28B). Statistical analyses (One-way ANOVA) followed by Dunnett's post-hoc comparisons revealed significant benefit of AAV treated groups comparing to the untreated group (*p≤0.05, **p≤0.01, ***p≤0.001, ****p<0.0001). For FIG. 28A, at 5 weeks post-TAC, the lines from top to bottom correspond to: FB (Neg CON), AAV9-hTnT-JPH2, AAVrh. 74-hTnt-JPH2, AAV9-MHCK7-JPH2, FB (POS CON), and AAVrh.74-MHCK7-JPH2.

FIGS. 29A-29F show left ventricle ejection fraction percentage (EF %) across time. FIG. 29A shows results for normal mice, and FIG. 29B shows results for untreated TAC mice. FIG. 29C shows results with the AAV9 vector and MHCK7 promoter (AAV9-MHCK7). FIG. 29D shows results with the AAVrh. 74 vector and MHCK7 promoter (AAVrh.74-MHCK7). FIG. 29E shows results with the AAV9 vector and hTnT promoter (AAV9-hTnT). FIG. 29F shows results with the AAVrh. 74 vector and hTnT promoter (AAVrh.74-hTnT).

### DETAILED DESCRIPTION OF THE DISCLOSURE

The present disclosure provided gene therapy vectors for JPH2 that deliver a polynucleotide encoding a JPH2 polypeptide or a functional variant thereof, along with methods of use, and other compositions and methods. In particular embodiments, the disclosure relates to a gene therapy vector comprising a promoter sequence operatively linked to a polynucleotide encoding a JPH2 polypeptide or a functional variant thereof. In some embodiments, the promoter is a Myosin Heavy-chain Creatine Kinase 7 (MHCK7) promoter. In some embodiments, the AAV vector is an AAV9 vector. In some embodiments, the promoter is an MHCK7 promoter and the AAV vector is an AAV9 vector. In some embodiments, the promoter is a hTNNT2 promoter. In some embodiments, the promoter is an hTNNT2 promoter and the AAV vector is an AAV9 vector. In some embodiments, the JPH2 is human JPH2. In some embodiments, the JPH2 is human JPH2 isoform 1 (SEQ ID NO:1). In some embodiments, the JPH2 is human JPH2 isoform 2 (SEQ ID NO:108). In some embodiments, the AAV vector is a rh74 vector. In some embodiments, the promoter is an MHCK7 promoter and the AAV vector is a rh74 vector. In some embodiments, the promoter is a hTNNT2 promoter. In some embodiments, the promoter is a hTNNT2 promoter and the AAV vector is a rh74 vector. In some embodiments, the JPH2 is human JPH2.

This disclosure further provides methods of treating a disorder or disorder in a subject by administering a gene therapy vector of the disclosure to the subject. In a certain embodiment, the disorder or disorder is atrial fibrillation. In a certain embodiment, the disorder or disorder is an arrhythmia. In a certain embodiment, the disorder or disorder is sinus node disease. In a certain embodiment, the disorder or disorder is familial hypertrophic cardiomyopathy 17. Mutations in JPH2 are associated with familial hypertrophic cardiomyopathy 17 and atrial fibrillation.

In certain embodiments, the subject being treated is a heart failure patient having one or more mutations or truncations in a JPH2 gene. The expression level of JPH2 is decreased in failing hearts of multiple etiologies including human heart failure. Heart failure patients carry a JPH2 fragment, generated during cardiac stress.

The gene JPH2 encodes the protein Junctophilin-2 (JPH2). JPH2 is a membrane-binding protein that provides a structural bridge between transverse (T)-tubule associated cardiac L-type Ca²⁺ channels in the plasma membrane and type-2 ryanodine receptors on the sarcoplasmic reticulum within junctional membrane complexes (JMCs) in cardiomyocytes. Its structure provides a structural foundation for functional cross-talk between the cell surface and intracellular Ca²⁺ release channels by maintaining the 12-15 nm gap between the sarcolemma and the sarcoplasmic reticulum membranes in the cardiac dyads. JPH2 is required for normal excitation-contraction coupling in cardiomyocytes and contributes to the construction of skeletal muscle triad junctions.

Following cardiac stress, JPH2 is cleaved by Ca²⁺-dependent protease calpain, which liberates an N-terminal fragment (JPH2NT) that translocates to the nucleus, binds to genomic DNA and controls expression of a spectrum of genes in cardiomyocytes. Stress-induced proteolysis of JPH2 disrupts the ultrastructural machinery and drives heart failure progression.

Cleavage by calpains is one of the main mechanisms underlying the loss of JPH2 levels in failing hearts. Because calpain-1 and calpain-2 activity are increased in myocardial tissue subjected to stress (i.e., ischemia, oxidative stress, HF), Ca²⁺-dependent proteolysis of JPH2 has been observed under pathological conditions. Human JPH2 contains three calpain cleavage sites. Calpain-1 and calpain-2 can cleave JPH2 at amino acids 572 and 573 of SEQ ID NO: 1. Calpain-1 can also cleave JPH2 at the sites found at amino acids 155 and 156, and at amino acids amino acids 204 and 205 of human JPH2, isoform 1, as set forth in SEQ ID NO: 1. Additional Calpain-2 cleavage sites are disclosed in Weninger et al. Sci Rep 12, 10387 (2022), which is incorporated by reference in its entirety.

In some embodiments of the instant disclosure, a polynucleotide encoding JPH2 for use in generating a gene therapy vector may comprise alanine substitutions for amino acids 155 and 156 of a JPH2 reference sequence as set forth in SEQ ID NO: 1. In some embodiments, a polynucleotide encoding JPH2 for use in generating a gene therapy vector may comprise alanine substitutions for amino acids 204 and 205 of a JPH2 reference sequence as set forth in SEQ ID NO: 1. In some embodiments, a polynucleotide encoding JPH2 for use in generating a gene therapy vector may comprise alanine substitutions for amino acids 573 and 573 of a JPH2 reference sequence as set forth in SEQ ID NO: 1. In some embodiments, a polynucleotide encoding JPH2 for use in generating a gene therapy vector may comprise alanine substitutions for amino acids 155, 156, 204, 205, 572, and 573 of a JPH2 reference sequence as set forth in SEQ ID NO: 1, or any combination thereof.

In some embodiments, at least one calpain cleavage site is removed from a polynucleotide encoding JPH2 by substituting the amino acids of the at least one cleavage site with alanine. In some embodiments, at least one calpain cleavage site is removed from a polynucleotide encoding JPH2 by substituting the amino acids of the at least one cleavage site with amino acids that have similar properties or a conservative amino acid substitution, e.g., alanine substituted for valine, lysine substituted for arginine, alanine substituted for leucine, and serine substituted for threonine. In accordance with the present disclosure, a polynucleotide encoding a JPH2 or functional variant thereof, wherein the JPH2 or functional variant thereof comprising at least 129, at least 600, at least 630, or at least 696 amino acids may be employed in generating a gene therapy vector. The resulting vector may be employed in treating diseases or disorders, e.g., a JPH2-related disease or disorder, e.g., atrial fibrillation, arrhythmia, sinus node disease, hypertensive heart disease, heart failure, cardiac hypertrophy, atrial fibrosis, myocardial infarction, symptomatic sick sinus syndrome, atrial disease, myocardial infarction, familial hypertrophic cardiomyopathy 17, and others.

Unless otherwise defined, all technical and scientific terms used herein have the same meaning as commonly understood by one of ordinary skill in the art to which this disclosure pertains. Although methods and materials similar or equivalent to those described herein can be used in the practice of the present disclosure, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

As used herein, the terms “identity” and “identical” refer, with respect to a polypeptide or polynucleotide sequence, to the percentage of exact matching residues in an alignment of that “query” sequence to a “subject” sequence, such as an alignment generated by the BLAST algorithm. Identity is calculated, unless specified otherwise, across the full length of the subject sequence. Thus, a query sequence “shares at least x % identity to” a subject sequence if, when the query sequence is aligned to the subject sequence, at least x % (rounded down) of the residues in the subject sequence are aligned as an exact match to a corresponding residue in the query sequence. Where the subject sequence has variable positions (e.g., residues denoted X), an alignment to any residue in the query sequence is counted as a match. Sequence alignments may be performed using the NCBI Blast service (BLAST+ version 2.12.0).

As used herein, the term “operatively linked” refers to a functional relationship between two or more nucleic acid (e.g., DNA) segments. Typically, it refers to the functional relationship of a transcriptional regulatory sequence to a transcribed sequence. For example, a promoter sequence is operatively linked to a coding sequence if it stimulates or modulates the transcription of the coding sequence in an appropriate host cell or other expression system. Generally, promoter transcriptional regulatory sequences that are operatively linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, an “AAV vector” or “rAAV vector” refers to a recombinant vector comprising one or more polynucleotides of interest (or transgenes) that are flanked by AAV inverted terminal repeat sequences (ITRs). Such AAV vectors can be replicated and packaged into infectious viral particles when present in a host cell that has been transfected with a plasmid encoding and expressing rep and cap gene products. Alternatively, AAV vectors can be packaged into infectious particles using a host cell that has been stably engineered to express rep and cap genes.

As used herein, an “AAV virion” or “AAV viral particle” or “AAV vector particle” refers to a viral particle composed of at least one AAV capsid protein and an encapsidated polynucleotide AAV vector. As used herein, if the particle comprises a heterologous polynucleotide (i.e., a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

As used herein, “promoter” refers to a polynucleotide sequence capable of promoting initiation of RNA transcription from a polynucleotide in a eukaryotic cell.

As used herein, “vector genome” refers to the polynucleotide sequence packaged by the vector (e.g., an rAAV virion), including flanking sequences (e.g., in AAV, inverted terminal repeats). In AAV, the terms “expression cassette” and “polynucleotide cassette” refer to the portion of the vector genome between the flanking ITR sequences. “Expression cassette” implies that the vector genome comprises at least one gene encoding a gene product operatively linked to an element that drives expression (e.g., a promoter), including any regulatory elements and/or enhancer elements. “Polynucleotide cassette” refers to the portion of the vector genome that comprises at least one gene encoding a gene product operatively linked to an element that drives expression (e.g., a promoter), including any regulatory elements and/or enhancer elements.

As used herein, the term “patient in need” or “subject in need” refers to a patient or subject at risk of, or suffering from, a disease, disorder or condition that is amenable to treatment or amelioration with a recombinant gene therapy vector or gene editing system disclosed herein. A patient or subject in need may, for instance, be a patient or subject diagnosed with a disorder associated with heart. A subject may have a mutation in an JPH2 gene or deletion of all or a part of JPH2 gene, or of gene regulatory sequences, that causes aberrant expression and/or nuclear translocation of the JPH2 protein. “Subject” and “patient” are used interchangeably herein. The subject treated by the methods described herein may be an adult or a child. Subjects may range in age.

As used herein, the term “variant” refers to a protein that has one or more amino-acid substitution, insertion, or deletion as compared to a parental protein. As used herein, the term “functional variant” refers to a protein that has one or more amino-acid substitution, insertion, or deletion as compared to a parental protein, and which retains one or more desired activities of the parental protein.

As used herein, “treating” refers to ameliorating one or more symptoms of a disease or disorder. The term “preventing” refers to delaying or interrupting the onset of one or more symptoms of a disease or disorder or slowing the progression of JPH2-related disease or disorder, e.g., familial hypertrophic cardiomyopathy 17.

In certain embodiments, “administration” may be performed by an injection, catheterization, and/or an infusion. In some embodiments, the vector is administered by intravenous infusion, intravenous injection, intracardiac infusion, intracardiac injection, intracoronary infusion, intracoronary injection, and/or cardiac catheterization.

Adeno-associated virus (AAV) is a replication-deficient parvovirus, the single-stranded DNA genome of which is about 4.7 kb in length including two ˜145-nucleotide inverted terminal repeat (ITRs). There are multiple known variants of AAV, also sometimes called serotypes when classified by antigenic epitopes. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45:555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Mol. Ther., 13 (1): 67-76 (2006); and the AAV-11 genome is provided in Virology, 330 (2): 375-383 (2004). The sequence of the AAVrh.74 genome is provided in U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep78, rep68, rep52, and rep40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Gene delivery viral vectors useful in the practice of the present disclosure can be constructed utilizing methodologies well known in the art of molecular biology. Typically, viral vectors carrying transgenes are assembled from polynucleotides encoding the transgene, suitable regulatory elements and elements necessary for production of viral proteins, which mediate cell transduction. Such recombinant viruses may be produced by techniques known in the art, e.g., by transfecting packaging cells or by transient transfection with helper plasmids or viruses. Typical examples of virus packaging cells include but are not limited to HeLa cells, SF9 cells (optionally with a baculovirus helper vector), HEK293 cells, etc. A Herpesvirus-based system can be used to produce AAV vectors, as described in US20170218395A1. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478, the complete contents of each of which is hereby incorporated by reference.

The present disclosure contemplates compositions and methods of use related to Junctophilin-2 (JPH2) proteins or polypeptides. Stress-induced cleavage of JPH2 is known to be associated with cardiomyopathy and heart failure, including diseases like those described in Beavers et al. Cardiovascular Research 103:198-205 (2014); and in other sources. Details regarding truncated variants of JPH2 proteins may be found for instance in U.S. Pat. App. No. 2019/0307899, the complete contents of each of which is hereby incorporated by reference. Viral vector-mediated delivery of the JPH2 gene may therefore serve as a viable therapeutic for JPH2-related human diseases such as cardiomyopathy and heart failure.

Mutations in the JPH2 gene have been identified in people with familial hypertrophic cardiomyopathy 17 (CMH17). (See “CMH17,” NCBI MedGen). This condition is a hereditary heart disorder characterized by ventricular hypertrophy, which is usually asymmetric and often involves the interventricular septum. The symptoms include dyspnea, syncope, collapse, palpitations, and chest pain and they can be readily provoked by exercise. The disorder has inter- and intrafamilial variability ranging from benign to malignant forms with high risk of cardiac failure and sudden cardiac death.

In some embodiments, JPH2 comprises one or more amino acid substitutions selected from: mutation of one or more residues in the predicted calpain 1 cleavage sites (V155A, R156K, L204A, L205A, R572K, or T573S), numbered relative to SEQ ID NO: 1. That is, the JPH2 protein may comprises one or more of, two or more of, three or more, or four or more amino acid substitutions selected from the group consisting of R572A or R572K, T573A or T573S, V155A, R156A or R156K, L204A, and L205A. Alternative conservative, or non-conservative mutations or substitutions at any of these sites may be used, including without limitation one or more of, two or more of, three one or more, or four or more amino acid substitutions selected from the group consisting of R572X, T573X, V155X, R156X, L204X, and L205X, where X represents any naturally or non-naturally occurring amino acid other than the amino acid present in the reference JPH2 protein.

The term “conservative substitution” as used herein denotes that one or more amino acid is replaced by another, biologically similar residue. Examples include substitution of amino acid residues with similar characteristics, e.g., small amino acids, acidic amino acids, polar amino acids, basic amino acids, hydrophobic amino acids and aromatic amino acids. In the scheme below, conservative substitutions of amino acids are grouped by physicochemical properties. I: neutral, hydrophilic, II: acids and amides, III: basic, IV: hydrophobic, V: aromatic, bulky amino acids.

| I | II | III | IV | V |
|---|---|---|---|---|
| A | N | H | M | F |
| S | D | R | L | Y |
| T | E | K | I | W |
| P | Q | | V | |
| G | | | C | |

In the scheme below, conservative substitutions of amino acids are grouped by physicochemical properties. VI: neutral or hydrophobic, VII: acidic, VIII: basic, IX: polar, X: aromatic.

| VI | VII | VIII | IX | X |
|---|---|---|---|---|
| A | E | H | M | F |
| L | D | R | S | Y |
| I | | K | T | W |
| P | | | C | |
| G | | | N | |
| V | | | Q | |

Particular mutations contemplated by the present disclosure include R572A or R572K; T573A or T573S; V155A; R156A or R156K; L204A; or L205A. In some embodiments, the amino acid substitution disrupts an intra-molecular or inter-molecular interface. In some embodiments, the amino acid substitution disrupts an intra-molecular or inter-molecular interface, while maintaining one or more characteristics of the residue, such as charge, size, and/or hydrophobicity.

The activated JPH2 may comprise one or more amino-acid substitutions, inserts, or deletions (collectively, mutations) that protect against truncation of JPH2 mediated by calpain 1, and thereby reduce calpain-induced cleavage of JPH2. For example, the JPH2 may comprise a mutation in one calpain 1 site that reduces binding and subsequent cleavage by calpain 1 or mutations in three calpain 1 sites that reduce binding and subsequent cleavage by calpain 1.

Various further embodiments of JPH2 are provided in Table 1.

TABLE 1
Illustrative Combinations of Amino Acid Substitutions

| | | | |
|---|---|---|---|
| R572A + | R572K + | V155A + R156A + | V155A + R156K + |
| T573A | T573S | L204A + L205A + | L204A + L205A + |
| | | R572A + T573A | R572K + T573S |

In some embodiments, the JPH2 protein comprises one or more amino acid substitutions at positions Arg-572 and Thr-573 relative to a reference JPH2 protein.

In some embodiments, the JPH2 protein comprises one or more amino acid substitutions at positions Val-155, Arg-156, Leu204, Leu205, Arg-572 and Thr-573 relative to a reference JPH2 protein.

In some embodiments, the JPH2 protein comprises one or more amino acid substitutions selected from R572A, R572K, T573A, T573S, V155A, R156A, R156K, L204A, and/or L205A relative to a reference JPH2 protein.

In some embodiments, the JPH2 protein comprises amino acid substitutions R572A and T573A relative to a reference JPH2 protein.

In some embodiments, the JPH2 protein comprises amino acid substitutions R572K and T573S relative to a reference JPH2 protein.

In some embodiments, the JPH2 protein comprises amino acid substitutions V155A, R156A, L204A, L205A, R572A, and T573A relative to a reference JPH2 protein.

In some embodiments, the JPH2 protein comprises amino acid substitutions V155A, R156K, L204A, L205A, R572K, and T573S relative to a reference JPH2 protein.

The native sequences of human JPH2, isoform 1 and isoform 2, protein and polynucleotide coding sequence are shown below:

| JPH2-wild type, Isoform 1-696 amino acids | |
|---|---|
| (SEQ ID NO: 1) | |
| 1 | MSGGRFDFDD GGAYCGGWEG GKAHGHGLCT GPKGQGEYSG |
| 41 | SWNFGFEVAG VYTWPSGNTF EGYWSQGKRH GLGIETKGRW |
| 81 | LYKGEWTHGF KGRYGIRQSS SSGAKYEGTW NNGLQDGYGT |
| 121 | ETYADGGTYQ GQFTNGMRHG YGVRQSVPYG MAVVVRSPLR |
| 161 | TSLSSLRSEH SNGTVAPDSP ASPASDGPAL PSPAIPRGGF |
| 201 | ALSLLANAEA AARAPKGGGL FQRGALLGKL RRAESRTSVG |
| 241 | SQRSRVSFLK SDLSSGASDA ASTASLGEAA EGADEAAPFE |
| 281 | ADIDATTTET YMGEWKNDKR SGFGVSERSS GLRYEGEWLD |
| 321 | NLRHGYGCTT LPDGHREEGK YRHNVLVKDT KRRMLQLKSN |
| 361 | KVRQKVEHSV EGAQRAAAIA RQKAEIAASR TSHAKAKAEA |
| 401 | AEQAALAANQ ESNIARTLAR ELAPDFYQPG PEYQKRRLLQ |
| 441 | EILENSESLL EPPDRGAGAA GLPQPPRESP QLHERETPRP |
| 481 | EGGSPSPAGT PPQPKRPRPG VSKDGLLSPG AWNGEPSGEG |
| 521 | SRSVTPSEGA GRRSPARPAT ERMAIEALQA PPAPSREPEV |
| 561 | ALYQGYHSYA VRTTPPEPPP FEDQPEPEVS GSESAPSSPA |
| 601 | TAPLQAPTLR GPEPARETPA KLEPKPIIPK AEPRAKARKT |
| 641 | EARGLTKAGA KKKARKEAAL AAEAEVEVEE VPNTILICMV |
| 681 | ILLNIGLAIL FVHLLT |

| JPH2-wild type, Transcript 1-2091 nucleotide bases | |
|---|---|
| (SEQ ID NO: 2) | |
| atgagtgggg gccgcttcga ctttgatgat ggaggggcgt actgcggggg ctgggagggg | 60 |
| ggaaaggccc atgggcatgg actgtgcaca ggccccaagg gccagggcga atactctggc | 120 |
| tcctggaact ttggctttga ggtggcaggt gtctacacct ggcccagcgg aaacaccttt | 180 |
| gagggatact ggagccaggg caaacggcat gggctgggca tagagaccaa ggggcgctgg | 240 |
| ctctacaagg gcgagtggac acatggcttc aagggacgct acggaatccg gcagagctca | 300 |
| agcagcggtg ccaagtatga gggcacctgg aacaatggcc tgcaagacgg ctatggcacc | 360 |
| gagacctatg ctgatggagg gacgtaccaa ggccagttca ccaacggcat gcgccatggc | 420 |
| tacggagtac gccagagcgt gccctacggg atggccgtgg tggtgcgctc gccgctgcgc | 480 |
| acgtcgctgt cgtccctgcg cagcgagcac agcaacggca cggtggcccc ggactctccc | 540 |
| gcctcgccgg cctccgacgg ccccgcgctg ccctcgcccg ccatcccgcg tggcggcttc | 600 |
| gcgctcagcc tcctggccaa tgccgaggcg gccgcgcggg cgcccaaggg cggcggcctc | 660 |
| ttccagcggg gcgcgctgct gggcaagctg cggcgcgcag agtcgcgcac gtccgtgggt | 720 |
| agccagcgca gccgtgtcag cttccttaag agcgacctca gctcgggcgc cagcgacgcc | 780 |
| gcgtccaccg ccagcctggg agaggccgcc gagggcgccg acgaggccgc acccttcgag | 840 |
| gccgatatcg acgccaccac caccgagacc tacatgggcg agtggaagaa cgacaaacgc | 900 |
| tcgggcttcg gcgtgagcga acgctccagt ggcctccgct acgagggcga gtggctggac | 960 |
| aacctgcgcc acggctatgg ctgcaccacg ctgcccgacg gccaccgcga ggagggcaag | 1020 |
| taccgccaca acgtgctggt caaggacacc aagcgccgca tgctgcagct caagagcaac | 1080 |
| aaggtccgcc agaaagtgga gcacagtgtg gagggtgccc agcgcgccgc tgctatcgcg | 1140 |
| cgccagaagg ccgagattgc cgcctccagg acaagccacg ccaaggccaa agctgaggca | 1200 |
| gcggaacagg ccgccctggc tgccaaccag gagtccaaca ttgctcgcac tttggccagg | 1260 |
| gagctggctc cggacttcta ccagccaggt ccggaatatc agaagcgccg gctgctgcag | 1320 |
| gagatcctgg agaactcgga gagcctgctg gagccccccg accggggcgc cggcgcagcg | 1380 |
| ggcctcccac agccgccccg cgagagcccg cagctgcacg agcgtgagac ccctcggccc | 1440 |
| gagggtggct ccccgtcacc ggccgggacg cccccgcagc ccaagcggcc caggcccggg | 1500 |
| gtgtccaagg acggcctgct gagcccaggc gcctggaacg gcgagcccag cggtgagggc | 1560 |
| agccggtcag tcactccgtc cgagggcgcg ggccgccgca gccccgcgcg tccagccacc | 1620 |
| gagcgcatgg ccatcgaggc tctgcaggca ccgcctgcgc cgtcgcggga gccggaggtg | 1680 |
| gcgctttacc agggctacca cagctatgct gtgcgcacca cgccgcccga gcccccaccc | 1740 |
| tttgaggacc agcccgagcc cgaggtctcc gggtccgagt ccgcgccctc gtccccggcc | 1800 |
| accgccccgc tgcaggcccc cacgctccga ggccccgagc ctgcacgcga gacccccgcc | 1860 |
| aagctggagc ccaagcccat catccccaaa gccgagccca gggccaaggc ccgcaagact | 1920 |
| gaggctcgag ggctgaccaa ggcgggggcc aagaagaagg cgcggaagga ggccgcactg | 1980 |
| gcggcagagg cggaggtgga ggtggaagag gtccccaaca ccatcctcat ctgcatggtg | 2040 |
| atcctgctga acatcggcct ggccatcctc tttgttcacc tcctgacctg a | 2091 |

| JPH2-wild type, Isoform 2-696 amino acids | |
|---|---|
| (SEQ ID NO: 108) | |
| 1 | MSGGRFDFDD GGAYCGGWEG GKAHGHGLCT GPKGQGEYSG |
| 41 | SWNFGFEVAG VYTWPSGNTF EGYWSQGKRH GLGIETKGRW |
| 81 | LYKGEWTHGF KGRYGIRQSS SSGAKYEGTW NNGLQDGYGT |
| 121 | ETYADGGMC |

| JPH2-wild type, Transcript 2-390 nucleotide bases | |
|---|---|
| (SEQ ID NO: 107) | |
| atgagtgggg gccgcttcga ctttgatgat ggaggggcgt actgcggggg ctgggagggg | 60 |
| ggaaaggccc atgggcatgg actgtgcaca ggccccaagg gccagggcga atactctggc | 120 |
| tcctggaact ttggctttga ggtggcaggt gtctacacct ggcccagcgg aaacaccttt | 180 |
| gagggatact ggagccaggg caaacggcat gggctgggca tagagaccaa ggggcgctgg | 240 |
| ctctacaagg gcgagtggac acatggcttc aagggacgct acggaatccg gcagagctca | 300 |
| agcagcggtg ccaagtatga gggcacctgg aacaatggcc tgcaagacgg ctatggcacc | 360 |
| gagacctatg ctgatggagg gatgtgttaa | 390 |

In some embodiments, the JPH2 protein comprises a polypeptide sequence at least 75%, 80%, 85%, 90%, 92%, 93%, 94%, 95%, 96%, 97%, 98%, 99%, or 100% identical to SEQ ID NO: 1. In some embodiments, the JPH2 polynucleotide comprises a polynucleotide sequence at least 75%, 80%, 85%, 90%, 92%, 93%, 94%, 95%, 96%, 97%, 98%, 99%, or 100% identical to SEQ ID NO: 2. In some embodiments, the JPH2 protein is a wild-type or native JPH2 protein, e.g., human JPH2.

In some embodiments, the JPH2 protein comprises a polypeptide sequence at least 75%, 80%, 85%, 90%, 92%, 93%, 94%, 95%, 96%, 97%, 98%, 99%, or 100% identical to SEQ ID NO: 108. In some embodiments, the JPH2 polynucleotide comprises a polynucleotide sequence at least 75%, 80%, 85%, 90%, 92%, 93%, 94%, 95%, 96%, 97%, 98%, 99%, or 100% identical to SEQ ID NO: 107. In some embodiments, the JPH2 protein is a wild-type or native JPH2 protein, e.g., human JPH2.

The present disclosure contemplates compositions and methods of use related to JPH2 proteins or polynucleotides with calpain 1 binding site mutations. The 1mutAA mutant of JPH2 comprises a polypeptide sequence comprising amino acid substitutions R572A and T573A (SEQ ID NO: 3). The 1mutAA mutant of JPH2 comprises a polynucleotide encoding amino acid substitutions R572A and T573A (SEQ ID NO: 4).

| JPH2-1mutAA-696 amino acids | |
|---|---|
| (SEQ ID NO: 3) | |
| 1 | MSGGRFDFDD GGAYCGGWEG GKAHGHGLCT GPKGQGEYSG |
| 41 | SWNFGFEVAG VYTWPSGNTF EGYWSQGKRH GLGIETKGRW |
| 81 | LYKGEWTHGF KGRYGIRQSS SSGAKYEGTW NNGLQDGYGT |
| 121 | ETYADGGTYQ GQFTNGMRHG YGVRQSVPYG MAVVVRSPLR |
| 161 | TSLSSLRSEH SNGTVAPDSP ASPASDGPAL PSPAIPRGGF |
| 201 | ALSLLANAEA AARAPKGGGL FQRGALLGKL RRAESRTSVG |
| 241 | SQRSRVSELK SDLSSGASDA ASTASLGEAA EGADEAAPFE |
| 281 | ADIDATTTET YMGEWKNDKR SGFGVSERSS GLRYEGEWLD |
| 321 | NLRHGYGCTT LPDGHREEGK YRHNVLVKDT KRRMLQLKSN |
| 361 | KVRQKVEHSV EGAQRAAAIA RQKAEIAASR TSHAKAKAEA |
| 401 | AEQAALAANQ ESNIARTLAR ELAPDFYQPG PEYQKRRLLQ |
| 441 | EILENSESLL EPPDRGAGAA GLPQPPRESP QLHERETPRP |
| 481 | EGGSPSPAGT PPQPKRPRPG VSKDGLLSPG AWNGEPSGEG |
| 521 | SRSVTPSEGA GRRSPARPAT ERMAIEALQA PPAPSREPEV |
| 561 | ALYQGYHSYA VAATPPEPPP FEDQPEPEVS GSESAPSSPA |
| 601 | TAPLQAPTLR GPEPARETPA KLEPKPIIPK AEPRAKARKT |
| 641 | EARGLTKAGA KKKARKEAAL AAEAEVEVEE VPNTILICMV |
| 681 | ILLNIGLAIL FVHLLT |

| JPH2-1mutAA-2091 nucleotide bases | |
|---|---|
| (SEQ ID NO: 4) | |
| atgagtgggg gccgcttcga ctttgatgat ggaggggcgt actgcggggg ctgggagggg | 60 |
| ggaaaggccc atgggcatgg actgtgcaca ggccccaagg gccagggcga atactctggc | 120 |
| tcctggaact ttggctttga ggtggcaggt gtctacacct ggcccagcgg aaacaccttt | 180 |
| gagggatact ggagccaggg caaacggcat gggctgggca tagagaccaa ggggcgctgg | 240 |
| ctctacaagg gcgagtggac acatggcttc aagggacgct acggaatccg gcagagctca | 300 |
| agcagcggtg ccaagtatga gggcacctgg aacaatggcc tgcaagacgg ctatggcacc | 360 |
| gagacctatg ctgatggagg gacgtaccaa ggccagttca ccaacggcat gcgccatggc | 420 |
| tacggagtac gccagagcgt gccctacggg atggccgtgg tggtgcgctc gccgctgcgc | 480 |
| acgtcgctgt cgtccctgcg cagcgagcac agcaacggca cggtggcccc ggactctccc | 540 |
| gcctcgccgg cctccgacgg ccccgcgctg ccctcgcccg ccatcccgcg tggcggcttc | 600 |
| gcgctcagcc tcctggccaa tgccgaggcg gccgcgcggg cgcccaaggg cggcggcctc | 660 |
| ttccagcggg gcgcgctgct gggcaagctg cggcgcgcag agtcgcgcac gtccgtgggt | 720 |
| agccagcgca gccgtgtcag cttccttaag agcgacctca gctcgggcgc cagcgacgcc | 780 |
| gcgtccaccg ccagcctggg agaggccgcc gagggcgccg acgaggccgc acccttcgag | 840 |
| gccgatatcg acgccaccac caccgagacc tacatgggcg agtggaagaa cgacaaacgc | 900 |
| tcgggcttcg gcgtgagcga acgctccagt ggcctccgct acgagggcga gtggctggac | 960 |
| aacctgcgcc acggctatgg ctgcaccacg ctgcccgacg gccaccgcga ggagggcaag | 1020 |
| taccgccaca acgtgctggt caaggacacc aagcgccgca tgctgcagct caagagcaac | 1080 |
| aaggtccgcc agaaagtgga gcacagtgtg gagggtgccc agcgcgccgc tgctatcgcg | 1140 |
| cgccagaagg ccgagattgc cgcctccagg acaagccacg ccaaggccaa agctgaggca | 1200 |
| gcggaacagg ccgccctggc tgccaaccag gagtccaaca ttgctcgcac tttggccagg | 1260 |
| gagctggctc cggacttcta ccagccaggt ccggaatatc agaagcgccg gctgctgcag | 1320 |
| gagatcctgg agaactcgga gagcctgctg gagccccccg accggggcgc cggcgcagcg | 1380 |
| ggcctcccac agccgccccg cgagagcccg cagctgcacg agcgtgagac ccctcggccc | 1440 |
| gagggtggct ccccgtcacc ggccgggacg cccccgcagc ccaagcggcc caggcccggg | 1500 |
| gtgtccaagg acggcctgct gagcccaggc gcctggaacg gcgagcccag cggtgagggc | 1560 |
| agccggtcag tcactccgtc cgagggcgcg ggccgccgca gccccgcgcg tccagccacc | 1620 |
| gagcgcatgg ccatcgaggc tctgcaggca ccgcctgcgc cgtcgcggga gccggaggtg | 1680 |
| gcgctttacc agggctacca cagctatgct gtggccgcca cgccgcccga gcccccaccc | 1740 |
| tttgaggacc agcccgagcc cgaggtctcc gggtccgagt ccgcgccctc gtccccggcc | 1800 |
| accgccccgc tgcaggcccc cacgctccga ggccccgagc ctgcacgcga gacccccgcc | 1860 |
| aagctggagc ccaagcccat catccccaaa gccgagccca gggccaaggc ccgcaagact | 1920 |
| gaggctcgag ggctgaccaa ggcgggggcc aagaagaagg cgcggaagga ggccgcactg | 1980 |
| gcggcagagg cggaggtgga ggtggaagag gtccccaaca ccatcctcat ctgcatggtg | 2040 |
| atcctgctga acatcggcct ggccatcctc tttgttcacc tcctgacctg a | 2091 |

In some embodiments, the JPH2 protein comprises a polypeptide sequence at least 75%, 80%, 85%, 90%, 92%, 93%, 94%, 95%, 96%, 97%, 98%, 99%, or 100% identical to SEQ ID NO: 3. In some embodiments, the JPH2 protein comprises a polynucleotide sequence at least 75%, 80%, 85%, 90%, 92%, 93%, 94%, 95%, 96%, 97%, 98%, 99%, or 100% identical to SEQ ID NO: 4. In some embodiments, the JPH2 protein is a mutant JPH2 protein.

The present disclosure contemplates compositions and methods of use related to JPH2 proteins or polynucleotides with calpain 1 binding site mutations. The 1mutKS mutant of JPH2 comprises a polypeptide sequence comprising amino acid substitutions R572K and T573S (SEQ ID NO: 5). The 1mutKS mutant of JPH2 comprises a polynucleotide encoding amino acid substitutions R572K and T573S (SEQ ID NO: 6).

| JPH2-1mutKS-696 amino acids | |
|---|---|
| (SEQ ID NO: 5) | |
| 1 | MSGGREDEDD GGAYCGGWEG GKAHGHGLCT GPKGQGEYSG |
| 41 | SWNFGFEVAG VYTWPSGNTF EGYWSQGKRH GLGIETKGRW |
| 81 | LYKGEWTHGF KGRYGIRQSS SSGAKYEGTW NNGLQDGYGT |
| 121 | ETYADGGTYQ GQFTNGMRHG YGVRQSVPYG MAVVVRSPLR |
| 161 | TSLSSLRSEH SNGTVAPDSP ASPASDGPAL PSPAIPRGGF |
| 201 | ALSLLANAEA AARAPKGGGL FQRGALLGKL RRAESRTSVG |
| 241 | SQRSRVSELK SDLSSGASDA ASTASLGEAA EGADEAAPFE |
| 281 | ADIDATTTET YMGEWKNDKR SGFGVSERSS GLRYEGEWLD |
| 321 | NLRHGYGCTT LPDGHREEGK YRHNVLVKDT KRRMLQLKSN |
| 361 | KVRQKVEHSV EGAQRAAAIA RQKAEIAASR TSHAKAKAEA |
| 401 | AEQAALAANQ ESNIARTLAR ELAPDFYQPG PEYQKRRLLQ |
| 441 | EILENSESLL EPPDRGAGAA GLPQPPRESP QLHERETPRP |
| 481 | EGGSPSPAGT PPQPKRPRPG VSKDGLLSPG AWNGEPSGEG |
| 521 | SRSVTPSEGA GRRSPARPAT ERMAIEALQA PPAPSREPEV |
| 561 | ALYQGYHSYA VKSTPPEPPP FEDQPEPEVS GSESAPSSPA |
| 601 | TAPLQAPTLR GPEPARETPA KLEPKPIIPK AEPRAKARKT |
| 641 | EARGLTKAGA KKKARKEAAL AAEAEVEVEE VPNTILICMV |
| 681 | ILLNIGLAIL FVHLLT |

| JPH2-1mutKS-2091 nucleotide bases | |
|---|---|
| (SEQ ID NO: 6) | |
| atgagtgggg gccgcttcga ctttgatgat ggaggggcgt actgcggggg ctgggagggg | 60 |
| ggaaaggccc atgggcatgg actgtgcaca ggccccaagg gccagggcga atactctggc | 120 |
| tcctggaact ttggctttga ggtggcaggt gtctacacct ggcccagcgg aaacaccttt | 180 |
| gagggatact ggagccaggg caaacggcat gggctgggca tagagaccaa ggggcgctgg | 240 |
| ctctacaagg gcgagtggac acatggcttc aagggacgct acggaatccg gcagagctca | 300 |
| agcagcggtg ccaagtatga gggcacctgg aacaatggcc tgcaagacgg ctatggcacc | 360 |
| gagacctatg ctgatggagg gacgtaccaa ggccagttca ccaacggcat gcgccatggc | 420 |
| tacggagtac gccagagcgt gccctacggg atggccgtgg tggtgcgctc gccgctgcgc | 480 |
| acgtcgctgt cgtccctgcg cagcgagcac agcaacggca cggtggcccc ggactctccc | 540 |
| gcctcgccgg cctccgacgg ccccgcgctg ccctcgcccg ccatcccgcg tggcggcttc | 600 |
| gcgctcagcc tcctggccaa tgccgaggcg gccgcgcggg cgcccaaggg cggcggcctc | 660 |
| ttccagcggg gcgcgctgct gggcaagctg cggcgcgcag agtcgcgcac gtccgtgggt | 720 |
| agccagcgca gccgtgtcag cttccttaag agcgacctca gctcgggcgc cagcgacgcc | 780 |
| gcgtccaccg ccagcctggg agaggccgcc gagggcgccg acgaggccgc acccttcgag | 840 |
| gccgatatcg acgccaccac caccgagacc tacatgggcg agtggaagaa cgacaaacgc | 900 |
| tcgggcttcg gcgtgagcga acgctccagt ggcctccgct acgagggcga gtggctggac | 960 |
| aacctgcgcc acggctatgg ctgcaccacg ctgcccgacg gccaccgcga ggagggcaag | 1020 |
| taccgccaca acgtgctggt caaggacacc aagcgccgca tgctgcagct caagagcaac | 1080 |
| aaggtccgcc agaaagtgga gcacagtgtg gagggtgccc agcgcgccgc tgctatcgcg | 1140 |
| cgccagaagg ccgagattgc cgcctccagg acaagccacg ccaaggccaa agctgaggca | 1200 |
| gcggaacagg ccgccctggc tgccaaccag gagtccaaca ttgctcgcac tttggccagg | 1260 |
| gagctggctc cggacttcta ccagccaggt ccggaatatc agaagcgccg gctgctgcag | 1320 |
| gagatcctgg agaactcgga gagcctgctg gagccccccg accggggcgc cggcgcagcg | 1380 |
| ggcctcccac agccgccccg cgagagcccg cagctgcacg agcgtgagac ccctcggccc | 1440 |
| gagggtggct ccccgtcacc ggccgggacg cccccgcagc ccaagcggcc caggcccggg | 1500 |
| gtgtccaagg acggcctgct gagcccaggc gcctggaacg gcgagcccag cggtgagggc | 1560 |
| agccggtcag tcactccgtc cgagggcgcg ggccgccgca gccccgcgcg tccagccacc | 1620 |
| gagcgcatgg ccatcgaggc tctgcaggca ccgcctgcgc cgtcgcggga gccggaggtg | 1680 |
| gcgctttacc agggctacca cagctatgct gtgaagagca cgccgcccga gcccccaccc | 1740 |
| tttgaggacc agcccgagcc cgaggtctcc gggtccgagt ccgcgccctc gtccccggcc | 1800 |
| accgccccgc tgcaggcccc cacgctccga ggccccgagc ctgcacgcga gacccccgcc | 1860 |
| aagctggagc ccaagcccat catccccaaa gccgagccca gggccaaggc ccgcaagact | 1920 |
| gaggctcgag ggctgaccaa ggcgggggcc aagaagaagg cgcggaagga ggccgcactg | 1980 |
| gcggcagagg cggaggtgga ggtggaagag gtccccaaca ccatcctcat ctgcatggtg | 2040 |
| atcctgctga acatcggcct ggccatcctc tttgttcacc tcctgacctg a | 2091 |

In some embodiments, the JPH2 protein comprises a polypeptide sequence at least 75%, 80%, 85%, 90%, 92%, 93%, 94%, 95%, 96%, 97%, 98%, 99%, or 100% identical to SEQ ID NO: 5. In some embodiments, the JPH2 protein comprises a polynucleotide sequence at least 75%, 80%, 85%, 90%, 92%, 93%, 94%, 95%, 96%, 97%, 98%, 99%, or 100% identical to SEQ ID NO: 6.

The present disclosure contemplates compositions and methods of use related to JPH2 proteins or polynucleotides with calpain 1 binding site mutations. The 3mutAA mutant of JPH2 comprises a polypeptide sequence comprising amino acid substitutions V155A, R156A, L204A, L205A, R572A, and T573A (SEQ ID NO: 7). The 3mutAA mutant of JPH2 comprises a polynucleotide encoding amino acid substitutions V155A, R156A, L204A, L205A, R572A, and T573A (SEQ ID NO: 8).

| JPH2-3mutAA-696 amino acids | |
|---|---|
| (SEQ ID NO: 7) | |
| 1 | MSGGRFDFDD GGAYCGGWEG GKAHGHGLCT GPKGQGEYSG |
| 41 | SWNFGFEVAG VYTWPSGNTF EGYWSQGKRH GLGIETKGRW |
| 81 | LYKGEWTHGF KGRYGIRQSS SSGAKYEGTW NNGLQDGYGT |
| 121 | ETYADGGTYQ GQFTNGMRHG YGVRQSVPYG MAVVAASPLR |
| 161 | TSLSSLRSEH SNGTVAPDSP ASPASDGPAL PSPAIPRGGF |
| 201 | ALSAAANAEA AARAPKGGGL FQRGALLGKL RRAESRTSVG |
| 241 | SQRSRVSFLK SDLSSGASDA ASTASLGEAA EGADEAAPFE |
| 281 | ADIDATTTET YMGEWKNDKR SGFGVSERSS GLRYEGEWLD |
| 321 | NLRHGYGCTT LPDGHREEGK YRHNVLVKDT KRRMLQLKSN |
| 361 | KVRQKVEHSV EGAQRAAAIA RQKAEIAASR TSHAKAKAEA |
| 401 | AEQAALAANQ ESNIARTLAR ELAPDFYQPG PEYQKRRLLQ |
| 441 | EILENSESLL EPPDRGAGAA GLPQPPRESP QLHERETPRP |
| 481 | EGGSPSPAGT PPQPKRPRPG VSKDGLLSPG AWNGEPSGEG |
| 521 | SRSVTPSEGA GRRSPARPAT ERMAIEALQA PPAPSREPEV |
| 561 | ALYQGYHSYA VAATPPEPPP FEDQPEPEVS GSESAPSSPA |
| 601 | TAPLQAPTLR GPEPARETPA KLEPKPIIPK AEPRAKARKT |
| 641 | EARGLTKAGA KKKARKEAAL AAEAEVEVEE VPNTILICMV |
| 681 | ILLNIGLAIL FVHLLT |

| JPH2-3mutAA-2091 nucleotide bases | |
|---|---|
| (SEQ ID NO: 8) | |
| atgagtgggg gccgcttcga ctttgatgat ggaggggcgt actgcggggg ctgggagggg | 60 |
| ggaaaggccc atgggcatgg actgtgcaca ggccccaagg gccagggcga atactctggc | 120 |
| tcctggaact ttggctttga ggtggcaggt gtctacacct ggcccagcgg aaacaccttt | 180 |
| gagggatact ggagccaggg caaacggcat gggctgggca tagagaccaa ggggcgctgg | 240 |
| ctctacaagg gcgagtggac acatggcttc aagggacgct acggaatccg gcagagctca | 300 |
| agcagcggtg ccaagtatga gggcacctgg aacaatggcc tgcaagacgg ctatggcacc | 360 |
| gagacctatg ctgatggagg gacgtaccaa ggccagttca ccaacggcat gcgccatggc | 420 |
| tacggagtac gccagagcgt gccctacggg atggccgtgg tggccgcctc gccgctgcgc | 480 |
| acgtcgctgt cgtccctgcg cagcgagcac agcaacggca cggtggcccc ggactctccc | 540 |
| gcctcgccgg cctccgacgg ccccgcgctg ccctcgcccg ccatcccgcg tggcggcttc | 600 |
| gcgctcagcg ccgccgccaa tgccgaggcg gccgcgcggg cgcccaaggg cggcggcctc | 660 |
| ttccagcggg gcgcgctgct gggcaagctg cggcgcgcag agtcgcgcac gtccgtgggt | 720 |
| agccagcgca gccgtgtcag cttccttaag agcgacctca gctcgggcgc cagcgacgcc | 780 |
| gcgtccaccg ccagcctggg agaggccgcc gagggcgccg acgaggccgc acccttcgag | 840 |
| gccgatatcg acgccaccac caccgagacc tacatgggcg agtggaagaa cgacaaacgc | 900 |
| tcgggcttcg gcgtgagcga acgctccagt ggcctccgct acgagggcga gtggctggac | 960 |
| aacctgcgcc acggctatgg ctgcaccacg ctgcccgacg gccaccgcga ggagggcaag | 1020 |
| taccgccaca acgtgctggt caaggacacc aagcgccgca tgctgcagct caagagcaac | 1080 |
| aaggtccgcc agaaagtgga gcacagtgtg gagggtgccc agcgcgccgc tgctatcgcg | 1140 |
| cgccagaagg ccgagattgc cgcctccagg acaagccacg ccaaggccaa agctgaggca | 1200 |
| gcggaacagg ccgccctggc tgccaaccag gagtccaaca ttgctcgcac tttggccagg | 1260 |
| gagctggctc cggacttcta ccagccaggt ccggaatatc agaagcgccg gctgctgcag | 1320 |
| gagatcctgg agaactcgga gagcctgctg gagccccccg accggggcgc cggcgcagcg | 1380 |
| ggcctcccac agccgccccg cgagagcccg cagctgcacg agcgtgagac ccctcggccc | 1440 |
| gagggtggct ccccgtcacc ggccgggacg cccccgcagc ccaagcggcc caggcccggg | 1500 |
| gtgtccaagg acggcctgct gagcccaggc gcctggaacg gcgagcccag cggtgagggc | 1560 |
| agccggtcag tcactccgtc cgagggcgcg ggccgccgca gccccgcgcg tccagccacc | 1620 |
| gagcgcatgg ccatcgaggc tctgcaggca ccgcctgcgc cgtcgcggga gccggaggtg | 1680 |
| gcgctttacc agggctacca cagctatgct gtggccgcca cgccgcccga gcccccaccc | 1740 |
| tttgaggacc agcccgagcc cgaggtctcc gggtccgagt ccgcgccctc gtccccggcc | 1800 |
| accgccccgc tgcaggcccc cacgctccga ggccccgagc ctgcacgcga gacccccgcc | 1860 |
| aagctggagc ccaagcccat catccccaaa gccgagccca gggccaaggc ccgcaagact | 1920 |
| gaggctcgag ggctgaccaa ggcgggggcc aagaagaagg cgcggaagga ggccgcactg | 1980 |
| gcggcagagg cggaggtgga ggtggaagag gtccccaaca ccatcctcat ctgcatggtg | 2040 |
| atcctgctga acatcggcct ggccatcctc tttgttcacc tcctgacctg a | 2091 |

In some embodiments, the JPH2 protein comprises a polypeptide sequence at least 75%, 80%, 85%, 90%, 92%, 93%, 94%, 95%, 96%, 97%, 98%, 99%, or 100% identical to SEQ ID NO: 7. In some embodiments, the JPH2 protein comprises a polynucleotide sequence at least 75%, 80%, 85%, 90%, 92%, 93%, 94%, 95%, 96%, 97%, 98%, 99%, or 100% identical to SEQ ID NO: 8.

The present disclosure contemplates compositions and methods of use related to JPH2 proteins or polynucleotides with calpain 1 binding site mutations. The 3mutAKAAKS mutant of JPH2 comprises a polypeptide sequence comprising amino acid substitutions V155A, R156K, L204A, L205A, R572K, and T573S (SEQ ID NO: 9). The 3mutAKAAKS mutant of JPH2 comprises a polynucleotide encoding amino acid substitutions V155A, R156K, L204A, L205A, R572K, and T573S (SEQ ID NO: 10).

| JPH2-3mutAKAAKS-696 amino acids | |
|---|---|
| (SEQ ID NO: 9) | |
| 1 | MSGGREDEDD GGAYCGGWEG GKAHGHGLCT GPKGQGEYSG |
| 41 | SWNFGFEVAG VYTWPSGNTF EGYWSQGKRH GLGIETKGRW |
| 81 | LYKGEWTHGF KGRYGIRQSS SSGAKYEGTW NNGLQDGYGT |
| 121 | ETYADGGTYQ GQFTNGMRHG YGVRQSVPYG MAVVAKSPLR |
| 161 | TSLSSLRSEH SNGTVAPDSP ASPASDGPAL PSPAIPRGGF |
| 201 | ALSAAANAEA AARAPKGGGL FQRGALLGKL RRAESRTSVG |
| 241 | SQRSRVSELK SDLSSGASDA ASTASLGEAA EGADEAAPFE |
| 281 | ADIDATTTET YMGEWKNDKR SGFGVSERSS GLRYEGEWLD |
| 321 | NLRHGYGCTT LPDGHREEGK YRHNVLVKDT KRRMLQLKSN |
| 361 | KVRQKVEHSV EGAQRAAAIA RQKAEIAASR TSHAKAKAEA |
| 401 | AEQAALAANQ ESNIARTLAR ELAPDFYQPG PEYQKRRLLQ |
| 441 | EILENSESLL EPPDRGAGAA GLPQPPRESP QLHERETPRP |
| 481 | EGGSPSPAGT PPQPKRPRPG VSKDGLLSPG AWNGEPSGEG |
| 521 | SRSVTPSEGA GRRSPARPAT ERMAIEALQA PPAPSREPEV |
| 561 | ALYQGYHSYA VKSTPPEPPP FEDQPEPEVS GSESAPSSPA |
| 601 | TAPLQAPTLR GPEPARETPA KLEPKPIIPK AEPRAKARKT |
| 641 | EARGLTKAGA KKKARKEAAL AAEAEVEVEE VPNTILICMV |
| 681 | ILLNIGLAIL FVHLLT |

| JPH2-3mutAKAAKS-2091 nucleotide bases | |
|---|---|
| (SEQ ID NO: 10) | |
| atgagtgggg gccgcttcga ctttgatgat ggaggggcgt actgcggggg ctgggagggg | 60 |
| ggaaaggccc atgggcatgg actgtgcaca ggccccaagg gccagggcga atactctggc | 120 |
| tcctggaact ttggctttga ggtggcaggt gtctacacct ggcccagcgg aaacaccttt | 180 |
| gagggatact ggagccaggg caaacggcat gggctgggca tagagaccaa ggggcgctgg | 240 |
| ctctacaagg gcgagtggac acatggcttc aagggacgct acggaatccg gcagagctca | 300 |
| agcagcggtg ccaagtatga gggcacctgg aacaatggcc tgcaagacgg ctatggcacc | 360 |
| gagacctatg ctgatggagg gacgtaccaa ggccagttca ccaacggcat gcgccatggc | 420 |
| tacggagtac gccagagcgt gccctacggg atggccgtgg tggccaagtc gccgctgcgc | 480 |
| acgtcgctgt cgtccctgcg cagcgagcac agcaacggca cggtggcccc ggactctccc | 540 |
| gcctcgccgg cctccgacgg ccccgcgctg ccctcgcccg ccatcccgcg tggcggcttc | 600 |
| gcgctcagcg ccgccgccaa tgccgaggcg gccgcgcggg cgcccaaggg cggcggcctc | 660 |
| ttccagcggg gcgcgctgct gggcaagctg cggcgcgcag agtcgcgcac gtccgtgggt | 720 |
| agccagcgca gccgtgtcag cttccttaag agcgacctca gctcgggcgc cagcgacgcc | 780 |
| gcgtccaccg ccagcctggg agaggccgcc gagggcgccg acgaggccgc acccttcgag | 840 |
| gccgatatcg acgccaccac caccgagacc tacatgggcg agtggaagaa cgacaaacgc | 900 |
| tcgggcttcg gcgtgagcga acgctccagt ggcctccgct acgagggcga gtggctggac | 960 |
| aacctgcgcc acggctatgg ctgcaccacg ctgcccgacg gccaccgcga ggagggcaag | 1020 |
| taccgccaca acgtgctggt caaggacacc aagcgccgca tgctgcagct caagagcaac | 1080 |
| aaggtccgcc agaaagtgga gcacagtgtg gagggtgccc agcgcgccgc tgctatcgcg | 1140 |
| cgccagaagg ccgagattgc cgcctccagg acaagccacg ccaaggccaa agctgaggca | 1200 |
| gcggaacagg ccgccctggc tgccaaccag gagtccaaca ttgctcgcac tttggccagg | 1260 |
| gagctggctc cggacttcta ccagccaggt ccggaatatc agaagcgccg gctgctgcag | 1320 |
| gagatcctgg agaactcgga gagcctgctg gagccccccg accggggcgc cggcgcagcg | 1380 |
| ggcctcccac agccgccccg cgagagcccg cagctgcacg agcgtgagac ccctcggccc | 1440 |
| gagggtggct ccccgtcacc ggccgggacg cccccgcagc ccaagcggcc caggcccggg | 1500 |
| gtgtccaagg acggcctgct gagcccaggc gcctggaacg gcgagcccag cggtgagggc | 1560 |
| agccggtcag tcactccgtc cgagggcgcg ggccgccgca gccccgcgcg tccagccacc | 1620 |
| gagcgcatgg ccatcgaggc tctgcaggca ccgcctgcgc cgtcgcggga gccggaggtg | 1680 |
| gcgctttacc agggctacca cagctatgct gtgaagagca cgccgcccga gcccccaccc | 1740 |
| tttgaggacc agcccgagcc cgaggtctcc gggtccgagt ccgcgccctc gtccccggcc | 1800 |
| accgccccgc tgcaggcccc cacgctccga ggccccgagc ctgcacgcga gacccccgcc | 1860 |
| aagctggagc ccaagcccat catccccaaa gccgagccca gggccaaggc ccgcaagact | 1920 |
| gaggctcgag ggctgaccaa ggcgggggcc aagaagaagg cgcggaagga ggccgcactg | 1980 |
| gcggcagagg cggaggtgga ggtggaagag gtccccaaca ccatcctcat ctgcatggtg | 2040 |
| atcctgctga acatcggcct ggccatcctc tttgttcacc tcctgacctg a | 2091 |

In some embodiments, the JPH2 protein comprises a polypeptide sequence at least 75%, 80%, 85%, 90%, 92%, 93%, 94%, 95%, 96%, 97%, 98%, 99%, or 100% identical to SEQ ID NO: 9. In some embodiments, the JPH2 protein comprises a polynucleotide sequence at least 75%, 80%, 85%, 90%, 92%, 93%, 94%, 95%, 96%, 97%, 98%, 99%, or 100% identical to SEQ ID NO: 10.

In some embodiments, the disclosure provides a recombinant adeno-associated virus (rAAV) virion, comprising a capsid and a vector genome, wherein the vector genome comprises a polynucleotide sequence encoding an JPH2 or a functional variant thereof, operatively linked to a promoter. In some embodiments, the disclosure provides a recombinant adeno-associated virus (rAAV) virion, comprising a capsid and a vector genome, wherein the vector genome comprises a polynucleotide sequence encoding an JPH2, operatively linked to a promoter. In some embodiments, the JPH2 protein comprises a polypeptide sequence at least 75%, 80%, 85%, 90%, 92%, 93%, 94%, 95%, 96%, 97%, 98%, 99%, or 100% identical to SEQ ID NO: 1. The polynucleotide encoding the JPH2 may comprise a polynucleotide sequence at least 75%, 80%, 85%, 90%, 92%, 93%, 94%, 95%, 96%, 97%, 98%, 99%, or 100% identical to SEQ ID NO: 2.

Optionally, the polynucleotide sequence encoding the vector genome may comprise a Kozak sequence, including but not limited to GCCACCATGG (SEQ ID NO: 11). Kozak sequence may overlap the polynucleotide sequence encoding an JPH2 protein or a functional variant thereof. For example, the vector genome may comprise a polynucleotide sequence (with first ten nucleotides constituting the Kozak sequence) at least 75%, 80%, 85%, 90%, 92%, 93%, 94%, 95%, 96%, 97%, 98%, 99%, or 100% identical to SEQ ID NO: 12.

| SEQ ID NO: 12 | |
|---|---|
| 60 | gccaccatga gtgggggccg cttcgacttt gatgatggag gggcgtactg cgggggctgg |
| 120 | gaggggggaa aggcccatgg gcatggactg tgcacaggcc ccaagggcca gggcgaatac |
| 180 | tctggctcct ggaactttgg ctttgaggtg gcaggtgtct acacctggcc cagcggaaac |
| 240 | acctttgagg gatactggag ccagggcaaa cggcatgggc tgggcataga gaccaagggg |
| 300 | cgctggctct acaagggcga gtggacacat ggcttcaagg gacgctacgg aatccggcag |
| 360 | agctcaagca gcggtgccaa gtatgagggc acctggaaca atggcctgca agacggctat |
| 420 | ggcaccgaga cctatgctga tggagggacg taccaaggcc agttcaccaa cggcatgcgc |
| 480 | catggctacg gagtacgcca gagcgtgccc tacgggatgg ccgtggtggt gcgctcgccg |
| 540 | ctgcgcacgt cgctgtcgtc cctgcgcagc gagcacagca acggcacggt ggccccggac |
| 600 | tctcccgcct cgccggcctc cgacggcccc gcgctgccct cgcccgccat cccgcgtggc |
| 660 | ggcttcgcgc tcagcctcct ggccaatgcc gaggcggccg cgcgggcgcc caagggcggc |
| 720 | ggcctcttcc agcggggcgc gctgctgggc aagctgcggc gcgcagagtc gcgcacgtcc |
| 780 | gtgggtagcc agcgcagccg tgtcagcttc cttaagagcg acctcagctc gggcgccagc |
| 840 | gacgccgcgt ccaccgccag cctgggagag gccgccgagg gcgccgacga ggccgcaccc |
| 900 | ttcgaggccg atatcgacgc caccaccacc gagacctaca tgggcgagtg gaagaacgac |
| 960 | aaacgctcgg gcttcggcgt gagcgaacgc tccagtggcc tccgctacga gggcgagtgg |
| 1020 | ctggacaacc tgcgccacgg ctatggctgc accacgctgc ccgacggcca ccgcgaggag |
| 1080 | ggcaagtacc gccacaacgt gctggtcaag gacaccaagc gccgcatgct gcagctcaag |
| 1140 | agcaacaagg tccgccagaa agtggagcac agtgtggagg gtgcccagcg cgccgctgct |
| 1200 | atcgcgcgcc agaaggccga gattgccgcc tccaggacaa gccacgccaa ggccaaagct |
| 1260 | gaggcagcgg aacaggccgc cctggctgcc aaccaggagt ccaacattgc tcgcactttg |
| 1320 | gccagggagc tggctccgga cttctaccag ccaggtccgg aatatcagaa gcgccggctg |
| 1380 | ctgcaggaga tcctggagaa ctcggagagc ctgctggagc cccccgaccg gggcgccggc |
| 1440 | gcagcgggcc tcccacagcc gccccgcgag agcccgcagc tgcacgagcg tgagacccct |
| 1500 | cggcccgagg gtggctcccc gtcaccggcc gggacgcccc cgcagcccaa gcggcccagg |
| 1560 | cccggggtgt ccaaggacgg cctgctgagc ccaggcgcct ggaacggcga gcccagcggt |
| 1620 | gagggcagcc ggtcagtcac tccgtccgag ggcgcgggcc gccgcagccc cgcgcgtcca |
| 1680 | gccaccgagc gcatggccat cgaggctctg caggcaccgc ctgcgccgtc gcgggagccg |
| 1740 | gaggtggcgc tttaccaggg ctaccacagc tatgctgtgc gcaccacgcc gcccgagccc |
| 1800 | ccaccctttg aggaccagcc cgagcccgag gtctccgggt ccgagtccgc gccctcgtcc |
| 1860 | ccggccaccg ccccgctgca ggcccccacg ctccgaggcc ccgagcctgc acgcgagacc |
| 1920 | cccgccaagc tggagcccaa gcccatcatc cccaaagccg agcccagggc caaggcccgc |
| 1980 | aagactgagg ctcgagggct gaccaaggcg ggggccaaga agaaggcgcg gaaggaggcc |
| 2040 | gcactggcgg cagaggcgga ggtggaggtg gaagaggtcc ccaacaccat cctcatctgc |
| 2094 | atggtgatcc tgctgaacat cggcctggcc atcctctttg ttcacctcct gacc |

In some embodiments, the Kozak sequence is an alternative Kozak sequence comprising or consisting of any one of:

(SEQ ID NO: 13)
(gcc)gccRccAUGG;
(SEQ ID NO: 45)
AGNNAUGN;
(SEQ ID NO: 46)
ANNAUGG;
(SEQ ID NO: 47)
ANNAUGC;
(SEQ ID NO: 49),
ACCAUGG;
and
(SEQ ID NO: 14)
GACACCAUGG.

In some embodiments, the vector genome comprises no Kozak sequence.

The AAV virions of the disclosure comprise a vector genome. The vector genome may comprise an expression cassette (or a polynucleotide cassette for gene-editing applications not requiring expression of the polynucleotide sequence). Any suitable inverted terminal repeats (ITRs) may be used. The ITRs may be AAV ITRs from the same serotype as the capsid present in the AAV virion, or a different serotype from the capsid (e.g., AAV2 ITRs may be used with an AAV virion having an AAV9 capsid or an AAVrh74 capsid). In each case, the serotype of the capsid determines the name applied to the virion. The ITR are generally the most 5′ and most 3′ elements of the vector genome. The vector genome will also generally contain, in 5′ to 3′ order, a promoter, a transgene, 3′ untranslated region (UTR) sequences (e.g., a WPRE element), and a polyadenylation sequence. In variations, the vector genome includes an enhancer element (generally 5′ to the promoter) and/or an exon (generally 3′ to the promoter). In variations, the vector genome includes a Green Fluorescence Protein (GFP) protein, generally 3′ to the transgene. In variations, the vector genomes of the disclosure encode a partial or complete transgene sequence used as a repair template in a gene editing system. In such variations, the vector genome may comprise an exogenous promoter, or the gene editing system may insert the transgene into a locus in the genome having an endogenous promoter, such as a cardiac- or myocyte-specific promoter.

In some embodiments, the 5′ ITR comprises an AAV2 ITR. In some embodiments, the 5′ ITR comprises a polynucleotide sequence at least 75%, 80%, 85%, 90%, 92%, 93%, 94%, 95%, 96%, 97%, 98%, 99%, or 100% identical to SEQ ID NO: 15.

In some embodiments, the 5′ ITR comprises an AAV2 ITR. In some embodiments, the 5′ ITR comprises a polynucleotide sequence at least 75%, 80%, 85%, 90%, 92%, 93%, 94%, 95%, 96%, 97%, 98%, 99%, or 100% identical to SEQ ID NO: 16.

In some embodiments, the 5′ ITR comprises an AAV2 ITR. In some embodiments, the 5′ ITR comprises a polynucleotide sequence at least 75%, 80%, 85%, 90%, 92%, 93%, 94%, 95%, 96%, 97%, 98%, 99%, or 100% identical to SEQ ID NO: 17)

In some embodiments, the 5′ ITR comprises an AAV2 ITR. In some embodiments, the 5′ ITR comprises a polynucleotide sequence at least 75%, 80%, 85%, 90%, 92%, 93%, 94%, 95%, 96%, 97%, 98%, 99%, or 100% identical to SEQ ID NO: 18.

In some embodiments, the 3′ ITR comprises an AAV2 ITR. In some embodiments, the 5′ ITR comprises a polynucleotide sequence at least 75%, 80%, 85%, 90%, 92%, 93%, 94%, 95%, 96%, 97%, 98%, 99%, or 100% identical to SEQ ID NO: 19.

In some embodiments, the 3′ ITR comprises an AAV2 ITR. In some embodiments, the 5′ ITR comprises a polynucleotide sequence at least 75%, 80%, 85%, 90%, 92%, 93%, 94%, 95%, 96%, 97%, 98%, 99%, or 100% identical to SEQ ID NO: 20.

In some embodiments, the 3′ ITR comprises an AAV2 ITR. In some embodiments, the 5′ ITR comprises a polynucleotide sequence at least 75%, 80%, 85%, 90%, 92%, 93%, 94%, 95%, 96%, 97%, 98%, 99%, or 100% identical to SEQ ID NO: 21.

In some embodiments the vector genome comprises one or more filler sequences, e.g., at least 75%, 80%, 85%, 90%, 92%, 93%, 94%, 95%, 96%, 97%, 98%, 99%, or 100% identical to SEQ ID NO: 22; SEQ ID NO: 23; or SEQ ID NO: 24.

In some embodiments, the polynucleotide sequence encoding an JPH2 protein or functional variant thereof is operatively linked to a promoter. In certain embodiments, the promoter is an MHCK7 promoter. In certain embodiments, the promoter is an TNNT2 promoter.

The present disclosure contemplates use of various promoters. Promoters useful in embodiments of the present disclosure include, without limitation, a cytomegalovirus (CMV) promoter, phosphoglycerate kinase (PGK) promoter, or a promoter sequence comprised of the CMV enhancer and portions of the chicken beta-actin promoter and the rabbit beta-globin gene (CAG). In some cases, the promoter may be a synthetic promoter. Exemplary synthetic promoters are provided by Schlabach et al. PNAS USA. 107 (6): 2538-43 (2010). In some embodiments, the promoter comprises a polynucleotide sequence at least 75%, 80%, 85%, 90%, 92%, 93%, 94%, 95%, 96%, 97%, 98%, 99%, or 100% identical to SEQ ID NO: 25.

In some embodiments, a polynucleotide sequence encoding an JPH2 protein or functional variant thereof is operatively linked to an inducible promoter. An inducible promoter may be configured to cause the polynucleotide sequence to be transcriptionally expressed or not transcriptionally expressed in response to addition or accumulation of an agent or in response to removal, degradation, or dilution of an agent. The agent may be a drug. The agent may be tetracycline or one of its derivatives, including, without limitation, doxycycline. In some cases, the inducible promoter is a tet-on promoter, a tet-off promoter, a chemically-regulated promoter, a physically-regulated promoter (i.e., a promoter that responds to presence or absence of light or to low or high temperature). Inducible promoters include heavy metal ion inducible promoters (such as the mouse mammary tumor virus (mMTV) promoter or various growth hormone promoters), and the promoters from T7 phage which are active in the presence of T7 RNA polymerase. This list of inducible promoters is non-limiting.

In some cases, the promoter is a tissue-specific promoter, such as a promoter capable of driving expression in a cardiac cell to a greater extent than in a non-cardiac cell. In some embodiments, tissue-specific promoter is a selected from any various cardiac cell-specific promoters including but not limited to, desmin (Des), alpha-myosin heavy chain (α-MHC), myosin light chain 2 (MLC-2), cardiac troponin C (cTnC), cardiac troponin T (hTNNT2), muscle creatine kinase (CK) and combinations of promoter/enhancer regions thereof, such as MHCK7. In some cases, the promoter is a ubiquitous promoter. A “ubiquitous promoter” refers to a promoter that is not tissue-specific under experimental or clinical conditions. In some cases, the ubiquitous promoter is any one of Cytomegalovirus (CMV), Cytomegalovirus early enhancer element chicken beta-Actin gene intron with the splice acceptor of the rabbit beta-Globin gene (CAG), ubiquitin C (UBC), Phosphoglycerate Kinase (PGK), Eukaryotic translation elongation factor 1 alpha 1 (EF1-alpha), Glyceraldehyde 3-phosphate dehydrogenase (GAPDH), simian virus 40 (SV40), Hepatitis B virus (HBV), chicken beta-actin, and human beta-actin promoters.

In some embodiments, the promoter sequence is selected from Table 3. In some embodiments, the promoter comprises a polynucleotide sequence at least 75%, 80%, 85%, 90%, 92%, 93%, 94%, 95%, 96%, 97%, 98%, 99%, or 100% identical to any one of SEQ ID NOs: 31-51. In some embodiments, the promoter comprises a fragment of a polynucleotide sequence of any one of SEQ ID NOs: 31-51, e.g., a fragment comprising at least 25%, at least 50%, at least 75%, 80%, 85%, 90%, 92%, 93%, 94%, 95%, 96%, 97%, 98%, 99%, or 100% of any one of SEQ ID NOs: 31-51.

TABLE 3

| | | SEQ ID |
|---|---|---|
| PROMOTER | SEQUENCE | NO: |
| MHCK7 | ACCCTTCAGATTAAAAATAACTGAGGTAAGGGCCTGGGTAG | 31 |
| | GGGAGGTGGTGTGAGACGCTCCTGTCTCTCCTCTATCTGCCC | |
| | ATCGGCCCTTTGGGGAGGAGGAATGTGCCCAAGGACTAAAA | |
| | AAAGGCCATGGAGCCAGAGGGGCGAGGGCAACAGACCTTTC | |
| | ATGGGCAAACCTTGGGGCCCTGCTGTCTAGCATGCCCCACTA | |
| | CGGGTCTAGGCTGCCCATGTAAGGAGGCAAGGCCTGGGGAC | |
| | ACCCGAGATGCCTGGTTATAATTAACCCAGACATGTGGCTGC | |
| | CCCCCCCCCCCCAACACCTGCTGCCTCTAAAAATAACCCTGT | |
| | CCCTGGTGGATCCCCTGCATGCGAAGATCTTCGAACAAGGCT | |
| | GTGGGGGACTGAGGGCAGGCTGTAACAGGCTTGGGGGCCAG | |
| | GGCTTATACGTGCCTGGGACTCCCAAAGTATTACTGTTCCAT | |
| | GTTCCCGGCGAAGGGCCAGCTGTCCCCCGCCAGCTAGACTCA | |
| | GCACTTAGTTTAGGAACCAGTGAGCAAGTCAGCCCTTGGGGC | |
| | AGCCCATACAAGGCCATGGGGCTGGGCAAGCTGCACGCCTG | |
| | GGTCCGGGGTGGGCACGGTGCCCGGGCAACGAGCTGAAAGC | |
| | TCATCTGCTCTCAGGGGCCCCTCCCTGGGGACAGCCCCTCCT | |
| | GGCTAGTCACACCCTGTAGGCTCCTCTATATAACCCAGGGGC | |
| | ACAGGGGCTGCCCTCATTCTACCACCACCTCCACAGCACAGA | |
| | CAGACACTCAGGAGCCAGCCAG | |
| Human cardiac | CTCAGTCCATTAGGAGCCAGTAGCCTGGAAGATGTCTTTACC | 33 |
| troponin T | CCCAGCATCAGTTCAAGTGGAGCAGCACATAACTCTTGCCCT | |
| promoter | CTGCCTTCCAAGATTCTGGTGCTGAGACTTATGGAGTGTCTT | |
| (without exon 1) | GGAGGTTGCCTTCTGCCCCCCAACCCTGCTCCCAGCTGGCCC | |
| hTnnT2/ | TCCCAGGCCTGGGTTGCTGGCCTCTGCTTTATCAGGATTCTCA | |
| HTNNT2 | AGAGGGACAGCTGGTTTATGTTGCATGACTGTTCCCTGCATA | |
| | TCTGCTCTGGTTTTAAATAGCTTATCTGAGCAGCTGGAGGAC | |
| | CACATGGGCTTATATGGCGTGGGGTACATGTTCCTGTAGCCT | |
| | TGTCCCTGGCACCTGCCAAAATAGCAGCCAACACCCCCCACC | |
| | CCCACCGCCATCCCCCTGCCCCACCCGTCCCCTGTCGCACAT | |
| | TCCTCCCTCCGCAGGGCTGGCTCACCAGGCCCCAGCCCACAT | |
| | GCCTGCTTAAAGCCCTCTCCATCCTCTGCCTCACCCAGT | |
| Human cardiac | CTCAGTCCATTAGGAGCCAGTAGCCTGGAAGATGTCTTTACC | 32 |
| troponin T | CCCAGCATCAGTTCAAGTGGAGCAGCACATAACTCTTGCCCT | |
| promoter (with | CTGCCTTCCAAGATTCTGGTGCTGAGACTTATGGAGTGTCTT | |
| exon 1, | GGAGGTTGCCTTCTGCCCCCCAACCCTGCTCCCAGCTGGCCC | |
| underlined) | TCCCAGGCCTGGGTTGCTGGCCTCTGCTTTATCAGGATTCTCA | |
| hTnnT2/ | AGAGGGACAGCTGGTTTATGTTGCATGACTGTTCCCTGCATA | |
| HTNNT2 | TCTGCTCTGGTTTTAAATAGCTTATCTGAGCAGCTGGAGGAC | |
| | CACATGGGCTTATATGGCGTGGGGTACATGTTCCTGTAGCCT | |
| | TGTCCCTGGCACCTGCCAAAATAGCAGCCAACACCCCCCACC | |
| | CCCACCGCCATCCCCCTGCCCCACCCGTCCCCTGTCGCACAT | |
| | TCCTCCCTCCGCAGGGCTGGCTCACCAGGCCCCAGCCCACAT | |
| | GCCTGCTTAAAGCCCTCTCCATCCTCTGCCTCACCCAGTCCCC | |
| | GCTGAGACTGAGCAGACGCCTCCAGGATCTGTCGGCAG | |
| Mouse α- | GGTACCGGATCCTGCAAGGTCACACAAGGGTCTCCACCCACC | 34 |
| cardiac myosin | AGGTGCCCTAGTCTCAATTTCAGTTTCCATGCCTTGTTCTCAC | |
| heavy chain | AATGCTGGCCTCCCCAGAGCTAATTTGGACTTTGTTTTTATTT | |
| promoter | CAAAAGGGCCTGAATGAGGAGTAGATCTTGTGCTACCCAGC | |
| (αMHC) | TCTAAGGGTGCCCGTGAAGCCCTCAGACCTGGAGCCTTTGCA | |
| | ACAGCCCTTTAGGTGGAAGCAGAATAAAGCAATTTTCCTTAA | |
| | AGCCAAAATCCTGCCTCTAGACTCTTCTTCTCTGACCTCGGTC | |
| | CCTGGGCTCTAGGGTGGGGAGGTGGGGCTTGGAAGAAGAAG | |
| | GTGGGGAAGTGGCAAAAGCCGATCCCTAGGGCCCTGTGAAG | |
| | TTCGGAGCCTTCCCTGTACAGCACTGGCTCATAGATCCTCCT | |
| | CCAGCCAAACATAGCAAGAAGTGATACCTCCTTTGTGACTTC | |
| | CCCAGGCCCAGTACCTGTCAGGTTGAAACAGGATTTAGAGA | |
| | AGCCTCTGAACTCACCTGAACTCTGAAGCTCATCCACCAAGC | |
| | AAGCACCTAGGTGCCACTGCTAGTTAGTATCCTACGCTGATA | |
| | ATATGCAGAGCTGGGCCACAGAAGTCCTGGGGTGTAGGAAC | |
| | TGACCAGTGACTTTTCAGTCGGCAAAGGTATGACCCCCTCAG | |
| | CAGATGTAGTAATGTCCCCTTAGATCCCATCCCAGGCAGGTC | |
| | TCTAAGAGGACATGGGATGAGAGATGTAGTCATGTGGCATT | |
| | CCAAACACAGCTATCCACAGTGTCCCTTGCCCCTTCCACTTA | |
| | GCCAGGAGGACAGTAACCTTAGCCTATCTTTCTTCCTCCCCA | |
| | TCCTCCCAGGACACACCCCCTGGTCTGCAGTATTCATTTCTTC | |
| | CTTCACGTCCCCTCTGTGACTTCCATTTGCAAGGCTTTTGACC | |
| | TCTGCAGCTGCTGGAAGATAGAGTTTGGCCCTAGGTGTGGCA | |
| | AGCCATCTCAAGAGAAAGCAGACAACAGGGGGACCAGATTT | |
| | TGGAAGGATCAGGAACTAAATCACTGGCGGGCCTGGGGGTA | |
| | GAAAAAAGAGTGAGTGAGTCCGCTCCAGCTAAGCCAAGCTA | |
| | GTCCCCGAGATACTCTGCCACAGCTGGGCTGCTCGGGGTAGC | |
| | TTTAGGAATGTGGGTCTGAAAGACAATGGGATTGGAAGACA | |
| | TCTCTTTGAGTCTCCCCTCAACCCCACCTACAGACACACTCGT | |
| | GTGTGGCCAGACTCCTGTTCAACAGCCCTCTGTGTTCTGACC | |
| | ACTGAGCTAGGCAACCAGAGCATGGGCCCTGTGCTGAGGAT | |
| | GAAGAGITGGTTACCAATAGCAAAAACAGCAGGGGAGGGAG | |
| | AACAGAGAACGAAATAAGGAAGGAAGAAGGAAAGGCCAGT | |
| | CAATCAGATGCAGTCAGAAGAGATGGGAAGCCAACACACAG | |
| | CTTGAGCAGAGGAAACAGAAAAGGGAGAGATTCTGGGCATA | |
| | AGGAGGCCACAGAAAGAAGAGCCCAGGCCCCCCAAGTCTCC | |
| | TCTTTATACCCTCATCCCGTCTCCCAATTAAGCCCACTCTTCT | |
| | TCCTAGATCAGACCTGAGCTGCAGCGAAGAGACCCGTAGGG | |
| | AGGATCACACTGGATGAAGGAGATGTGTGGAGAAGTCCAGG | |
| | GAACCTAAGAGCCAGAGCCTAAAAGAGCAAGAGATAAAGGT | |
| | GCTTCAAAGGTGGCCAGGCTGTGCACACAGAGGGTCGAGGA | |
| | CTGGTGGTAGAGCCTCAAGATAAGGATGATGCTCAGAATGG | |
| | GCGGGGGGGGGGATTCTGGGGGGGGGAGAGAGAAGGTGAG | |
| | AAGGAGCCTGGAACAGAGAATCTGGAAGCGCTGGAAACGAT | |
| | ACCATAAAGGGAAGAACCCAGGCTACCTTTAGATGTAAATC | |
| | ATGAAAGACAGGGAGAAGGGAAGCTGGAGAGAGTAGAAGG | |
| | ACCCCGGGGCAAGACATTGAAGCAAGGACAAGCCAGGTTGA | |
| | GCGCTCCGTGAAATCAGCCTGCTGAAGGCAGAGCCCTGGTAT | |
| | GAGCACCAGAACAGCAGAGGCTAGGGTTAATGTCGAGACAG | |
| | GGAACAGAAGGTAGACACAGGAACAGACAGAGACGGGGGA | |
| | GCCAGGTAACAAAGGAATGGTCCTTCTCACCTGTGGCCAGA | |
| | GCGTCCATCTGTGTCCACATACTCTAGAATGTTCATCAGACT | |
| | GCAGGGCTGGCTTGGGAGGCAGCTGGAAAGAGTATGTGAGA | |
| | GCCAGGGGAGACAAGGGGGCCTAGGAAAGGAAGAAGAGGG | |
| | CAAACCAGGCCACACAAGAGGGCAGAGCCCAGAACTGAGTT | |
| | AACTCCTTCCTTGTTGCATCTTCCATAGGAGGCAGTGGGAAC | |
| | TCTGTGACCACCATCCCCCATGAGCCCCCACTACCCATACCA | |
| | AGTTTGGCCTGAGTGGCATTCTAGGTTCCCTGAGGACAGAGC | |
| | CTGGCCTTTGTCTCTTGGACCTGACCCAAGCTGACCCAATGT | |
| | TCTCAGTACCTTATCATGCCCTCAAGAGCTTGAGAACCAGGC | |
| | AGTGACATATTAGGCCATGGGCTAACCCTGGAGCTTGCACAC | |
| | AGGAGCCTCAAGTGACCTCCAGGGACACAGCTGCAGACAGG | |
| | TGGCCTTTATCCCCAAAGAGCAACCATTTGGCATAGGTGGCT | |
| | GCAAATGGGAATGCAAGGTTGAATCAGGTCCCTTCAAGAAT | |
| | ACTGCATGCAAGACCTAAGACCCCTGGAGAGAGGGGTATGC | |
| | TCCTGCCCCCACCCACCATAAGGGGAGTGAACTATCCTAGGG | |
| | GGCTGGCGACCTTGGGGAGACACCACATTACTGAGAGTGCT | |
| | GAGCCCAGAAAAACTGACCGCCCTGTGTCCTGCCCACCTCCA | |
| | CACTCTAGAGCTATATTGAGAGGTGACAGTAGATAGGGTGG | |
| | GAGCTGGTAGCAGGGAGAGTGTTCCTGGGTGTGAGGGTGTA | |
| | GGGGAAAGCCAGAGCAGGGGAGTCTGGCTTTGTCTCCTGAA | |
| | CACAATGTCTACTTAGTTATAACAGGCATGACCTGCTAAAGA | |
| | CCCAACATCTACGACCTCTGAAAAGACAGCAGCCCTGGAGG | |
| | ACAGGGGTTGTCTCTGAGCCTTGGGTGCTTGATGGTGCCACA | |
| | AAGGAGGGCATGAGTGTGAGTATAAGGCCCCAGGAGCGTTA | |
| | GAGAAGGGCACTTGGGAAGGGGTCAGTCTGCAGAGCCCCTA | |
| | TCCATGGAATCTGGAGCCTGGGGCCAACTGGTGTAAATCTCT | |
| | GGGCCTGCCAGGCATTCAAAGCAGCACCTGCATCCTCTGGCA | |
| | GCCTGGGGAGGCGGAAGGGAGCAACCCCCCACTTATACCCT | |
| | TTCTCCCTCAGCCCCAGGATTAACACCTCTGGCCTTCCCCCTT | |
| | CCCACCTCCCATCAGGAGTGGAGGGTTGCAGAGGGAGGGTA | |
| | AAAACCTACATGTCCAAACATCATGGTGCACGATATATGGAT | |
| | CAGTATGTGTAGAGGCAAGAAAGGAAATCTGCAGGCTTAAC | |
| | TGGGTTAATGTGTAAAGTCTGTGTGCATGTGTGTGTGTCTGA | |
| | CTGAAAACGGGCATGGCTGTGCAGCTGTTCAGTTCTGTGCGT | |
| | GAGGTTACCAGACTGCAGGTTTGTGTGTAAATTGCCCAAGGC | |
| | AAAGTGGGTGAATCCCTTCCATGGTTTAAAGAGATTGGATGA | |
| | TGGCCTGCATCTCAAGGACCATGGAAAATAGAATGGACACT | |
| | CTATATGTGTCTCTAAGCTAAGGTAGCAAGGTCTTTGGAGGA | |
| | CACCTGTCTAGAGATGTGGGCAACAGAGACTACAGACAGTA | |
| | TCTGTACAGAGTAAGGAGAGAGAGGAGGGGGTGTAGAATTC | |
| | TCTTACTATCAAAGGGAAACTGAGTCGTGCACCTGCAAAGTG | |
| | GATGCTCTCCCTAGACATCATGACTTTGTCTCTGGGGAGCCA | |
| | GCACTGTGGAACTTCAGGTCTGAGAGAGTAGGAGGCTCCCCT | |
| | CAGCCTGAAGCTATGCAGATAGCCAGGGTTGAAAGGGGGAA | |
| | GGGAGAGCCTGGGATGGGAGCTTGTGTGTTGGAGGCAGGGG | |
| | ACAGATATTAAGCCTGGAAGAGAAGGTGACCCTTACCCAGT | |
| | TGTTCAACTCACCCTTCAGATTAAAAATAACTGAGGTAAGGG | |
| | CCTGGGTAGGGGAGGTGGTGTGAGACGCTCCTGTCTCTCCTC | |
| | TATCTGCCCATCGGCCCTTTGGGGAGGAGGAATGTGCCCAAG | |
| | GACTAAAAAAAGGCCATGGAGCCAGAGGGGCGAGGGCAAC | |
| | AGACCTTTCATGGGCAAACCTTGGGGCCCTGCTGTCCTCCTG | |
| | TCACCTCCAGAGCCAAGGGATCAAAGGAGGAGGAGCCAGGA | |
| | CAGGAGGGAAGTGGGAGGGAGGGTCCCAGCAGAGGACTCC | |
| | AAATTTAGGCAGCAGGCATATGGGATGGGATATAAAGGGGC | |
| | TGGAGCACTGAGAGCTGTCAGAGATTTCTCCAACCCAGGTAA | |
| | GAGGGAGTTTCGGGTGGGGGCTCTTCACCCACACCAGACCTC | |
| | TCCCCACCTAGAAGGAAACTGCCTTTCCTGGAAGTGGGGTTC | |
| | AGGCCGGTCAGAGATCTGACAGGGTGGCCTTCCACCAGCCT | |
| | GGGAAGTTCTCAGTGGCAGGAGGTTTCCACAAGAAACACTG | |
| | GATGCCCCTTCCCTTACGCTGTCTTCTCCATCTTCCTCCTGGG | |
| | GATGCTCCTCCCCGTCTTGGTTTATCTTGGCTCTTCGTCTTCA | |
| | GCAAGATTTGCCCTGTGCTGTCCACTCCATCTTTCTCTACTGT | |
| | CTCCGTGCCTTGCCTTGCCTTCTTGCGTGTCCTTCCTTTCCAC | |
| | CCATTTCTCACTTCACCTTTTCTCCCCTTCTCATTTGTATTCAT | |
| | CCTTCCTTCCTTCCTTCCTTCCTTCCTTCCTTCCTTCCTTCCTTC | |
| | CTTTCTCCCTTCCTTCCTTCCTTCCTTCCTTCCTTCCTTCCTTCC | |
| | TTCCTGTGTCAGAGTGCTGAGAATCACACCTGGGGTTCCCAC | |
| | CCTTATGTAAACAATCTTCCAGTGAGCCACAGCTTCAGTGCT | |
| | GCTGGGTGCTCTCTTACCTTCCTCACCCCCTGGCTTGTCCTGT | |
| | TCCATCCTGGTCAGGATCTCTAGATTGGTCTCCCAGCCTCTGC | |
| | TACTCCTCTTCCTGCCTGTTCCTCTCTCTGTCCAGCTGCGCCA | |
| | CTGTGGTGCCTCGTTCCAGCTGTGGTCCACATTCTTCAGGATT | |
| | CTCTGAAAAGTTAACCAGGTGAGAATGTTTCCCCTGTAGACA | |
| | GCAGATCACGATTCTCCCGGAAGTCAGGCTTCCAGCCCTCTC | |
| | TTTCTCTGCCCAGCTGCCCGGCACTCTTAGCAAACCTCAGGC | |
| | ACCCTTACCCCACATAGACCTCTGACAGAGAAGCAGGCACTT | |
| | TACATGGAGTCCTGGTGGGAGAGCCATAGGCTACGGTGTAA | |
| | AAGAGGCAGGGAAGTGGTGGTGTAGGAAAGTCAGGACTTCA | |
| | CATAGAAGCCTAGCCCACACCAGAAATGACAGACAGATCCC | |
| | TCCTATCTCCCCCATAAGAGTTTGAGTCGACCCGCGGCCCCG | |
| | AATTG | |
| Chicken cardiac | GGGATAAAAGCAGTCTGGGCTTTCACATGACAGCATCTGGG | 35 |
| troponin T | GCTGCGGCAGAGGGTCGGGTCCGAAGCGCTGCCTTATCAGC | |
| promoter | GTCCCCAGCCCTGGGAGGTGACAGCTGGCTGGCTTGTGTCAG | |
| (cTnT) | CCCCTCGGGCACTCACGTATCTCCGTCCGACGGGTTTAAAAT | |
| | AGCAAAACTCTGAGGCCACACAATAGCTTGGGCTTATATGG | |
| | GCTCCTGTGGGGGAAGGGGGAGCACGGAGGGGGCCGGGGCC | |
| | GCTGCTGCCAAAATAGCAGCTCACAAGTGTTGCATTCCTCTC | |
| | TGGGCGCCGGGCACATTCCTGCTGGCTCTGCCCGCCCCGGGG | |
| | TGGGCGCCGGGGGGACCTTAAAGCCTCTGCCCCCCAAGGAG | |
| | CCCTTCCCAGACAGCCGCCGGCACCCACCGCTCCGTGGGA | |
| Human Creatine | CTCTCAGCCCTGGAAGTCCTTGCTCACAGCCGAGGCGCCGAG | 36 |
| Kinase M | AGCGCTTGCTCTGCCCAGATCTGCGCGAGTCTGGCGCCCGCG | |
| (hCKM) | CTCTGAACGGCGTCGCTGCCCAGCCCCCTTCCCCGGGAGGTG | |
| | GGAGCGGCCACCCAGGGCCCCGTGGCTGCCCTTGTAAGGAG | |
| | GCGAGGCCCGAGGACACCCGAGACGCCCGGTTATAATTAAC | |
| | CAGGACACGTGGCGAACCCCCCTCCAACACCTGCCCCCGAA | |
| | CCCCCCCATACCCAGCGCCTCGGGTCTCGGCCTTTGCGGCAG | |
| | AGGAGACAGCAAAGCGCCCTCTAAAAATAACTCCTTTCCCG | |
| | GCGACCGAGACCCTCCCTGTCCCCCGCACAGCGGAAATCTCC | |
| | CAGTGGCACCGAGGGGGCGAGGGTTAAGTGGGGGGGAGGGT | |
| | GACCACCGCCTCCCACCCTTGCCCTGAGTTTGAATCTCTCCA | |
| | ACTCAGCCAGCCTCAGTTTCCCCTCCACTCAGTCCCTAGGAG | |
| | GAAGGGGCGCCCAAGCGCGGGTTTCTGGGGTTAGACTGCCC | |
| | TCCATTGCAATTGGTCCTTCTCCCGGCCTCTGCTTCCTCCAGC | |
| | TCACAGGGTATCTGCTCCTCCTGGAGCCACACCTTGGTTCCC | |
| | CGAGGTGCCGCTGGGACTCGGGTAGGGGTGAGGGCCCAGGG | |
| | GGCACAGGGGGAGCCGAGGGCCACAGGAAGGGCTGGTGGCT | |
| | GAAGGAGACTCAGGGGCCAGGGGACGGTGGCTTCTACGTGC | |
| | TTGGGACGTTCCCAGCCACCGTCCCATGTTCCCGGCGGGGGG | |
| | CCAGCTGTCCCCACCGCCAGCCCAACTCAGCACTTGGTCAGG | |
| | GTATCAGCTTGGTGGGGGGGCGTGAGCCCAGCCCCTGGGGC | |
| | GGCTCAGCCCATACAAGGCCATGGGGCTGGGCGCAAAGCAT | |
| | GCCTGGGTTCAGGGTGGGTATGGTGCGGGAGCAGGGAGGTG | |
| | AGAGGCTCAGCTGCCCTCCAGAACTCCTCCCTGGGGACAACC | |
| | CCTCCCAGCCAATAGCACAGCCTAGGTCCCCCTATATAAGGC | |
| | CACGGCTGCTGGCCCTTCCTTTGGGTCAGTGTCACCTCCAGG | |
| | ATACAGACA | |
| Human beta- | GCCCAGCACCCCAAGGCGGCCAACGCCAAAACTCTCCCTCCT | 37 |
| actin (HuBa) | CCTCTTCCTCAATCTCGCTCTCGCTCTTTTTTTTTTTCGCAAAA | |
| | GGAGGGGAGAGGGGGTAAAAAAATGCTGCACTGTGCGGCGA | |
| | AGCCGGTGAGTGAGCGGCGCGGGGCCAATCAGCGTGCGCCG | |
| | TTCCGAAAGTTGCCTTTTATGGCTCGAGCGGCCGCGGCGGCG | |
| | CCCTATAAAACCCAGCGGCGCGACGCGCCACCACCGCCGAG | |
| | TC | |
| Chicken beta- | GGTCGAGGTGAGCCCCACGTTCTGCTTCACTCTCCCCATCTC | 38 |
| actin (CBA) | CCCCCCCTCCCCACCCCCAATTTTGTATTTATTTATTTTTTAAT | |
| | TATTTTGTGCAGCGATGGGGGCGGGGGGGGGGGGGGCGCGC | |
| | GCCAGGCGGGGCGGGGCGGGGCGAGGGGCGGGGCGGGGCG | |
| | AGGCGGAGAGGTGCGGCGGCAGCCAATCAGAGCGGCGCGCT | |
| | CCGAAAGTTTCCTTTTATGGCGAGGCGGCGGCGGCGGCGGCC | |
| | CTATAAAAAGCGAAGCGCGCGGCGGGCGGGA | |
| Cytomegalovirus | TGGTGATGCGGTTTTGGCAGTACACCAATGGGCGTGGATAGC | 39 |
| (CMV) | GGTTTGACTCACGGGGATTTCCAAGTCTCCACCCCATTGACG | |
| | TCAATGGGAGTTTGTTTTGGCACCAAAATCAACGGGACTTTC | |
| | CAAAATGTCGTAATAACCCCGCCCCGTTGACGCAAATGGGC | |
| | GGTAGGCGTGTACGGTGGGAGGTCTATATAAGCAGAGCTCG | |
| | TTTAGTGAACCG | |
| Cytomegalovirus | TAGTTATTAATAGTAATCAATTACGGGGTCATTAGTTCATAG | 40 |
| (CMV) | CCCATATATGGAGTTCCGCGTTACATAACTTACGGTAAATGG | |
| (second version) | CCCGCCTGGCTGACCGCCCAACGACCCCCGCCCATTGACGTC | |
| | AATAATGACGTATGTTCCCATAGTAACGCCAATAGGGACTTT | |
| | CCATTGACGTCAATGGGTGGAGTATTTACGGTAAACTGCCCA | |
| | CTTGGCAGTACATCAAGTGTATCATATGCCAAGTACGCCCCC | |
| | TATTGACGTCAATGACGGTAAATGGCCCGCCTGGCATTATGC | |
| | CCAGTACATGACCTTATGGGACTTTCCTACTTGGCAGTACAT | |
| | CTACGTATTAGTCATCGCTATTACCATGGTGATGCGGTTTTG | |
| | GCAGTACATCAATGGGCGTGGATAGCGGTTTGACTCACGGG | |
| | GATTTCCAAGTCTCCACCCCATTGACGTCAATGGGAGTTTGT | |
| | TTTGGCACCAAAATCAACGGGACTTTCCAAAATGTCGTAACA | |
| | ACTCCGCCCCATTGACGCAAATGGGCGGTAGGCGTGTACGGT | |
| | GGGAGGTCTATATAAGCAGAGCTGGTTTAGTGAACCGT | |
| Cytomegalovirus | CGTTACATAACTTACGGTAAATGGCCCGCCTGGCTGACCGCC | 41 |
| (CMV) (third | CAACGACCCCCGCCCATTGACGTCAATAATGACGTATGTTCC | |
| version) | CATAGTAACGCCAATAGGGACTTTCCATTGACGTCAATGGGT | |
| | GGAGTATTTACGGTAAACTGCCCACTTGGCAGTACATCAAGT | |
| | GTATCATATGCCAAGTACGCCCCCTATTGACGTCAATGACGG | |
| | TAAATGGCCCGCCTGGCATTATGCCCAGTACATGACCTTATG | |
| | GGACTTTCCTACTTGGCAGTACATCTACGTATTAGTCATCGCT | |
| | ATTACCATGGTGATGCGGTTTTGGCAGTACATCAATGGGCGT | |
| | GGATAGCGGTTTGACTCACGGGGATTTCCAAGTCTCCACCCC | |
| | ATTGACGTCAATGGGAGTTTGTTTTGGCACCAAAATCAACGG | |
| | GACTTTCCAAAATGTCGTAACAACTCCGCCCCATTGACGCAA | |
| | ATGGGCGGTAGGCGTGTACGGTGGGAGGTCTATATAAGCAG | |
| | AGCT | |
| CAG promoter | ACTTACGGTAAATGGCCCGCCTGGCTGACCGCCCAACGACCC | 42 |
| (first version) | CCGCCCATTGACGTCAATAATGACGTATGTTCCCATAGTAAC | |
| | GCCAATAGGGACTTTCCATTGACGTCAATGGGTGGAGTATTT | |
| | ACGGTAAACTGCCCACTTGGCAGTACATCAAGTGTATCATAT | |
| | GCCAAGTACGCCCCCTATTGACGTCAATGACGGTAAATGGCC | |
| | CGCCTGGCATTATGCCCAGTACATGACCTTATGGGACTTTCC | |
| | TACTTGGCAGTACATCTACGTATTAGTCATCGCTATTACCAT | |
| | GGTCGAGGTGAGCCCCACGTTCTGCTTCACTCTCCCCATCTC | |
| | CCCCCCCTCCCCACCCCCAATTTTGTATTTATTTATTTTTTAAT | |
| | TATTTTGTGCAGCGATGGGGGCGGGGGGGGGGGGGGCGCGC | |
| | GCCAGGCGGGGCGGGGCGGGGCGAGGGGCGGGGCGGGGCG | |
| | AGGCGGAGAGGTGCGGCGGCAGCCAATCAGAGCGGCGCGCT | |
| | CCGAAAGTTTCCTTTTATGGCGAGGCGGCGGCGGCGGCGGCC | |
| | CTATAAAAAGCGAAGCGCGCGGCGGGCGG | |
| CAG promoter | CGTTACATAACTTACGGTAAATGGCCCGCCTGGCTGACCGCC | 43 |
| (second version) | CAACGACCCCCGCCCATTGACGTCAATAATGACGTATGTTCC | |
| | CATAGTAACGCCAATAGGGACTTTCCATTGACGTCAATGGGT | |
| | GGAGTATTTACGGTAAACTGCCCACTTGGCAGTACATCAAGT | |
| | GTATCATATGCCAAGTACGCCCCCTATTGACGTCAATGACGG | |
| | TAAATGGCCCGCCTGGCATTATGCCCAGTACATGACCTTATG | |
| | GGACTTTCCTACTTGGCAGTACATCTACGTATTAGTCATCGCT | |
| | ATTACCATGTCGAGGTGAGCCCCACGTTCTGCTTCACTCTCC | |
| | CCATCTCCCCCCCCTCCCCACCCCCAATTTTGTATTTATTTAT | |
| | TTTTTAATTATTTTGTGCAGCGATGGGGGCGGGGGGGGGGGG | |
| | GGCGCGCGCCAGGCGGGGCGGGGCGGGGCGAGGGGCGGGG | |
| | CGGGGCGAGGCGGAGAGGTGCGGCGGCAGCCAATCAGAGC | |
| | GGCGCGCTCCGAAAGTTTCCTTTTATGGCGAGGCGGCGGCGG | |
| | CGGCGGCCCTATAAAAAGCGAAGCGCGCGGCGGGCG | |
| Human EF1- | CAACCTTTGGAGCTAAGCCAGCAATGGTAGAGGGAAGATTC | 44 |
| alpha (EF1-α) | TGCACGTCCCTTCCAGGCGGCCTCCCCGTCACCACCCCCCCC | |
| | AACCCGCCCCGACCGGAGCTGAGAGTAATTCATACAAAAGG | |
| | ACTCGCCCCTGCCTTGGGGAATCCCAGGGACCGTCGTTAAAC | |
| | TCCCACTAACGTAGAACCCAGAGATCGCTGCGTTCCCGCCCC | |
| | CTCACCCGCCCGCTCTCGTCATCACTGAGGTGGAGAATAGCA | |
| | TGCGTGAGGCTCCGGTGCCCGTCAGTGGGCAGAGCGCACAT | |
| | CGCCCACAGTCCCCGAGAAGTTGGGGGGAGGGGTCGGCAAT | |
| | TGAACGGGTGCCTAGAGAAGGTGGCGCGGGGTAAACTGGGA | |
| | AAGTGATGTCGTGTACTGGCTCCGCCTTTTTCCCGAGGGTGG | |
| | GGGAGAACCGTATATAAGTGCAGTAGTCGCCGTGAACGTT | |
| Human | ACTTGTGGACAAAGTTTGCTCTATTCCACCTCCTCCAGGCCCT | 48 |
| CamKIIa | CCTTGGGTCCATCACCCCAGGGGTGCTGGGTCCATCCCACCC | |
| (CaMKIIa) | CCAGGCCCACACAGGCTTGCAGTATTGTGTGCGGTATGGTCA | |
| | GGGCGTCCGAGAGCAGGTTTCGCAGTGGAAGGCAGGCAGGT | |
| | GTTGGGGAGGCAGTTACCGGGGCAACGGGAACAGGGCGTTT | |
| | TGGAGGTGGTTGCCATGGGGACCTGGATGCTGACGAAGGCT | |
| | CGCGAGGCTGTGAGCAGCCACAGTGCCCTGC | |

In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 31. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 32. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 33.

Further illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. A large variety of other promoters are known and generally available in the art, and the sequences of many such promoters are available in sequence databases such as the GenBank database.

In some cases, vectors of the present disclosure further comprise one or more regulatory elements selected from the group consisting of an enhancer, an intron, a poly-A signal, a 2A peptide encoding sequence, a WPRE (Woodchuck hepatitis virus posttranscriptional regulatory element), and a HPRE (Hepatitis B posttranscriptional regulatory element).

In some embodiments, the vector comprises a CMV enhancer.

In certain embodiments, the vectors comprise one or more enhancers. In particular embodiments, the enhancer is a CMV enhancer sequence, a GAPDH enhancer sequence, a β-actin enhancer sequence, or an EF1-α enhancer sequence. Sequences of the foregoing are known in the art. For example, the sequence of the CMV immediate early (IE) enhancer is SEQ ID NO: 50.

In certain embodiments, the vectors comprise one or more introns. In particular embodiments, the intron is a rabbit globin intron sequence, a chicken β-actin intron sequence, a synthetic intron sequence, an SV40 intron, or an EF1-α intron sequence.

In certain embodiments, the vectors comprise a polyA sequence. In particular embodiments, the polyA sequence is a rabbit globin polyA sequence, a human growth hormone polyA sequence, a bovine growth hormone polyA sequence, a PGK polyA sequence, an SV40 polyA sequence, or a TK polyA sequence. In some embodiments, the poly-A signal may be a bovine growth hormone polyadenylation signal (bGHpA).

In certain embodiments, the vectors comprise one or more transcript stabilizing element. In particular embodiments, the transcript stabilizing element is a WPRE sequence, a HPRE sequence, a scaffold-attachment region, a 3′ UTR, or a 5′ UTR. In particular embodiments, the vectors comprise both a 5′ UTR and a 3′ UTR.

In some embodiments, the vector comprises a 5′ untranslated region (UTR) selected from Table 4. In some embodiments, the vector genome comprises a polynucleotide sequence at least 75%, 80%, 85%, 90%, 92%, 93%, 94%, 95%, 96%, 97%, 98%, 99%, or 100% identical to any one of SEQ ID NOS 51-61.

TABLE 4

| 5′ | | SEQ |
|---|---|---|
| UNTRANSLATED | | ID |
| REGION | SEQUENCE | NO: |
| Human beta-actin | CGCGTCCGCCCGCGAGCACAGAGCCTCGCCTTTGCCGATC | 51 |
| exon/intron | CGCCGCCCGTCCACACCCGCCGCCAGGTAAGCCCGGCCAG | |
| | CCGACCGGGGCATGCGGCCGCGGCCCTTCGCCCGTGCAGA | |
| | GCCGCCGTCTGGGCCGCAGCGGGGGGCGCATGGGGCGGA | |
| | ACCGGACCGCCGTGGGGGGCGCGGGAGAAGCCCCTGGGC | |
| | CTCCGGAGATGGGGGACACCCCACGCCAGTTCGCAGGCG | |
| | CGAGGCCGCGCTCGGGCGGGCGCGCTCCGGGGGTGCCGC | |
| | TCTCGGGGCGGGGGCAACCGGCGGGGTCTTTGTCTGAGCC | |
| | GGGCTCTTGCCAATGGGGATCGCACGGTGGGCGCGGCGTA | |
| | GCCCCCGTCAGGCCCGGTGGGGGCTGGGGCGCCATGCGC | |
| | GTGCGCGCTGGTCCTTTGGGCGCTAACTGCGTGCGCGCTG | |
| | GGAATTGGCGCTAATTGCGCGTGCGCGCTGGGACTCAATG | |
| | GCGCTAATCGCGCGTGCGTTCTGGGGCCCGGGCGCTTGCG | |
| | CCACTTCCTGCCCGAGCCGCTGGCGCCCGAGGGTGTGGCC | |
| | GCTGCGTGCGCGCGCGCGACCCGGTCGCTGTTTGAACCGG | |
| | GCGGAGGCGGGGCTGGCGCCCGGTTGGGAGGGGGTTGGG | |
| | GCCTGGCTTCCTGCCGCGCGCCGCGGGGACGCCTCCGACC | |
| | AGTGTTTGCCTTTTATGGTAATAACGCGGCCGGCCCGGCT | |
| | TCCTTTGTCCCCAATCTGGGCGCGCGCCGGCGCCCCCTGG | |
| | CGGCCTAAGGACTCGGCGCGCCGGAAGTGGCCAGGGCGG | |
| | CAGCGGCTGCTCTTGGCGGCCCCGAGGTGACTATAGCCTT | |
| | CTTTTGTGTCTTGATAGTTCGCCAGCCTCTGCTAACCATGT | |
| | TCATGCCTTCTTCTTTTTCCTACAGCTCCTGGGCAACGTGC | |
| | TGGTTATTGTGCTGTCTCATCATTTTGGCAAAGAATTC | |
| Chicken beta-actin | GTCGCTGCGCGCTGCCTTCGCCCCGTGCCCCGCTCCGCCG | 52 |
| exon/intron + rabbit | CCGCCTCGCGCCGCCCGCCCCGGCTCTGACTGACCGCGTT | |
| globin intron | ACTCCCACAGGTGAGCGGGCGGGACGGCCCTTCTCCTCCG | |
| | GGCTGTAATTAGCGCTTGGTTTAATGACGGCTTGTTTCTTT | |
| | TCTGTGGCTGCGTGAAAGCCTTGAGGGGCTCCGGGAGGGC | |
| | CCTTTGTGCGGGGGGAGCGGCTCGGGGGGTGCGTGCGTGT | |
| | GTGTGTGCGTGGGGAGCGCCGCGTGCGGCTCCGCGCTGCC | |
| | CGGCGGCTGTGAGCGCTGCGGGCGCGGCGCGGGGCTTTGT | |
| | GCGCTCCGCAGTGTGCGCGAGGGGAGCGCGGCCGGGGGC | |
| | GGTGCCCCGCGGTGCGGGGGGGGCTGCGAGGGGAACAAA | |
| | GGCTGCGTGCGGGGTGTGTGCGTGGGGGGGTGAGCAGGG | |
| | GGTGTGGGCGCGTCGGTCGGGCTGCAACCCCCCCTGCACC | |
| | CCCCTCCCCGAGTTGCTGAGCACGGCCCGGCTTCGGGTGC | |
| | GGGGCTCCGTACGGGGCGTGGCGCGGGGCTCGCCGTGCC | |
| | GGGCGGGGGGTGGCGGCAGGTGGGGGTGCCGGGCGGGGC | |
| | GGGGCCGCCTCGGGCCGGGGAGGGCTCGGGGGAGGGGCG | |
| | CGGCGGCCCCCGGAGCGCCGGCGGCTGTCGAGGCGCGGC | |
| | GAGCCGCAGCCATTGCCTTTTATGGTAATCGTGCGAGAGG | |
| | GCGCAGGGACTTCCTTTGTCCCAAATCTGTGCGGAGCCGA | |
| | AATCTGGGAGGCGCCGCCGCACCCCCTCTAGCGGGCGCGG | |
| | GGCGAAGCGGTGCGGCGCCGGCAGGAAGGAAATGGGCGG | |
| | GGAGGGCCTTCGTGCGTCGCCGCGCCGCCGTCCCCTTCTC | |
| | CCTCTCCAGCCTCGGGGCTGTCCGCGGGGGGACGGCTGCC | |
| | TTCGGGGGGGACGGGGCAGGGCGGGGTTCGGCTTCTGGC | |
| | GTGTGACCGGCGGCTCTAGAGCCTCTGCTAACCATGTTCA | |
| | TGCCTTCTTCTTTTTCCTACAGCTCCTGGGCAACGTGCTGG | |
| | TTATTGTGCTGTCTCATCATTTTGGCAAAGAATTC | |
| SV40 intron | GGTAAGTTTAGTCTTTTTGTCTTTTATTTCAGGTCCCGGAT | 53 |
| (Chimeric intron | CCGGTGGTGGTGCAAATCAAAGAACTGCTCCTCAGTGGAT | |
| sequence) | GTTGCCTTTACTTCTAGGCCTGTACGGAAGTGTTACTTCTG | |
| | CTCTAAAAGCTGCGGAATTGTACCCGC | |
| 5′ UTR-Syn1 Hs | AGTCTGCGGTGGGCAGCGGAGGAGTCGTGTCGTGCCTGAG | 54 |
| | AGCGCAGCTGTGCTCCTGGGCACCGCGCAGTCCGCCCCCG | |
| | CGGCTCCTGGCCAGACCACCCCTAGGACCCCCTGCCCCAA | |
| | GTCGCA | |
| CMV IE exon | TCAGATCGCCTGGAGAGGCCATCCACGCTGTTTTGACCTC | 55 |
| | CATAGTGGACACCGGGACCGATCCAGCCTCCGCGGCCGG | |
| | GAACGGTGCATTGGAACGCGGATTCCCCGTGCCAAGAGTG | |
| | AC | |
| TPL-eJPH2 | CTCACTCTCTTCCGCATCGCTGTCTGCGAGGGCCAGCTGTT | 56 |
| (adenovirus derived | GGGCTCGCGGTTGAGGACAAACTCTTCGCGGTCTTTCCAG | |
| enhancer element) | TACTCTTGGATCGGAAACCCGTCGGCCTCCGAACGGTACT | |
| | CCGCCACCGAGGGACCTGAGCGAGTCCGCATCGACCGGA | |
| | TCGGAAAACCTCTCGAGAAAGGCGTCTAACCAGTCACAGT | |
| | CGCAAGGTAGGCTGAGCACCGTGGCGGGCGGCAGCGGGT | |
| | GGCGGTCGGGGTTGTTTCTGGCGGAGGTGCTGCTGATGAT | |
| | GTAATTAAAGTAGGCGGTCTTGAGACGGCGGATGGTCGA | |
| | GGTGAGGTGTGGCAGGCTTGAGATCCAGCTGTTGGGGTGA | |
| | GTACTCCCTCTCAAAAGCGGGCATTACTTCTGCGCTAAGA | |
| | TTGTCAGTTTCCAAAAACGAGGAGGATTTGATATTCACCT | |
| | GGCCCGATCTGGCCATACACTTGAGTGACAATGACATCCA | |
| | CTTTGCCTTTCTCTCCACAGGTGTCCACTCCCAG | |
| Human EF1-α | CTTTTTCGCAACGGGTTTGCCGCCAGAACACAGGTAAGTG | 57 |
| intron/exon | CCGTGTGTGGTTCCCGCGGGCCTGGCCTCTTTACGGGTTAT | |
| | GGCCCTTGCGTGCCTTGAATTACTTCCACCTGGCTCCAGTA | |
| | CGTGATTCTTGATCCCGAGCTGGAGCCAGGGGCGGGCCTT | |
| | GCGCTTTAGGAGCCCCTTCGCCTCGTGCTTGAGTTGAGGC | |
| | CTGGCCTGGGCGCTGGGGCCGCCGCGTGCGAATCTGGTGG | |
| | CACCTTCGCGCCTGTCTCGCTGCTTTCGATAAGTCTCTAGC | |
| | CATTTAAAATTTTTGATGACGTGCTGCGACGCTTTTTTTCT | |
| | GGCAAGATAGTCTTGTAAATGCGGGCCAGGATCTGCACAC | |
| | TGGTATTTCGGTTTTTGGGCCCGCGGCCGGCGACGGGGCC | |
| | CGTGCGTCCCAGCGCACATGTTCGGCGAGGCGGGGCCTGC | |
| | GAGCGCGGCCACCGAGAATCGGACGGGGGTAGTCTCAAG | |
| | CTGGCCGGCCTGCTCTGGTGCCTGGCCTCGCGCCGCCGTG | |
| | TATCGCCCCGCCCTGGGCGGCAAGGCTGGCCCGGTCGGCA | |
| | CCAGTTGCGTGAGCGGAAAGATGGCCGCTTCCCGGCCCTG | |
| | CTCCAGGGGGCTCAAAATGGAGGACGCGGCGCTCGGGAG | |
| | AGCGGGCGGGTGAGTCACCCACACAAAGGAAAAGGGCCT | |
| | TTCCGTCCTCAGCCGTCGCTTCATGTGACTCCACGGAGTAC | |
| | CGGGCGCCGTCCAGGCACCTCGATTAGTTCTGGAGCTTTT | |
| | GGAGTACGTCGTCTTTAGGTTGGGGGGAGGGGTTTTATGC | |
| | GATGGAGTTTCCCCACACTGAGTGGGTGGAGACTGAAGTT | |
| | AGGCCAGCTTGGCACTTGATGTAATTCTCCTTGGAATTTG | |
| | GCCTTTTTGAGTTTGGATCTTGGTTCATTCTCAAGCCTCAG | |
| | ACAGTGGTTCAAAGTTTTTTTCTTCCATTTCAG | |
| Human EF1-α, intron | GTAAGTGCCGTGTGTGGTTCCCGCGGGCCTGGCCTCTTTA | 58 |
| A | CGGGTTATGGCCCTTGCGTGCCTTGAATTACTTCCACCTGG | |
| | CTGCAGTACGTGATTCTTGATCCCGAGCTTCGGGTTGGAA | |
| | GTGGGTGGGAGAGTTCGAGGCCTTGCGCTTAAGGAGCCCC | |
| | TTCGCCTCGTGCTTGAGTTGAGGCCTGGCCTGGGCGCTGG | |
| | GGCCGCCGCGTGCGAATCTGGTGGCACCTTCGCGCCTGTC | |
| | TCGCTGCTTTCGATAAGTCTCTAGCCATTTAAAATTTTTGA | |
| | TGACCTGCTGCGACGCTTTTTTTCTGGCAAGATAGTCTTGT | |
| | AAATGCGGGCCAAGATCTGCACACTGGTATTTCGGTTTTT | |
| | GGGGCCGCGGGCGGCGACGGGGCCCGTGCGTCCCAGCGC | |
| | ACATGTTCGGCGAGGCGGGGCCTGCGAGCGCGGCCACCG | |
| | AGAATCGGACGGGGGTAGTCTCAAGCTGGCCGGCCTGCTC | |
| | TGGTGCCTGGCCTCGCGCCGCCGTGTATCGCCCCGCCCTG | |
| | GGCGGCAAGGCTGGCCCGGTCGGCACCAGTTGCGTGAGC | |
| | GGAAAGATGGCCGCTTCCCGGCCCTGCTGCAGGGAGCTCA | |
| | AAATGGAGGACGCGGCGCTCGGGAGAGCGGGCGGGTGAG | |
| | TCACCCACACAAAGGAAAAGGGCCTTTCCGTCCTCAGCCG | |
| | TCGCTTCATGTGACTCCACGGAGTACCGGGCGCCGTCCAG | |
| | GCACCTCGATTAGTTCTCGAGCTTTTGGAGTACGTCGTCTT | |
| | TAGGTTGGGGGGAGGGGTTTTATGCGATGGAGTTTCCCCA | |
| | CACTGAGTGGGTGGAGACTGAAGTTAGGCCAGCTTGGCAC | |
| | TTGATGTAATTCTCCTTGGAATTTGCCCTTTTTGAGTTTGG | |
| | ATCTTGGTTCATTCTCAAGCCTCAGACAGTGGTTCAAAGTT | |
| | TTTTTCTTCCATTTCAG | |
| 5′ UTR human | TCAGAAGCCCCGGGCTCGTCAGTCAAACCGGTTCTCTGTT | 59 |
| CamKIIa | TGCACTCGGCAGCACGGGCAGGCAAGTGGTCCCTAGGTTC | |
| | GGG | |
| B-globin intron | GTGAGTCTATGGGACCCTTGATGTTTTCTTTCCCCTTCTTTT | 60 |
| | CTATGGTTAAGTTCATGTCATAGGAAGGGGAGAAGTAACA | |
| | GGGTACACATATTGACCAAATCAGGGTAATTTTGCATTTG | |
| | TAATTTTAAAAAATGCTTTCTTCTTTTAATATACTTTTTTGT | |
| | TTATCTTATTTCTAATACTTTCCCTAATCTCTTTCTTTCAGG | |
| | GCAATAATGATACAATGTATCATGCCTCTTTGCACCATTCT | |
| | AAAGAATAACAGTGATAATTTCTGGGTTAAGGCAATAGCA | |
| | ATATTTCTGCATATAAATATTTCTGCATATAAATTGTAACT | |
| | GATGTAAGAGGTTTCATATTGCTAATAGCAGCTACAATCC | |
| | AGCTACCATTCTGCTTTTATTTTATGGTTGGGATAAGGCTG | |
| | GATTATTCTGAGTCCAAGCTAGGCCCTTTTGCTAATCATGT | |
| | TCATACCTCTTATCTTCCTCCCACAG | |
| SV40 intron (long | TCTAGAGGATCCGGTACTCGAGGAACTGAAAAACCAGAA | 61 |
| form; underlined 5′ | AGTTAACTGGTAAGTTTAGTCTTTTTGTCTTTTATTTCAGG | |
| and 3′ extensions) | TCCCGGATCCGGTGGTGGTGCAAATCAAAGAACTGCTCCT | |
| | CAGTGGATGTTGCCTTTACTTCTAGGCCTGTACGGAAGTG | |
| | TTACTTCTGCTCTAAAAGCTGCGGAATTGTACCCGC | |

In some embodiments, the vector comprises a 3′ untranslated region selected from Table 5. In some embodiments, the vector genome comprises a polynucleotide sequence at least 75%, 80%, 85%, 90%, 92%, 93%, 94%, 95%, 96%, 97%, 98%, 99%, or 100% identical to any one of SEQ ID NOS 62-70.

TABLE 5

| | | SEQ |
|---|---|---|
| 3′ UNTRANSLATED | | ID |
| REGION | SEQUENCE | NO: |
| WPRE(x) (mutated | AATCAACCTCTGGATTACAAAATTTGTGAAAGATTGACT | 62 |
| woodchuck hepatitis | GGTATTCTTAACTATGTTGCTCCTTTTACGCTATGTGGAT | |
| regulatory element- | ACGCTGCTTTAATGCCTTTGTATCATGCTATTGCTTCCCGT | |
| version 1) | ATGGCTTTCATTTTCTCCTCCTTGTATAAATCCTGGTTGCT | |
| | GTCTCTTTATGAGGAGTTGTGGCCCGTTGTCAGGCAACGT | |
| | GGCGTGGTGTGCACTGTGTTTGCTGACGCAACCCCCACTG | |
| | GTTGGGGCATTGCCACCACCTGTCAGCTCCTTTCCGGGAC | |
| | TTTCGCTTTCCCCCTCCCTATTGCCACGGCGGAACTCATC | |
| | GCCGCCTGCCTTGCCCGCTGCTGGACAGGGGCTCGGCTGT | |
| | TGGGCACTGACAATTCCGTGGTGTTGTCGGGGAAATCAT | |
| | CGTCCTTTCCTTGGCTGCTCGCCTGTGTTGCCACCTGGATT | |
| | CTGCGCGGGACGTCCTTCTGCTACGTCCCTTCGGCCCTCA | |
| | ATCCAGCGGACCTTCCTTCCCGCGGCCTGCTGCCGGCTCT | |
| | GCGGCCTCTTCCGCGTCTTCGCCTTCGCCCTCAGACGAGT | |
| | CGGATCTCCCTTTGGGCCGCCTCCCCGC | |
| WPRE(x) (mutated | TCAACCTCTGGATTACAAAATTTGTGAAAGATTGACTGGT | 63 |
| woodchuck hepatitis | ATTCTTAACTATGTTGCTCCTTTTACGCTATGTGGATACG | |
| regulatory element- | CTGCTTTAATGCCTTTGTATCATGCTATTGCTTCCCGTATG | |
| version 2) | GCTTTCATTTTCTCCTCCTTGTATAAATCCTGGTTGCTGTC | |
| | TCTTTATGAGGAGTTGTGGCCCGTTGTCAGGCAACGTGGC | |
| | GTGGTGTGCACTGTGTTTGCTGACGCAACCCCCACTGGTT | |
| | GGGGCATTGCCACCACCTGTCAGCTCCTTTCCGGGACTTT | |
| | CGCTTTCCCCCTCCCTATTGCCACGGCGGAACTCATCGCC | |
| | GCCTGCCTTGCCCGCTGCTGGACAGGGGCTCGGCTGTTGG | |
| | GCACTGACAATTCCGTGGTGTTGTCGGGGAAATCATCGTC | |
| | CTTTCCTTGGCTGCTCGCCTGTGTTGCCACCTGGATTCTGC | |
| | GCGGGACGTCCTTCTGCTACGTCCCTTCGGCCCTCAATCC | |
| | AGCGGACCTTCCTTCCCGCGGCCTGCTGCCGGCTCTGCGG | |
| | CCTCTTCCGCGTCTTCGCCTTCGCCCTCAGACGAGTCGGA | |
| | TCTCCCTTTGGGCCGCCTCCCCGCA | |
| WPRE(x) (mutated | TTCCTGTTAATCAACCTCTGGATTACAAAATTTGTGAAAG | 64 |
| woodchuck hepatitis | ATTGACTGGTATTCTTAACTATGTTGCTCCTTTTACGCTAT | |
| regulatory element- | GTGGATACGCTGCTTTAATGCCTTTGTATCATGCTATTGC | |
| version 3) | TTCCCGTATGGCTTTCATTTTCTCCTCCTTGTATAAATCCT | |
| | GGTTGCTGTCTCTTTATGAGGAGTTGTGGCCCGTTGTCAG | |
| | GCAACGTGGCGTGGTGTGCACTGTGTTTGCTGACGCAAC | |
| | CCCCACTGGTTGGGGCATTGCCACCACCTGTCAGCTCCTT | |
| | TCCGGGACTTTCGCTTTCCCCCTCCCTATTGCCACGGCGG | |
| | AACTCATCGCCGCCTGCCTTGCCCGCTGCTGGACAGGGG | |
| | CTCGGCTGTTGGGCACTGACAATTCCGTGGTGTTGTCGGG | |
| | GAAGCTGACGTCCTTTCCGCGGCTGCTCGCCTGTGTTGCC | |
| | ACCTGGATTCTGCGCGGGACGTCCTTCTGCTACGTCCCTT | |
| | CGGCCCTCAATCCAGCGGACCTTCCTTCCCGCGGCCTGCT | |
| | GCCGGCTCTGCGGCCTCTTCCGCCTCTTCGCCTTCGCCCT | |
| | CAGACGAGTCGGATCTCCCTTTGGGCCGCCTCCCCGCCCA | |
| | TGTATCTTTTTCACCTGTGCCTTGTTTTTGCCTGTGTTCCG | |
| | CGTCCTACTTTTCAAGCCTCCAAGCTGTGCCTTGGGCGGC | |
| | TTTGGGGCATGGACATAGATCCCTATAAAGAATTTGGTTC | |
| | ATCTTATCAGTTGTTGAATTTTCTTCCTTTGGAC | |
| CAAX | TGTGTGATAATG | 65 |
| EES | CTGTTCTCATCACATCATATCAAGGTTATATACCATCAAT | 66 |
| HPRE | ATTGCCACAGATGTTACTTAGCCTTTTAATATTTCTCTAAT | 67 |
| | TTAGTGTATATGCAATGATAGTTCTCTGATTTCTGAGATT | |
| | GAGTTTCTCATGTGTAATGATTATTTAGAGTTTCTCTTTCA | |
| | TCTGTTCAAATTTTTGTCTAGTTTTATTTTTTACTGATTTG | |
| | TAAGACTTCTTTTTATAATCTGCATATTACAATTCTCTTTA | |
| | CTGGGGTGTTGCAAATATTTTCTGTCATTCTATGGCCTGA | |
| | CTTTTCTTAATGGTTTTTTAATTTTAAAAATAAGTCTTAAT | |
| | ATTCATGCAATCTAATTAACAATCTTTTCTTTGTGGTTAG | |
| | GACTTTGAGTCATAAGAAATTTTTCTCTACACTGAAGTCA | |
| | TGATGGCATGCTTCTATATTATTTTCTAAAAGATTTAAAG | |
| | TTTTGCCTTCTCCATTTAGACTTATAATTCACTGGAATTTT | |
| | TTTGTGTGTATGGTATGACATATGGGTTCCCTTTTATTTTT | |
| | TACATATAAATATATTTCCCTGTTTTTCTAAAAAAGAAAA | |
| | AGATCATCATTTTCCCATTGTAAAATGCCATATTTTTTTCA | |
| | TAGGTCACTTACATATATCAATGGGTCTGTTTCTGAGCTC | |
| | TACTCTATTTTATCAGCCTCACTGTCTATCCCCACACATCT | |
| | CATGCTTTGCTCTAAATCTTGATATTTAGTGGAACATTCT | |
| | TTCCCATTTTGTTCTACAAGAATATTTTTGTTATTGTCTTT | |
| | GGGCTTTCTATATACATTTTGAAATGAGGTTGACAAGTTA | |
| | ATAACAGGCCTATTGATTGGAAAGTTTGTCAACGAATTGT | |
| | GGGTCTTTTGGGGTTTGCTGCCCCTTTTACGCAATGTGGA | |
| | TATCCTGCTTTAATGCCTTTATATGCATGTATACAAGCAA | |
| | AACAGGCTTTTACTTTCTCGCCAACTTACAAGGCCTTTCT | |
| | CAGTAAACAGTATATGACCCTTTACCCCGTTGCTCGGCAA | |
| | CGGCCTGGTCTGTGCCAAGTGTTTGCTGACGCAACCCCCA | |
| | CTGGTTGGGGCTTGGCCATAGGCCATCAGCGCATGCGTG | |
| | GAACCTTTGTGTCTCCTCTGCCGATCCATACTGCGGAACT | |
| | CCTAGCCGCTTGTTTTGCTCGCAGCAGGTCTGGAGCAAAC | |
| | CTCATCGGGACCGACAATTCTGTCGTACTCTCCCGCAAGT | |
| | ATACATCGTTTCCATGGCTGCTAGGCTGTGCTGCCAACTG | |
| | GATCCTGCGCGGGACGTCCTTTGTTTACGTCCCGTCGGCG | |
| | CTGAATCCCGCGGACGACCCCTCCCGGGGCCGCTTGGGG | |
| | CTCTACCGCCCGCTTCTCCGTCTGCCGTACCGTCCGACCA | |
| | CGGGGCGCACCTCTCTTTACGCGGACTCCCCGTCTGTGCC | |
| | TTCTCATCTGCCGGACCGTGTGCACTTCGCTTCACCTCTG | |
| | CACGTCGCATGGAGGCCACCGTGAACGCCCACCGGAACC | |
| | TGCCCAAGGTCTTGCATAAGAGGACTCTTGGACTTTCAGC | |
| | AATGTCATC | |
| R2V17 (HepB derived | TTCCTGTAAACAGGCCTATTGATTGGAAAGTTTGTCAACG | 68 |
| enhancer element) | AATTGTGGGTCTTTTGGGGTTTGCTGCCCCTTTTACGCAA | |
| | TGTGGATATCCTGCTTTAATGCCTTTATATGCATGTATAC | |
| | AAGCAAAACAGGCTTTTACTTTCTCGCCAACTTACAAGGC | |
| | CTTTCTCAGTAAACAGTATATGACCCTTTACCCCGTTGCT | |
| | CGGCAACGGCCTGGTCTGTGCCAAGTGTTTGCTGACGCA | |
| | ACCCCCACTGGTTGGGGCTTGGCCATAGGCCATCAGCGC | |
| | ATGCGTGGAACCTTTGTGTCTCCTCTGCCGATCCATACTG | |
| | CGGAACTCCTAGCCGCTTGTTTTGCTCGCAGCTGGACTGG | |
| | AGCAAACCTCATCGGGACCGACAATTCTGTCGTACTCTCC | |
| | CGCAAGCACTCACCGTTTCCGCGGCTGCTCGCCTGTGTTG | |
| | CCACCTGGATTCTGCGCGGGACGTCCTTCTGCTACGTCCC | |
| | TTCGGCCCTCAATCCAGCGGACCTTCCTTCCCGCGGCCTG | |
| | CTGCCGGCTCTGCGGCCTCTTCCGCCTCTTCGCCTTCGCC | |
| | CTCAGACGAGTCGGATCTCCCTTTGGGCCGCCTCCCCGCC | |
| | CATGTATCTTTTTCACCTGTGCCTTGTTTTTGCCTGTGTTC | |
| | CGCGTCCTACTTTTCAAGCCTCCAAGCTGTGCCTTGGGCG | |
| | GCTTTGGGGCATGGACATAGATCCCTATAAAGAATTTGG | |
| | TTCATCTTATCAGTTGTTGAATTTTCTTCCTTTGGAC | |
| 3”UTR(globin) | GCTGGAGCCTCGGTAGCCGTTCCTCCTGCCCGCTGGGCCT | 69 |
| | CCCAACGGGCCCTCCTCCCCTCCTTGCACCGGCCCTTCCT | |
| | GGTCTTTGAATAAA | |
| WPRE(r) | ATTCGAGCATCTTACCGCCATTTATTCCCATATTTGTTCTG | 70 |
| | TTTTTCTTGATTTGGGTATACATTTAAATGTTAATAAAAC | |
| | AAAATGGTGGGGCAATCATTTACATTTTTAGGGATATGTA | |
| | ATTACTAGTTCAGGTGTATTGCCACAAGACAAACATGTTA | |
| | AGAAACTTTCCCGTTATTTACGCTCTGTTCCTGTTAATCA | |
| | ACCTCTGGATTACAAAATTTGTGAAAGATTGACTGATATT | |
| | CTTAACTATGTTGCTCCTTTTACGCTGTGTGGATATGCTG | |
| | CTTTAATGCCTCTGTATCATGCTATTGCTTCCCGTACGGCT | |
| | TTCGTTTTCTCCTCCTTGTATAAATCCTGGTTGCTGTCTCT | |
| | TTATGAGGAGTTGTGGCCCGTTGTCCGTCAACGTGGCGTG | |
| | GTGTGCTCTGTGTTTGCTGACGCAACCCCCACTGGCTGGG | |
| | GCATTGCCACCACCTGTCAACTCCTTTCTGGGACTTTCGC | |
| | TTTCCCCCTCCCGATCGCCACGGCAGAACTCATCGCCGCC | |
| | TGCCTTGCCCGCTGCTGGACAGGGGCTAGGTTGCTGGGC | |
| | ACTGATAATTCCGTGGTGTTGTCGGGGAAGGGCC | |

In some embodiments, the vector comprises a polyadenylation (polyA) signal selected from Table 6. In some embodiments, the polyA signal comprises a polynucleotide sequence at least 75%, 80%, 85%, 90%, 92%, 93%, 94%, 95%, 96%, 97%, 98%, 99%, or 100% identical to any one of SEQ ID NOS 71-75.

TABLE 6

| POLY- | | SEQ |
|---|---|---|
| ADENYLATION | | ID |
| SITE | SEQUENCE | NO: |
| Rabbit globin | TGGCTAATAAAGGAAATTTATTTTCATTGCAATAGTGTG | 71 |
| (pAGlobin-Oc) | TTGGAATTTTTTGTGTCTCTCACTCGGAAGAACATATGG | |
| | GAGGGCAAATCATTTAAAACATCAGAATGAGTATTTGGT | |
| | TTAGAGTTTGGCAACATATGCCCATATGCTGGCTGCCAT | |
| | GAACAAAGGTTGGCTATAAAGAGGTCATCAGTATATGA | |
| | AACAGCCCCCTGCTGTCCATTCCTTATTCCATAGAAAAG | |
| | CCTTGACTTGAGGTTAGATTTTTTTTATATTTTGTTTTGTG | |
| | TTATTTTTTTCTTTAACATCCCTAAAATTTTCCTTACATGT | |
| | TTTACTAGCCAGATTTTTCCTCCTCTCCTGACTACTCCCA | |
| | GTCATAGCTGTCCCTCTTCTCTTATGGAGATC | |
| Bovine growth | TTGCCAGCCATCTGTTGTTTGCCCCTCCCCCGTGCCTTCC | 72 |
| hormone (pAGH-Bt- | TTGACCCTGGAAGGTGCCACTCCCACTGTCCTTTCCTAA | |
| version 1) | TAAAATGAGGAAATTGCATCGCATTGTCTGAGTAGGTGT | |
| | CATTCTATTCTGGGGGGTGGGGTGGGGCAGGACAGCAA | |
| | GGGGGAGGATTGGGAATACAATAGCAGGCATGCTGGGG | |
| | ATGCGGTGGGCTCTATGGGTACCCAGGTGCTGAAGAATT | |
| | GACCCGGTTCCTCCTGGG | |
| Bovine growth | TTGCCAGCCATCTGTTGTTTGCCCCTCCCCCGTGCCTTCC | 73 |
| hormone (pAGH-Bt- | TTGACCCTGGAAGGTGCCACTCCCACTGTCCTTTCCTAA | |
| version 2) | TAAAATGAGGAAATTGCATCGCATTGTCTGAGTAGGTGT | |
| | CATTCTATTCTGGGGGGTGGGGTGGGGCAGGACAGCAA | |
| | GGGGGAGGATTGGGAAGACAATAGCAGGCATGCTGGGG | |
| | ATGCGGTGGGCTCTATGGGTACCCAGGTGCTGAAGAATT | |
| | GACCCGGTTCCTCCTGGG | |
| Bovine growth | CTGTGCCTTCTAGTTGCCAGCCATCTGTTGTTTGCCCCTC | 74 |
| hormone (pAGH-Bt- | CCCCGTGCCTTCCTTGACCCTGGAAGGTGCCACTCCCAC | |
| version 3) | TGTCCTTTCCTAATAAAATGAGGAAATTGCATCGCATTG | |
| | TCTGAGTAGGTGTCATTCTATTCTGGGGGGTGGGGTGGG | |
| | GCAGGACAGCAAGGGGGAGGATTGGGAAGACAATAGC | |
| | AGGCATGCTGGGGATGCGGTGGGCTCTATGG | |
| Human growth | CTGCCCGGGTGGCATCCCTGTGACCCCTCCCCAGTGCCT | 75 |
| hormone (pAGH-Hs) | CTCCTGGCCCTGGAAGTTGCCACTCCAGTGCCCACCAGC | |
| | CTTGTCCTAATAAAATTAAGTTGCATCATTTTGTCTGACT | |
| | AGGTGTCCTTCTATAATATTATGGGGTGGAGGGGGGTGG | |
| | TATGGAGCAAGGGGCCCAAGTTGGGAAGAAACCTGTAG | |
| | GGCCTGC | |

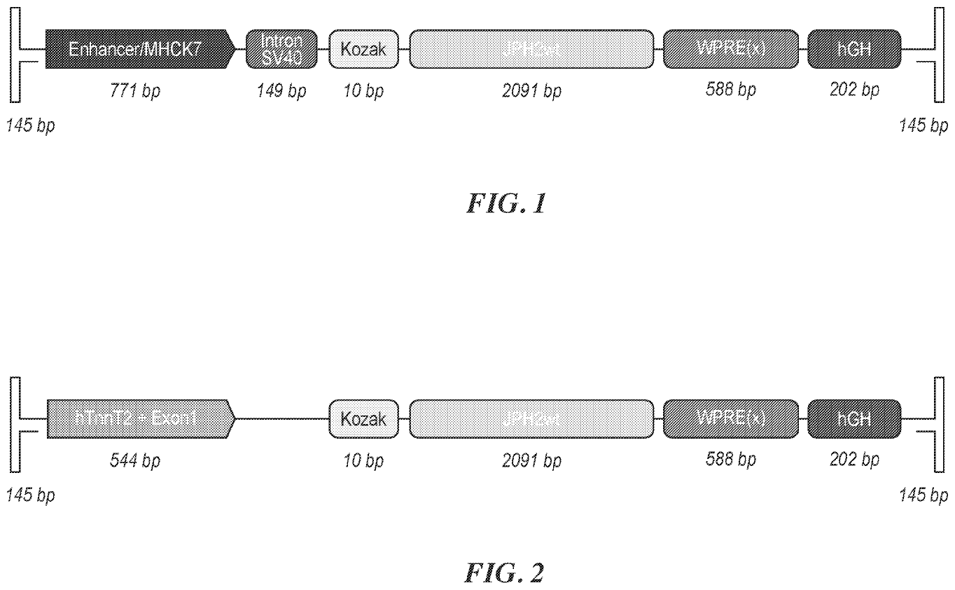

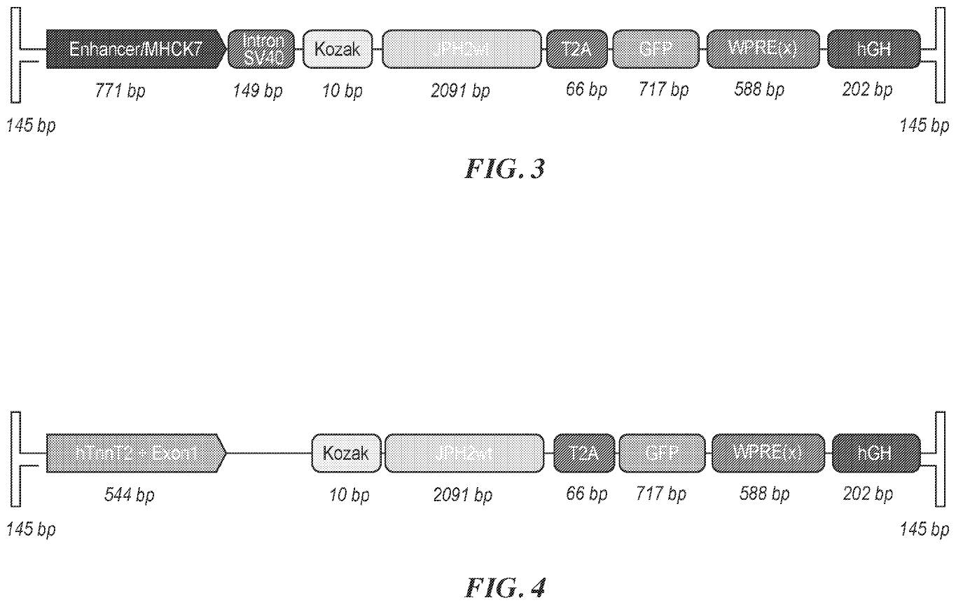

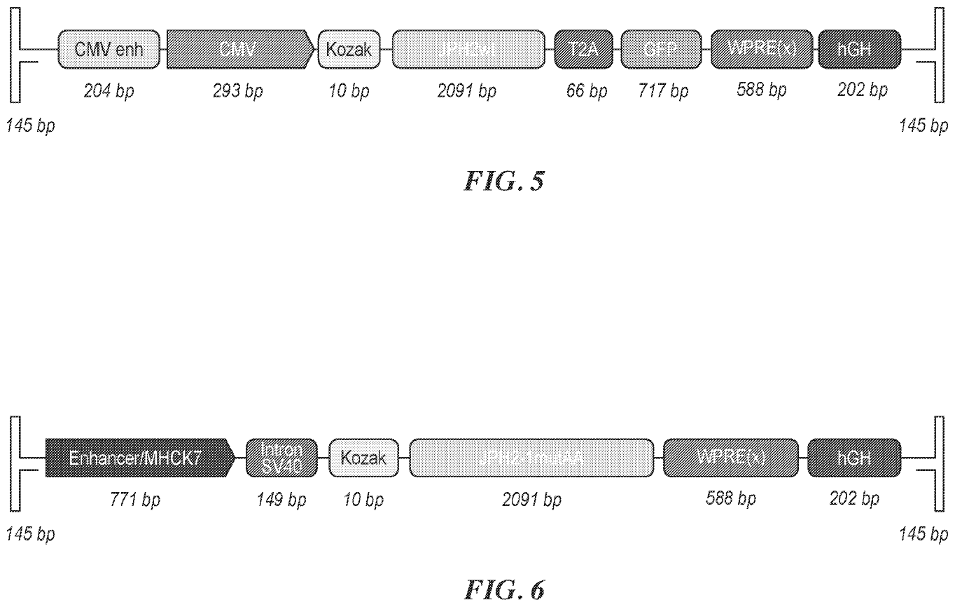

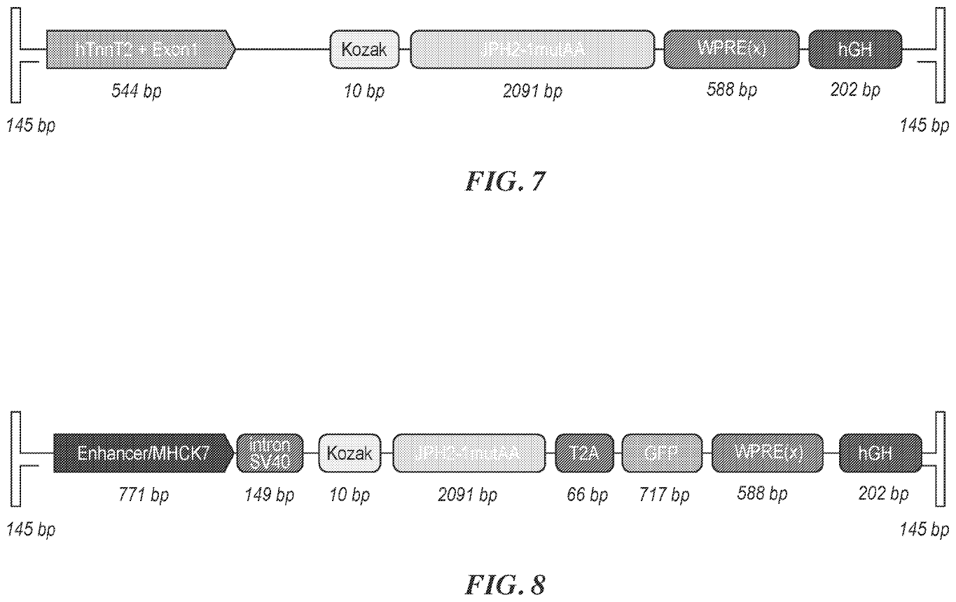

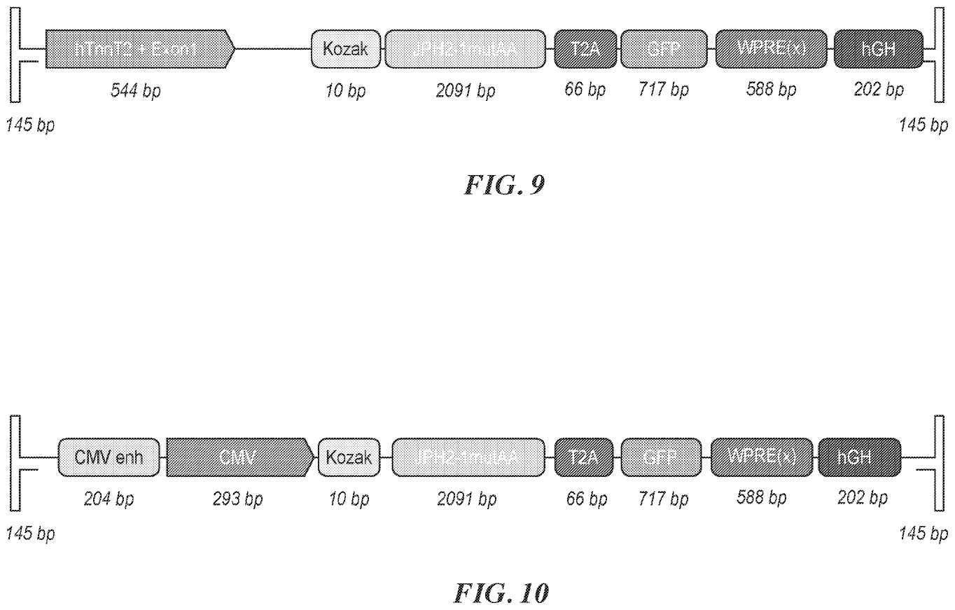

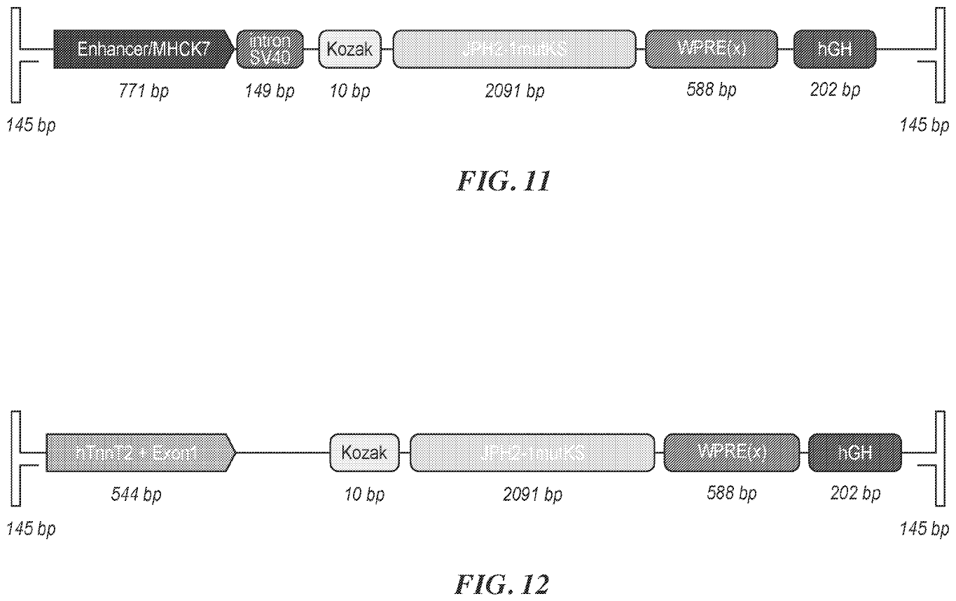

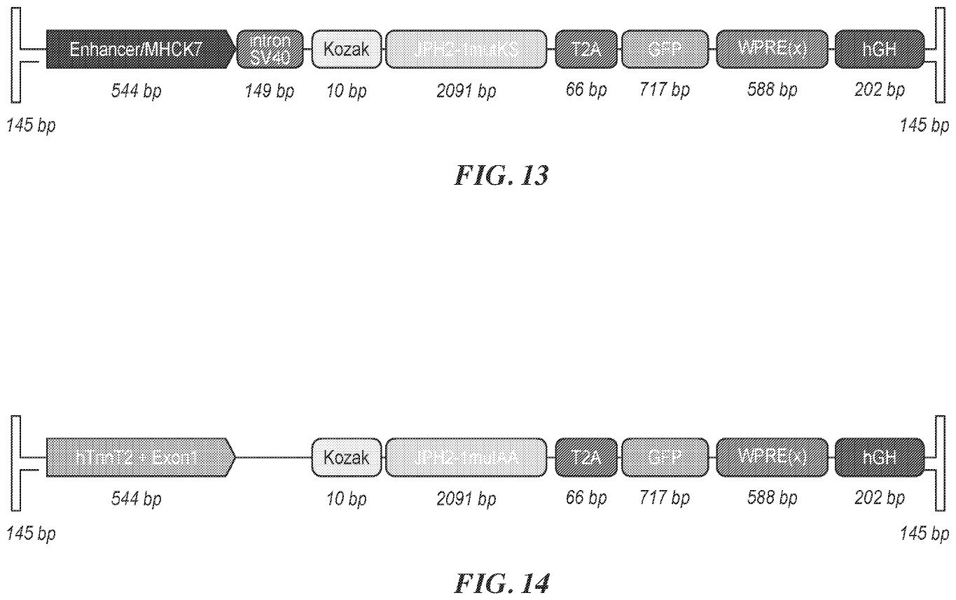

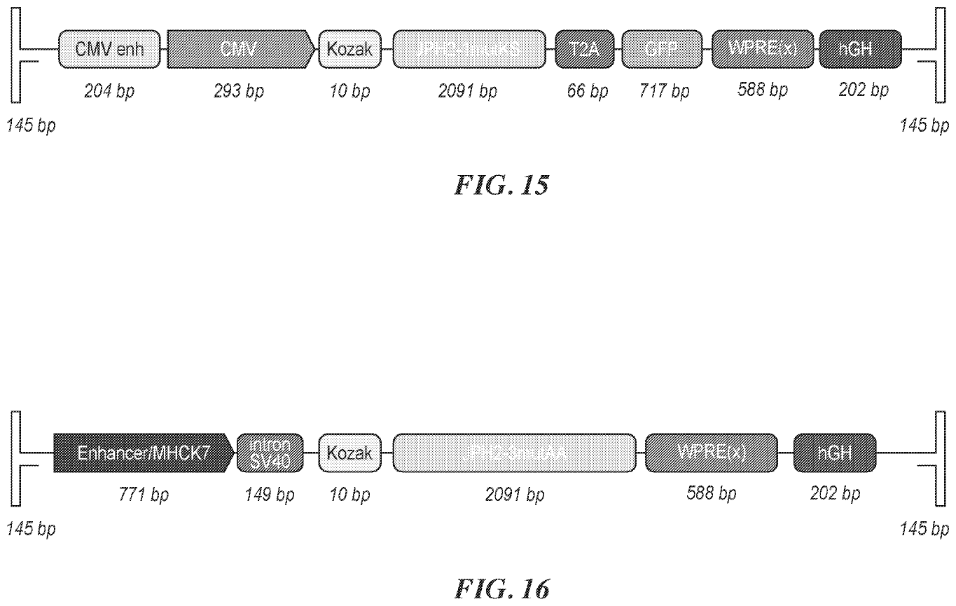

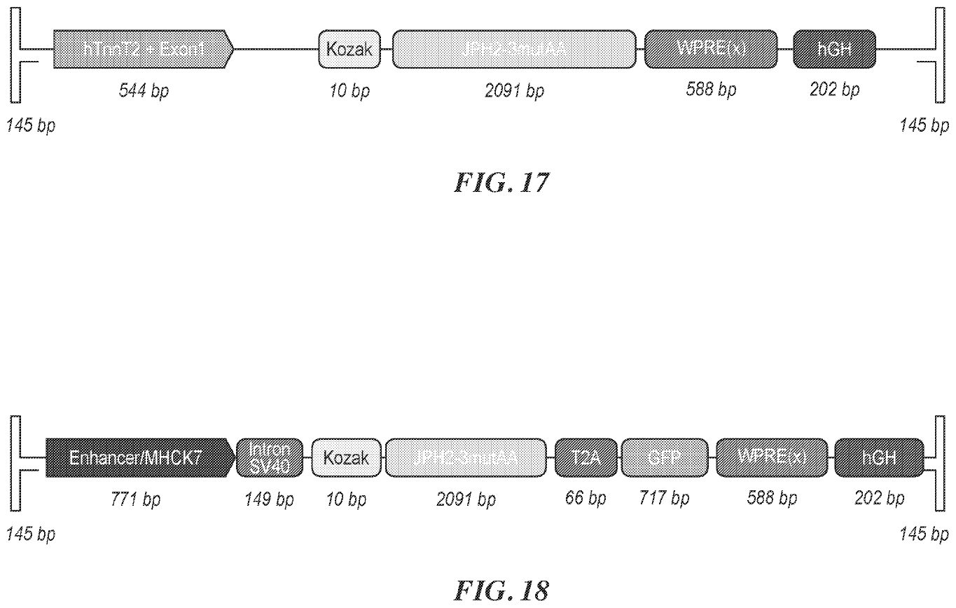

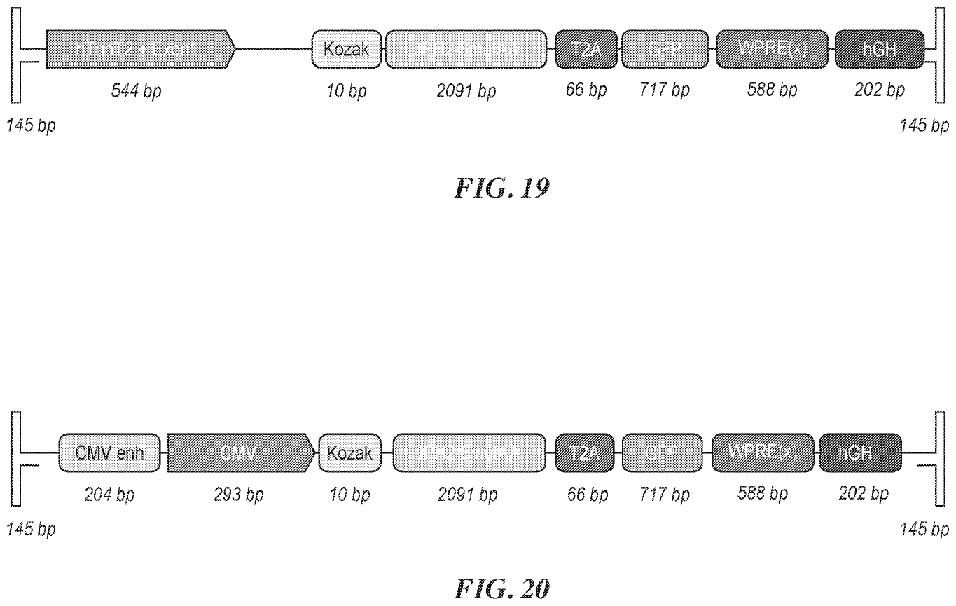

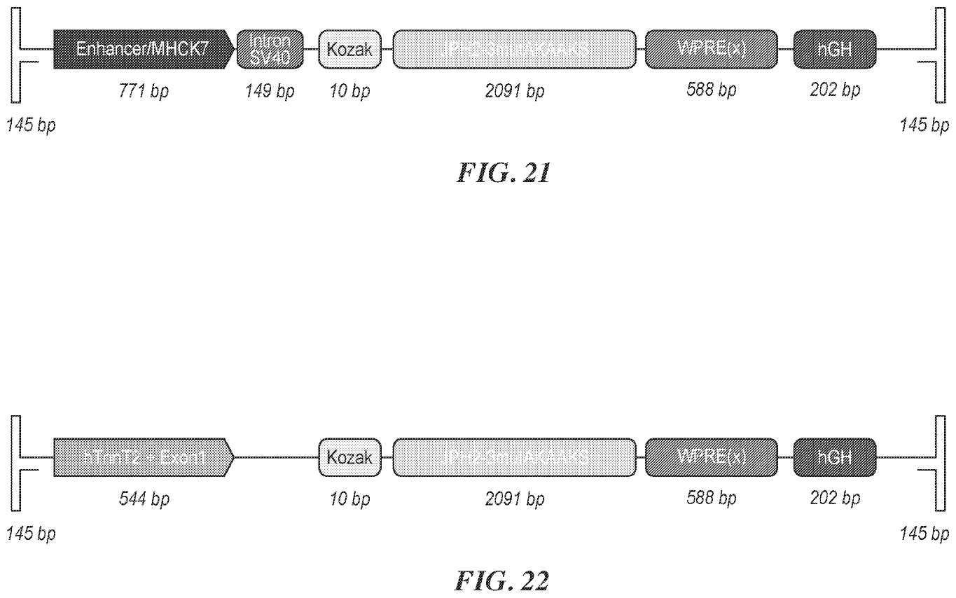

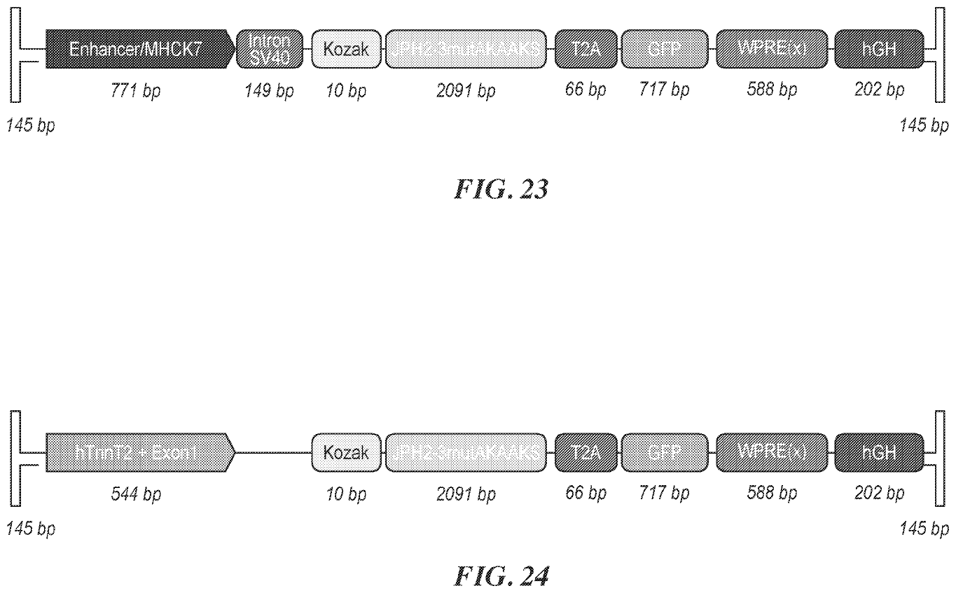

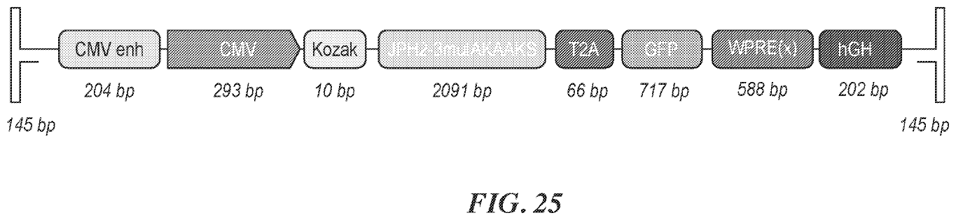

Illustrative vector genomes are depicted in FIGS. 1-25; and provided as SEQ ID NOs: 26-30 and 76-95. In some embodiments, the vector genome comprises, consists essentially of, or consists of a polynucleotide sequence that shares at least 90%, 91%, 92%, 93%, 94%, 95%, 96%, 97%, 98%, 99%, or 100% identity to any one of SEQ ID NOs: 26-30 and 76-95, optionally with or without the ITR sequences. The disclosure also contemplates expression cassettes of the illustrative vector genomes depicted in FIGS. 1-25 and sequences comprising these, e.g., the sequences set forth in SEQ ID NOs: 26-30 and 76-95, but lacking the 5′ and 3′ ITRs, and variants thereof sharing 90%, 91%, 92%, 93%, 94%, 95%, 96%, 97%, 98%, 99%, or 100% identity to any of the foregoing.

In a certain embodiment, the vector genome comprises, in 5′ to 3′ order, a 5′ ITR; an MHCK7 promoter; a JPH2 transgene; an WPRE(x) element; a Human GH poly (A) signal (hGH) sequence; and a 3′ ITR. The vector genome may comprise, in 5′ to 3′ order, the polynucleotide sequences SEQ ID NO: 26; or polynucleotide sequences sharing 90%, 91%, 92%, 93%, 94%, 95%, 96%, 97%, 98%, 99%, or 100% identity to any of the foregoing. In certain embodiments, this vector genome is packaged in an AAV9 or AAVrh74 vector. The JPH2 transgene of this embodiment is a full length wild type transgene, i.e., a transgene encoding a JPH2 of at least 600 or at least 630 amino acids.

| SEQ ID NO: 26 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtac ccttcagatt aaaaataact | 180 |
| gaggtaaggg cctgggtagg ggaggtggtg tgagacgctc ctgtctctcc tctatctgcc | 240 |
| catcggccct ttggggagga ggaatgtgcc caaggactaa aaaaaggcca tggagccaga | 300 |
| ggggcgaggg caacagacct ttcatgggca aaccttgggg ccctgctgtc tagcatgccc | 360 |
| cactacgggt ctaggctgcc catgtaagga ggcaaggcct ggggacaccc gagatgcctg | 420 |
| gttataatta acccagacat gtggctgccc cccccccccc aacacctgct gcctctaaaa | 480 |
| ataaccctgt ccctggtgga tcccctgcat gcgaagatct tcgaacaagg ctgtggggga | 540 |
| ctgagggcag gctgtaacag gcttgggggc cagggcttat acgtgcctgg gactcccaaa | 600 |
| gtattactgt tccatgttcc cggcgaaggg ccagctgtcc cccgccagct agactcagca | 660 |
| cttagtttag gaaccagtga gcaagtcagc ccttggggca gcccatacaa ggccatgggg | 720 |
| ctgggcaagc tgcacgcctg ggtccggggt gggcacggtg cccgggcaac gagctgaaag | 780 |
| ctcatctgct ctcaggggcc cctccctggg gacagcccct cctggctagt cacaccctgt | 840 |
| aggctcctct atataaccca ggggcacagg ggctgccctc attctaccac cacctccaca | 900 |
| gcacagacag acactcagga gccagccagg gtaagtttag tctttttgtc ttttatttca | 960 |
| ggtcccggat ccggtggtgg tgcaaatcaa agaactgctc ctcagtggat gttgccttta | 1020 |
| cttctaggcc tgtacggaag tgttacttct gctctaaaag ctgcggaatt gtacccgcgc | 1080 |
| caccatgagt gggggccgct tcgactttga tgatggaggg gcgtactgcg ggggctggga | 1140 |
| ggggggaaag gcccatgggc atggactgtg cacaggcccc aagggccagg gcgaatactc | 1200 |
| tggctcctgg aactttggct ttgaggtggc aggtgtctac acctggccca gcggaaacac | 1260 |
| ctttgaggga tactggagcc agggcaaacg gcatgggctg ggcatagaga ccaaggggcg | 1320 |
| ctggctctac aagggcgagt ggacacatgg cttcaaggga cgctacggaa tccggcagag | 1380 |
| ctcaagcagc ggtgccaagt atgagggcac ctggaacaat ggcctgcaag acggctatgg | 1440 |
| caccgagacc tatgctgatg gagggacgta ccaaggccag ttcaccaacg gcatgcgcca | 1500 |
| tggctacgga gtacgccaga gcgtgcccta cgggatggcc gtggtggtgc gctcgccgct | 1560 |
| gcgcacgtcg ctgtcgtccc tgcgcagcga gcacagcaac ggcacggtgg ccccggactc | 1620 |
| tcccgcctcg ccggcctccg acggccccgc gctgccctcg cccgccatcc cgcgtggcgg | 1680 |
| cttcgcgctc agcctcctgg ccaatgccga ggcggccgcg cgggcgccca agggcggcgg | 1740 |
| cctcttccag cggggcgcgc tgctgggcaa gctgcggcgc gcagagtcgc gcacgtccgt | 1800 |
| gggtagccag cgcagccgtg tcagcttcct taagagcgac ctcagctcgg gcgccagcga | 1860 |
| cgccgcgtcc accgccagcc tgggagaggc cgccgagggc gccgacgagg ccgcaccctt | 1920 |
| cgaggccgat atcgacgcca ccaccaccga gacctacatg ggcgagtgga agaacgacaa | 1980 |
| acgctcgggc ttcggcgtga gcgaacgctc cagtggcctc cgctacgagg gcgagtggct | 2040 |
| ggacaacctg cgccacggct atggctgcac cacgctgccc gacggccacc gcgaggaggg | 2100 |
| caagtaccgc cacaacgtgc tggtcaagga caccaagcgc cgcatgctgc agctcaagag | 2160 |
| caacaaggtc cgccagaaag tggagcacag tgtggagggt gcccagcgcg ccgctgctat | 2220 |
| cgcgcgccag aaggccgaga ttgccgcctc caggacaagc cacgccaagg ccaaagctga | 2280 |
| ggcagcggaa caggccgccc tggctgccaa ccaggagtcc aacattgctc gcactttggc | 2340 |
| cagggagctg gctccggact tctaccagcc aggtccggaa tatcagaagc gccggctgct | 2400 |
| gcaggagatc ctggagaact cggagagcct gctggagccc cccgaccggg gcgccggcgc | 2460 |
| agcgggcctc ccacagccgc cccgcgagag cccgcagctg cacgagcgtg agacccctcg | 2520 |
| gcccgagggt ggctccccgt caccggccgg gacgcccccg cagcccaagc ggcccaggcc | 2580 |
| cggggtgtcc aaggacggcc tgctgagccc aggcgcctgg aacggcgagc ccagcggtga | 2640 |
| gggcagccgg tcagtcactc cgtccgaggg cgcgggccgc cgcagccccg cgcgtccagc | 2700 |
| caccgagcgc atggccatcg aggctctgca ggcaccgcct gcgccgtcgc gggagccgga | 2760 |
| ggtggcgctt taccagggct accacagcta tgctgtgcgc accacgccgc ccgagccccc | 2820 |
| accctttgag gaccagcccg agcccgaggt ctccgggtcc gagtccgcgc cctcgtcccc | 2880 |
| ggccaccgcc ccgctgcagg cccccacgct ccgaggcccc gagcctgcac gcgagacccc | 2940 |
| cgccaagctg gagcccaagc ccatcatccc caaagccgag cccagggcca aggcccgcaa | 3000 |
| gactgaggct cgagggctga ccaaggcggg ggccaagaag aaggcgcgga aggaggccgc | 3060 |
| actggcggca gaggcggagg tggaggtgga agaggtcccc aacaccatcc tcatctgcat | 3120 |
| ggtgatcctg ctgaacatcg gcctggccat cctctttgtt cacctcctga cctgatcaac | 3180 |
| ctctggatta caaaatttgt gaaagattga ctggtattct taactatgtt gctcctttta | 3240 |
| cgctatgtgg atacgctgct ttaatgcctt tgtatcatgc tattgcttcc cgtatggctt | 3300 |
| tcattttctc ctccttgtat aaatcctggt tgctgtctct ttatgaggag ttgtggcccg | 3360 |
| ttgtcaggca acgtggcgtg gtgtgcactg tgtttgctga cgcaaccccc actggttggg | 3420 |
| gcattgccac cacctgtcag ctcctttccg ggactttcgc tttccccctc cctattgcca | 3480 |
| cggcggaact catcgccgcc tgccttgccc gctgctggac aggggctcgg ctgttgggca | 3540 |
| ctgacaattc cgtggtgttg tcggggaaat catcgtcctt tccttggctg ctcgcctgtg | 3600 |
| ttgccacctg gattctgcgc gggacgtcct tctgctacgt cccttcggcc ctcaatccag | 3660 |
| cggaccttcc ttcccgcggc ctgctgccgg ctctgcggcc tcttccgcgt cttcgccttc | 3720 |
| gccctcagac gagtcggatc tccctttggg ccgcctcccc gcactgcccg ggtggcatcc | 3780 |
| ctgtgacccc tccccagtgc ctctcctggc cctggaagtt gccactccag tgcccaccag | 3840 |
| ccttgtccta ataaaattaa gttgcatcat tttgtctgac taggtgtcct tctataatat | 3900 |
| tatggggtgg aggggggtgg tatggagcaa ggggcccaag ttgggaagaa acctgtaggg | 3960 |
| cctgccctaa ggaggaaccc ctagtgatgg agttggccac tccctctctg cgcgctcgct | 4020 |
| cgctcactga ggccgggcga ccaaaggtcg cccgacgccc gggctttgcc cgggcggcct | 4080 |
| cagtgagcga gcgagcgcgc agagagggag tggccaa | 4117 |

In a certain embodiment, the vector genome comprises, in 5′ to 3′ order, a 5′ITR; a hTnnT2 promoter; a JPH2 transgene; an WPRE(x) element; a hGH sequence; and a 3′ ITR. The vector genome may comprise, in 5′ to 3′ order, any one of the polynucleotide sequences SEQ ID NO: 27; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length wild type transgene, i.e., a transgene encoding a JPH2 of at least 600 or at least 630 amino acids.

| SEQ ID NO: 27 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtct cagtccatta ggagccagta | 180 |
| gcctggaaga tgtctttacc cccagcatca gttcaagtgg agcagcacat aactcttgcc | 240 |
| ctctgccttc caagattctg gtgctgagac ttatggagtg tcttggaggt tgccttctgc | 300 |
| cccccaaccc tgctcccagc tggccctccc aggcctgggt tgctggcctc tgctttatca | 360 |
| ggattctcaa gagggacagc tggtttatgt tgcatgactg ttccctgcat atctgctctg | 420 |
| gttttaaata gcttatctga gcagctggag gaccacatgg gcttatatgg cgtggggtac | 480 |
| atgttcctgt agccttgtcc ctggcacctg ccaaaatagc agccaacacc ccccaccccc | 540 |
| accgccatcc ccctgcccca cccgtcccct gtcgcacatt cctccctccg cagggctggc | 600 |
| tcaccaggcc ccagcccaca tgcctgctta aagccctctc catcctctgc ctcacccagt | 660 |
| ccccgctgag actgagcaga cgcctccagg atctgtcggc aggccaccat gagtgggggc | 720 |
| cgcttcgact ttgatgatgg aggggcgtac tgcgggggct gggagggggg aaaggcccat | 780 |
| gggcatggac tgtgcacagg ccccaagggc cagggcgaat actctggctc ctggaacttt | 840 |
| ggctttgagg tggcaggtgt ctacacctgg cccagcggaa acacctttga gggatactgg | 900 |
| agccagggca aacggcatgg gctgggcata gagaccaagg ggcgctggct ctacaagggc | 960 |
| gagtggacac atggcttcaa gggacgctac ggaatccggc agagctcaag cagcggtgcc | 1020 |
| aagtatgagg gcacctggaa caatggcctg caagacggct atggcaccga gacctatgct | 1080 |
| gatggaggga cgtaccaagg ccagttcacc aacggcatgc gccatggcta cggagtacgc | 1140 |
| cagagcgtgc cctacgggat ggccgtggtg gtgcgctcgc cgctgcgcac gtcgctgtcg | 1200 |
| tccctgcgca gcgagcacag caacggcacg gtggccccgg actctcccgc ctcgccggcc | 1260 |
| tccgacggcc ccgcgctgcc ctcgcccgcc atcccgcgtg gcggcttcgc gctcagcctc | 1320 |
| ctggccaatg ccgaggcggc cgcgcgggcg cccaagggcg gcggcctctt ccagcggggc | 1380 |
| gcgctgctgg gcaagctgcg gcgcgcagag tcgcgcacgt ccgtgggtag ccagcgcagc | 1440 |
| cgtgtcagct tccttaagag cgacctcagc tcgggcgcca gcgacgccgc gtccaccgcc | 1500 |
| agcctgggag aggccgccga gggcgccgac gaggccgcac ccttcgaggc cgatatcgac | 1560 |
| gccaccacca ccgagaccta catgggcgag tggaagaacg acaaacgctc gggcttcggc | 1620 |
| gtgagcgaac gctccagtgg cctccgctac gagggcgagt ggctggacaa cctgcgccac | 1680 |
| ggctatggct gcaccacgct gcccgacggc caccgcgagg agggcaagta ccgccacaac | 1740 |
| gtgctggtca aggacaccaa gcgccgcatg ctgcagctca agagcaacaa ggtccgccag | 1800 |
| aaagtggagc acagtgtgga gggtgcccag cgcgccgctg ctatcgcgcg ccagaaggcc | 1860 |
| gagattgccg cctccaggac aagccacgcc aaggccaaag ctgaggcagc ggaacaggcc | 1920 |
| gccctggctg ccaaccagga gtccaacatt gctcgcactt tggccaggga gctggctccg | 1980 |
| gacttctacc agccaggtcc ggaatatcag aagcgccggc tgctgcagga gatcctggag | 2040 |
| aactcggaga gcctgctgga gccccccgac cggggcgccg gcgcagcggg cctcccacag | 2100 |
| ccgccccgcg agagcccgca gctgcacgag cgtgagaccc ctcggcccga gggtggctcc | 2160 |
| ccgtcaccgg ccgggacgcc cccgcagccc aagcggccca ggcccggggt gtccaaggac | 2220 |
| ggcctgctga gcccaggcgc ctggaacggc gagcccagcg gtgagggcag ccggtcagtc | 2280 |
| actccgtccg agggcgcggg ccgccgcagc cccgcgcgtc cagccaccga gcgcatggcc | 2340 |
| atcgaggctc tgcaggcacc gcctgcgccg tcgcgggagc cggaggtggc gctttaccag | 2400 |
| ggctaccaca gctatgctgt gcgcaccacg ccgcccgagc ccccaccctt tgaggaccag | 2460 |
| cccgagcccg aggtctccgg gtccgagtcc gcgccctcgt ccccggccac cgccccgctg | 2520 |
| caggccccca cgctccgagg ccccgagcct gcacgcgaga cccccgccaa gctggagccc | 2580 |
| aagcccatca tccccaaagc cgagcccagg gccaaggccc gcaagactga ggctcgaggg | 2640 |
| ctgaccaagg cgggggccaa gaagaaggcg cggaaggagg ccgcactggc ggcagaggcg | 2700 |
| gaggtggagg tggaagaggt ccccaacacc atcctcatct gcatggtgat cctgctgaac | 2760 |
| atcggcctgg ccatcctctt tgttcacctc ctgacctgat caacctctgg attacaaaat | 2820 |
| ttgtgaaaga ttgactggta ttcttaacta tgttgctcct tttacgctat gtggatacgc | 2880 |
| tgctttaatg cctttgtatc atgctattgc ttcccgtatg gctttcattt tctcctcctt | 2940 |
| gtataaatcc tggttgctgt ctctttatga ggagttgtgg cccgttgtca ggcaacgtgg | 3000 |
| cgtggtgtgc actgtgtttg ctgacgcaac ccccactggt tggggcattg ccaccacctg | 3060 |
| tcagctcctt tccgggactt tcgctttccc cctccctatt gccacggcgg aactcatcgc | 3120 |
| cgcctgcctt gcccgctgct ggacaggggc tcggctgttg ggcactgaca attccgtggt | 3180 |
| gttgtcgggg aaatcatcgt cctttccttg gctgctcgcc tgtgttgcca cctggattct | 3240 |
| gcgcgggacg tccttctgct acgtcccttc ggccctcaat ccagcggacc ttccttcccg | 3300 |
| cggcctgctg ccggctctgc ggcctcttcc gcgtcttcgc cttcgccctc agacgagtcg | 3360 |
| gatctccctt tgggccgcct ccccgcactg cccgggtggc atccctgtga cccctcccca | 3420 |
| gtgcctctcc tggccctgga agttgccact ccagtgccca ccagccttgt cctaataaaa | 3480 |
| ttaagttgca tcattttgtc tgactaggtg tccttctata atattatggg gtggaggggg | 3540 |
| gtggtatgga gcaaggggcc caagttggga agaaacctgt agggcctgcc ctaaggagga | 3600 |
| acccctagtg atggagttgg ccactccctc tctgcgcgct cgctcgctca ctgaggccgg | 3660 |
| gcgaccaaag gtcgcccgac gcccgggctt tgcccgggcg gcctcagtga gcgagcgagc | 3720 |
| gcgcagagag ggagtggcca a | 3741 |

In a certain embodiment, the vector genome comprises, in 5′ to 3′ order, a 5′ITR; a hTnnT2 promoter; a JPH2 transgene; a GFP tag; an WPRE(x) element; a hGH sequence; and a 3′ ITR. The vector genome may comprise, in 5′ to 3′ order, any one of the polynucleotide sequences SEQ ID NO: 28; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length wild type transgene, i.e. a transgene encoding a JPH2 of at least 600 or at least 630 amino acids.

| SEQ ID NO: 28 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtac ccttcagatt aaaaataact | 180 |
| gaggtaaggg cctgggtagg ggaggtggtg tgagacgctc ctgtctctcc tctatctgcc | 240 |
| catcggccct ttggggagga ggaatgtgcc caaggactaa aaaaaggcca tggagccaga | 300 |
| ggggcgaggg caacagacct ttcatgggca aaccttgggg ccctgctgtc tagcatgccc | 360 |
| cactacgggt ctaggctgcc catgtaagga ggcaaggcct ggggacaccc gagatgcctg | 420 |
| gttataatta acccagacat gtggctgccc cccccccccc aacacctgct gcctctaaaa | 480 |
| ataaccctgt ccctggtgga tcccctgcat gcgaagatct tcgaacaagg ctgtggggga | 540 |
| ctgagggcag gctgtaacag gcttgggggc cagggcttat acgtgcctcg gactcccaaa | 600 |
| gtattactgt tccatgttcc cggcgaaggg ccagctgtcc cccgccagct agactcagca | 660 |
| cttagtttag gaaccagtga gcaagtcagc ccttggggca gcccatacaa ggccatgggg | 720 |
| ctgggcaagc tgcacgcctg ggtccggggt gggcacggtg cccgggcaac gagctgaaag | 780 |
| ctcatctgct ctcaggggcc cctccctggg gacagcccct cctggctagt cacaccctgt | 840 |
| aggctcctct atataaccca ggggcacagg ggctgccctc attctaccac cacctccaca | 900 |
| gcacagacag acactcagga gccagccagg ccaccatgag tgggggccgc ttcgactttg | 960 |
| atgatggagg ggcgtactgc gggggctggg aggggggaaa ggcccatggg catggactgt | 1020 |
| gcacaggccc caagggccag ggcgaatact ctggctcctg gaactttggc tttgaggtgg | 1080 |
| caggtgtcta cacctggccc agcggaaaca cctttgaggg atactggagc cagggcaaac | 1140 |
| ggcatgggct gggcatagag accaaggggc gctggctcta caagggcgag tggacacatg | 1200 |
| gcttcaaggg acgctacgga atccggcaga gctcaagcag cggtgccaag tatgagggca | 1260 |
| cctggaacaa tggcctgcaa gacggctatg gcaccgagac ctatgctgat ggagggacgt | 1320 |
| accaaggcca gttcaccaac ggcatgcgcc atggctacgg agtacgccag agcgtgccct | 1380 |
| acgggatggc cgtggtggtg cgctcgccgc tgcgcacgtc gctgtcgtcc ctgcgcagcg | 1440 |
| agcacagcaa cggcacggtg gccccggact ctcccgcctc gccggcctcc gacggccccg | 1500 |
| cgctgccctc gcccgccatc ccgcgtggcg gcttcgcgct cagcctcctg gccaatgccg | 1560 |
| aggcggccgc gcgggcgccc aagggcggcg gcctcttcca gcggggcgcg ctgctgggca | 1620 |
| agctgcggcg cgcagagtcg cgcacgtccg tgggtagcca gcgcagccgt gtcagcttcc | 1680 |
| ttaagagcga cctcagctcg ggcgccagcg acgccgcgtc caccgccagc ctgggagagg | 1740 |
| ccgccgaggg cgccgacgag gccgcaccct tcgaggccga tatcgacgcc accaccaccg | 1800 |
| agacctacat gggcgagtgg aagaacgaca aacgctcggg cttcggcgtg agcgaacgct | 1860 |
| ccagtggcct ccgctacgag ggcgagtggc tggacaacct gcgccacggc tatggctgca | 1920 |
| ccacgctgcc cgacggccac cgcgaggagg gcaagtaccg ccacaacgtg ctggtcaagg | 1980 |
| acaccaagcg ccgcatgctg cagctcaaga gcaacaaggt ccgccagaaa gtggagcaca | 2040 |
| gtgtggaggg tgcccagcgc gccgctgcta tcgcgcgcca gaaggccgag attgccgcct | 2100 |
| ccaggacaag ccacgccaag gccaaagctg aggcagcgga acaggccgcc ctggctgcca | 2160 |
| accaggagtc caacattgct cgcactttgg ccagggagct ggctccggac ttctaccagc | 2220 |
| caggtccgga atatcagaag cgccggctgc tgcaggagat cctggagaac tcggagagcc | 2280 |
| tgctggagcc ccccgaccgg ggcgccggcg cagcgggcct cccacagccg ccccgcgaga | 2340 |
| gcccgcagct gcacgagcgt gagacccctc ggcccgaggg tggctccccg tcaccggccg | 2400 |
| ggacgccccc gcagcccaag cggcccaggc ccggggtgtc caaggacggc ctgctgagcc | 2460 |
| caggcgcctg gaacggcgag cccagcggtg agggcagccg gtcagtcact ccgtccgagg | 2520 |
| gcgcgggccg ccgcagcccc gcgcgtccag ccaccgagcg catggccatc gaggctctgc | 2580 |
| aggcaccgcc tgcgccgtcg cgggagccgg aggtggcgct ttaccagggc taccacagct | 2640 |
| atgctgtgcg caccacgccg cccgagcccc caccctttga ggaccagccc gagcccgagg | 2700 |
| tctccgggtc cgagtccgcg ccctcgtccc cggccaccgc cccgctgcag gcccccacgc | 2760 |
| tccgaggccc cgagcctgca cgcgagaccc ccgccaagct ggagcccaag cccatcatcc | 2820 |
| ccaaagccga gcccagggcc aaggcccgca agactgaggc tcgagggctg accaaggcgg | 2880 |
| gggccaagaa gaaggcgcgg aaggaggccg cactggcggc agaggcggag gtggaggtgg | 2940 |
| aagaggtccc caacaccatc ctcatctgca tggtgatcct gctgaacatc ggcctggcca | 3000 |
| tcctctttgt tcacctcctg accggatccg gcagtggaga gggcagagga agtctgctaa | 3060 |
| catgcggtga cgtcgaggag aatcctggcc caatgagcaa gggcgaggag ctgttcaccg | 3120 |
| gcgtggtgcc catcctggtg gagctggacg gcgacgtgaa cggccacaag ttcagcgtga | 3180 |
| gaggcgaggg cgagggcgac gccaccaacg gcaagctgac cctgaagttc atctgcacca | 3240 |
| ccggcaagct gcccgtgccc tggcccaccc tggtgaccac cctgacctac ggcgtgctgt | 3300 |
| gcttcagcag ataccccgac cacatgaaga gacacgactt cttcaagagc gccatgcccg | 3360 |
| agggctacgt gcaggagaga accatcagct tcaaggacga cggcacctac aagaccagag | 3420 |
| ccgaggtgaa gttcgagggc gacaccctgg tgaacagaat cgagctgaag ggcatcgact | 3480 |
| tcaaggagga cggcaacatc ctgggccaca agctggagta caacttcaac agccacaacg | 3540 |
| tgtacatcac cgccgacaag cagaagaacg gcatcaaggc ctacttcaag atcagacaca | 3600 |
| acgtggagga cggcagcgtg cagctggccg accactacca gcagaacacc cccatcggcg | 3660 |
| acggccccgt gctgctgccc gacaaccact acctgagcac ccagagcgtg ctgagcaagg | 3720 |
| accccaacga gaagagagac cacatggtgc tgctggagga cgtgaccgcc gccggcatca | 3780 |
| cccacggcat ggacgagctg tacaagtgat caacctctgg attacaaaat ttgtgaaaga | 3840 |
| ttgactggta ttcttaacta tgttgctcct tttacgctat gtggatacgc tgctttaatg | 3900 |
| cctttgtatc atgctattgc ttcccgtatg gctttcattt tctcctcctt gtataaatcc | 3960 |
| tggttgctgt ctctttatga ggagttgtgg cccgttgtca ggcaacgtgg cgtggtgtgc | 4020 |
| actgtgtttg ctgacgcaac ccccactggt tggggcattg ccaccacctg tcagctcctt | 4080 |
| tccgggactt tcgctttccc cctccctatt gccacggcgg aactcatcgc cgcctgcctt | 4140 |
| gcccgctgct ggacaggggc tcggctgttg ggcactgaca attccgtggt gttgtcgggg | 4200 |
| aaatcatcgt cctttccttg gctgctcgcc tgtgttgcca cctggattct gcgcgggacg | 4260 |
| tccttctgct acgtcccttc ggccctcaat ccagcggacc ttccttcccg cggcctgctg | 4320 |
| ccggctctgc ggcctcttcc gcgtcttcgc cttcgccctc agacgagtcg gatctccctt | 4380 |
| tgggccgcct ccccgcactg cccgggtggc atccctgtga cccctcccca gtgcctctcc | 4440 |
| tggccctgga agttgccact ccagtgccca ccagccttgt cctaataaaa ttaagttgca | 4500 |
| tcattttgtc tgactaggtg tccttctata atattatggg gtggaggggg gtggtatgga | 4560 |
| gcaaggggcc caagttggga agaaacctgt agggcctgcc ctaaggagga acccctagtg | 4620 |
| atggagttgg ccactccctc tctgcgcgct cgctcgctca ctgaggccgg gcgaccaaag | 4680 |
| gtcgcccgac gcccgggctt tgcccgggcg gcctcagtga gcgagcgagc gcgcagagag | 4740 |
| ggagtggcca a | 4751 |

In a certain embodiment, the vector genome comprises, in 5′ to 3′ order, a 5′ITR; a hTnnT2 promoter; a JPH2 transgene; a GFP tag; an WPRE(x) element; a hGH sequence; and a 3′ ITR. The vector genome may comprise, in 5′ to 3′ order, any one of the polynucleotide sequences SEQ ID NO: 29; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length wild type transgene, i.e. a transgene encoding a JPH2 of at least 600 or at least 630 amino acids.

| SEQ ID NO: 29 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtct cagtccatta ggagccagta | 180 |
| gcctggaaga tgtctttacc cccagcatca gttcaagtgg agcagcacat aactcttgcc | 240 |
| ctctgccttc caagattctg gtgctgagac ttatggagtg tcttggaggt tgccttctgc | 300 |
| cccccaaccc tgctcccagc tggccctccc aggcctgggt tgctggcctc tgctttatca | 360 |
| ggattctcaa gagggacagc tggtttatgt tgcatgactg ttccctgcat atctgctctg | 420 |
| gttttaaata gcttatctga gcagctggag gaccacatgg gcttatatgg cgtggggtac | 480 |
| atgttcctgt agccttgtcc ctggcacctg ccaaaatagc agccaacacc ccccaccccc | 540 |
| accgccatcc ccctgcccca cccgtcccct gtcgcacatt cctccctccg cagggctggc | 600 |
| tcaccaggcc ccagcccaca tgcctgctta aagccctctc catcctctgc ctcacccagt | 660 |
| ccccgctgag actgagcaga cgcctccagg atctgtcggc aggccaccat gagtgggggc | 720 |
| cgcttcgact ttgatgatgg aggggcgtac tgcgggggct gggagggggg aaaggcccat | 780 |
| gggcatggac tgtgcacagg ccccaagggc cagggcgaat actctggctc ctggaacttt | 840 |
| ggctttgagg tggcaggtgt ctacacctgg cccagcggaa acacctttga gggatactgg | 900 |
| agccagggca aacggcatgg gctgggcata gagaccaagg ggcgctggct ctacaagggc | 960 |
| gagtggacac atggcttcaa gggacgctac ggaatccggc agagctcaag cagcggtgcc | 1020 |
| aagtatgagg gcacctggaa caatggcctg caagacggct atggcaccga gacctatgct | 1080 |
| gatggaggga cgtaccaagg ccagttcacc aacggcatgc gccatggcta cggagtacgc | 1140 |
| cagagcgtgc cctacgggat ggccgtggtg gtgcgctcgc cgctgcgcac gtcgctgtcg | 1200 |
| tccctgcgca gcgagcacag caacggcacg gtggccccgg actctcccgc ctcgccggcc | 1260 |
| tccgacggcc ccgcgctgcc ctcgcccgcc atcccgcgtg gcggcttcgc gctcagcctc | 1320 |
| ctggccaatg ccgaggcggc cgcgcgggcg cccaagggcg gcggcctctt ccagcggggc | 1380 |
| gcgctgctgg gcaagctgcg gcgcgcagag tcgcgcacgt ccgtgggtag ccagcgcagc | 1440 |
| cgtgtcagct tccttaagag cgacctcagc tcgggcgcca gcgacgccgc gtccaccgcc | 1500 |
| agcctgggag aggccgccga gggcgccgac gaggccgcac ccttcgaggc cgatatcgac | 1560 |
| gccaccacca ccgagaccta catgggcgag tggaagaacg acaaacgctc gggcttcggc | 1620 |
| gtgagcgaac gctccagtgg cctccgctac gagggcgagt ggctggacaa cctgcgccac | 1680 |
| ggctatggct gcaccacgct gcccgacggc caccgcgagg agggcaagta ccgccacaac | 1740 |
| gtgctggtca aggacaccaa gcgccgcatg ctgcagctca agagcaacaa ggtccgccag | 1800 |
| aaagtggagc acagtgtgga gggtgcccag cgcgccgctg ctatcgcgcg ccagaaggcc | 1860 |
| gagattgccg cctccaggac aagccacgcc aaggccaaag ctgaggcagc ggaacaggcc | 1920 |
| gccctggctg ccaaccagga gtccaacatt gctcgcactt tggccaggga gctggctccg | 1980 |
| gacttctacc agccaggtcc ggaatatcag aagcgccggc tgctgcagga gatcctggag | 2040 |
| aactcggaga gcctgctgga gccccccgac cggggcgccg gcgcagcggg cctcccacag | 2100 |
| ccgccccgcg agagcccgca gctgcacgag cgtgagaccc ctcggcccga gggtggctcc | 2160 |
| ccgtcaccgg ccgggacgcc cccgcagccc aagcggccca ggcccggggt gtccaaggac | 2220 |
| ggcctgctga gcccaggcgc ctggaacggc gagcccagcg gtgagggcag ccggtcagtc | 2280 |
| actccgtccg agggcgcggg ccgccgcagc cccgcgcgtc cagccaccga gcgcatggcc | 2340 |
| atcgaggctc tgcaggcacc gcctgcgccg tcgcgggagc cggaggtggc gctttaccag | 2400 |
| ggctaccaca gctatgctgt gcgcaccacg ccgcccgagc ccccaccctt tgaggaccag | 2460 |
| cccgagcccg aggtctccgg gtccgagtcc gcgccctcgt ccccggccac cgccccgctg | 2520 |
| caggccccca cgctccgagg ccccgagcct gcacgcgaga cccccgccaa gctggagccc | 2580 |
| aagcccatca tccccaaagc cgagcccagg gccaaggccc gcaagactga ggctcgaggg | 2640 |
| ctgaccaagg cgggggccaa gaagaaggcg cggaaggagg ccgcactggc ggcagaggcg | 2700 |
| gaggtggagg tggaagaggt ccccaacacc atcctcatct gcatggtgat cctgctgaac | 2760 |
| atcggcctgg ccatcctctt tgttcacctc ctgaccggat ccggcagtgg agagggcaga | 2820 |
| ggaagtctgc taacatgcgg tgacgtcgag gagaatcctg gcccaatgag caagggcgag | 2880 |
| gagctgttca ccggcgtggt gcccatcctg gtggagctgg acggcgacgt gaacggccac | 2940 |
| aagttcagcg tgagaggcga gggcgagggc gacgccacca acggcaagct gaccctgaag | 3000 |
| ttcatctgca ccaccggcaa gctgcccgtg ccctggccca ccctggtgac caccctgacc | 3060 |
| tacggcgtgc tgtgcttcag cagatacccc gaccacatga agagacacga cttcttcaag | 3120 |
| agcgccatgc ccgagggcta cgtgcaggag agaaccatca gcttcaagga cgacggcacc | 3180 |
| tacaagacca gagccgaggt gaagttcgag ggcgacaccc tggtgaacag aatcgagctg | 3240 |
| aagggcatcg acttcaagga ggacggcaac atcctgggcc acaagctgga gtacaacttc | 3300 |
| aacagccaca acgtgtacat caccgccgac aagcagaaga acggcatcaa ggcctacttc | 3360 |
| aagatcagac acaacgtgga ggacggcagc gtgcagctgg ccgaccacta ccagcagaac | 3420 |
| acccccatcg gcgacggccc cgtgctgctg cccgacaacc actacctgag cacccagagc | 3480 |
| gtgctgagca aggaccccaa cgagaagaga gaccacatgg tgctgctgga ggacgtgacc | 3540 |
| gccgccggca tcacccacgg catggacgag ctgtacaagt gatcaacctc tggattacaa | 3600 |
| aatttgtgaa agattgactg gtattcttaa ctatgttgct ccttttacgc tatgtggata | 3660 |
| cgctgcttta atgcctttgt atcatgctat tgcttcccgt atggctttca ttttctcctc | 3720 |
| cttgtataaa tcctggttgc tgtctcttta tgaggagttg tggcccgttg tcaggcaacg | 3780 |
| tggcgtggtg tgcactgtgt ttgctgacgc aacccccact ggttggggca ttgccaccac | 3840 |
| ctgtcagctc ctttccggga ctttcgcttt ccccctccct attgccacgg cggaactcat | 3900 |
| cgccgcctgc cttgcccgct gctggacagg ggctcggctg ttgggcactg acaattccgt | 3960 |
| ggtgttgtcg gggaaatcat cgtcctttcc ttggctgctc gcctgtgttg ccacctggat | 4020 |
| tctgcgcggg acgtccttct gctacgtccc ttcggccctc aatccagcgg accttccttc | 4080 |
| ccgcggcctg ctgccggctc tgcggcctct tccgcgtctt cgccttcgcc ctcagacgag | 4140 |
| tcggatctcc ctttgggccg cctccccgca ctgcccgggt ggcatccctg tgacccctcc | 4200 |
| ccagtgcctc tcctggccct ggaagttgcc actccagtgc ccaccagcct tgtcctaata | 4260 |
| aaattaagtt gcatcatttt gtctgactag gtgtccttct ataatattat ggggtggagg | 4320 |
| ggggtggtat ggagcaaggg gcccaagttg ggaagaaacc tgtagggcct gccctaagga | 4380 |
| ggaaccccta gtgatggagt tggccactcc ctctctgcgc gctcgctcgc tcactgaggc | 4440 |
| cgggcgacca aaggtcgccc gacgcccggg ctttgcccgg gcggcctcag tgagcgagcg | 4500 |
| agcgcgcaga gagggagtgg ccaa | 4524 |

In a certain embodiment, the vector genome comprises, in 5′ to 3′ order, a 5′ITR; a CMV enhancer element; a CMV promoter; a JPH2 transgene; a GFP tag; an WPRE(x) element; a hGH sequence; and a 3′ ITR. The vector genome may comprise, in 5′ to 3′ order, any one of the polynucleotide sequences SEQ ID NO: 30; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length wild type transgene, i.e. a transgene encoding a JPH2 of at least 600 or at least 630 amino acids.

| SEQ ID NO: 30 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtta catacgttac ataacttacg | 180 |
| gtaaatggcc cgcctggctg accgcccaac gacccccgcc cattgacgtc aataatgacg | 240 |
| tatgttccca tagtaacgcc aatagggact ttccattgac gtcaatgggt ggagtattta | 300 |
| cggtaaactg cccacttggc agtacatcaa gtgtatcata tgccaagtac gccccctatt | 360 |
| gacgtcaatg acggtaaatg gcccgcctgg cattatgccc agtacatgac cttatgggac | 420 |
| tttcctactt ggcagtacat ctacgtatta gtcatcgcta ttaccatggt gatgcggttt | 480 |
| tggcagtaca tcaatgggcg tggatagcgg tttgactcac ggggatttcc aagtctccac | 540 |
| cccattgacg tcaatgggag tttgttttgg caccaaaatc aacgggactt tccaaaatgt | 600 |
| cgtaacaact ccgccccatt gacgcaaatg ggcggtaggc gtgtacggtg ggaggtctat | 660 |
| ataagcagag ctcgtttagt gaaccgtcag atcgcctgga gacgccatcc acgctgtttt | 720 |
| gacctccata gaagacaccg ggaccgatcc agcctccgcg ggccaccatg agtgggggcc | 780 |
| gcttcgactt tgatgatgga ggggcgtact gcgggggctg ggagggggga aaggcccatg | 840 |
| ggcatggact gtgcacaggc cccaagggcc agggcgaata ctctggctcc tggaactttg | 900 |
| gctttgaggt ggcaggtgtc tacacctggc ccagcggaaa cacctttgag ggatactgga | 960 |
| gccagggcaa acggcatggg ctgggcatag agaccaaggg gcgctggctc tacaagggcg | 1020 |
| agtggacaca tggcttcaag ggacgctacg gaatccggca gagctcaagc agcggtgcca | 1080 |
| agtatgaggg cacctggaac aatggcctgc aagacggcta tggcaccgag acctatgctg | 1140 |
| atggagggac gtaccaaggc cagttcacca acggcatgcg ccatggctac ggagtacgcc | 1200 |
| agagcgtgcc ctacgggatg gccgtggtgg tgcgctcgcc gctgcgcacg tcgctgtcgt | 1260 |
| ccctgcgcag cgagcacagc aacggcacgg tggccccgga ctctcccgcc tcgccggcct | 1320 |
| ccgacggccc cgcgctgccc tcgcccgcca tcccgcgtgg cggcttcgcg ctcagcctcc | 1380 |
| tggccaatgc cgaggcggcc gcgcgggcgc ccaagggcgg cggcctcttc cagcggggcg | 1440 |
| cgctgctggg caagctgcgg cgcgcagagt cgcgcacgtc cgtgggtagc cagcgcagcc | 1500 |
| gtgtcagctt ccttaagagc gacctcagct cgggcgccag cgacgccgcg tccaccgcca | 1560 |
| gcctgggaga ggccgccgag ggcgccgacg aggccgcacc cttcgaggcc gatatcgacg | 1620 |
| ccaccaccac cgagacctac atgggcgagt ggaagaacga caaacgctcg ggcttcggcg | 1680 |
| tgagcgaacg ctccagtggc ctccgctacg agggcgagtg gctggacaac ctgcgccacg | 1740 |
| gctatggctg caccacgctg cccgacggcc accgcgagga gggcaagtac cgccacaacg | 1800 |
| tgctggtcaa ggacaccaag cgccgcatgc tgcagctcaa gagcaacaag gtccgccaga | 1860 |
| aagtggagca cagtgtggag ggtgcccagc gcgccgctgc tatcgcgcgc cagaaggccg | 1920 |
| agattgccgc ctccaggaca agccacgcca aggccaaagc tgaggcagcg gaacaggccg | 1980 |
| ccctggctgc caaccaggag tccaacattg ctcgcacttt ggccagggag ctggctccgg | 2040 |
| acttctacca gccaggtccg gaatatcaga agcgccggct gctgcaggag atcctggaga | 2100 |
| actcggagag cctgctggag ccccccgacc ggggcgccgg cgcagcgggc ctcccacagc | 2160 |
| cgccccgcga gagcccgcag ctgcacgagc gtgagacccc tcggcccgag ggtggctccc | 2220 |
| cgtcaccggc cgggacgccc ccgcagccca agcggcccag gcccggggtg tccaaggacg | 2280 |
| gcctgctgag cccaggcgcc tggaacggcg agcccagcgg tgagggcagc cggtcagtca | 2340 |
| ctccgtccga gggcgcgggc cgccgcagcc ccgcgcgtcc agccaccgag cgcatggcca | 2400 |
| tcgaggctct gcaggcaccg cctgcgccgt cgcgggagcc ggaggtggcg ctttaccagg | 2460 |
| gctaccacag ctatgctgtg cgcaccacgc cgcccgagcc cccacccttt gaggaccagc | 2520 |
| ccgagcccga ggtctccggg tccgagtccg cgccctcgtc cccggccacc gccccgctgc | 2580 |
| aggcccccac gctccgaggc cccgagcctg cacgcgagac ccccgccaag ctggagccca | 2640 |
| agcccatcat ccccaaagcc gagcccaggg ccaaggcccg caagactgag gctcgagggc | 2700 |
| tgaccaaggc gggggccaag aagaaggcgc ggaaggaggc cgcactggcg gcagaggcgg | 2760 |
| aggtggaggt ggaagaggtc cccaacacca tcctcatctg catggtgatc ctgctgaaca | 2820 |
| tcggcctggc catcctcttt gttcacctcc tgaccggatc cggcagtgga gagggcagag | 2880 |
| gaagtctgct aacatgcggt gacgtcgagg agaatcctgg cccaatgagc aagggcgagg | 2940 |
| agctgttcac cggcgtggtg cccatcctgg tggagctgga cggcgacgtg aacggccaca | 3000 |
| agttcagcgt gagaggcgag ggcgagggcg acgccaccaa cggcaagctg accctgaagt | 3060 |
| tcatctgcac caccggcaag ctgcccgtgc cctggcccac cctggtgacc accctgacct | 3120 |
| acggcgtgct gtgcttcagc agataccccg accacatgaa gagacacgac ttcttcaaga | 3180 |
| gcgccatgcc cgagggctac gtgcaggaga gaaccatcag cttcaaggac gacggcacct | 3240 |
| acaagaccag agccgaggtg aagttcgagg gcgacaccct ggtgaacaga atcgagctga | 3300 |
| agggcatcga cttcaaggag gacggcaaca tcctgggcca caagctggag tacaacttca | 3360 |
| acagccacaa cgtgtacatc accgccgaca agcagaagaa cggcatcaag gcctacttca | 3420 |
| agatcagaca caacgtggag gacggcagcg tgcagctggc cgaccactac cagcagaaca | 3480 |
| cccccatcgg cgacggcccc gtgctgctgc ccgacaacca ctacctgagc acccagagcg | 3540 |
| tgctgagcaa ggaccccaac gagaagagag accacatggt gctgctggag gacgtgaccg | 3600 |
| ccgccggcat cacccacggc atggacgagc tgtacaagtg atcaacctct ggattacaaa | 3660 |
| atttgtgaaa gattgactgg tattcttaac tatgttgctc cttttacgct atgtggatac | 3720 |
| gctgctttaa tgcctttgta tcatgctatt gcttcccgta tggctttcat tttctcctcc | 3780 |
| ttgtataaat cctggttgct gtctctttat gaggagttgt ggcccgttgt caggcaacgt | 3840 |
| ggcgtggtgt gcactgtgtt tgctgacgca acccccactg gttggggcat tgccaccacc | 3900 |
| tgtcagctcc tttccgggac tttcgctttc cccctcccta ttgccacggc ggaactcatc | 3960 |
| gccgcctgcc ttgcccgctg ctggacaggg gctcggctgt tgggcactga caattccgtg | 4020 |
| gtgttgtcgg ggaaatcatc gtcctttcct tggctgctcg cctgtgttgc cacctggatt | 4080 |
| ctgcgcggga cgtccttctg ctacgtccct tcggccctca atccagcgga ccttccttcc | 4140 |
| cgcggcctgc tgccggctct gcggcctctt ccgcgtcttc gccttcgccc tcagacgagt | 4200 |
| cggatctccc tttgggccgc ctccccgcac tgcccgggtg gcatccctgt gacccctccc | 4260 |
| cagtgcctct cctggccctg gaagttgcca ctccagtgcc caccagcctt gtcctaataa | 4320 |
| aattaagttg catcattttg tctgactagg tgtccttcta taatattatg gggtggaggg | 4380 |
| gggtggtatg gagcaagggg cccaagttgg gaagaaacct gtagggcctg ccctaaggag | 4440 |
| gaacccctag tgatggagtt ggccactccc tctctgcgcg ctcgctcgct cactgaggcc | 4500 |
| gggcgaccaa aggtcgcccg acgcccgggc tttgcccggg cggcctcagt gagcgagcga | 4560 |
| gcgcgcagag agggagtggc caa | 4583 |

In a certain embodiment, the vector genome comprises, in 5′ to 3′ order, a 5′ITR; an MHCK7 promoter; a JPH2 1mutAA (R572A and T573A) transgene; an WPRE(x) element; a hGH sequence; and a 3′ ITR. The vector genome may comprise, in 5′ to 3′ order, the polynucleotide sequences SEQ ID NO: 76; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length 1mutAA (R572A and T573A) transgene, i.e. a transgene encoding a JPH2 of at least 600 or at least 630 amino acids.

| SEQ ID NO: 76 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtac ccttcagatt aaaaataact | 180 |
| gaggtaaggg cctgggtagg ggaggtggtg tgagacgctc ctgtctctcc tctatctgcc | 240 |
| catcggccct ttggggagga ggaatgtgcc caaggactaa aaaaaggcca tggagccaga | 300 |
| ggggcgaggg caacagacct ttcatgggca aaccttgggg ccctgctgtc tagcatgccc | 360 |
| cactacgggt ctaggctgcc catgtaagga ggcaaggcct ggggacaccc gagatgcctg | 420 |
| gttataatta acccagacat gtggctgccc cccccccccc aacacctgct gcctctaaaa | 480 |
| ataaccctgt ccctggtgga tcccctgcat gcgaagatct tcgaacaagg ctgtggggga | 540 |
| ctgagggcag gctgtaacag gcttgggggc cagggcttat acgtgcctgg gactcccaaa | 600 |
| gtattactgt tccatgttcc cggcgaaggg ccagctgtcc cccgccagct agactcagca | 660 |
| cttagtttag gaaccagtga gcaagtcagc ccttggggca gcccatacaa ggccatgggg | 720 |
| ctgggcaagc tgcacgcctg ggtccggggt gggcacggtg cccgggcaac gagctgaaag | 780 |
| ctcatctgct ctcaggggcc cctccctggg gacagcccct cctggctagt cacaccctgt | 840 |
| aggctcctct atataaccca ggggcacagg ggctgccctc attctaccac cacctccaca | 900 |
| gcacagacag acactcagga gccagccagg gtaagtttag tctttttgtc ttttatttca | 960 |
| ggtcccggat ccggtggtgg tgcaaatcaa agaactgctc ctcagtggat gttgccttta | 1020 |
| cttctaggcc tgtacggaag tgttacttct gctctaaaag ctgcggaatt gtacccgcgc | 1080 |
| caccatgagt gggggccgct tcgactttga tgatggaggg gcgtactgcg ggggctggga | 1140 |
| ggggggaaag gcccatgggc atggactgtg cacaggcccc aagggccagg gcgaatactc | 1200 |
| tggctcctgg aactttggct ttgaggtggc aggtgtctac acctggccca gcggaaacac | 1260 |
| ctttgaggga tactggagcc agggcaaacg gcatgggctg ggcatagaga ccaaggggcg | 1320 |
| ctggctctac aagggcgagt ggacacatgg cttcaaggga cgctacggaa tccggcagag | 1380 |
| ctcaagcagc ggtgccaagt atgagggcac ctggaacaat ggcctgcaag acggctatgg | 1440 |
| caccgagacc tatgctgatg gagggacgta ccaaggccag ttcaccaacg gcatgcgcca | 1500 |
| tggctacgga gtacgccaga gcgtgcccta cgggatggcc gtggtggtgc gctcgccgct | 1560 |
| gcgcacgtcg ctgtcgtccc tgcgcagcga gcacagcaac ggcacggtgg ccccggactc | 1620 |
| tcccgcctcg ccggcctccg acggccccgc gctgccctcg cccgccatcc cgcgtggcgg | 1680 |
| cttcgcgctc agcctcctgg ccaatgccga ggcggccgcg cgggcgccca agggcggcgg | 1740 |
| cctcttccag cggggcgcgc tgctgggcaa gctgcggcgc gcagagtcgc gcacgtccgt | 1800 |
| gggtagccag cgcagccgtg tcagcttcct taagagcgac ctcagctcgg gcgccagcga | 1860 |
| cgccgcgtcc accgccagcc tgggagaggc cgccgagggc gccgacgagg ccgcaccctt | 1920 |
| cgaggccgat atcgacgcca ccaccaccga gacctacatg ggcgagtgga agaacgacaa | 1980 |
| acgctcgggc ttcggcgtga gcgaacgctc cagtggcctc cgctacgagg gcgagtggct | 2040 |
| ggacaacctg cgccacggct atggctgcac cacgctgccc gacggccacc gcgaggaggg | 2100 |
| caagtaccgc cacaacgtgc tggtcaagga caccaagcgc cgcatgctgc agctcaagag | 2160 |
| caacaaggtc cgccagaaag tggagcacag tgtggagggt gcccagcgcg ccgctgctat | 2220 |
| cgcgcgccag aaggccgaga ttgccgcctc caggacaagc cacgccaagg ccaaagctga | 2280 |
| ggcagcggaa caggccgccc tggctgccaa ccaggagtcc aacattgctc gcactttggc | 2340 |
| cagggagctg gctccggact tctaccagcc aggtccggaa tatcagaagc gccggctgct | 2400 |
| gcaggagatc ctggagaact cggagagcct gctggagccc cccgaccggg gcgccggcgc | 2460 |
| agcgggcctc ccacagccgc cccgcgagag cccgcagctg cacgagcgtg agacccctcg | 2520 |
| gcccgagggt ggctccccgt caccggccgg gacgcccccg cagcccaagc ggcccaggcc | 2580 |
| cggggtgtcc aaggacggcc tgctgagccc aggcgcctgg aacggcgagc ccagcggtga | 2640 |
| gggcagccgg tcagtcactc cgtccgaggg cgcgggccgc cgcagccccg cgcgtccagc | 2700 |
| caccgagcgc atggccatcg aggctctgca ggcaccgcct gcgccgtcgc gggagccgga | 2760 |
| ggtggcgctt taccagggct accacagcta tgctgtggcc gccacgccgc ccgagccccc | 2820 |
| accctttgag gaccagcccg agcccgaggt ctccgggtcc gagtccgcgc cctcgtcccc | 2880 |
| ggccaccgcc ccgctgcagg cccccacgct ccgaggcccc gagcctgcac gcgagacccc | 2940 |
| cgccaagctg gagcccaagc ccatcatccc caaagccgag cccagggcca aggcccgcaa | 3000 |
| gactgaggct cgagggctga ccaaggcggg ggccaagaag aaggcgcgga aggaggccgc | 3060 |
| actggcggca gaggcggagg tggaggtgga agaggtcccc aacaccatcc tcatctgcat | 3120 |
| ggtgatcctg ctgaacatcg gcctggccat cctctttgtt cacctcctga cctgatcaac | 3180 |
| ctctggatta caaaatttgt gaaagattga ctggtattct taactatgtt gctcctttta | 3240 |
| cgctatgtgg atacgctgct ttaatgcctt tgtatcatgc tattgcttcc cgtatggctt | 3300 |
| tcattttctc ctccttgtat aaatcctggt tgctgtctct ttatgaggag ttgtggcccg | 3360 |
| ttgtcaggca acgtggcgtg gtgtgcactg tgtttgctga cgcaaccccc actggttggg | 3420 |
| gcattgccac cacctgtcag ctcctttccg ggactttcgc tttccccctc cctattgcca | 3480 |
| cggcggaact catcgccgcc tgccttgccc gctgctggac aggggctcgg ctgttgggca | 3540 |
| ctgacaattc cgtggtgttg tcggggaaat catcgtcctt tccttggctg ctcgcctgtg | 3600 |
| ttgccacctg gattctgcgc gggacgtcct tctgctacgt cccttcggcc ctcaatccag | 3660 |
| cggaccttcc ttcccgcggc ctgctgccgg ctctgcggcc tcttccgcgt cttcgccttc | 3720 |
| gccctcagac gagtcggatc tccctttggg ccgcctcccc gcactgcccg ggtggcatcc | 3780 |
| ctgtgacccc tccccagtgc ctctcctggc cctggaagtt gccactccag tgcccaccag | 3840 |
| ccttgtccta ataaaattaa gttgcatcat tttgtctgac taggtgtcct tctataatat | 3900 |
| tatggggtgg aggggggtgg tatggagcaa ggggcccaag ttgggaagaa acctgtaggg | 3960 |
| cctgccctaa ggaggaaccc ctagtgatgg agttggccac tccctctctg cgcgctcgct | 4020 |
| cgctcactga ggccgggcga ccaaaggtcg cccgacgccc gggctttgcc cgggcggcct | 4080 |
| cagtgagcga gcgagcgcgc agagagggag tggccaa | 4117 |

In a certain embodiment, the vector genome comprises, in 5′ to 3′ order, a 5′ ITR; a hTnnT2 promoter; a JPH2 1mutAA (R572A and T573A) transgene; an WPRE(x) element; a hGH sequence; and a 3′ ITR. The vector genome may comprise, in 5′ to 3′ order, any one of the polynucleotide sequences SEQ ID NO: 77; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length JPH2 1mutAA (R572A and T573A) transgene, i.e. a transgene encoding a JPH2 of at least 600 or at least 630 amino acids.

| SEQ ID NO: 77 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtct cagtccatta ggagccagta | 180 |
| gcctggaaga tgtctttacc cccagcatca gttcaagtgg agcagcacat aactcttgcc | 240 |
| ctctgccttc caagattctg gtgctgagac ttatggagtg tcttggaggt tgccttctgc | 300 |
| cccccaaccc tgctcccagc tggccctccc aggcctgggt tgctggcctc tgctttatca | 360 |
| ggattctcaa gagggacagc tggtttatgt tgcatgactg ttccctgcat atctgctctg | 420 |
| gttttaaata gcttatctga gcagctggag gaccacatgg gcttatatgg cgtggggtac | 480 |
| atgttcctgt agccttgtcc ctggcacctg ccaaaatagc agccaacacc ccccaccccc | 540 |
| accgccatcc ccctgcccca cccgtcccct gtcgcacatt cctccctccg cagggctggc | 600 |
| tcaccaggcc ccagcccaca tgcctgctta aagccctctc catcctctgc ctcacccagt | 660 |
| ccccgctgag actgagcaga cgcctccagg atctgtcggc aggccaccat gagtgggggc | 720 |
| cgcttcgact ttgatgatgg aggggcgtac tgcgggggct gggagggggg aaaggcccat | 780 |
| gggcatggac tgtgcacagg ccccaagggc cagggcgaat actctggctc ctggaacttt | 840 |
| ggctttgagg tggcaggtgt ctacacctgg cccagcggaa acacctttga gggatactgg | 900 |
| agccagggca aacggcatgg gctgggcata gagaccaagg ggcgctggct ctacaagggc | 960 |
| gagtggacac atggcttcaa gggacgctac ggaatccggc agagctcaag cagcggtgcc | 1020 |
| aagtatgagg gcacctggaa caatggcctg caagacggct atggcaccga gacctatgct | 1080 |
| gatggaggga cgtaccaagg ccagttcacc aacggcatgc gccatggcta cggagtacgc | 1140 |
| cagagcgtgc cctacgggat ggccgtggtg gtgcgctcgc cgctgcgcac gtcgctgtcg | 1200 |
| tccctgcgca gcgagcacag caacggcacg gtggccccgg actctcccgc ctcgccggcc | 1260 |
| tccgacggcc ccgcgctgcc ctcgcccgcc atcccgcgtg gcggcttcgc gctcagcctc | 1320 |
| ctggccaatg ccgaggcggc cgcgcgggcg cccaagggcg gcggcctctt ccagcggggc | 1380 |
| gcgctgctgg gcaagctgcg gcgcgcagag tcgcgcacgt ccgtgggtag ccagcgcagc | 1440 |
| cgtgtcagct tccttaagag cgacctcagc tcgggcgcca gcgacgccgc gtccaccgcc | 1500 |
| agcctgggag aggccgccga gggcgccgac gaggccgcac ccttcgaggc cgatatcgac | 1560 |
| gccaccacca ccgagaccta catgggcgag tggaagaacg acaaacgctc gggcttcggc | 1620 |
| gtgagcgaac gctccagtgg cctccgctac gagggcgagt ggctggacaa cctgcgccac | 1680 |
| ggctatggct gcaccacgct gcccgacggc caccgcgagg agggcaagta ccgccacaac | 1740 |
| gtgctggtca aggacaccaa gcgccgcatg ctgcagctca agagcaacaa ggtccgccag | 1800 |
| aaagtggagc acagtgtgga gggtgcccag cgcgccgctg ctatcgcgcg ccagaaggcc | 1860 |
| gagattgccg cctccaggac aagccacgcc aaggccaaag ctgaggcagc ggaacaggcc | 1920 |
| gccctggctg ccaaccagga gtccaacatt gctcgcactt tggccaggga gctggctccg | 1980 |
| gacttctacc agccaggtcc ggaatatcag aagcgccggc tgctgcagga gatcctggag | 2040 |
| aactcggaga gcctgctgga gccccccgac cggggcgccg gcgcagcggg cctcccacag | 2100 |
| ccgccccgcg agagcccgca gctgcacgag cgtgagaccc ctcggcccga gggtggctcc | 2160 |
| ccgtcaccgg ccgggacgcc cccgcagccc aagcggccca ggcccggggt gtccaaggac | 2220 |
| ggcctgctga gcccaggcgc ctggaacggc gagcccagcg gtgagggcag ccggtcagtc | 2280 |
| actccgtccg agggcgcggg ccgccgcagc cccgcgcgtc cagccaccga gcgcatggcc | 2340 |
| atcgaggctc tgcaggcacc gcctgcgccg tcgcgggagc cggaggtggc gctttaccag | 2400 |
| ggctaccaca gctatgctgt ggccgccacg ccgcccgagc ccccaccctt tgaggaccag | 2460 |
| cccgagcccg aggtctccgg gtccgagtcc gcgccctcgt ccccggccac cgccccgctg | 2520 |
| caggccccca cgctccgagg ccccgagcct gcacgcgaga cccccgccaa gctggagccc | 2580 |
| aagcccatca tccccaaagc cgagcccagg gccaaggccc gcaagactga ggctcgaggg | 2640 |
| ctgaccaagg cgggggccaa gaagaaggcg cggaaggagg ccgcactggc ggcagaggcg | 2700 |
| gaggtggagg tggaagaggt ccccaacacc atcctcatct gcatggtgat cctgctgaac | 2760 |
| atcggcctgg ccatcctctt tgttcacctc ctgacctgat caacctctgg attacaaaat | 2820 |
| ttgtgaaaga ttgactggta ttcttaacta tgttgctcct tttacgctat gtggatacgc | 2880 |
| tgctttaatg cctttgtatc atgctattgc ttcccgtatg gctttcattt tctcctcctt | 2940 |
| gtataaatcc tggttgctgt ctctttatga ggagttgtgg cccgttgtca ggcaacgtgg | 3000 |
| cgtggtgtgc actgtgtttg ctgacgcaac ccccactggt tggggcattg ccaccacctg | 3060 |
| tcagctcctt tccgggactt tcgctttccc cctccctatt gccacggcgg aactcatcgc | 3120 |
| cgcctgcctt gcccgctgct ggacaggggc tcggctgttg ggcactgaca attccgtggt | 3180 |
| gttgtcgggg aaatcatcgt cctttccttg gctgctcgcc tgtgttgcca cctggattct | 3240 |
| gcgcgggacg tccttctgct acgtcccttc ggccctcaat ccagcggacc ttccttcccg | 3300 |
| cggcctgctg ccggctctgc ggcctcttcc gcgtcttcgc cttcgccctc agacgagtcg | 3360 |
| gatctccctt tgggccgcct ccccgcactg cccgggtggc atccctgtga cccctcccca | 3420 |
| gtgcctctcc tggccctgga agttgccact ccagtgccca ccagccttgt cctaataaaa | 3480 |
| ttaagttgca tcattttgtc tgactaggtg tccttctata atattatggg gtggaggggg | 3540 |
| gtggtatgga gcaaggggcc caagttggga agaaacctgt agggcctgcc ctaaggagga | 3600 |
| acccctagtg atggagttgg ccactccctc tctgcgcgct cgctcgctca ctgaggccgg | 3660 |
| gcgaccaaag gtcgcccgac gcccgggctt tgcccgggcg gcctcagtga gcgagcgagc | 3720 |
| gcgcagagag ggagtggcca a | 3741 |

In a certain embodiment, the vector genome comprises, in 5′ to 3′ order, a 5′ITR; an MHCK7 promoter; a JPH2 1mutAA (R572A and T573A) transgene; a GFP tag; an WPRE(x) element; a hGH sequence; and a 3′ ITR. The vector genome may comprise, in 5′ to 3′ order, the polynucleotide sequences SEQ ID NO: 78; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length 1mutAA (R572A and T573A) transgene, i.e. a transgene encoding a JPH2 of at least 600 or at least 630 amino acids.

| SEQ ID NO: 78 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtac ccttcagatt aaaaataact | 180 |
| gaggtaaggg cctgggtagg ggaggtggtg tgagacgctc ctgtctctcc tctatctgcc | 240 |
| catcggccct ttggggagga ggaatgtgcc caaggactaa aaaaaggcca tggagccaga | 300 |
| ggggcgaggg caacagacct ttcatgggca aaccttgggg ccctgctgtc tagcatgccc | 360 |
| cactacgggt ctaggctgcc catgtaagga ggcaaggcct ggggacaccc gagatgcctg | 420 |
| gttataatta acccagacat gtggctgccc cccccccccc aacacctgct gcctctaaaa | 480 |
| ataaccctgt ccctggtgga tcccctgcat gcgaagatct tcgaacaagg ctgtggggga | 540 |
| ctgagggcag gctgtaacag gcttgggggc cagggcttat acgtgcctgg gactcccaaa | 600 |
| gtattactgt tccatgttcc cggcgaaggg ccagctgtcc cccgccagct agactcagca | 660 |
| cttagtttag gaaccagtga gcaagtcagc ccttggggca gcccatacaa ggccatgggg | 720 |
| ctgggcaagc tgcacgcctg ggtccggggt gggcacggtg cccgggcaac gagctgaaag | 780 |
| ctcatctgct ctcaggggcc cctccctggg gacagcccct cctggctagt cacaccctgt | 840 |
| aggctcctct atataaccca ggggcacagg ggctgccctc attctaccac cacctccaca | 900 |
| gcacagacag acactcagga gccagccagg ccaccatgag tgggggccgc ttcgactttg | 960 |
| atgatggagg ggcgtactgc gggggctggg aggggggaaa ggcccatggg catggactgt | 1020 |
| gcacaggccc caagggccag ggcgaatact ctggctcctg gaactttggc tttgaggtgg | 1080 |
| caggtgtcta cacctggccc agcggaaaca cctttgaggg atactggagc cagggcaaac | 1140 |
| ggcatgggct gggcatagag accaaggggc gctggctcta caagggcgag tggacacatg | 1200 |
| gcttcaaggg acgctacgga atccggcaga gctcaagcag cggtgccaag tatgagggca | 1260 |
| cctggaacaa tggcctgcaa gacggctatg gcaccgagac ctatgctgat ggagggacgt | 1320 |
| accaaggcca gttcaccaac ggcatgcgcc atggctacgg agtacgccag agcgtgccct | 1380 |
| acgggatggc cgtggtggtg cgctcgccgc tgcgcacgtc gctgtcgtcc ctgcgcagcg | 1440 |
| agcacagcaa cggcacggtg gccccggact ctcccgcctc gccggcctcc gacggccccg | 1500 |
| cgctgccctc gcccgccatc ccgcgtggcg gcttcgcgct cagcctcctg gccaatgccg | 1560 |
| aggcggccgc gcgggcgccc aagggcggcg gcctcttcca gcggggcgcg ctgctgggca | 1620 |
| agctgcggcg cgcagagtcg cgcacgtccg tgggtagcca gcgcagccgt gtcagcttcc | 1680 |
| ttaagagcga cctcagctcg ggcgccagcg acgccgcgtc caccgccagc ctgggagagg | 1740 |
| ccgccgaggg cgccgacgag gccgcaccct tcgaggccga tatcgacgcc accaccaccg | 1800 |
| agacctacat gggcgagtgg aagaacgaca aacgctcggg cttcggcgtg agcgaacgct | 1860 |
| ccagtggcct ccgctacgag ggcgagtggc tggacaacct gcgccacggc tatggctgca | 1920 |
| ccacgctgcc cgacggccac cgcgaggagg gcaagtaccg ccacaacgtg ctggtcaagg | 1980 |
| acaccaagcg ccgcatgctg cagctcaaga gcaacaaggt ccgccagaaa gtggagcaca | 2040 |
| gtgtggaggg tgcccagcgc gccgctgcta tcgcgcgcca gaaggccgag attgccgcct | 2100 |
| ccaggacaag ccacgccaag gccaaagctg aggcagcgga acaggccgcc ctggctgcca | 2160 |
| accaggagtc caacattgct cgcactttgg ccagggagct ggctccggac ttctaccagc | 2220 |
| caggtccgga atatcagaag cgccggctgc tgcaggagat cctggagaac tcggagagcc | 2280 |
| tgctggagcc ccccgaccgg ggcgccggcg cagcgggcct cccacagccg ccccgcgaga | 2340 |
| gcccgcagct gcacgagcgt gagacccctc ggcccgaggg tggctccccg tcaccggccg | 2400 |
| ggacgccccc gcagcccaag cggcccaggc ccggggtgtc caaggacggc ctgctgagcc | 2460 |
| caggcgcctg gaacggcgag cccagcggtg agggcagccg gtcagtcact ccgtccgagg | 2520 |
| gcgcgggccg ccgcagcccc gcgcgtccag ccaccgagcg catggccatc gaggctctgc | 2580 |
| aggcaccgcc tgcgccgtcg cgggagccgg aggtggcgct ttaccagggc taccacagct | 2640 |
| atgctgtggc cgccacgccg cccgagcccc caccctttga ggaccagccc gagcccgagg | 2700 |
| tctccgggtc cgagtccgcg ccctcgtccc cggccaccgc cccgctgcag gcccccacgc | 2760 |
| tccgaggccc cgagcctgca cgcgagaccc ccgccaagct ggagcccaag cccatcatcc | 2820 |
| ccaaagccga gcccagggcc aaggcccgca agactgaggc tcgagggctg accaaggcgg | 2880 |
| gggccaagaa gaaggcgcgg aaggaggccg cactggcggc agaggcggag gtggaggtgg | 2940 |
| aagaggtccc caacaccatc ctcatctgca tggtgatcct gctgaacatc ggcctggcca | 3000 |
| tcctctttgt tcacctcctg accggatccg gcagtggaga gggcagagga agtctgctaa | 3060 |
| catgcggtga cgtcgaggag aatcctggcc caatgagcaa gggcgaggag ctgttcaccg | 3120 |
| gcgtggtgcc catcctggtg gagctggacg gcgacgtgaa cggccacaag ttcagcgtga | 3180 |
| gaggcgaggg cgagggcgac gccaccaacg gcaagctgac cctgaagttc atctgcacca | 3240 |
| ccggcaagct gcccgtgccc tggcccaccc tggtgaccac cctgacctac ggcgtgctgt | 3300 |
| gcttcagcag ataccccgac cacatgaaga gacacgactt cttcaagagc gccatgcccg | 3360 |
| agggctacgt gcaggagaga accatcagct tcaaggacga cggcacctac aagaccagag | 3420 |
| ccgaggtgaa gttcgagggc gacaccctgg tgaacagaat cgagctgaag ggcatcgact | 3480 |
| tcaaggagga cggcaacatc ctgggccaca agctggagta caacttcaac agccacaacg | 3540 |
| tgtacatcac cgccgacaag cagaagaacg gcatcaaggc ctacttcaag atcagacaca | 3600 |
| acgtggagga cggcagcgtg cagctggccg accactacca gcagaacacc cccatcggcg | 3660 |
| acggccccgt gctgctgccc gacaaccact acctgagcac ccagagcgtg ctgagcaagg | 3720 |
| accccaacga gaagagagac cacatggtgc tgctggagga cgtgaccgcc gccggcatca | 3780 |
| cccacggcat ggacgagctg tacaagtgat caacctctgg attacaaaat ttgtgaaaga | 3840 |
| ttgactggta ttcttaacta tgttgctcct tttacgctat gtggatacgc tgctttaatg | 3900 |
| cctttgtatc atgctattgc ttcccgtatg gctttcattt tctcctcctt gtataaatcc | 3960 |
| tggttgctgt ctctttatga ggagttgtgg cccgttgtca ggcaacgtgg cgtggtgtgc | 4020 |
| actgtgtttg ctgacgcaac ccccactggt tggggcattg ccaccacctg tcagctcctt | 4080 |
| tccgggactt tcgctttccc cctccctatt gccacggcgg aactcatcgc cgcctgcctt | 4140 |
| gcccgctgct ggacaggggc tcggctgttg ggcactgaca attccgtggt gttgtcgggg | 4200 |
| aaatcatcgt cctttccttg gctgctcgcc tgtgttgcca cctggattct gcgcgggacg | 4260 |
| tccttctgct acgtcccttc ggccctcaat ccagcggacc ttccttcccg cggcctgctg | 4320 |
| ccggctctgc ggcctcttcc gcgtcttcgc cttcgccctc agacgagtcg gatctccctt | 4380 |
| tgggccgcct ccccgcactg cccgggtggc atccctgtga cccctcccca gtgcctctcc | 4440 |
| tggccctgga agttgccact ccagtgccca ccagccttgt cctaataaaa ttaagttgca | 4500 |
| tcattttgtc tgactaggtg tccttctata atattatggg gtggaggggg gtggtatgga | 4560 |
| gcaaggggcc caagttggga agaaacctgt agggcctgcc ctaaggagga acccctagtg | 4620 |
| atggagttgg ccactccctc tctgcgcgct cgctcgctca ctgaggccgg gcgaccaaag | 4680 |
| gtcgcccgac gcccgggctt tgcccgggcg gcctcagtga gcgagcgagc gcgcagagag | 4740 |
| ggagtggcca a | 4751 |

In a certain embodiment, the vector genome comprises, in 5′ to 3′ order, a 5′ITR; a hTnnT2 promoter; a JPH2 1mutAA (R572A and T573A) transgene; a GFP tag; an WPRE(x) element; a hGH sequence; and a 3′ ITR. The vector genome may comprise, in 5′ to 3′ order, any one of the polynucleotide sequences SEQ ID NO: 79; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length JPH2 1mutAA (R572A and T573A) transgene, i.e. a transgene encoding a JPH2 of at least 600 or at least 630 amino acids.

| SEQ ID NO: 79 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtct cagtccatta ggagccagta | 180 |
| gcctggaaga tgtctttacc cccagcatca gttcaagtgg agcagcacat aactcttgcc | 240 |
| ctctgccttc caagattctg gtgctgagac ttatggagtg tcttggaggt tgccttctgc | 300 |
| cccccaaccc tgctcccagc tggccctccc aggcctgggt tgctggcctc tgctttatca | 360 |
| ggattctcaa gagggacagc tggtttatgt tgcatgactg ttccctgcat atctgctctg | 420 |
| gttttaaata gcttatctga gcagctggag gaccacatgg gcttatatgg cgtggggtac | 480 |
| atgttcctgt agccttgtcc ctggcacctg ccaaaatagc agccaacacc ccccaccccc | 540 |
| accgccatcc ccctgcccca cccgtcccct gtcgcacatt cctccctccg cagggctggc | 600 |
| tcaccaggcc ccagcccaca tgcctgctta aagccctctc catcctctgc ctcacccagt | 660 |
| ccccgctgag actgagcaga cgcctccagg atctgtcggc aggccaccat gagtgggggc | 720 |
| cgcttcgact ttgatgatgg aggggcgtac tgcgggggct gggagggggg aaaggcccat | 780 |
| gggcatggac tgtgcacagg ccccaagggc cagggcgaat actctggctc ctggaacttt | 840 |
| ggctttgagg tggcaggtgt ctacacctgg cccagcggaa acacctttga gggatactgg | 900 |
| agccagggca aacggcatgg gctgggcata gagaccaagg ggcgctggct ctacaagggc | 960 |
| gagtggacac atggcttcaa gggacgctac ggaatccggc agagctcaag cagcggtgcc | 1020 |
| aagtatgagg gcacctggaa caatggcctg caagacggct atggcaccga gacctatgct | 1080 |
| gatggaggga cgtaccaagg ccagttcacc aacggcatgc gccatggcta cggagtacgc | 1140 |
| cagagcgtgc cctacgggat ggccgtggtg gtgcgctcgc cgctgcgcac gtcgctgtcg | 1200 |
| tccctgcgca gcgagcacag caacggcacg gtggccccgg actctcccgc ctcgccggcc | 1260 |
| tccgacggcc ccgcgctgcc ctcgcccgcc atcccgcgtg gcggcttcgc gctcagcctc | 1320 |
| ctggccaatg ccgaggcggc cgcgcgggcg cccaagggcg gcggcctctt ccagcggggc | 1380 |
| gcgctgctgg gcaagctgcg gcgcgcagag tcgcgcacgt ccgtgggtag ccagcgcagc | 1440 |
| cgtgtcagct tccttaagag cgacctcagc tcgggcgcca gcgacgccgc gtccaccgcc | 1500 |
| agcctgggag aggccgccga gggcgccgac gaggccgcac ccttcgaggc cgatatcgac | 1560 |
| gccaccacca ccgagaccta catgggcgag tggaagaacg acaaacgctc gggcttcggc | 1620 |
| gtgagcgaac gctccagtgg cctccgctac gagggcgagt ggctggacaa cctgcgccac | 1680 |
| ggctatggct gcaccacgct gcccgacggc caccgcgagg agggcaagta ccgccacaac | 1740 |
| gtgctggtca aggacaccaa gcgccgcatg ctgcagctca agagcaacaa ggtccgccag | 1800 |
| aaagtggagc acagtgtgga gggtgcccag cgcgccgctg ctatcgcgcg ccagaaggcc | 1860 |
| gagattgccg cctccaggac aagccacgcc aaggccaaag ctgaggcagc ggaacaggcc | 1920 |
| gccctggctg ccaaccagga gtccaacatt gctcgcactt tggccaggga gctggctccg | 1980 |
| gacttctacc agccaggtcc ggaatatcag aagcgccggc tgctgcagga gatcctggag | 2040 |
| aactcggaga gcctgctgga gccccccgac cggggcgccg gcgcagcggg cctcccacag | 2100 |
| ccgccccgcg agagcccgca gctgcacgag cgtgagaccc ctcggcccga gggtggctcc | 2160 |
| ccgtcaccgg ccgggacgcc cccgcagccc aagcggccca ggcccggggt gtccaaggac | 2220 |
| ggcctgctga gcccaggcgc ctggaacggc gagcccagcg gtgagggcag ccggtcagtc | 2280 |
| actccgtccg agggcgcggg ccgccgcagc cccgcgcgtc cagccaccga gcgcatggcc | 2340 |
| atcgaggctc tgcaggcacc gcctgcgccg tcgcgggagc cggaggtggc gctttaccag | 2400 |
| ggctaccaca gctatgctgt ggccgccacg ccgcccgagc ccccaccctt tgaggaccag | 2460 |
| cccgagcccg aggtctccgg gtccgagtcc gcgccctcgt ccccggccac cgccccgctg | 2520 |
| caggccccca cgctccgagg ccccgagcct gcacgcgaga cccccgccaa gctggagccc | 2580 |
| aagcccatca tccccaaagc cgagcccagg gccaaggccc gcaagactga ggctcgaggg | 2640 |
| ctgaccaagg cgggggccaa gaagaaggcg cggaaggagg ccgcactggc ggcagaggcg | 2700 |
| gaggtggagg tggaagaggt ccccaacacc atcctcatct gcatggtgat cctgctgaac | 2760 |
| atcggcctgg ccatcctctt tgttcacctc ctgaccggat ccggcagtgg agagggcaga | 2820 |
| ggaagtctgc taacatgcgg tgacgtcgag gagaatcctg gcccaatgag caagggcgag | 2880 |
| gagctgttca ccggcgtggt gcccatcctg gtggagctgg acggcgacgt gaacggccac | 2940 |
| aagttcagcg tgagaggcga gggcgagggc gacgccacca acggcaagct gaccctgaag | 3000 |
| ttcatctgca ccaccggcaa gctgcccgtg ccctggccca ccctggtgac caccctgacc | 3060 |
| tacggcgtgc tgtgcttcag cagatacccc gaccacatga agagacacga cttcttcaag | 3120 |
| agcgccatgc ccgagggcta cgtgcaggag agaaccatca gcttcaagga cgacggcacc | 3180 |
| tacaagacca gagccgaggt gaagttcgag ggcgacaccc tggtgaacag aatcgagctg | 3240 |
| aagggcatcg acttcaagga ggacggcaac atcctgggcc acaagctgga gtacaacttc | 3300 |
| aacagccaca acgtgtacat caccgccgac aagcagaaga acggcatcaa ggcctacttc | 3360 |
| aagatcagac acaacgtgga ggacggcagc gtgcagctgg ccgaccacta ccagcagaac | 3420 |
| acccccatcg gcgacggccc cgtgctgctg cccgacaacc actacctgag cacccagagc | 3480 |
| gtgctgagca aggaccccaa cgagaagaga gaccacatgg tgctgctgga ggacgtgacc | 3540 |
| gccgccggca tcacccacgg catggacgag ctgtacaagt gatcaacctc tggattacaa | 3600 |
| aatttgtgaa agattgactg gtattcttaa ctatgttgct ccttttacgc tatgtggata | 3660 |
| cgctgcttta atgcctttgt atcatgctat tgcttcccgt atggctttca ttttctcctc | 3720 |
| cttgtataaa tcctggttgc tgtctcttta tgaggagttg tggcccgttg tcaggcaacg | 3780 |
| tggcgtggtg tgcactgtgt ttgctgacgc aacccccact ggttggggca ttgccaccac | 3840 |
| ctgtcagctc ctttccggga ctttcgcttt ccccctccct attgccacgg cggaactcat | 3900 |
| cgccgcctgc cttgcccgct gctggacagg ggctcggctg ttgggcactg acaattccgt | 3960 |
| ggtgttgtcg gggaaatcat cgtcctttcc ttggctgctc gcctgtgttg ccacctggat | 4020 |
| tctgcgcggg acgtccttct gctacgtccc ttcggccctc aatccagcgg accttccttc | 4080 |
| ccgcggcctg ctgccggctc tgcggcctct tccgcgtctt cgccttcgcc ctcagacgag | 4140 |
| tcggatctcc ctttgggccg cctccccgca ctgcccgggt ggcatccctg tgacccctcc | 4200 |
| ccagtgcctc tcctggccct ggaagttgcc actccagtgc ccaccagcct tgtcctaata | 4260 |
| aaattaagtt gcatcatttt gtctgactag gtgtccttct ataatattat ggggtggagg | 4320 |
| ggggtggtat ggagcaaggg gcccaagttg ggaagaaacc tgtagggcct gccctaagga | 4380 |
| ggaaccccta gtgatggagt tggccactcc ctctctgcgc gctcgctcgc tcactgaggc | 4440 |
| cgggcgacca aaggtcgccc gacgcccggg ctttgcccgg gcggcctcag tgagcgagcg | 4500 |
| agcgcgcaga gagggagtgg ccaa | 4524 |

In a certain embodiment, the vector genome comprises, in 5′ to 3′ order, a 5′ITR; a CMV enhancer element; a CMV promoter; a JPH2 1mutAA (R572A and T573A) transgene; a GFP tag; an WPRE(x) element; a hGH sequence; and a 3′ ITR. The vector genome may comprise, in 5′ to 3′ order, any one of the polynucleotide sequences SEQ ID NO: 80; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length 1mutAA (R572A and T573A) transgene, i.e. a transgene encoding a JPH2 of at least 600 or at least 630 amino acids.

| SEQ ID NO: 80 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtta catacgttac ataacttacg | 180 |
| gtaaatggcc cgcctggctg accgcccaac gacccccgcc cattgacgtc aataatgacg | 240 |
| tatgttccca tagtaacgcc aatagggact ttccattgac gtcaatgggt ggagtattta | 300 |
| cggtaaactg cccacttggc agtacatcaa gtgtatcata tgccaagtac gccccctatt | 360 |
| gacgtcaatg acggtaaatg gcccgcctgg cattatgccc agtacatgac cttatgggac | 420 |
| tttcctactt ggcagtacat ctacgtatta gtcatcgcta ttaccatggt gatgcggttt | 480 |
| tggcagtaca tcaatgggcg tggatagcgg tttgactcac ggggatttcc aagtctccac | 540 |
| cccattgacg tcaatgggag tttgttttgg caccaaaatc aacgggactt tccaaaatgt | 600 |
| cgtaacaact ccgccccatt gacgcaaatg ggcggtaggc gtgtacggtg ggaggtctat | 660 |
| ataagcagag ctcgtttagt gaaccgtcag atcgcctgga gacgccatcc acgctgtttt | 720 |
| gacctccata gaagacaccg ggaccgatcc agcctccgcg ggccaccatg agtgggggcc | 780 |
| gcttcgactt tgatgatgga ggggcgtact gcgggggctg ggagggggga aaggcccatg | 840 |
| ggcatggact gtgcacaggc cccaagggcc agggcgaata ctctggctcc tggaactttg | 900 |
| gctttgaggt ggcaggtgtc tacacctggc ccagcggaaa cacctttgag ggatactgga | 960 |
| gccagggcaa acggcatggg ctgggcatag agaccaaggg gcgctggctc tacaagggcg | 1020 |
| agtggacaca tggcttcaag ggacgctacg gaatccggca gagctcaagc agcggtgcca | 1080 |
| agtatgaggg cacctggaac aatggcctgc aagacggcta tggcaccgag acctatgctg | 1140 |
| atggagggac gtaccaaggc cagttcacca acggcatgcg ccatggctac ggagtacgcc | 1200 |
| agagcgtgcc ctacgggatg gccgtggtgg tgcgctcgcc gctgcgcacg tcgctgtcgt | 1260 |
| ccctgcgcag cgagcacagc aacggcacgg tggccccgga ctctcccgcc tcgccggcct | 1320 |
| ccgacggccc cgcgctgccc tcgcccgcca tcccgcgtgg cggcttcgcg ctcagcctcc | 1380 |
| tggccaatgc cgaggcggcc gcgcgggcgc ccaagggcgg cggcctcttc cagcggggcg | 1440 |
| cgctgctggg caagctgcgg cgcgcagagt cgcgcacgtc cgtgggtagc cagcgcagcc | 1500 |
| gtgtcagctt ccttaagagc gacctcagct cgggcgccag cgacgccgcg tccaccgcca | 1560 |
| gcctgggaga ggccgccgag ggcgccgacg aggccgcacc cttcgaggcc gatatcgacg | 1620 |
| ccaccaccac cgagacctac atgggcgagt ggaagaacga caaacgctcg ggcttcggcg | 1680 |
| tgagcgaacg ctccagtggc ctccgctacg agggcgagtg gctggacaac ctgcgccacg | 1740 |
| gctatggctg caccacgctg cccgacggcc accgcgagga gggcaagtac cgccacaacg | 1800 |
| tgctggtcaa ggacaccaag cgccgcatgc tgcagctcaa gagcaacaag gtccgccaga | 1860 |
| aagtggagca cagtgtggag ggtgcccagc gcgccgctgc tatcgcgcgc cagaaggccg | 1920 |
| agattgccgc ctccaggaca agccacgcca aggccaaagc tgaggcagcg gaacaggccg | 1980 |
| ccctggctgc caaccaggag tccaacattg ctcgcacttt ggccagggag ctggctccgg | 2040 |
| acttctacca gccaggtccg gaatatcaga agcgccggct gctgcaggag atcctggaga | 2100 |
| actcggagag cctgctggag ccccccgacc ggggcgccgg cgcagcgggc ctcccacagc | 2160 |
| cgccccgcga gagcccgcag ctgcacgagc gtgagacccc tcggcccgag ggtggctccc | 2220 |
| cgtcaccggc cgggacgccc ccgcagccca agcggcccag gcccggggtg tccaaggacg | 2280 |
| gcctgctgag cccaggcgcc tggaacggcg agcccagcgg tgagggcagc cggtcagtca | 2340 |
| ctccgtccga gggcgcgggc cgccgcagcc ccgcgcgtcc agccaccgag cgcatggcca | 2400 |
| tcgaggctct gcaggcaccg cctgcgccgt cgcgggagcc ggaggtggcg ctttaccagg | 2460 |
| gctaccacag ctatgctgtg gccgccacgc cgcccgagcc cccacccttt gaggaccagc | 2520 |
| ccgagcccga ggtctccggg tccgagtccg cgccctcgtc cccggccacc gccccgctgc | 2580 |
| aggcccccac gctccgaggc cccgagcctg cacgcgagac ccccgccaag ctggagccca | 2640 |
| agcccatcat ccccaaagcc gagcccaggg ccaaggcccg caagactgag gctcgagggc | 2700 |
| tgaccaaggc gggggccaag aagaaggcgc ggaaggaggc cgcactggcg gcagaggcgg | 2760 |
| aggtggaggt ggaagaggtc cccaacacca tcctcatctg catggtgatc ctgctgaaca | 2820 |
| tcggcctggc catcctcttt gttcacctcc tgaccggatc cggcagtgga gagggcagag | 2880 |
| gaagtctgct aacatgcggt gacgtcgagg agaatcctgg cccaatgagc aagggcgagg | 2940 |
| agctgttcac cggcgtggtg cccatcctgg tggagctgga cggcgacgtg aacggccaca | 3000 |
| agttcagcgt gagaggcgag ggcgagggcg acgccaccaa cggcaagctg accctgaagt | 3060 |
| tcatctgcac caccggcaag ctgcccgtgc cctggcccac cctggtgacc accctgacct | 3120 |
| acggcgtgct gtgcttcagc agataccccg accacatgaa gagacacgac ttcttcaaga | 3180 |
| gcgccatgcc cgagggctac gtgcaggaga gaaccatcag cttcaaggac gacggcacct | 3240 |
| acaagaccag agccgaggtg aagttcgagg gcgacaccct ggtgaacaga atcgagctga | 3300 |
| agggcatcga cttcaaggag gacggcaaca tcctgggcca caagctggag tacaacttca | 3360 |
| acagccacaa cgtgtacatc accgccgaca agcagaagaa cggcatcaag gcctacttca | 3420 |
| agatcagaca caacgtggag gacggcagcg tgcagctggc cgaccactac cagcagaaca | 3480 |
| cccccatcgg cgacggcccc gtgctgctgc ccgacaacca ctacctgagc acccagagcg | 3540 |
| tgctgagcaa ggaccccaac gagaagagag accacatggt gctgctggag gacgtgaccg | 3600 |
| ccgccggcat cacccacggc atggacgagc tgtacaagtg atcaacctct ggattacaaa | 3660 |
| atttgtgaaa gattgactgg tattcttaac tatgttgctc cttttacgct atgtggatac | 3720 |
| gctgctttaa tgcctttgta tcatgctatt gcttcccgta tggctttcat tttctcctcc | 3780 |
| ttgtataaat cctggttgct gtctctttat gaggagttgt ggcccgttgt caggcaacgt | 3840 |
| ggcgtggtgt gcactgtgtt tgctgacgca acccccactg gttggggcat tgccaccacc | 3900 |
| tgtcagctcc tttccgggac tttcgctttc cccctcccta ttgccacggc ggaactcatc | 3960 |
| gccgcctgcc ttgcccgctg ctggacaggg gctcggctgt tgggcactga caattccgtg | 4020 |
| gtgttgtcgg ggaaatcatc gtcctttcct tggctgctcg cctgtgttgc cacctggatt | 4080 |
| ctgcgcggga cgtccttctg ctacgtccct tcggccctca atccagcgga ccttccttcc | 4140 |
| cgcggcctgc tgccggctct gcggcctctt ccgcgtcttc gccttcgccc tcagacgagt | 4200 |
| cggatctccc tttgggccgc ctccccgcac tgcccgggtg gcatccctgt gacccctccc | 4260 |
| cagtgcctct cctggccctg gaagttgcca ctccagtgcc caccagcctt gtcctaataa | 4320 |
| aattaagttg catcattttg tctgactagg tgtccttcta taatattatg gggtggaggg | 4380 |
| gggtggtatg gagcaagggg cccaagttgg gaagaaacct gtagggcctg ccctaaggag | 4440 |
| gaacccctag tgatggagtt ggccactccc tctctgcgcg ctcgctcgct cactgaggcc | 4500 |
| gggcgaccaa aggtcgcccg acgcccgggc tttgcccggg cggcctcagt gagcgagcga | 4560 |
| gcgcgcagag agggagtggc caa | 4583 |

In a certain embodiment, the vector genome comprises, in 5′ to 3′ order, a 5′ITR; an MHCK7 promoter; a JPH2 1mutKS (R572K and T573S) transgene; an WPRE(x) element; a hGH sequence; and a 3′ ITR. The vector genome may comprise, in 5′ to 3′ order, the polynucleotide sequences SEQ ID NO: 81; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length 1mutKS (R572K and T573S) transgene, i.e. a transgene encoding a JPH2 of at least 600 or at least 630 amino acids.

| SEQ ID NO: 81 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtac ccttcagatt aaaaataact | 180 |
| gaggtaaggg cctgggtagg ggaggtggtg tgagacgctc ctgtctctcc tctatctgcc | 240 |
| catcggccct ttggggagga ggaatgtgcc caaggactaa aaaaaggcca tggagccaga | 300 |
| ggggcgaggg caacagacct ttcatgggca aaccttgggg ccctgctgtc tagcatgccc | 360 |
| cactacgggt ctaggctgcc catgtaagga ggcaaggcct ggggacaccc gagatgcctg | 420 |
| gttataatta acccagacat gtggctgccc cccccccccc aacacctgct gcctctaaaa | 480 |
| ataaccctgt ccctggtgga tcccctgcat gcgaagatct tcgaacaagg ctgtggggga | 540 |
| ctgagggcag gctgtaacag gcttgggggc cagggcttat acgtgcctgg gactcccaaa | 600 |
| gtattactgt tccatgttcc cggcgaaggg ccagctgtcc cccgccagct agactcagca | 660 |
| cttagtttag gaaccagtga gcaagtcagc ccttggggca gcccatacaa ggccatgggg | 720 |
| ctgggcaagc tgcacgcctg ggtccggggt gggcacggtg cccgggcaac gagctgaaag | 780 |
| ctcatctgct ctcaggggcc cctccctggg gacagcccct cctggctagt cacaccctgt | 840 |
| aggctcctct atataaccca ggggcacagg ggctgccctc attctaccac cacctccaca | 900 |
| gcacagacag acactcagga gccagccagg gtaagtttag tctttttgtc ttttatttca | 960 |
| ggtcccggat ccggtggtgg tgcaaatcaa agaactgctc ctcagtggat gttgccttta | 1020 |
| cttctaggcc tgtacggaag tgttacttct gctctaaaag ctgcggaatt gtacccgcgc | 1080 |
| caccatgagt gggggccgct tcgactttga tgatggaggg gcgtactgcg ggggctggga | 1140 |
| ggggggaaag gcccatgggc atggactgtg cacaggcccc aagggccagg gcgaatactc | 1200 |
| tggctcctgg aactttggct ttgaggtggc aggtgtctac acctggccca gcggaaacac | 1260 |
| ctttgaggga tactggagcc agggcaaacg gcatgggctg ggcatagaga ccaaggggcg | 1320 |
| ctggctctac aagggcgagt ggacacatgg cttcaaggga cgctacggaa tccggcagag | 1380 |
| ctcaagcagc ggtgccaagt atgagggcac ctggaacaat ggcctgcaag acggctatgg | 1440 |
| caccgagacc tatgctgatg gagggacgta ccaaggccag ttcaccaacg gcatgcgcca | 1500 |
| tggctacgga gtacgccaga gcgtgcccta cgggatggcc gtggtggtgc gctcgccgct | 1560 |
| gcgcacgtcg ctgtcgtccc tgcgcagcga gcacagcaac ggcacggtgg ccccggactc | 1620 |
| tcccgcctcg ccggcctccg acggccccgc gctgccctcg cccgccatcc cgcgtggcgg | 1680 |
| cttcgcgctc agcctcctgg ccaatgccga ggcggccgcg cgggcgccca agggcggcgg | 1740 |
| cctcttccag cggggcgcgc tgctgggcaa gctgcggcgc gcagagtcgc gcacgtccgt | 1800 |
| gggtagccag cgcagccgtg tcagcttcct taagagcgac ctcagctcgg gcgccagcga | 1860 |
| cgccgcgtcc accgccagcc tgggagaggc cgccgagggc gccgacgagg ccgcaccctt | 1920 |
| cgaggccgat atcgacgcca ccaccaccga gacctacatg ggcgagtgga agaacgacaa | 1980 |
| acgctcgggc ttcggcgtga gcgaacgctc cagtggcctc cgctacgagg gcgagtggct | 2040 |
| ggacaacctg cgccacggct atggctgcac cacgctgccc gacggccacc gcgaggaggg | 2100 |
| caagtaccgc cacaacgtgc tggtcaagga caccaagcgc cgcatgctgc agctcaagag | 2160 |
| caacaaggtc cgccagaaag tggagcacag tgtggagggt gcccagcgcg ccgctgctat | 2220 |
| cgcgcgccag aaggccgaga ttgccgcctc caggacaagc cacgccaagg ccaaagctga | 2280 |
| ggcagcggaa caggccgccc tggctgccaa ccaggagtcc aacattgctc gcactttggc | 2340 |
| cagggagctg gctccggact tctaccagcc aggtccggaa tatcagaagc gccggctgct | 2400 |
| gcaggagatc ctggagaact cggagagcct gctggagccc cccgaccggg gcgccggcgc | 2460 |
| agcgggcctc ccacagccgc cccgcgagag cccgcagctg cacgagcgtg agacccctcg | 2520 |
| gcccgagggt ggctccccgt caccggccgg gacgcccccg cagcccaagc ggcccaggcc | 2580 |
| cggggtgtcc aaggacggcc tgctgagccc aggcgcctgg aacggcgagc ccagcggtga | 2640 |
| gggcagccgg tcagtcactc cgtccgaggg cgcgggccgc cgcagccccg cgcgtccagc | 2700 |
| caccgagcgc atggccatcg aggctctgca ggcaccgcct gcgccgtcgc gggagccgga | 2760 |
| ggtggcgctt taccagggct accacagcta tgctgtgaag agcacgccgc ccgagccccc | 2820 |
| accctttgag gaccagcccg agcccgaggt ctccgggtcc gagtccgcgc cctcgtcccc | 2880 |
| ggccaccgcc ccgctgcagg cccccacgct ccgaggcccc gagcctgcac gcgagacccc | 2940 |
| cgccaagctg gagcccaagc ccatcatccc caaagccgag cccagggcca aggcccgcaa | 3000 |
| gactgaggct cgagggctga ccaaggcggg ggccaagaag aaggcgcgga aggaggccgc | 3060 |
| actggcggca gaggcggagg tggaggtgga agaggtcccc aacaccatcc tcatctgcat | 3120 |
| ggtgatcctg ctgaacatcg gcctggccat cctctttgtt cacctcctga cctgatcaac | 3180 |
| ctctggatta caaaatttgt gaaagattga ctggtattct taactatgtt gctcctttta | 3240 |
| cgctatgtgg atacgctgct ttaatgcctt tgtatcatgc tattgcttcc cgtatggctt | 3300 |
| tcattttctc ctccttgtat aaatcctggt tgctgtctct ttatgaggag ttgtggcccg | 3360 |
| ttgtcaggca acgtggcgtg gtgtgcactg tgtttgctga cgcaaccccc actggttggg | 3420 |
| gcattgccac cacctgtcag ctcctttccg ggactttcgc tttccccctc cctattgcca | 3480 |
| cggcggaact catcgccgcc tgccttgccc gctgctggac aggggctcgg ctgttgggca | 3540 |
| ctgacaattc cgtggtgttg tcggggaaat catcgtcctt tccttggctg ctcgcctgtg | 3600 |
| ttgccacctg gattctgcgc gggacgtcct tctgctacgt cccttcggcc ctcaatccag | 3660 |
| cggaccttcc ttcccgcggc ctgctgccgg ctctgcggcc tcttccgcgt cttcgccttc | 3720 |
| gccctcagac gagtcggatc tccctttggg ccgcctcccc gcactgcccg ggtggcatcc | 3780 |
| ctgtgacccc tccccagtgc ctctcctggc cctggaagtt gccactccag tgcccaccag | 3840 |
| ccttgtccta ataaaattaa gttgcatcat tttgtctgac taggtgtcct tctataatat | 3900 |
| tatggggtgg aggggggtgg tatggagcaa ggggcccaag ttgggaagaa acctgtaggg | 3960 |
| cctgccctaa ggaggaaccc ctagtgatgg agttggccac tccctctctg cgcgctcgct | 4020 |
| cgctcactga ggccgggcga ccaaaggtcg cccgacgccc gggctttgcc cgggcggcct | 4080 |
| cagtgagcga gcgagcgcgc agagagggag tggccaa | 4117 |

In a certain embodiment, the vector genome comprises, in 5′ to 3′ order, a 5′ITR; a hTnnT2 promoter; a JPH2 1mutKS (R572K and T573S) transgene; an WPRE(x) element; a hGH sequence; and a 3′ ITR. The vector genome may comprise, in 5′ to 3′ order, any one of the polynucleotide sequences SEQ ID NO: 82; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length JPH2 1mutKS (R572K and T573S) transgene, i.e. a transgene encoding a JPH2 of at least 600 or at least 630 amino acids.

| SEQ ID NO: 82 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtct cagtccatta ggagccagta | 180 |
| gcctggaaga tgtctttacc cccagcatca gttcaagtgg agcagcacat aactcttgcc | 240 |
| ctctgccttc caagattctg gtgctgagac ttatggagtg tcttggaggt tgccttctgc | 300 |
| cccccaaccc tgctcccagc tggccctccc aggcctgggt tgctggcctc tgctttatca | 360 |
| ggattctcaa gagggacagc tggtttatgt tgcatgactg ttccctgcat atctgctctg | 420 |
| gttttaaata gcttatctga gcagctggag gaccacatgg gcttatatgg cgtggggtac | 480 |
| atgttcctgt agccttgtcc ctggcacctg ccaaaatagc agccaacacc ccccaccccc | 540 |
| accgccatcc ccctgcccca cccgtcccct gtcgcacatt cctccctccg cagggctggc | 600 |
| tcaccaggcc ccagcccaca tgcctgctta aagccctctc catcctctgc ctcacccagt | 660 |
| ccccgctgag actgagcaga cgcctccagg atctgtcggc aggccaccat gagtgggggc | 720 |
| cgcttcgact ttgatgatgg aggggcgtac tgcgggggct gggagggggg aaaggcccat | 780 |
| gggcatggac tgtgcacagg ccccaagggc cagggcgaat actctggctc ctggaacttt | 840 |
| ggctttgagg tggcaggtgt ctacacctgg cccagcggaa acacctttga gggatactgg | 900 |
| agccagggca aacggcatgg gctgggcata gagaccaagg ggcgctggct ctacaagggc | 960 |
| gagtggacac atggcttcaa gggacgctac ggaatccggc agagctcaag cagcggtgcc | 1020 |
| aagtatgagg gcacctggaa caatggcctg caagacggct atggcaccga gacctatgct | 1080 |
| gatggaggga cgtaccaagg ccagttcacc aacggcatgc gccatggcta cggagtacgc | 1140 |
| cagagcgtgc cctacgggat ggccgtggtg gtgcgctcgc cgctgcgcac gtcgctgtcg | 1200 |
| tccctgcgca gcgagcacag caacggcacg gtggccccgg actctcccgc ctcgccggcc | 1260 |
| tccgacggcc ccgcgctgcc ctcgcccgcc atcccgcgtg gcggcttcgc gctcagcctc | 1320 |
| ctggccaatg ccgaggcggc cgcgcgggcg cccaagggcg gcggcctctt ccagcggggc | 1380 |
| gcgctgctgg gcaagctgcg gcgcgcagag tcgcgcacgt ccgtgggtag ccagcgcagc | 1440 |
| cgtgtcagct tccttaagag cgacctcagc tcgggcgcca gcgacgccgc gtccaccgcc | 1500 |
| agcctgggag aggccgccga gggcgccgac gaggccgcac ccttcgaggc cgatatcgac | 1560 |
| gccaccacca ccgagaccta catgggcgag tggaagaacg acaaacgctc gggcttcggc | 1620 |
| gtgagcgaac gctccagtgg cctccgctac gagggcgagt ggctggacaa cctgcgccac | 1680 |
| ggctatggct gcaccacgct gcccgacggc caccgcgagg agggcaagta ccgccacaac | 1740 |
| gtgctggtca aggacaccaa gcgccgcatg ctgcagctca agagcaacaa ggtccgccag | 1800 |
| aaagtggagc acagtgtgga gggtgcccag cgcgccgctg ctatcgcgcg ccagaaggcc | 1860 |
| gagattgccg cctccaggac aagccacgcc aaggccaaag ctgaggcagc ggaacaggcc | 1920 |
| gccctggctg ccaaccagga gtccaacatt gctcgcactt tggccaggga gctggctccg | 1980 |
| gacttctacc agccaggtcc ggaatatcag aagcgccggc tgctgcagga gatcctggag | 2040 |
| aactcggaga gcctgctgga gccccccgac cggggcgccg gcgcagcggg cctcccacag | 2100 |
| ccgccccgcg agagcccgca gctgcacgag cgtgagaccc ctcggcccga gggtggctcc | 2160 |
| ccgtcaccgg ccgggacgcc cccgcagccc aagcggccca ggcccggggt gtccaaggac | 2220 |
| ggcctgctga gcccaggcgc ctggaacggc gagcccagcg gtgagggcag ccggtcagtc | 2280 |
| actccgtccg agggcgcggg ccgccgcagc cccgcgcgtc cagccaccga gcgcatggcc | 2340 |
| atcgaggctc tgcaggcacc gcctgcgccg tcgcgggagc cggaggtggc gctttaccag | 2400 |
| ggctaccaca gctatgctgt gaagagcacg ccgcccgagc ccccaccctt tgaggaccag | 2460 |
| cccgagcccg aggtctccgg gtccgagtcc gcgccctcgt ccccggccac cgccccgctg | 2520 |
| caggccccca cgctccgagg ccccgagcct gcacgcgaga cccccgccaa gctggagccc | 2580 |
| aagcccatca tccccaaagc cgagcccagg gccaaggccc gcaagactga ggctcgaggg | 2640 |
| ctgaccaagg cgggggccaa gaagaaggcg cggaaggagg ccgcactggc ggcagaggcg | 2700 |
| gaggtggagg tggaagaggt ccccaacacc atcctcatct gcatggtgat cctgctgaac | 2760 |
| atcggcctgg ccatcctctt tgttcacctc ctgacctgat caacctctgg attacaaaat | 2820 |
| ttgtgaaaga ttgactggta ttcttaacta tgttgctcct tttacgctat gtggatacgc | 2880 |
| tgctttaatg cctttgtatc atgctattgc ttcccgtatg gctttcattt tctcctcctt | 2940 |
| gtataaatcc tggttgctgt ctctttatga ggagttgtgg cccgttgtca ggcaacgtgg | 3000 |
| cgtggtgtgc actgtgtttg ctgacgcaac ccccactggt tggggcattg ccaccacctg | 3060 |
| tcagctcctt tccgggactt tcgctttccc cctccctatt gccacggcgg aactcatcgc | 3120 |
| cgcctgcctt gcccgctgct ggacaggggc tcggctgttg ggcactgaca attccgtggt | 3180 |
| gttgtcgggg aaatcatcgt cctttccttg gctgctcgcc tgtgttgcca cctggattct | 3240 |
| gcgcgggacg tccttctgct acgtcccttc ggccctcaat ccagcggacc ttccttcccg | 3300 |
| cggcctgctg ccggctctgc ggcctcttcc gcgtcttcgc cttcgccctc agacgagtcg | 3360 |
| gatctccctt tgggccgcct ccccgcactg cccgggtggc atccctgtga cccctcccca | 3420 |
| gtgcctctcc tggccctgga agttgccact ccagtgccca ccagccttgt cctaataaaa | 3480 |
| ttaagttgca tcattttgtc tgactaggtg tccttctata atattatggg gtggaggggg | 3540 |
| gtggtatgga gcaaggggcc caagttggga agaaacctgt agggcctgcc ctaaggagga | 3600 |
| acccctagtg atggagttgg ccactccctc tctgcgcgct cgctcgctca ctgaggccgg | 3660 |
| gcgaccaaag gtcgcccgac gcccgggctt tgcccgggcg gcctcagtga gcgagcgagc | 3720 |
| gcgcagagag ggagtggcca a | 3741 |

In a certain embodiment, the vector genome comprises, in 5′ to 3′ order, a 5′ITR; an MHCK7 promoter; a JPH2 1mutKS (R572K and T573S) transgene; a GFP tag; an WPRE(x) element; a hGH sequence; and a 3′ ITR. The vector genome may comprise, in 5′ to 3′ order, the polynucleotide sequences SEQ ID NO: 83; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length 1mutKS (R572K and T573S) transgene, i.e., a transgene encoding a JPH2 of at least 600 or at least 630 amino acids.

| SEQ ID NO: 83 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtac ccttcagatt aaaaataact | 180 |
| gaggtaaggg cctgggtagg ggaggtggtg tgagacgctc ctgtctctcc tctatctgcc | 240 |
| catcggccct ttggggagga ggaatgtgcc caaggactaa aaaaaggcca tggagccaga | 300 |
| ggggcgaggg caacagacct ttcatgggca aaccttgggg ccctgctgtc tagcatgccc | 360 |
| cactacgggt ctaggctgcc catgtaagga ggcaaggcct ggggacaccc gagatgcctg | 420 |
| gttataatta acccagacat gtggctgccc cccccccccc aacacctgct gcctctaaaa | 480 |
| ataaccctgt ccctggtgga tcccctgcat gcgaagatct tcgaacaagg ctgtggggga | 540 |
| ctgagggcag gctgtaacag gcttgggggc cagggcttat acgtgcctgg gactcccaaa | 600 |
| gtattactgt tccatgttcc cggcgaaggg ccagctgtcc cccgccagct agactcagca | 660 |
| cttagtttag gaaccagtga gcaagtcagc ccttggggca gcccatacaa ggccatgggg | 720 |
| ctgggcaagc tgcacgcctg ggtccggggt gggcacggtg cccgggcaac gagctgaaag | 780 |
| ctcatctgct ctcaggggcc cctccctggg gacagcccct cctggctagt cacaccctgt | 840 |
| aggctcctct atataaccca ggggcacagg ggctgccctc attctaccac cacctccaca | 900 |
| gcacagacag acactcagga gccagccagg ccaccatgag tgggggccgc ttcgactttg | 960 |
| atgatggagg ggcgtactgc gggggctggg aggggggaaa ggcccatggg catggactgt | 1020 |
| gcacaggccc caagggccag ggcgaatact ctggctcctg gaactttggc tttgaggtgg | 1080 |
| caggtgtcta cacctggccc agcggaaaca cctttgaggg atactggagc cagggcaaac | 1140 |
| ggcatgggct gggcatagag accaaggggc gctggctcta caagggcgag tggacacatg | 1200 |
| gcttcaaggg acgctacgga atccggcaga gctcaagcag cggtgccaag tatgagggca | 1260 |
| cctggaacaa tggcctgcaa gacggctatg gcaccgagac ctatgctgat ggagggacgt | 1320 |
| accaaggcca gttcaccaac ggcatgcgcc atggctacgg agtacgccag agcgtgccct | 1380 |
| acgggatggc cgtggtggtg cgctcgccgc tgcgcacgtc gctgtcgtcc ctgcgcagcg | 1440 |
| agcacagcaa cggcacggtg gccccggact ctcccgcctc gccggcctcc gacggccccg | 1500 |
| cgctgccctc gcccgccatc ccgcgtggcg gcttcgcgct cagcctcctg gccaatgccg | 1560 |
| aggcggccgc gcgggcgccc aagggcggcg gcctcttcca gcggggcgcg ctgctgggca | 1620 |
| agctgcggcg cgcagagtcg cgcacgtccg tgggtagcca gcgcagccgt gtcagcttcc | 1680 |
| ttaagagcga cctcagctcg ggcgccagcg acgccgcgtc caccgccagc ctgggagagg | 1740 |
| ccgccgaggg cgccgacgag gccgcaccct tcgaggccga tatcgacgcc accaccaccg | 1800 |
| agacctacat gggcgagtgg aagaacgaca aacgctcggg cttcggcgtg agcgaacgct | 1860 |
| ccagtggcct ccgctacgag ggcgagtggc tggacaacct gcgccacggc tatggctgca | 1920 |
| ccacgctgcc cgacggccac cgcgaggagg gcaagtaccg ccacaacgtg ctggtcaagg | 1980 |
| acaccaagcg ccgcatgctg cagctcaaga gcaacaaggt ccgccagaaa gtggagcaca | 2040 |
| gtgtggaggg tgcccagcgc gccgctgcta tcgcgcgcca gaaggccgag attgccgcct | 2100 |
| ccaggacaag ccacgccaag gccaaagctg aggcagcgga acaggccgcc ctggctgcca | 2160 |
| accaggagtc caacattgct cgcactttgg ccagggagct ggctccggac ttctaccagc | 2220 |
| caggtccgga atatcagaag cgccggctgc tgcaggagat cctggagaac tcggagagcc | 2280 |
| tgctggagcc ccccgaccgg ggcgccggcg cagcgggcct cccacagccg ccccgcgaga | 2340 |
| gcccgcagct gcacgagcgt gagacccctc ggcccgaggg tggctccccg tcaccggccg | 2400 |
| ggacgccccc gcagcccaag cggcccaggc ccggggtgtc caaggacggc ctgctgagcc | 2460 |
| caggcgcctg gaacggcgag cccagcggtg agggcagccg gtcagtcact ccgtccgagg | 2520 |
| gcgcgggccg ccgcagcccc gcgcgtccag ccaccgagcg catggccatc gaggctctgc | 2580 |
| aggcaccgcc tgcgccgtcg cgggagccgg aggtggcgct ttaccagggc taccacagct | 2640 |
| atgctgtgaa gagcacgccg cccgagcccc caccctttga ggaccagccc gagcccgagg | 2700 |
| tctccgggtc cgagtccgcg ccctcgtccc cggccaccgc cccgctgcag gcccccacgc | 2760 |
| tccgaggccc cgagcctgca cgcgagaccc ccgccaagct ggagcccaag cccatcatcc | 2820 |
| ccaaagccga gcccagggcc aaggcccgca agactgaggc tcgagggctg accaaggcgg | 2880 |
| gggccaagaa gaaggcgcgg aaggaggccg cactggcggc agaggcggag gtggaggtgg | 2940 |
| aagaggtccc caacaccatc ctcatctgca tggtgatcct gctgaacatc ggcctggcca | 3000 |
| tcctctttgt tcacctcctg accggatccg gcagtggaga gggcagagga agtctgctaa | 3060 |
| catgcggtga cgtcgaggag aatcctggcc caatgagcaa gggcgaggag ctgttcaccg | 3120 |
| gcgtggtgcc catcctggtg gagctggacg gcgacgtgaa cggccacaag ttcagcgtga | 3180 |
| gaggcgaggg cgagggcgac gccaccaacg gcaagctgac cctgaagttc atctgcacca | 3240 |
| ccggcaagct gcccgtgccc tggcccaccc tggtgaccac cctgacctac ggcgtgctgt | 3300 |
| gcttcagcag ataccccgac cacatgaaga gacacgactt cttcaagagc gccatgcccg | 3360 |
| agggctacgt gcaggagaga accatcagct tcaaggacga cggcacctac aagaccagag | 3420 |
| ccgaggtgaa gttcgagggc gacaccctgg tgaacagaat cgagctgaag ggcatcgact | 3480 |
| tcaaggagga cggcaacatc ctgggccaca agctggagta caacttcaac agccacaacg | 3540 |
| tgtacatcac cgccgacaag cagaagaacg gcatcaaggc ctacttcaag atcagacaca | 3600 |
| acgtggagga cggcagcgtg cagctggccg accactacca gcagaacacc cccatcggcg | 3660 |
| acggccccgt gctgctgccc gacaaccact acctgagcac ccagagcgtg ctgagcaagg | 3720 |
| accccaacga gaagagagac cacatggtgc tgctggagga cgtgaccgcc gccggcatca | 3780 |
| cccacggcat ggacgagctg tacaagtgat caacctctgg attacaaaat ttgtgaaaga | 3840 |
| ttgactggta ttcttaacta tgttgctcct tttacgctat gtggatacgc tgctttaatg | 3900 |
| cctttgtatc atgctattgc ttcccgtatg gctttcattt tctcctcctt gtataaatcc | 3960 |
| tggttgctgt ctctttatga ggagttgtgg cccgttgtca ggcaacgtgg cgtggtgtgc | 4020 |
| actgtgtttg ctgacgcaac ccccactggt tggggcattg ccaccacctg tcagctcctt | 4080 |
| tccgggactt tcgctttccc cctccctatt gccacggcgg aactcatcgc cgcctgcctt | 4140 |
| gcccgctgct ggacaggggc tcggctgttg ggcactgaca attccgtggt gttgtcgggg | 4200 |
| aaatcatcgt cctttccttg gctgctcgcc tgtgttgcca cctggattct gcgcgggacg | 4260 |
| tccttctgct acgtcccttc ggccctcaat ccagcggacc ttccttcccg cggcctgctg | 4320 |
| ccggctctgc ggcctcttcc gcgtcttcgc cttcgccctc agacgagtcg gatctccctt | 4380 |
| tgggccgcct ccccgcactg cccgggtggc atccctgtga cccctcccca gtgcctctcc | 4440 |
| tggccctgga agttgccact ccagtgccca ccagccttgt cctaataaaa ttaagttgca | 4500 |
| tcattttgtc tgactaggtg tccttctata atattatggg gtggaggggg gtggtatgga | 4560 |
| gcaaggggcc caagttggga agaaacctgt agggcctgcc ctaaggagga acccctagtg | 4620 |
| atggagttgg ccactccctc tctgcgcgct cgctcgctca ctgaggccgg gcgaccaaag | 4680 |
| gtcgcccgac gcccgggctt tgcccgggcg gcctcagtga gcgagcgagc gcgcagagag | 4740 |
| ggagtggcca a | 4751 |

In a certain embodiment, the vector genome comprises, in 5′ to 3′ order, a 5′ITR; a hTnnT2 promoter; a JPH2 1mutKS (R572K and T573S) transgene; a GFP tag; an WPRE(x) element; a hGH sequence; and a 3′ ITR. The vector genome may comprise, in 5′ to 3′ order, any one of the polynucleotide sequences SEQ ID NO: 84; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length JPH2 1mutKS (R572K and T573S) transgene, i.e. a transgene encoding a JPH2 of at least 600 or at least 630 amino acids.

| SEQ ID NO: 84 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtct cagtccatta ggagccagta | 180 |
| gcctggaaga tgtctttacc cccagcatca gttcaagtgg agcagcacat aactcttgcc | 240 |
| ctctgccttc caagattctg gtgctgagac ttatggagtg tcttggaggt tgccttctgc | 300 |
| cccccaaccc tgctcccagc tggccctccc aggcctgggt tgctggcctc tgctttatca | 360 |
| ggattctcaa gagggacagc tggtttatgt tgcatgactg ttccctgcat atctgctctg | 420 |
| gttttaaata gcttatctga gcagctggag gaccacatgg gcttatatgg cgtggggtac | 480 |
| atgttcctgt agccttgtcc ctggcacctg ccaaaatagc agccaacacc ccccaccccc | 540 |
| accgccatcc ccctgcccca cccgtcccct gtcgcacatt cctccctccg cagggctggc | 600 |
| tcaccaggcc ccagcccaca tgcctgctta aagccctctc catcctctgc ctcacccagt | 660 |
| ccccgctgag actgagcaga cgcctccagg atctgtcggc aggccaccat gagtgggggc | 720 |
| cgcttcgact ttgatgatgg aggggcgtac tgcgggggct gggagggggg aaaggcccat | 780 |
| gggcatggac tgtgcacagg ccccaagggc cagggcgaat actctggctc ctggaacttt | 840 |
| ggctttgagg tggcaggtgt ctacacctgg cccagcggaa acacctttga gggatactgg | 900 |
| agccagggca aacggcatgg gctgggcata gagaccaagg ggcgctggct ctacaagggc | 960 |
| gagtggacac atggcttcaa gggacgctac ggaatccggc agagctcaag cagcggtgcc | 1020 |
| aagtatgagg gcacctggaa caatggcctg caagacggct atggcaccga gacctatgct | 1080 |
| gatggaggga cgtaccaagg ccagttcacc aacggcatgc gccatggcta cggagtacgc | 1140 |
| cagagcgtgc cctacgggat ggccgtggtg gtgcgctcgc cgctgcgcac gtcgctgtcg | 1200 |
| tccctgcgca gcgagcacag caacggcacg gtggccccgg actctcccgc ctcgccggcc | 1260 |
| tccgacggcc ccgcgctgcc ctcgcccgcc atcccgcgtg gcggcttcgc gctcagcctc | 1320 |
| ctggccaatg ccgaggcggc cgcgcgggcg cccaagggcg gcggcctctt ccagcggggc | 1380 |
| gcgctgctgg gcaagctgcg gcgcgcagag tcgcgcacgt ccgtgggtag ccagcgcagc | 1440 |
| cgtgtcagct tccttaagag cgacctcagc tcgggcgcca gcgacgccgc gtccaccgcc | 1500 |
| agcctgggag aggccgccga gggcgccgac gaggccgcac ccttcgaggc cgatatcgac | 1560 |
| gccaccacca ccgagaccta catgggcgag tggaagaacg acaaacgctc gggcttcggc | 1620 |
| gtgagcgaac gctccagtgg cctccgctac gagggcgagt ggctggacaa cctgcgccac | 1680 |
| ggctatggct gcaccacgct gcccgacggc caccgcgagg agggcaagta ccgccacaac | 1740 |
| gtgctggtca aggacaccaa gcgccgcatg ctgcagctca agagcaacaa ggtccgccag | 1800 |
| aaagtggagc acagtgtgga gggtgcccag cgcgccgctg ctatcgcgcg ccagaaggcc | 1860 |
| gagattgccg cctccaggac aagccacgcc aaggccaaag ctgaggcagc ggaacaggcc | 1920 |
| gccctggctg ccaaccagga gtccaacatt gctcgcactt tggccaggga gctggctccg | 1980 |
| gacttctacc agccaggtcc ggaatatcag aagcgccggc tgctgcagga gatcctggag | 2040 |
| aactcggaga gcctgctgga gccccccgac cggggcgccg gcgcagcggg cctcccacag | 2100 |
| ccgccccgcg agagcccgca gctgcacgag cgtgagaccc ctcggcccga gggtggctcc | 2160 |
| ccgtcaccgg ccgggacgcc cccgcagccc aagcggccca ggcccggggt gtccaaggac | 2220 |
| ggcctgctga gcccaggcgc ctggaacggc gagcccagcg gtgagggcag ccggtcagtc | 2280 |
| actccgtccg agggcgcggg ccgccgcagc cccgcgcgtc cagccaccga gcgcatggcc | 2340 |
| atcgaggctc tgcaggcacc gcctgcgccg tcgcgggagc cggaggtggc gctttaccag | 2400 |
| ggctaccaca gctatgctgt gaagagcacg ccgcccgagc ccccaccctt tgaggaccag | 2460 |
| cccgagcccg aggtctccgg gtccgagtcc gcgccctcgt ccccggccac cgccccgctg | 2520 |
| caggccccca cgctccgagg ccccgagcct gcacgcgaga cccccgccaa gctggagccc | 2580 |
| aagcccatca tccccaaagc cgagcccagg gccaaggccc gcaagactga ggctcgaggg | 2640 |
| ctgaccaagg cgggggccaa gaagaaggcg cggaaggagg ccgcactggc ggcagaggcg | 2700 |
| gaggtggagg tggaagaggt ccccaacacc atcctcatct gcatggtgat cctgctgaac | 2760 |
| atcggcctgg ccatcctctt tgttcacctc ctgaccggat ccggcagtgg agagggcaga | 2820 |
| ggaagtctgc taacatgcgg tgacgtcgag gagaatcctg gcccaatgag caagggcgag | 2880 |
| gagctgttca ccggcgtggt gcccatcctg gtggagctgg acggcgacgt gaacggccac | 2940 |
| aagttcagcg tgagaggcga gggcgagggc gacgccacca acggcaagct gaccctgaag | 3000 |
| ttcatctgca ccaccggcaa gctgcccgtg ccctggccca ccctggtgac caccctgacc | 3060 |
| tacggcgtgc tgtgcttcag cagatacccc gaccacatga agagacacga cttcttcaag | 3120 |
| agcgccatgc ccgagggcta cgtgcaggag agaaccatca gcttcaagga cgacggcacc | 3180 |
| tacaagacca gagccgaggt gaagttcgag ggcgacaccc tggtgaacag aatcgagctg | 3240 |
| aagggcatcg acttcaagga ggacggcaac atcctgggcc acaagctgga gtacaacttc | 3300 |
| aacagccaca acgtgtacat caccgccgac aagcagaaga acggcatcaa ggcctacttc | 3360 |
| aagatcagac acaacgtgga ggacggcagc gtgcagctgg ccgaccacta ccagcagaac | 3420 |
| acccccatcg gcgacggccc cgtgctgctg cccgacaacc actacctgag cacccagagc | 3480 |
| gtgctgagca aggaccccaa cgagaagaga gaccacatgg tgctgctgga ggacgtgacc | 3540 |
| gccgccggca tcacccacgg catggacgag ctgtacaagt gatcaacctc tggattacaa | 3600 |
| aatttgtgaa agattgactg gtattcttaa ctatgttgct ccttttacgc tatgtggata | 3660 |
| cgctgcttta atgcctttgt atcatgctat tgcttcccgt atggctttca ttttctcctc | 3720 |
| cttgtataaa tcctggttgc tgtctcttta tgaggagttg tggcccgttg tcaggcaacg | 3780 |
| tggcgtggtg tgcactgtgt ttgctgacgc aacccccact ggttggggca ttgccaccac | 3840 |
| ctgtcagctc ctttccggga ctttcgcttt ccccctccct attgccacgg cggaactcat | 3900 |
| cgccgcctgc cttgcccgct gctggacagg ggctcggctg ttgggcactg acaattccgt | 3960 |
| ggtgttgtcg gggaaatcat cgtcctttcc ttggctgctc gcctgtgttg ccacctggat | 4020 |
| tctgcgcggg acgtccttct gctacgtccc ttcggccctc aatccagcgg accttccttc | 4080 |
| ccgcggcctg ctgccggctc tgcggcctct tccgcgtctt cgccttcgcc ctcagacgag | 4140 |
| tcggatctcc ctttgggccg cctccccgca ctgcccgggt ggcatccctg tgacccctcc | 4200 |
| ccagtgcctc tcctggccct ggaagttgcc actccagtgc ccaccagcct tgtcctaata | 4260 |
| aaattaagtt gcatcatttt gtctgactag gtgtccttct ataatattat ggggtggagg | 4320 |
| ggggtggtat ggagcaaggg gcccaagttg ggaagaaacc tgtagggcct gccctaagga | 4380 |
| ggaaccccta gtgatggagt tggccactcc ctctctgcgc gctcgctcgc tcactgaggc | 4440 |
| cgggcgacca aaggtcgccc gacgcccggg ctttgcccgg gcggcctcag tgagcgagcg | 4500 |
| agcgcgcaga gagggagtgg ccaa | 4524 |

In a certain embodiment, the vector genome comprises, in 5′ to 3′ order, a 5′ITR; a CMV enhancer element; a CMV promoter; a JPH2 1mutKS (R572K and T573S) transgene; a GFP tag; an WPRE(x) element; a hGH sequence; and a 3′ ITR. The vector genome may comprise, in 5′ to 3′ order, any one of the polynucleotide sequences SEQ ID NO: 85; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length 1mutKS (R572K and T573S) transgene, i.e. a transgene encoding a JPH2 of at least 600 or at least 630 amino acids.

| SEQ ID NO: 85 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtta catacgttac ataacttacg | 180 |
| gtaaatggcc cgcctggctg accgcccaac gacccccgcc cattgacgtc aataatgacg | 240 |
| tatgttccca tagtaacgcc aatagggact ttccattgac gtcaatgggt ggagtattta | 300 |
| cggtaaactg cccacttggc agtacatcaa gtgtatcata tgccaagtac gccccctatt | 360 |
| gacgtcaatg acggtaaatg gcccgcctgg cattatgccc agtacatgac cttatgggac | 420 |
| tttcctactt ggcagtacat ctacgtatta gtcatcgcta ttaccatggt gatgcggttt | 480 |
| tggcagtaca tcaatgggcg tggatagcgg tttgactcac ggggatttcc aagtctccac | 540 |
| cccattgacg tcaatgggag tttgttttgg caccaaaatc aacgggactt tccaaaatgt | 600 |
| cgtaacaact ccgccccatt gacgcaaatg ggcggtaggc gtgtacggtg ggaggtctat | 660 |
| ataagcagag ctcgtttagt gaaccgtcag atcgcctgga gacgccatcc acgctgtttt | 720 |
| gacctccata gaagacaccg ggaccgatcc agcctccgcg ggccaccatg agtgggggcc | 780 |
| gcttcgactt tgatgatgga ggggcgtact gcgggggctg ggagggggga aaggcccatg | 840 |
| ggcatggact gtgcacaggc cccaagggcc agggcgaata ctctggctcc tggaactttg | 900 |
| gctttgaggt ggcaggtgtc tacacctggc ccagcggaaa cacctttgag ggatactgga | 960 |
| gccagggcaa acggcatggg ctgggcatag agaccaaggg gcgctggctc tacaagggcg | 1020 |
| agtggacaca tggcttcaag ggacgctacg gaatccggca gagctcaagc agcggtgcca | 1080 |
| agtatgaggg cacctggaac aatggcctgc aagacggcta tggcaccgag acctatgctg | 1140 |
| atggagggac gtaccaaggc cagttcacca acggcatgcg ccatggctac ggagtacgcc | 1200 |
| agagcgtgcc ctacgggatg gccgtggtgg tgcgctcgcc gctgcgcacg tcgctgtcgt | 1260 |
| ccctgcgcag cgagcacagc aacggcacgg tggccccgga ctctcccgcc tcgccggcct | 1320 |
| ccgacggccc cgcgctgccc tcgcccgcca tcccgcgtgg cggcttcgcg ctcagcctcc | 1380 |
| tggccaatgc cgaggcggcc gcgcgggcgc ccaagggcgg cggcctcttc cagcggggcg | 1440 |
| cgctgctggg caagctgcgg cgcgcagagt cgcgcacgtc cgtgggtagc cagcgcagcc | 1500 |
| gtgtcagctt ccttaagagc gacctcagct cgggcgccag cgacgccgcg tccaccgcca | 1560 |
| gcctgggaga ggccgccgag ggcgccgacg aggccgcacc cttcgaggcc gatatcgacg | 1620 |
| ccaccaccac cgagacctac atgggcgagt ggaagaacga caaacgctcg ggcttcggcg | 1680 |
| tgagcgaacg ctccagtggc ctccgctacg agggcgagtg gctggacaac ctgcgccacg | 1740 |
| gctatggctg caccacgctg cccgacggcc accgcgagga gggcaagtac cgccacaacg | 1800 |
| tgctggtcaa ggacaccaag cgccgcatgc tgcagctcaa gagcaacaag gtccgccaga | 1860 |
| aagtggagca cagtgtggag ggtgcccagc gcgccgctgc tatcgcgcgc cagaaggccg | 1920 |
| agattgccgc ctccaggaca agccacgcca aggccaaagc tgaggcagcg gaacaggccg | 1980 |
| ccctggctgc caaccaggag tccaacattg ctcgcacttt ggccagggag ctggctccgg | 2040 |
| acttctacca gccaggtccg gaatatcaga agcgccggct gctgcaggag atcctggaga | 2100 |
| actcggagag cctgctggag ccccccgacc ggggcgccgg cgcagcgggc ctcccacagc | 2160 |
| cgccccgcga gagcccgcag ctgcacgagc gtgagacccc tcggcccgag ggtggctccc | 2220 |
| cgtcaccggc cgggacgccc ccgcagccca agcggcccag gcccggggtg tccaaggacg | 2280 |
| gcctgctgag cccaggcgcc tggaacggcg agcccagcgg tgagggcagc cggtcagtca | 2340 |
| ctccgtccga gggcgcgggc cgccgcagcc ccgcgcgtcc agccaccgag cgcatggcca | 2400 |
| tcgaggctct gcaggcaccg cctgcgccgt cgcgggagcc ggaggtggcg ctttaccagg | 2460 |
| gctaccacag ctatgctgtg aagagcacgc cgcccgagcc cccacccttt gaggaccagc | 2520 |
| ccgagcccga ggtctccggg tccgagtccg cgccctcgtc cccggccacc gccccgctgc | 2580 |
| aggcccccac gctccgaggc cccgagcctg cacgcgagac ccccgccaag ctggagccca | 2640 |
| agcccatcat ccccaaagcc gagcccaggg ccaaggcccg caagactgag gctcgagggc | 2700 |
| tgaccaaggc gggggccaag aagaaggcgc ggaaggaggc cgcactggcg gcagaggcgg | 2760 |
| aggtggaggt ggaagaggtc cccaacacca tcctcatctg catggtgatc ctgctgaaca | 2820 |
| tcggcctggc catcctcttt gttcacctcc tgaccggatc cggcagtgga gagggcagag | 2880 |
| gaagtctgct aacatgcggt gacgtcgagg agaatcctgg cccaatgagc aagggcgagg | 2940 |
| agctgttcac cggcgtggtg cccatcctgg tggagctgga cggcgacgtg aacggccaca | 3000 |
| agttcagcgt gagaggcgag ggcgagggcg acgccaccaa cggcaagctg accctgaagt | 3060 |
| tcatctgcac caccggcaag ctgcccgtgc cctggcccac cctggtgacc accctgacct | 3120 |
| acggcgtgct gtgcttcagc agataccccg accacatgaa gagacacgac ttcttcaaga | 3180 |
| gcgccatgcc cgagggctac gtgcaggaga gaaccatcag cttcaaggac gacggcacct | 3240 |
| acaagaccag agccgaggtg aagttcgagg gcgacaccct ggtgaacaga atcgagctga | 3300 |
| agggcatcga cttcaaggag gacggcaaca tcctgggcca caagctggag tacaacttca | 3360 |
| acagccacaa cgtgtacatc accgccgaca agcagaagaa cggcatcaag gcctacttca | 3420 |
| agatcagaca caacgtggag gacggcagcg tgcagctggc cgaccactac cagcagaaca | 3480 |
| cccccatcgg cgacggcccc gtgctgctgc ccgacaacca ctacctgagc acccagagcg | 3540 |
| tgctgagcaa ggaccccaac gagaagagag accacatggt gctgctggag gacgtgaccg | 3600 |
| ccgccggcat cacccacggc atggacgagc tgtacaagtg atcaacctct ggattacaaa | 3660 |
| atttgtgaaa gattgactgg tattcttaac tatgttgctc cttttacgct atgtggatac | 3720 |
| gctgctttaa tgcctttgta tcatgctatt gcttcccgta tggctttcat tttctcctcc | 3780 |
| ttgtataaat cctggttgct gtctctttat gaggagttgt ggcccgttgt caggcaacgt | 3840 |
| ggcgtggtgt gcactgtgtt tgctgacgca acccccactg gttggggcat tgccaccacc | 3900 |
| tgtcagctcc tttccgggac tttcgctttc cccctcccta ttgccacggc ggaactcatc | 3960 |
| gccgcctgcc ttgcccgctg ctggacaggg gctcggctgt tgggcactga caattccgtg | 4020 |
| gtgttgtcgg ggaaatcatc gtcctttcct tggctgctcg cctgtgttgc cacctggatt | 4080 |
| ctgcgcggga cgtccttctg ctacgtccct tcggccctca atccagcgga ccttccttcc | 4140 |
| cgcggcctgc tgccggctct gcggcctctt ccgcgtcttc gccttcgccc tcagacgagt | 4200 |
| cggatctccc tttgggccgc ctccccgcac tgcccgggtg gcatccctgt gacccctccc | 4260 |
| cagtgcctct cctggccctg gaagttgcca ctccagtgcc caccagcctt gtcctaataa | 4320 |
| aattaagttg catcattttg tctgactagg tgtccttcta taatattatg gggtggaggg | 4380 |
| gggtggtatg gagcaagggg cccaagttgg gaagaaacct gtagggcctg ccctaaggag | 4440 |
| gaacccctag tgatggagtt ggccactccc tctctgcgcg ctcgctcgct cactgaggcc | 4500 |
| gggcgaccaa aggtcgcccg acgcccgggc tttgcccggg cggcctcagt gagcgagcga | 4560 |
| gcgcgcagag agggagtggc caa | 4583 |

In a certain embodiment, the vector genome comprises, in 5′ to 3′ order, a 5′ITR; an MHCK7 promoter; a JPH2 3mutAA (V155A, R156A, L204A, L205A, R572A, and T573A) transgene; an WPRE(x) element; a hGH sequence; and a 3′ ITR. The vector genome may comprise, in 5′ to 3′ order, the polynucleotide sequences SEQ ID NO: 86; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length 3mutAA (V155A, R156A, L204A, L205A, R572A, and T573A) transgene, i.e. a transgene encoding a JPH2 of at least 600 or at least 630 amino acids.

| SEQ ID NO: 86 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtac ccttcagatt aaaaataact | 180 |
| gaggtaaggg cctgggtagg ggaggtggtg tgagacgctc ctgtctctcc tctatctgcc | 240 |
| catcggccct ttggggagga ggaatgtgcc caaggactaa aaaaaggcca tggagccaga | 300 |
| ggggcgaggg caacagacct ttcatgggca aaccttgggg ccctgctgtc tagcatgccc | 360 |
| cactacgggt ctaggctgcc catgtaagga ggcaaggcct ggggacaccc gagatgcctg | 420 |
| gttataatta acccagacat gtggctgccc cccccccccc aacacctgct gcctctaaaa | 480 |
| ataaccctgt ccctggtgga tcccctgcat gcgaagatct tcgaacaagg ctgtggggga | 540 |
| ctgagggcag gctgtaacag gcttgggggc cagggcttat acgtgcctgg gactcccaaa | 600 |
| gtattactgt tccatgttcc cggcgaaggg ccagctgtcc cccgccagct agactcagca | 660 |
| cttagtttag gaaccagtga gcaagtcagc ccttggggca gcccatacaa ggccatgggg | 720 |
| ctgggcaagc tgcacgcctg ggtccggggt gggcacggtg cccgggcaac gagctgaaag | 780 |
| ctcatctgct ctcaggggcc cctccctggg gacagcccct cctggctagt cacaccctgt | 840 |
| aggctcctct atataaccca ggggcacagg ggctgccctc attctaccac cacctccaca | 900 |
| gcacagacag acactcagga gccagccagg gtaagtttag tctttttgtc ttttatttca | 960 |
| ggtcccggat ccggtggtgg tgcaaatcaa agaactgctc ctcagtggat gttgccttta | 1020 |
| cttctaggcc tgtacggaag tgttacttct gctctaaaag ctgcggaatt gtacccgcgc | 1080 |
| caccatgagt gggggccgct tcgactttga tgatggaggg gcgtactgcg ggggctggga | 1140 |
| ggggggaaag gcccatgggc atggactgtg cacaggcccc aagggccagg gcgaatactc | 1200 |
| tggctcctgg aactttggct ttgaggtggc aggtgtctac acctggccca gcggaaacac | 1260 |
| ctttgaggga tactggagcc agggcaaacg gcatgggctg ggcatagaga ccaaggggcg | 1320 |
| ctggctctac aagggcgagt ggacacatgg cttcaaggga cgctacggaa tccggcagag | 1380 |
| ctcaagcagc ggtgccaagt atgagggcac ctggaacaat ggcctgcaag acggctatgg | 1440 |
| caccgagacc tatgctgatg gagggacgta ccaaggccag ttcaccaacg gcatgcgcca | 1500 |
| tggctacgga gtacgccaga gcgtgcccta cgggatggcc gtggtggccg cctcgccgct | 1560 |
| gcgcacgtcg ctgtcgtccc tgcgcagcga gcacagcaac ggcacggtgg ccccggactc | 1620 |
| tcccgcctcg ccggcctccg acggccccgc gctgccctcg cccgccatcc cgcgtggcgg | 1680 |
| cttcgcgctc agcgccgccg ccaatgccga ggcggccgcg cgggcgccca agggcggcgg | 1740 |
| cctcttccag cggggcgcgc tgctgggcaa gctgcggcgc gcagagtcgc gcacgtccgt | 1800 |
| gggtagccag cgcagccgtg tcagcttcct taagagcgac ctcagctcgg gcgccagcga | 1860 |
| cgccgcgtcc accgccagcc tgggagaggc cgccgagggc gccgacgagg ccgcaccctt | 1920 |
| cgaggccgat atcgacgcca ccaccaccga gacctacatg ggcgagtgga agaacgacaa | 1980 |
| acgctcgggc ttcggcgtga gcgaacgctc cagtggcctc cgctacgagg gcgagtggct | 2040 |
| ggacaacctg cgccacggct atggctgcac cacgctgccc gacggccacc gcgaggaggg | 2100 |
| caagtaccgc cacaacgtgc tggtcaagga caccaagcgc cgcatgctgc agctcaagag | 2160 |
| caacaaggtc cgccagaaag tggagcacag tgtggagggt gcccagcgcg ccgctgctat | 2220 |
| cgcgcgccag aaggccgaga ttgccgcctc caggacaagc cacgccaagg ccaaagctga | 2280 |
| ggcagcggaa caggccgccc tggctgccaa ccaggagtcc aacattgctc gcactttggc | 2340 |
| cagggagctg gctccggact tctaccagcc aggtccggaa tatcagaagc gccggctgct | 2400 |
| gcaggagatc ctggagaact cggagagcct gctggagccc cccgaccggg gcgccggcgc | 2460 |
| agcgggcctc ccacagccgc cccgcgagag cccgcagctg cacgagcgtg agacccctcg | 2520 |
| gcccgagggt ggctccccgt caccggccgg gacgcccccg cagcccaagc ggcccaggcc | 2580 |
| cggggtgtcc aaggacggcc tgctgagccc aggcgcctgg aacggcgagc ccagcggtga | 2640 |
| gggcagccgg tcagtcactc cgtccgaggg cgcgggccgc cgcagccccg cgcgtccagc | 2700 |
| caccgagcgc atggccatcg aggctctgca ggcaccgcct gcgccgtcgc gggagccgga | 2760 |
| ggtggcgctt taccagggct accacagcta tgctgtggcc gccacgccgc ccgagccccc | 2820 |
| accctttgag gaccagcccg agcccgaggt ctccgggtcc gagtccgcgc cctcgtcccc | 2880 |
| ggccaccgcc ccgctgcagg cccccacgct ccgaggcccc gagcctgcac gcgagacccc | 2940 |
| cgccaagctg gagcccaagc ccatcatccc caaagccgag cccagggcca aggcccgcaa | 3000 |
| gactgaggct cgagggctga ccaaggcggg ggccaagaag aaggcgcgga aggaggccgc | 3060 |
| actggcggca gaggcggagg tggaggtgga agaggtcccc aacaccatcc tcatctgcat | 3120 |
| ggtgatcctg ctgaacatcg gcctggccat cctctttgtt cacctcctga cctgatcaac | 3180 |
| ctctggatta caaaatttgt gaaagattga ctggtattct taactatgtt gctcctttta | 3240 |
| cgctatgtgg atacgctgct ttaatgcctt tgtatcatgc tattgcttcc cgtatggctt | 3300 |
| tcattttctc ctccttgtat aaatcctggt tgctgtctct ttatgaggag ttgtggcccg | 3360 |
| ttgtcaggca acgtggcgtg gtgtgcactg tgtttgctga cgcaaccccc actggttggg | 3420 |
| gcattgccac cacctgtcag ctcctttccg ggactttcgc tttccccctc cctattgcca | 3480 |
| cggcggaact catcgccgcc tgccttgccc gctgctggac aggggctcgg ctgttgggca | 3540 |
| ctgacaattc cgtggtgttg tcggggaaat catcgtcctt tccttggctg ctcgcctgtg | 3600 |
| ttgccacctg gattctgcgc gggacgtcct tctgctacgt cccttcggcc ctcaatccag | 3660 |
| cggaccttcc ttcccgcggc ctgctgccgg ctctgcggcc tcttccgcgt cttcgccttc | 3720 |
| gccctcagac gagtcggatc tccctttggg ccgcctcccc gcactgcccg ggtggcatcc | 3780 |
| ctgtgacccc tccccagtgc ctctcctggc cctggaagtt gccactccag tgcccaccag | 3840 |
| ccttgtccta ataaaattaa gttgcatcat tttgtctgac taggtgtcct tctataatat | 3900 |
| tatggggtgg aggggggtgg tatggagcaa ggggcccaag ttgggaagaa acctgtaggg | 3960 |
| cctgccctaa ggaggaaccc ctagtgatgg agttggccac tccctctctg cgcgctcgct | 4020 |
| cgctcactga ggccgggcga ccaaaggtcg cccgacgccc gggctttgcc cgggcggcct | 4080 |
| cagtgagcga gcgagcgcgc agagagggag tggccaa | 4117 |

In a certain embodiment, the vector genome comprises, in 5′ to 3′ order, a 5′ITR; a hTnnT2 promoter; a JPH2 3mutAA (V155A, R156A, L204A, L205A, R572A, and T573A) transgene; an WPRE(x) element; a hGH sequence; and a 3′ ITR. The vector genome may comprise, in 5′ to 3′ order, any one of the polynucleotide sequences SEQ ID NO: 87; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length JPH2 3mutAA (V155A, R156A, L204A, L205A, R572A, and T573A) transgene, i.e., a transgene encoding a JPH2 of at least 600 or at least 630 amino acids.

| SEQ ID NO: 87 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtct cagtccatta ggagccagta | 180 |
| gcctggaaga tgtctttacc cccagcatca gttcaagtgg agcagcacat aactcttgcc | 240 |
| ctctgccttc caagattctg gtgctgagac ttatggagtg tcttggaggt tgccttctgc | 300 |
| cccccaaccc tgctcccagc tggccctccc aggcctgggt tgctggcctc tgctttatca | 360 |
| ggattctcaa gagggacagc tggtttatgt tgcatgactg ttccctgcat atctgctctg | 420 |
| gttttaaata gcttatctga gcagctggag gaccacatgg gcttatatgg cgtggggtac | 480 |
| atgttcctgt agccttgtcc ctggcacctg ccaaaatagc agccaacacc ccccaccccc | 540 |
| accgccatcc ccctgcccca cccgtcccct gtcgcacatt cctccctccg cagggctggc | 600 |
| tcaccaggcc ccagcccaca tgcctgctta aagccctctc catcctctgc ctcacccagt | 660 |
| ccccgctgag actgagcaga cgcctccagg atctgtcggc aggccaccat gagtgggggc | 720 |
| cgcttcgact ttgatgatgg aggggcgtac tgcgggggct gggagggggg aaaggcccat | 780 |
| gggcatggac tgtgcacagg ccccaagggc cagggcgaat actctggctc ctggaacttt | 840 |
| ggctttgagg tggcaggtgt ctacacctgg cccagcggaa acacctttga gggatactgg | 900 |
| agccagggca aacggcatgg gctgggcata gagaccaagg ggcgctggct ctacaagggc | 960 |
| gagtggacac atggcttcaa gggacgctac ggaatccggc agagctcaag cagcggtgcc | 1020 |
| aagtatgagg gcacctggaa caatggcctg caagacggct atggcaccga gacctatgct | 1080 |
| gatggaggga cgtaccaagg ccagttcacc aacggcatgc gccatggcta cggagtacgc | 1140 |
| cagagcgtgc cctacgggat ggccgtggtg gccgcctcgc cgctgcgcac gtcgctgtcg | 1200 |
| tccctgcgca gcgagcacag caacggcacg gtggccccgg actctcccgc ctcgccggcc | 1260 |
| tccgacggcc ccgcgctgcc ctcgcccgcc atcccgcgtg gcggcttcgc gctcagcgcc | 1320 |
| gccgccaatg ccgaggcggc cgcgcgggcg cccaagggcg gcggcctctt ccagcggggc | 1380 |
| gcgctgctgg gcaagctgcg gcgcgcagag tcgcgcacgt ccgtgggtag ccagcgcagc | 1440 |
| cgtgtcagct tccttaagag cgacctcagc tcgggcgcca gcgacgccgc gtccaccgcc | 1500 |
| agcctgggag aggccgccga gggcgccgac gaggccgcac ccttcgaggc cgatatcgac | 1560 |
| gccaccacca ccgagaccta catgggcgag tggaagaacg acaaacgctc gggcttcggc | 1620 |
| gtgagcgaac gctccagtgg cctccgctac gagggcgagt ggctggacaa cctgcgccac | 1680 |
| ggctatggct gcaccacgct gcccgacggc caccgcgagg agggcaagta ccgccacaac | 1740 |
| gtgctggtca aggacaccaa gcgccgcatg ctgcagctca agagcaacaa ggtccgccag | 1800 |
| aaagtggagc acagtgtgga gggtgcccag cgcgccgctg ctatcgcgcg ccagaaggcc | 1860 |
| gagattgccg cctccaggac aagccacgcc aaggccaaag ctgaggcagc ggaacaggcc | 1920 |
| gccctggctg ccaaccagga gtccaacatt gctcgcactt tggccaggga gctggctccg | 1980 |
| gacttctacc agccaggtcc ggaatatcag aagcgccggc tgctgcagga gatcctggag | 2040 |
| aactcggaga gcctgctgga gccccccgac cggggcgccg gcgcagcggg cctcccacag | 2100 |
| ccgccccgcg agagcccgca gctgcacgag cgtgagaccc ctcggcccga gggtggctcc | 2160 |
| ccgtcaccgg ccgggacgcc cccgcagccc aagcggccca ggcccggggt gtccaaggac | 2220 |
| ggcctgctga gcccaggcgc ctggaacggc gagcccagcg gtgagggcag ccggtcagtc | 2280 |
| actccgtccg agggcgcggg ccgccgcagc cccgcgcgtc cagccaccga gcgcatggcc | 2340 |
| atcgaggctc tgcaggcacc gcctgcgccg tcgcgggagc cggaggtggc gctttaccag | 2400 |
| ggctaccaca gctatgctgt ggccgccacg ccgcccgagc ccccaccctt tgaggaccag | 2460 |
| cccgagcccg aggtctccgg gtccgagtcc gcgccctcgt ccccggccac cgccccgctg | 2520 |
| caggccccca cgctccgagg ccccgagcct gcacgcgaga cccccgccaa gctggagccc | 2580 |
| aagcccatca tccccaaagc cgagcccagg gccaaggccc gcaagactga ggctcgaggg | 2640 |
| ctgaccaagg cgggggccaa gaagaaggcg cggaaggagg ccgcactggc ggcagaggcg | 2700 |
| gaggtggagg tggaagaggt ccccaacacc atcctcatct gcatggtgat cctgctgaac | 2760 |
| atcggcctgg ccatcctctt tgttcacctc ctgacctgat caacctctgg attacaaaat | 2820 |
| ttgtgaaaga ttgactggta ttcttaacta tgttgctcct tttacgctat gtggatacgc | 2880 |
| tgctttaatg cctttgtatc atgctattgc ttcccgtatg gctttcattt tctcctcctt | 2940 |
| gtataaatcc tggttgctgt ctctttatga ggagttgtgg cccgttgtca ggcaacgtgg | 3000 |
| cgtggtgtgc actgtgtttg ctgacgcaac ccccactggt tggggcattg ccaccacctg | 3060 |
| tcagctcctt tccgggactt tcgctttccc cctccctatt gccacggcgg aactcatcgc | 3120 |
| cgcctgcctt gcccgctgct ggacaggggc tcggctgttg ggcactgaca attccgtggt | 3180 |
| gttgtcgggg aaatcatcgt cctttccttg gctgctcgcc tgtgttgcca cctggattct | 3240 |
| gcgcgggacg tccttctgct acgtcccttc ggccctcaat ccagcggacc ttccttcccg | 3300 |
| cggcctgctg ccggctctgc ggcctcttcc gcgtcttcgc cttcgccctc agacgagtcg | 3360 |
| gatctccctt tgggccgcct ccccgcactg cccgggtggc atccctgtga cccctcccca | 3420 |
| gtgcctctcc tggccctgga agttgccact ccagtgccca ccagccttgt cctaataaaa | 3480 |
| ttaagttgca tcattttgtc tgactaggtg tccttctata atattatggg gtggaggggg | 3540 |
| gtggtatgga gcaaggggcc caagttggga agaaacctgt agggcctgcc ctaaggagga | 3600 |
| acccctagtg atggagttgg ccactccctc tctgcgcgct cgctcgctca ctgaggccgg | 3660 |
| gcgaccaaag gtcgcccgac gcccgggctt tgcccgggcg gcctcagtga gcgagcgagc | 3720 |
| gcgcagagag ggagtggcca a | 3741 |

In a certain embodiment, the vector genome comprises, in 5′ to 3′ order, a 5′ITR; an MHCK7 promoter; a JPH2 3mutAA (V155A, R156A, L204A, L205A, R572A, and T573A) transgene; a GFP tag; an WPRE(x) element; a hGH sequence; and a 3′ ITR. The vector genome may comprise, in 5′ to 3′ order, the polynucleotide sequences SEQ ID NO: 88; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length 3mutAA (V155A, R156A, L204A, L205A, R572A, and T573A) transgene, i.e., a transgene encoding a JPH2 of at least 600 or at least 630 amino acids.

| SEQ ID NO: 88 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtac ccttcagatt aaaaataact | 180 |
| gaggtaaggg cctgggtagg ggaggtggtg tgagacgctc ctgtctctcc tctatctgcc | 240 |
| catcggccct ttggggagga ggaatgtgcc caaggactaa aaaaaggcca tggagccaga | 300 |
| ggggcgaggg caacagacct ttcatgggca aaccttgggg ccctgctgtc tagcatgccc | 360 |
| cactacgggt ctaggctgcc catgtaagga ggcaaggcct ggggacaccc gagatgcctg | 420 |
| gttataatta acccagacat gtggctgccc cccccccccc aacacctgct gcctctaaaa | 480 |
| ataaccctgt ccctggtgga tcccctgcat gcgaagatct tcgaacaagg ctgtggggga | 540 |
| ctgagggcag gctgtaacag gcttgggggc cagggcttat acgtgcctgg gactcccaaa | 600 |
| gtattactgt tccatgttcc cggcgaaggg ccagctgtcc cccgccagct agactcagca | 660 |
| cttagtttag gaaccagtga gcaagtcagc ccttggggca gcccatacaa ggccatgggg | 720 |
| ctgggcaagc tgcacgcctg ggtccggggt gggcacggtg cccgggcaac gagctgaaag | 780 |
| ctcatctgct ctcaggggcc cctccctggg gacagcccct cctggctagt cacaccctgt | 840 |
| aggctcctct atataaccca ggggcacagg ggctgccctc attctaccac cacctccaca | 900 |
| gcacagacag acactcagga gccagccagg ccaccatgag tgggggccgc ttcgactttg | 960 |
| atgatggagg ggcgtactgc gggggctggg aggggggaaa ggcccatggg catggactgt | 1020 |
| gcacaggccc caagggccag ggcgaatact ctggctcctg gaactttggc tttgaggtgg | 1080 |
| caggtgtcta cacctggccc agcggaaaca cctttgaggg atactggagc cagggcaaac | 1140 |
| ggcatgggct gggcatagag accaaggggc gctggctcta caagggcgag tggacacatg | 1200 |
| gcttcaaggg acgctacgga atccggcaga gctcaagcag cggtgccaag tatgagggca | 1260 |
| cctggaacaa tggcctgcaa gacggctatg gcaccgagac ctatgctgat ggagggacgt | 1320 |
| accaaggcca gttcaccaac ggcatgcgcc atggctacgg agtacgccag agcgtgccct | 1380 |
| acgggatggc cgtggtggcc gcctcgccgc tgcgcacgtc gctgtcgtcc ctgcgcagcg | 1440 |
| agcacagcaa cggcacggtg gccccggact ctcccgcctc gccggcctcc gacggccccg | 1500 |
| cgctgccctc gcccgccatc ccgcgtggcg gcttcgcgct cagcgccgcc gccaatgccg | 1560 |
| aggcggccgc gcgggcgccc aagggcggcg gcctcttcca gcggggcgcg ctgctgggca | 1620 |
| agctgcggcg cgcagagtcg cgcacgtccg tgggtagcca gcgcagccgt gtcagcttcc | 1680 |
| ttaagagcga cctcagctcg ggcgccagcg acgccgcgtc caccgccagc ctgggagagg | 1740 |
| ccgccgaggg cgccgacgag gccgcaccct tcgaggccga tatcgacgcc accaccaccg | 1800 |
| agacctacat gggcgagtgg aagaacgaca aacgctcggg cttcggcgtg agcgaacgct | 1860 |
| ccagtggcct ccgctacgag ggcgagtggc tggacaacct gcgccacggc tatggctgca | 1920 |
| ccacgctgcc cgacggccac cgcgaggagg gcaagtaccg ccacaacgtg ctggtcaagg | 1980 |
| acaccaagcg ccgcatgctg cagctcaaga gcaacaaggt ccgccagaaa gtggagcaca | 2040 |
| gtgtggaggg tgcccagcgc gccgctgcta tcgcgcgcca gaaggccgag attgccgcct | 2100 |
| ccaggacaag ccacgccaag gccaaagctg aggcagcgga acaggccgcc ctggctgcca | 2160 |
| accaggagtc caacattgct cgcactttgg ccagggagct ggctccggac ttctaccagc | 2220 |
| caggtccgga atatcagaag cgccggctgc tgcaggagat cctggagaac tcggagagcc | 2280 |
| tgctggagcc ccccgaccgg ggcgccggcg cagcgggcct cccacagccg ccccgcgaga | 2340 |
| gcccgcagct gcacgagcgt gagacccctc ggcccgaggg tggctccccg tcaccggccg | 2400 |
| ggacgccccc gcagcccaag cggcccaggc ccggggtgtc caaggacggc ctgctgagcc | 2460 |
| caggcgcctg gaacggcgag cccagcggtg agggcagccg gtcagtcact ccgtccgagg | 2520 |
| gcgcgggccg ccgcagcccc gcgcgtccag ccaccgagcg catggccatc gaggctctgc | 2580 |
| aggcaccgcc tgcgccgtcg cgggagccgg aggtggcgct ttaccagggc taccacagct | 2640 |
| atgctgtggc cgccacgccg cccgagcccc caccctttga ggaccagccc gagcccgagg | 2700 |
| tctccgggtc cgagtccgcg ccctcgtccc cggccaccgc cccgctgcag gcccccacgc | 2760 |
| tccgaggccc cgagcctgca cgcgagaccc ccgccaagct ggagcccaag cccatcatcc | 2820 |
| ccaaagccga gcccagggcc aaggcccgca agactgaggc tcgagggctg accaaggcgg | 2880 |
| gggccaagaa gaaggcgcgg aaggaggccg cactggcggc agaggcggag gtggaggtgg | 2940 |
| aagaggtccc caacaccatc ctcatctgca tggtgatcct gctgaacatc ggcctggcca | 3000 |
| tcctctttgt tcacctcctg accggatccg gcagtggaga gggcagagga agtctgctaa | 3060 |
| catgcggtga cgtcgaggag aatcctggcc caatgagcaa gggcgaggag ctgttcaccg | 3120 |
| gcgtggtgcc catcctggtg gagctggacg gcgacgtgaa cggccacaag ttcagcgtga | 3180 |
| gaggcgaggg cgagggcgac gccaccaacg gcaagctgac cctgaagttc atctgcacca | 3240 |
| ccggcaagct gcccgtgccc tggcccaccc tggtgaccac cctgacctac ggcgtgctgt | 3300 |
| gcttcagcag ataccccgac cacatgaaga gacacgactt cttcaagagc gccatgcccg | 3360 |
| agggctacgt gcaggagaga accatcagct tcaaggacga cggcacctac aagaccagag | 3420 |
| ccgaggtgaa gttcgagggc gacaccctgg tgaacagaat cgagctgaag ggcatcgact | 3480 |
| tcaaggagga cggcaacatc ctgggccaca agctggagta caacttcaac agccacaacg | 3540 |
| tgtacatcac cgccgacaag cagaagaacg gcatcaaggc ctacttcaag atcagacaca | 3600 |
| acgtggagga cggcagcgtg cagctggccg accactacca gcagaacacc cccatcggcg | 3660 |
| acggccccgt gctgctgccc gacaaccact acctgagcac ccagagcgtg ctgagcaagg | 3720 |
| accccaacga gaagagagac cacatggtgc tgctggagga cgtgaccgcc gccggcatca | 3780 |
| cccacggcat ggacgagctg tacaagtgat caacctctgg attacaaaat ttgtgaaaga | 3840 |
| ttgactggta ttcttaacta tgttgctcct tttacgctat gtggatacgc tgctttaatg | 3900 |
| cctttgtatc atgctattgc ttcccgtatg gctttcattt tctcctcctt gtataaatcc | 3960 |
| tggttgctgt ctctttatga ggagttgtgg cccgttgtca ggcaacgtgg cgtggtgtgc | 4020 |
| actgtgtttg ctgacgcaac ccccactggt tggggcattg ccaccacctg tcagctcctt | 4080 |
| tccgggactt tcgctttccc cctccctatt gccacggcgg aactcatcgc cgcctgcctt | 4140 |
| gcccgctgct ggacaggggc tcggctgttg ggcactgaca attccgtggt gttgtcgggg | 4200 |
| aaatcatcgt cctttccttg gctgctcgcc tgtgttgcca cctggattct gcgcgggacg | 4260 |
| tccttctgct acgtcccttc ggccctcaat ccagcggacc ttccttcccg cggcctgctg | 4320 |
| ccggctctgc ggcctcttcc gcgtcttcgc cttcgccctc agacgagtcg gatctccctt | 4380 |
| tgggccgcct ccccgcactg cccgggtggc atccctgtga cccctcccca gtgcctctcc | 4440 |
| tggccctgga agttgccact ccagtgccca ccagccttgt cctaataaaa ttaagttgca | 4500 |
| tcattttgtc tgactaggtg tccttctata atattatggg gtggaggggg gtggtatgga | 4560 |
| gcaaggggcc caagttggga agaaacctgt agggcctgcc ctaaggagga acccctagtg | 4620 |
| atggagttgg ccactccctc tctgcgcgct cgctcgctca ctgaggccgg gcgaccaaag | 4680 |
| gtcgcccgac gcccgggctt tgcccgggcg gcctcagtga gcgagcgagc gcgcagagag | 4740 |
| ggagtggcca a | 4751 |

In a certain embodiment, the vector genome comprises, in 5′ to 3′ order, a 5′ITR; a hTnnT2 promoter; a JPH2 3mutAA (V155A, R156A, L204A, L205A, R572A, and T573A) transgene; a GFP tag; an WPRE(x) element; a hGH sequence; and a 3′ ITR. The vector genome may comprise, in 5′ to 3′ order, any one of the polynucleotide sequences SEQ ID NO: 89; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length JPH2 3mutAA (V155A, R156A, L204A, L205A, R572A, and T573A) transgene, i.e. a transgene encoding a JPH2 of at least 600 or at least 630 amino acids.

| SEQ ID NO: 89 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtct cagtccatta ggagccagta | 180 |
| gcctggaaga tgtctttacc cccagcatca gttcaagtgg agcagcacat aactcttgcc | 240 |
| ctctgccttc caagattctg gtgctgagac ttatggagtg tcttggaggt tgccttctgc | 300 |
| cccccaaccc tgctcccagc tggccctccc aggcctgggt tgctggcctc tgctttatca | 360 |
| ggattctcaa gagggacagc tggtttatgt tgcatgactg ttccctgcat atctgctctg | 420 |
| gttttaaata gcttatctga gcagctggag gaccacatgg gcttatatgg cgtggggtac | 480 |
| atgttcctgt agccttgtcc ctggcacctg ccaaaatagc agccaacacc ccccaccccc | 540 |
| accgccatcc ccctgcccca cccgtcccct gtcgcacatt cctccctccg cagggctggc | 600 |
| tcaccaggcc ccagcccaca tgcctgctta aagccctctc catcctctgc ctcacccagt | 660 |
| ccccgctgag actgagcaga cgcctccagg atctgtcggc aggccaccat gagtgggggc | 720 |
| cgcttcgact ttgatgatgg aggggcgtac tgcgggggct gggagggggg aaaggcccat | 780 |
| gggcatggac tgtgcacagg ccccaagggc cagggcgaat actctggctc ctggaacttt | 840 |
| ggctttgagg tggcaggtgt ctacacctgg cccagcggaa acacctttga gggatactgg | 900 |
| agccagggca aacggcatgg gctgggcata gagaccaagg ggcgctggct ctacaagggc | 960 |
| gagtggacac atggcttcaa gggacgctac ggaatccggc agagctcaag cagcggtgcc | 1020 |
| aagtatgagg gcacctggaa caatggcctg caagacggct atggcaccga gacctatgct | 1080 |
| gatggaggga cgtaccaagg ccagttcacc aacggcatgc gccatggcta cggagtacgc | 1140 |
| cagagcgtgc cctacgggat ggccgtggtg gccgcctcgc cgctgcgcac gtcgctgtcg | 1200 |
| tccctgcgca gcgagcacag caacggcacg gtggccccgg actctcccgc ctcgccggcc | 1260 |
| tccgacggcc ccgcgctgcc ctcgcccgcc atcccgcgtg gcggcttcgc gctcagcgcc | 1320 |
| gccgccaatg ccgaggcggc cgcgcgggcg cccaagggcg gcggcctctt ccagcggggc | 1380 |
| gcgctgctgg gcaagctgcg gcgcgcagag tcgcgcacgt ccgtgggtag ccagcgcagc | 1440 |
| cgtgtcagct tccttaagag cgacctcagc tcgggcgcca gcgacgccgc gtccaccgcc | 1500 |
| agcctgggag aggccgccga gggcgccgac gaggccgcac ccttcgaggc cgatatcgac | 1560 |
| gccaccacca ccgagaccta catgggcgag tggaagaacg acaaacgctc gggcttcggc | 1620 |
| gtgagcgaac gctccagtgg cctccgctac gagggcgagt ggctggacaa cctgcgccac | 1680 |
| ggctatggct gcaccacgct gcccgacggc caccgcgagg agggcaagta ccgccacaac | 1740 |
| gtgctggtca aggacaccaa gcgccgcatg ctgcagctca agagcaacaa ggtccgccag | 1800 |
| aaagtggagc acagtgtgga gggtgcccag cgcgccgctg ctatcgcgcg ccagaaggcc | 1860 |
| gagattgccg cctccaggac aagccacgcc aaggccaaag ctgaggcagc ggaacaggcc | 1920 |
| gccctggctg ccaaccagga gtccaacatt gctcgcactt tggccaggga gctggctccg | 1980 |
| gacttctacc agccaggtcc ggaatatcag aagcgccggc tgctgcagga gatcctggag | 2040 |
| aactcggaga gcctgctgga gccccccgac cggggcgccg gcgcagcggg cctcccacag | 2100 |
| ccgccccgcg agagcccgca gctgcacgag cgtgagaccc ctcggcccga gggtggctcc | 2160 |
| ccgtcaccgg ccgggacgcc cccgcagccc aagcggccca ggcccggggt gtccaaggac | 2220 |
| ggcctgctga gcccaggcgc ctggaacggc gagcccagcg gtgagggcag ccggtcagtc | 2280 |
| actccgtccg agggcgcggg ccgccgcagc cccgcgcgtc cagccaccga gcgcatggcc | 2340 |
| atcgaggctc tgcaggcacc gcctgcgccg tcgcgggagc cggaggtggc gctttaccag | 2400 |
| ggctaccaca gctatgctgt ggccgccacg ccgcccgagc ccccaccctt tgaggaccag | 2460 |
| cccgagcccg aggtctccgg gtccgagtcc gcgccctcgt ccccggccac cgccccgctg | 2520 |
| caggccccca cgctccgagg ccccgagcct gcacgcgaga cccccgccaa gctggagccc | 2580 |
| aagcccatca tccccaaagc cgagcccagg gccaaggccc gcaagactga ggctcgaggg | 2640 |
| ctgaccaagg cgggggccaa gaagaaggcg cggaaggagg ccgcactggc ggcagaggcg | 2700 |
| gaggtggagg tggaagaggt ccccaacacc atcctcatct gcatggtgat cctgctgaac | 2760 |
| atcggcctgg ccatcctctt tgttcacctc ctgaccggat ccggcagtgg agagggcaga | 2820 |
| ggaagtctgc taacatgcgg tgacgtcgag gagaatcctg gcccaatgag caagggcgag | 2880 |
| gagctgttca ccggcgtggt gcccatcctg gtggagctgg acggcgacgt gaacggccac | 2940 |
| aagttcagcg tgagaggcga gggcgagggc gacgccacca acggcaagct gaccctgaag | 3000 |
| ttcatctgca ccaccggcaa gctgcccgtg ccctggccca ccctggtgac caccctgacc | 3060 |
| tacggcgtgc tgtgcttcag cagatacccc gaccacatga agagacacga cttcttcaag | 3120 |
| agcgccatgc ccgagggcta cgtgcaggag agaaccatca gcttcaagga cgacggcacc | 3180 |
| tacaagacca gagccgaggt gaagttcgag ggcgacaccc tggtgaacag aatcgagctg | 3240 |
| aagggcatcg acttcaagga ggacggcaac atcctgggcc acaagctgga gtacaacttc | 3300 |
| aacagccaca acgtgtacat caccgccgac aagcagaaga acggcatcaa ggcctacttc | 3360 |
| aagatcagac acaacgtgga ggacggcagc gtgcagctgg ccgaccacta ccagcagaac | 3420 |
| acccccatcg gcgacggccc cgtgctgctg cccgacaacc actacctgag cacccagagc | 3480 |
| gtgctgagca aggaccccaa cgagaagaga gaccacatgg tgctgctgga ggacgtgacc | 3540 |
| gccgccggca tcacccacgg catggacgag ctgtacaagt gatcaacctc tggattacaa | 3600 |
| aatttgtgaa agattgactg gtattcttaa ctatgttgct ccttttacgc tatgtggata | 3660 |
| cgctgcttta atgcctttgt atcatgctat tgcttcccgt atggctttca ttttctcctc | 3720 |
| cttgtataaa tcctggttgc tgtctcttta tgaggagttg tggcccgttg tcaggcaacg | 3780 |
| tggcgtggtg tgcactgtgt ttgctgacgc aacccccact ggttggggca ttgccaccac | 3840 |
| ctgtcagctc ctttccggga ctttcgcttt ccccctccct attgccacgg cggaactcat | 3900 |
| cgccgcctgc cttgcccgct gctggacagg ggctcggctg ttgggcactg acaattccgt | 3960 |
| ggtgttgtcg gggaaatcat cgtcctttcc ttggctgctc gcctgtgttg ccacctggat | 4020 |
| tctgcgcggg acgtccttct gctacgtccc ttcggccctc aatccagcgg accttccttc | 4080 |
| ccgcggcctg ctgccggctc tgcggcctct tccgcgtctt cgccttcgcc ctcagacgag | 4140 |
| tcggatctcc ctttgggccg cctccccgca ctgcccgggt ggcatccctg tgacccctcc | 4200 |
| ccagtgcctc tcctggccct ggaagttgcc actccagtgc ccaccagcct tgtcctaata | 4260 |
| aaattaagtt gcatcatttt gtctgactag gtgtccttct ataatattat ggggtggagg | 4320 |
| ggggtggtat ggagcaaggg gcccaagttg ggaagaaacc tgtagggcct gccctaagga | 4380 |
| ggaaccccta gtgatggagt tggccactcc ctctctgcgc gctcgctcgc tcactgaggc | 4440 |
| cgggcgacca aaggtcgccc gacgcccggg ctttgcccgg gcggcctcag tgagcgagcg | 4500 |
| agcgcgcaga gagggagtgg ccaa | 4524 |

In a certain embodiment, the vector genome comprises, in 5′ to 3′ order, a 5′ITR; a CMV enhancer element; a CMV promoter; a JPH2 3mutAA (V155A, R156A, L204A, L205A, R572A, and T573A) transgene; a GFP tag; an WPRE(x) element; a hGH sequence; and a 3′ ITR. The vector genome may comprise, in 5′ to 3′ order, any one of the polynucleotide sequences SEQ ID NO: 90; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The transgene of this embodiment is a full length JPH2 3mutAA (V155A, R156A, L204A, L205A, R572A, and T573A) transgene, i.e., a transgene encoding a JPH2 of at least 600 or at least 630 amino acids.

| SEQ ID NO: 90 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtta catacgttac ataacttacg | 180 |
| gtaaatggcc cgcctggctg accgcccaac gacccccgcc cattgacgtc aataatgacg | 240 |
| tatgttccca tagtaacgcc aatagggact ttccattgac gtcaatgggt ggagtattta | 300 |
| cggtaaactg cccacttggc agtacatcaa gtgtatcata tgccaagtac gccccctatt | 360 |
| gacgtcaatg acggtaaatg gcccgcctgg cattatgccc agtacatgac cttatgggac | 420 |
| tttcctactt ggcagtacat ctacgtatta gtcatcgcta ttaccatggt gatgcggttt | 480 |
| tggcagtaca tcaatgggcg tggatagcgg tttgactcac ggggatttcc aagtctccac | 540 |
| cccattgacg tcaatgggag tttgttttgg caccaaaatc aacgggactt tccaaaatgt | 600 |
| cgtaacaact ccgccccatt gacgcaaatg ggcggtaggc gtgtacggtg ggaggtctat | 660 |
| ataagcagag ctcgtttagt gaaccgtcag atcgcctgga gacgccatcc acgctgtttt | 720 |
| gacctccata gaagacaccg ggaccgatcc agcctccgcg ggccaccatg agtgggggcc | 780 |
| gcttcgactt tgatgatgga ggggcgtact gcgggggctg ggagggggga aaggcccatg | 840 |
| ggcatggact gtgcacaggc cccaagggcc agggcgaata ctctggctcc tggaactttg | 900 |
| gctttgaggt ggcaggtgtc tacacctggc ccagcggaaa cacctttgag ggatactgga | 960 |
| gccagggcaa acggcatggg ctgggcatag agaccaaggg gcgctggctc tacaagggcg | 1020 |
| agtggacaca tggcttcaag ggacgctacg gaatccggca gagctcaagc agcggtgcca | 1080 |
| agtatgaggg cacctggaac aatggcctgc aagacggcta tggcaccgag acctatgctg | 1140 |
| atggagggac gtaccaaggc cagttcacca acggcatgcg ccatggctac ggagtacgcc | 1200 |
| agagcgtgcc ctacgggatg gccgtggtgg ccgcctcgcc gctgcgcacg tcgctgtcgt | 1260 |
| ccctgcgcag cgagcacagc aacggcacgg tggccccgga ctctcccgcc tcgccggcct | 1320 |
| ccgacggccc cgcgctgccc tcgcccgcca tcccgcgtgg cggcttcgcg ctcagcgccg | 1380 |
| ccgccaatgc cgaggcggcc gcgcgggcgc ccaagggcgg cggcctcttc cagcggggcg | 1440 |
| cgctgctggg caagctgcgg cgcgcagagt cgcgcacgtc cgtgggtagc cagcgcagcc | 1500 |
| gtgtcagctt ccttaagagc gacctcagct cgggcgccag cgacgccgcg tccaccgcca | 1560 |
| gcctgggaga ggccgccgag ggcgccgacg aggccgcacc cttcgaggcc gatatcgacg | 1620 |
| ccaccaccac cgagacctac atgggcgagt ggaagaacga caaacgctcg ggcttcggcg | 1680 |
| tgagcgaacg ctccagtggc ctccgctacg agggcgagtg gctggacaac ctgcgccacg | 1740 |
| gctatggctg caccacgctg cccgacggcc accgcgagga gggcaagtac cgccacaacg | 1800 |
| tgctggtcaa ggacaccaag cgccgcatgc tgcagctcaa gagcaacaag gtccgccaga | 1860 |
| aagtggagca cagtgtggag ggtgcccagc gcgccgctgc tatcgcgcgc cagaaggccg | 1920 |
| agattgccgc ctccaggaca agccacgcca aggccaaagc tgaggcagcg gaacaggccg | 1980 |
| ccctggctgc caaccaggag tccaacattg ctcgcacttt ggccagggag ctggctccgg | 2040 |
| acttctacca gccaggtccg gaatatcaga agcgccggct gctgcaggag atcctggaga | 2100 |
| actcggagag cctgctggag ccccccgacc ggggcgccgg cgcagcgggc ctcccacagc | 2160 |
| cgccccgcga gagcccgcag ctgcacgagc gtgagacccc tcggcccgag ggtggctccc | 2220 |
| cgtcaccggc cgggacgccc ccgcagccca agcggcccag gcccggggtg tccaaggacg | 2280 |
| gcctgctgag cccaggcgcc tggaacggcg agcccagcgg tgagggcagc cggtcagtca | 2340 |
| ctccgtccga gggcgcgggc cgccgcagcc ccgcgcgtcc agccaccgag cgcatggcca | 2400 |
| tcgaggctct gcaggcaccg cctgcgccgt cgcgggagcc ggaggtggcg ctttaccagg | 2460 |
| gctaccacag ctatgctgtg gccgccacgc cgcccgagcc cccacccttt gaggaccagc | 2520 |
| ccgagcccga ggtctccggg tccgagtccg cgccctcgtc cccggccacc gccccgctgc | 2580 |
| aggcccccac gctccgaggc cccgagcctg cacgcgagac ccccgccaag ctggagccca | 2640 |
| agcccatcat ccccaaagcc gagcccaggg ccaaggcccg caagactgag gctcgagggc | 2700 |
| tgaccaaggc gggggccaag aagaaggcgc ggaaggaggc cgcactggcg gcagaggcgg | 2760 |
| aggtggaggt ggaagaggtc cccaacacca tcctcatctg catggtgatc ctgctgaaca | 2820 |
| tcggcctggc catcctcttt gttcacctcc tgaccggatc cggcagtgga gagggcagag | 2880 |
| gaagtctgct aacatgcggt gacgtcgagg agaatcctgg cccaatgagc aagggcgagg | 2940 |
| agctgttcac cggcgtggtg cccatcctgg tggagctgga cggcgacgtg aacggccaca | 3000 |
| agttcagcgt gagaggcgag ggcgagggcg acgccaccaa cggcaagctg accctgaagt | 3060 |
| tcatctgcac caccggcaag ctgcccgtgc cctggcccac cctggtgacc accctgacct | 3120 |
| acggcgtgct gtgcttcagc agataccccg accacatgaa gagacacgac ttcttcaaga | 3180 |
| gcgccatgcc cgagggctac gtgcaggaga gaaccatcag cttcaaggac gacggcacct | 3240 |
| acaagaccag agccgaggtg aagttcgagg gcgacaccct ggtgaacaga atcgagctga | 3300 |
| agggcatcga cttcaaggag gacggcaaca tcctgggcca caagctggag tacaacttca | 3360 |
| acagccacaa cgtgtacatc accgccgaca agcagaagaa cggcatcaag gcctacttca | 3420 |
| agatcagaca caacgtggag gacggcagcg tgcagctggc cgaccactac cagcagaaca | 3480 |
| cccccatcgg cgacggcccc gtgctgctgc ccgacaacca ctacctgagc acccagagcg | 3540 |
| tgctgagcaa ggaccccaac gagaagagag accacatggt gctgctggag gacgtgaccg | 3600 |
| ccgccggcat cacccacggc atggacgagc tgtacaagtg atcaacctct ggattacaaa | 3660 |
| atttgtgaaa gattgactgg tattcttaac tatgttgctc cttttacgct atgtggatac | 3720 |
| gctgctttaa tgcctttgta tcatgctatt gcttcccgta tggctttcat tttctcctcc | 3780 |
| ttgtataaat cctggttgct gtctctttat gaggagttgt ggcccgttgt caggcaacgt | 3840 |
| ggcgtggtgt gcactgtgtt tgctgacgca acccccactg gttggggcat tgccaccacc | 3900 |
| tgtcagctcc tttccgggac tttcgctttc cccctcccta ttgccacggc ggaactcatc | 3960 |
| gccgcctgcc ttgcccgctg ctggacaggg gctcggctgt tgggcactga caattccgtg | 4020 |
| gtgttgtcgg ggaaatcatc gtcctttcct tggctgctcg cctgtgttgc cacctggatt | 4080 |
| ctgcgcggga cgtccttctg ctacgtccct tcggccctca atccagcgga ccttccttcc | 4140 |
| cgcggcctgc tgccggctct gcggcctctt ccgcgtcttc gccttcgccc tcagacgagt | 4200 |
| cggatctccc tttgggccgc ctccccgcac tgcccgggtg gcatccctgt gacccctccc | 4260 |
| cagtgcctct cctggccctg gaagttgcca ctccagtgcc caccagcctt gtcctaataa | 4320 |
| aattaagttg catcattttg tctgactagg tgtccttcta taatattatg gggtggaggg | 4380 |
| gggtggtatg gagcaagggg cccaagttgg gaagaaacct gtagggcctg ccctaaggag | 4440 |
| gaacccctag tgatggagtt ggccactccc tctctgcgcg ctcgctcgct cactgaggcc | 4500 |
| gggcgaccaa aggtcgcccg acgcccgggc tttgcccggg cggcctcagt gagcgagcga | 4560 |
| gcgcgcagag agggagtggc caa | 4583 |

In a certain embodiment, the vector genome comprises, in 5′ to 3′ order, a 5′ITR; a MHCK7 promoter; a JPH2 3mutAKAAKS (V155A, R156K, L204A, L205A, R572K, and T573S) transgene; an WPRE(x) element; a hGH sequence; and a 3′ ITR. The vector genome may comprise, in 5′ to 3′ order, any one of the polynucleotide sequences SEQ ID NO: 91; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length JPH2 3mutAKAAKS (V155A, R156K, L204A, L205A, R572K, and T573S) transgene, i.e., a transgene encoding a JPH2 of at least 600 or at least 630 amino acids.

| SEQ ID NO: 91 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtac ccttcagatt aaaaataact | 180 |
| gaggtaaggg cctgggtagg ggaggtggtg tgagacgctc ctgtctctcc tctatctgcc | 240 |
| catcggccct ttggggagga ggaatgtgcc caaggactaa aaaaaggcca tggagccaga | 300 |
| ggggcgaggg caacagacct ttcatgggca aaccttgggg ccctgctgtc tagcatgccc | 360 |
| cactacgggt ctaggctgcc catgtaagga ggcaaggcct ggggacaccc gagatgcctg | 420 |
| gttataatta acccagacat gtggctgccc cccccccccc aacacctgct gcctctaaaa | 480 |
| ataaccctgt ccctggtgga tcccctgcat gcgaagatct tcgaacaagg ctgtggggga | 540 |
| ctgagggcag gctgtaacag gcttgggggc cagggcttat acgtgcctgg gactcccaaa | 600 |
| gtattactgt tccatgttcc cggcgaaggg ccagctgtcc cccgccagct agactcagca | 660 |
| cttagtttag gaaccagtga gcaagtcagc ccttggggca gcccatacaa ggccatgggg | 720 |
| ctgggcaagc tgcacgcctg ggtccggggt gggcacggtg cccgggcaac gagctgaaag | 780 |
| ctcatctgct ctcaggggcc cctccctggg gacagcccct cctggctagt cacaccctgt | 840 |
| aggctcctct atataaccca ggggcacagg ggctgccctc attctaccac cacctccaca | 900 |
| gcacagacag acactcagga gccagccagg gtaagtttag tctttttgtc ttttatttca | 960 |
| ggtcccggat ccggtggtgg tgcaaatcaa agaactgctc ctcagtggat gttgccttta | 1020 |
| cttctaggcc tgtacggaag tgttacttct gctctaaaag ctgcggaatt gtacccgcgc | 1080 |
| caccatgagt gggggccgct tcgactttga tgatggaggg gcgtactgcg ggggctggga | 1140 |
| ggggggaaag gcccatgggc atggactgtg cacaggcccc aagggccagg gcgaatactc | 1200 |
| tggctcctgg aactttggct ttgaggtggc aggtgtctac acctggccca gcggaaacac | 1260 |
| ctttgaggga tactggagcc agggcaaacg gcatgggctg ggcatagaga ccaaggggcg | 1320 |
| ctggctctac aagggcgagt ggacacatgg cttcaaggga cgctacggaa tccggcagag | 1380 |
| ctcaagcagc ggtgccaagt atgagggcac ctggaacaat ggcctgcaag acggctatgg | 1440 |
| caccgagacc tatgctgatg gagggacgta ccaaggccag ttcaccaacg gcatgcgcca | 1500 |
| tggctacgga gtacgccaga gcgtgcccta cgggatggcc gtggtggcca agtcgccgct | 1560 |
| gcgcacgtcg ctgtcgtccc tgcgcagcga gcacagcaac ggcacggtgg ccccggactc | 1620 |
| tcccgcctcg ccggcctccg acggccccgc gctgccctcg cccgccatcc cgcgtggcgg | 1680 |
| cttcgcgctc agcgccgccg ccaatgccga ggcggccgcg cgggcgccca agggcggcgg | 1740 |
| cctcttccag cggggcgcgc tgctgggcaa gctgcggcgc gcagagtcgc gcacgtccgt | 1800 |
| gggtagccag cgcagccgtg tcagcttcct taagagcgac ctcagctcgg gcgccagcga | 1860 |
| cgccgcgtcc accgccagcc tgggagaggc cgccgagggc gccgacgagg ccgcaccctt | 1920 |
| cgaggccgat atcgacgcca ccaccaccga gacctacatg ggcgagtgga agaacgacaa | 1980 |
| acgctcgggc ttcggcgtga gcgaacgctc cagtggcctc cgctacgagg gcgagtggct | 2040 |
| ggacaacctg cgccacggct atggctgcac cacgctgccc gacggccacc gcgaggaggg | 2100 |
| caagtaccgc cacaacgtgc tggtcaagga caccaagcgc cgcatgctgc agctcaagag | 2160 |
| caacaaggtc cgccagaaag tggagcacag tgtggagggt gcccagcgcg ccgctgctat | 2220 |
| cgcgcgccag aaggccgaga ttgccgcctc caggacaagc cacgccaagg ccaaagctga | 2280 |
| ggcagcggaa caggccgccc tggctgccaa ccaggagtcc aacattgctc gcactttggc | 2340 |
| cagggagctg gctccggact tctaccagcc aggtccggaa tatcagaagc gccggctgct | 2400 |
| gcaggagatc ctggagaact cggagagcct gctggagccc cccgaccggg gcgccggcgc | 2460 |
| agcgggcctc ccacagccgc cccgcgagag cccgcagctg cacgagcgtg agacccctcg | 2520 |
| gcccgagggt ggctccccgt caccggccgg gacgcccccg cagcccaagc ggcccaggcc | 2580 |
| cggggtgtcc aaggacggcc tgctgagccc aggcgcctgg aacggcgagc ccagcggtga | 2640 |
| gggcagccgg tcagtcactc cgtccgaggg cgcgggccgc cgcagccccg cgcgtccagc | 2700 |
| caccgagcgc atggccatcg aggctctgca ggcaccgcct gcgccgtcgc gggagccgga | 2760 |
| ggtggcgctt taccagggct accacagcta tgctgtgaag agcacgccgc ccgagccccc | 2820 |
| accctttgag gaccagcccg agcccgaggt ctccgggtcc gagtccgcgc cctcgtcccc | 2880 |
| ggccaccgcc ccgctgcagg cccccacgct ccgaggcccc gagcctgcac gcgagacccc | 2940 |
| cgccaagctg gagcccaagc ccatcatccc caaagccgag cccagggcca aggcccgcaa | 3000 |
| gactgaggct cgagggctga ccaaggcggg ggccaagaag aaggcgcgga aggaggccgc | 3060 |
| actggcggca gaggcggagg tggaggtgga agaggtcccc aacaccatcc tcatctgcat | 3120 |
| ggtgatcctg ctgaacatcg gcctggccat cctctttgtt cacctcctga cctgatcaac | 3180 |
| ctctggatta caaaatttgt gaaagattga ctggtattct taactatgtt gctcctttta | 3240 |
| cgctatgtgg atacgctgct ttaatgcctt tgtatcatgc tattgcttcc cgtatggctt | 3300 |
| tcattttctc ctccttgtat aaatcctggt tgctgtctct ttatgaggag ttgtggcccg | 3360 |
| ttgtcaggca acgtggcgtg gtgtgcactg tgtttgctga cgcaaccccc actggttggg | 3420 |
| gcattgccac cacctgtcag ctcctttccg ggactttcgc tttccccctc cctattgcca | 3480 |
| cggcggaact catcgccgcc tgccttgccc gctgctggac aggggctcgg ctgttgggca | 3540 |
| ctgacaattc cgtggtgttg tcggggaaat catcgtcctt tccttggctg ctcgcctgtg | 3600 |
| ttgccacctg gattctgcgc gggacgtcct tctgctacgt cccttcggcc ctcaatccag | 3660 |
| cggaccttcc ttcccgcggc ctgctgccgg ctctgcggcc tcttccgcgt cttcgccttc | 3720 |
| gccctcagac gagtcggatc tccctttggg ccgcctcccc gcactgcccg ggtggcatcc | 3780 |
| ctgtgacccc tccccagtgc ctctcctggc cctggaagtt gccactccag tgcccaccag | 3840 |
| ccttgtccta ataaaattaa gttgcatcat tttgtctgac taggtgtcct tctataatat | 3900 |
| tatggggtgg aggggggtgg tatggagcaa ggggcccaag ttgggaagaa acctgtaggg | 3960 |
| cctgccctaa ggaggaaccc ctagtgatgg agttggccac tccctctctg cgcgctcgct | 4020 |
| cgctcactga ggccgggcga ccaaaggtcg cccgacgccc gggctttgcc cgggcggcct | 4080 |
| cagtgagcga gcgagcgcgc agagagggag tggccaa | 4117 |

In a certain embodiment, the vector genome comprises, in 5′ to 3′ order, a 5′ITR; a hTnnT2 promoter; a JPH2 3mutAKAAKS (V155A, R156K, L204A, L205A, R572K, and T573S) transgene; an WPRE(x) element; a hGH sequence; and a 3′ ITR. The vector genome may comprise, in 5′ to 3′ order, any one of the polynucleotide sequences SEQ ID NO: 92; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length JPH2 3mutAKAAKS (V155A, R156K, L204A, L205A, R572K, and T573S) transgene, i.e. a transgene encoding a JPH2 of at least 600 or at least 630 amino acids.

| SEQ ID NO: 92 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtct cagtccatta ggagccagta | 180 |
| gcctggaaga tgtctttacc cccagcatca gttcaagtgg agcagcacat aactcttgcc | 240 |
| ctctgccttc caagattctg gtgctgagac ttatggagtg tcttggaggt tgccttctgc | 300 |
| cccccaaccc tgctcccagc tggccctccc aggcctgggt tgctggcctc tgctttatca | 360 |
| ggattctcaa gagggacagc tggtttatgt tgcatgactg ttccctgcat atctgctctg | 420 |
| gttttaaata gcttatctga gcagctggag gaccacatgg gcttatatgg cgtggggtac | 480 |
| atgttcctgt agccttgtcc ctggcacctg ccaaaatagc agccaacacc ccccaccccc | 540 |
| accgccatcc ccctgcccca cccgtcccct gtcgcacatt cctccctccg cagggctggc | 600 |
| tcaccaggcc ccagcccaca tgcctgctta aagccctctc catcctctgc ctcacccagt | 660 |
| ccccgctgag actgagcaga cgcctccagg atctgtcggc aggccaccat gagtgggggc | 720 |
| cgcttcgact ttgatgatgg aggggcgtac tgcgggggct gggagggggg aaaggcccat | 780 |
| gggcatggac tgtgcacagg ccccaagggc cagggcgaat actctggctc ctggaacttt | 840 |
| ggctttgagg tggcaggtgt ctacacctgg cccagcggaa acacctttga gggatactgg | 900 |
| agccagggca aacggcatgg gctgggcata gagaccaagg ggcgctggct ctacaagggc | 960 |
| gagtggacac atggcttcaa gggacgctac ggaatccggc agagctcaag cagcggtgcc | 1020 |
| aagtatgagg gcacctggaa caatggcctg caagacggct atggcaccga gacctatgct | 1080 |
| gatggaggga cgtaccaagg ccagttcacc aacggcatgc gccatggcta cggagtacgc | 1140 |
| cagagcgtgc cctacgggat ggccgtggtg gccaagtcgc cgctgcgcac gtcgctgtcg | 1200 |
| tccctgcgca gcgagcacag caacggcacg gtggccccgg actctcccgc ctcgccggcc | 1260 |
| tccgacggcc ccgcgctgcc ctcgcccgcc atcccgcgtg gcggcttcgc gctcagcgcc | 1320 |
| gccgccaatg ccgaggcggc cgcgcgggcg cccaagggcg gcggcctctt ccagcggggc | 1380 |
| gcgctgctgg gcaagctgcg gcgcgcagag tcgcgcacgt ccgtgggtag ccagcgcagc | 1440 |
| cgtgtcagct tccttaagag cgacctcagc tcgggcgcca gcgacgccgc gtccaccgcc | 1500 |
| agcctgggag aggccgccga gggcgccgac gaggccgcac ccttcgaggc cgatatcgac | 1560 |
| gccaccacca ccgagaccta catgggcgag tggaagaacg acaaacgctc gggcttcggc | 1620 |
| gtgagcgaac gctccagtgg cctccgctac gagggcgagt ggctggacaa cctgcgccac | 1680 |
| ggctatggct gcaccacgct gcccgacggc caccgcgagg agggcaagta ccgccacaac | 1740 |
| gtgctggtca aggacaccaa gcgccgcatg ctgcagctca agagcaacaa ggtccgccag | 1800 |
| aaagtggagc acagtgtgga gggtgcccag cgcgccgctg ctatcgcgcg ccagaaggcc | 1860 |
| gagattgccg cctccaggac aagccacgcc aaggccaaag ctgaggcagc ggaacaggcc | 1920 |
| gccctggctg ccaaccagga gtccaacatt gctcgcactt tggccaggga gctggctccg | 1980 |
| gacttctacc agccaggtcc ggaatatcag aagcgccggc tgctgcagga gatcctggag | 2040 |
| aactcggaga gcctgctgga gccccccgac cggggcgccg gcgcagcggg cctcccacag | 2100 |
| ccgccccgcg agagcccgca gctgcacgag cgtgagaccc ctcggcccga gggtggctcc | 2160 |
| ccgtcaccgg ccgggacgcc cccgcagccc aagcggccca ggcccggggt gtccaaggac | 2220 |
| ggcctgctga gcccaggcgc ctggaacggc gagcccagcg gtgagggcag ccggtcagtc | 2280 |
| actccgtccg agggcgcggg ccgccgcagc cccgcgcgtc cagccaccga gcgcatggcc | 2340 |
| atcgaggctc tgcaggcacc gcctgcgccg tcgcgggagc cggaggtggc gctttaccag | 2400 |
| ggctaccaca gctatgctgt gaagagcacg ccgcccgagc ccccaccctt tgaggaccag | 2460 |
| cccgagcccg aggtctccgg gtccgagtcc gcgccctcgt ccccggccac cgccccgctg | 2520 |
| caggccccca cgctccgagg ccccgagcct gcacgcgaga cccccgccaa gctggagccc | 2580 |
| aagcccatca tccccaaagc cgagcccagg gccaaggccc gcaagactga ggctcgaggg | 2640 |
| ctgaccaagg cgggggccaa gaagaaggcg cggaaggagg ccgcactggc ggcagaggcg | 2700 |
| gaggtggagg tggaagaggt ccccaacacc atcctcatct gcatggtgat cctgctgaac | 2760 |
| atcggcctgg ccatcctctt tgttcacctc ctgacctgat caacctctgg attacaaaat | 2820 |
| ttgtgaaaga ttgactggta ttcttaacta tgttgctcct tttacgctat gtggatacgc | 2880 |
| tgctttaatg cctttgtatc atgctattgc ttcccgtatg gctttcattt tctcctcctt | 2940 |
| gtataaatcc tggttgctgt ctctttatga ggagttgtgg cccgttgtca ggcaacgtgg | 3000 |
| cgtggtgtgc actgtgtttg ctgacgcaac ccccactggt tggggcattg ccaccacctg | 3060 |
| tcagctcctt tccgggactt tcgctttccc cctccctatt gccacggcgg aactcatcgc | 3120 |
| cgcctgcctt gcccgctgct ggacaggggc tcggctgttg ggcactgaca attccgtggt | 3180 |
| gttgtcgggg aaatcatcgt cctttccttg gctgctcgcc tgtgttgcca cctggattct | 3240 |
| gcgcgggacg tccttctgct acgtcccttc ggccctcaat ccagcggacc ttccttcccg | 3300 |
| cggcctgctg ccggctctgc ggcctcttcc gcgtcttcgc cttcgccctc agacgagtcg | 3360 |
| gatctccctt tgggccgcct ccccgcactg cccgggtggc atccctgtga cccctcccca | 3420 |
| gtgcctctcc tggccctgga agttgccact ccagtgccca ccagccttgt cctaataaaa | 3480 |
| ttaagttgca tcattttgtc tgactaggtg tccttctata atattatggg gtggaggggg | 3540 |
| gtggtatgga gcaaggggcc caagttggga agaaacctgt agggcctgcc ctaaggagga | 3600 |
| acccctagtg atggagttgg ccactccctc tctgcgcgct cgctcgctca ctgaggccgg | 3660 |
| gcgaccaaag gtcgcccgac gcccgggctt tgcccgggcg gcctcagtga gcgagcgagc | 3720 |
| gcgcagagag ggagtggcca a | 3741 |

In a certain embodiment, the vector genome comprises, in 5′ to 3′ order, a 5′ITR; a MHCK7 promoter, a JPH2 3mutAKAAKS (V155A, R156K, L204A, L205A, R572K, and T573S) transgene; a GFP tag; an WPRE(x) element; a hGH sequence; and a 3′ ITR. The vector genome may comprise, in 5′ to 3′ order, any one of the polynucleotide sequences SEQ ID NO: 93; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length JPH2 3mutAKAAKS (V155A, R156K, L204A, L205A, R572K, and T573S) transgene, i.e. a transgene encoding a JPH2 of at least 600 or at least 630 amino acids.

| SEQ ID NO: 93 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtac ccttcagatt aaaaataact | 180 |
| gaggtaaggg cctgggtagg ggaggtggtg tgagacgctc ctgtctctcc tctatctgcc | 240 |
| catcggccct ttggggagga ggaatgtgcc caaggactaa aaaaaggcca tggagccaga | 300 |
| ggggcgaggg caacagacct ttcatgggca aaccttgggg ccctgctgtc tagcatgccc | 360 |
| cactacgggt ctaggctgcc catgtaagga ggcaaggcct ggggacaccc gagatgcctg | 420 |
| gttataatta acccagacat gtggctgccc cccccccccc aacacctgct gcctctaaaa | 480 |
| ataaccctgt ccctggtgga tcccctgcat gcgaagatct tcgaacaagg ctgtggggga | 540 |
| ctgagggcag gctgtaacag gcttgggggc cagggcttat acgtgcctgg gactcccaaa | 600 |
| gtattactgt tccatgttcc cggcgaaggg ccagctgtcc cccgccagct agactcagca | 660 |
| cttagtttag gaaccagtga gcaagtcagc ccttggggca gcccatacaa ggccatgggg | 720 |
| ctgggcaagc tgcacgcctg ggtccggggt gggcacggtg cccgggcaac gagctgaaag | 780 |
| ctcatctgct ctcaggggcc cctccctggg gacagcccct cctggctagt cacaccctgt | 840 |
| aggctcctct atataaccca ggggcacagg ggctgccctc attctaccac cacctccaca | 900 |
| gcacagacag acactcagga gccagccagg ccaccatgag tgggggccgc ttcgactttg | 960 |
| atgatggagg ggcgtactgc gggggctggg aggggggaaa ggcccatggg catggactgt | 1020 |
| gcacaggccc caagggccag ggcgaatact ctggctcctg gaactttggc tttgaggtgg | 1080 |
| caggtgtcta cacctggccc agcggaaaca cctttgaggg atactggagc cagggcaaac | 1140 |
| ggcatgggct gggcatagag accaaggggc gctggctcta caagggcgag tggacacatg | 1200 |
| gcttcaaggg acgctacgga atccggcaga gctcaagcag cggtgccaag tatgagggca | 1260 |
| cctggaacaa tggcctgcaa gacggctatg gcaccgagac ctatgctgat ggagggacgt | 1320 |
| accaaggcca gttcaccaac ggcatgcgcc atggctacgg agtacgccag agcgtgccct | 1380 |
| acgggatggc cgtggtggcc aagtcgccgc tgcgcacgtc gctgtcgtcc ctgcgcagcg | 1440 |
| agcacagcaa cggcacggtg gccccggact ctcccgcctc gccggcctcc gacggccccg | 1500 |
| cgctgccctc gcccgccatc ccgcgtggcg gcttcgcgct cagcgccgcc gccaatgccg | 1560 |
| aggcggccgc gcgggcgccc aagggcggcg gcctcttcca gcggggcgcg ctgctgggca | 1620 |
| agctgcggcg cgcagagtcg cgcacgtccg tgggtagcca gcgcagccgt gtcagcttcc | 1680 |
| ttaagagcga cctcagctcg ggcgccagcg acgccgcgtc caccgccagc ctgggagagg | 1740 |
| ccgccgaggg cgccgacgag gccgcaccct tcgaggccga tatcgacgcc accaccaccg | 1800 |
| agacctacat gggcgagtgg aagaacgaca aacgctcggg cttcggcgtg agcgaacgct | 1860 |
| ccagtggcct ccgctacgag ggcgagtggc tggacaacct gcgccacggc tatggctgca | 1920 |
| ccacgctgcc cgacggccac cgcgaggagg gcaagtaccg ccacaacgtg ctggtcaagg | 1980 |
| acaccaagcg ccgcatgctg cagctcaaga gcaacaaggt ccgccagaaa gtggagcaca | 2040 |
| gtgtggaggg tgcccagcgc gccgctgcta tcgcgcgcca gaaggccgag attgccgcct | 2100 |
| ccaggacaag ccacgccaag gccaaagctg aggcagcgga acaggccgcc ctggctgcca | 2160 |
| accaggagtc caacattgct cgcactttgg ccagggagct ggctccggac ttctaccagc | 2220 |
| caggtccgga atatcagaag cgccggctgc tgcaggagat cctggagaac tcggagagcc | 2280 |
| tgctggagcc ccccgaccgg ggcgccggcg cagcgggcct cccacagccg ccccgcgaga | 2340 |
| gcccgcagct gcacgagcgt gagacccctc ggcccgaggg tggctccccg tcaccggccg | 2400 |
| ggacgccccc gcagcccaag cggcccaggc ccggggtgtc caaggacggc ctgctgagcc | 2460 |
| caggcgcctg gaacggcgag cccagcggtg agggcagccg gtcagtcact ccgtccgagg | 2520 |
| gcgcgggccg ccgcagcccc gcgcgtccag ccaccgagcg catggccatc gaggctctgc | 2580 |
| aggcaccgcc tgcgccgtcg cgggagccgg aggtggcgct ttaccagggc taccacagct | 2640 |
| atgctgtgaa gagcacgccg cccgagcccc caccctttga ggaccagccc gagcccgagg | 2700 |
| tctccgggtc cgagtccgcg ccctcgtccc cggccaccgc cccgctgcag gcccccacgc | 2760 |
| tccgaggccc cgagcctgca cgcgagaccc ccgccaagct ggagcccaag cccatcatcc | 2820 |
| ccaaagccga gcccagggcc aaggcccgca agactgaggc tcgagggctg accaaggcgg | 2880 |
| gggccaagaa gaaggcgcgg aaggaggccg cactggcggc agaggcggag gtggaggtgg | 2940 |
| aagaggtccc caacaccatc ctcatctgca tggtgatcct gctgaacatc ggcctggcca | 3000 |
| tcctctttgt tcacctcctg accggatccg gcagtggaga gggcagagga agtctgctaa | 3060 |
| catgcggtga cgtcgaggag aatcctggcc caatgagcaa gggcgaggag ctgttcaccg | 3120 |
| gcgtggtgcc catcctggtg gagctggacg gcgacgtgaa cggccacaag ttcagcgtga | 3180 |
| gaggcgaggg cgagggcgac gccaccaacg gcaagctgac cctgaagttc atctgcacca | 3240 |
| ccggcaagct gcccgtgccc tggcccaccc tggtgaccac cctgacctac ggcgtgctgt | 3300 |
| gcttcagcag ataccccgac cacatgaaga gacacgactt cttcaagagc gccatgcccg | 3360 |
| agggctacgt gcaggagaga accatcagct tcaaggacga cggcacctac aagaccagag | 3420 |
| ccgaggtgaa gttcgagggc gacaccctgg tgaacagaat cgagctgaag ggcatcgact | 3480 |
| tcaaggagga cggcaacatc ctgggccaca agctggagta caacttcaac agccacaacg | 3540 |
| tgtacatcac cgccgacaag cagaagaacg gcatcaaggc ctacttcaag atcagacaca | 3600 |
| acgtggagga cggcagcgtg cagctggccg accactacca gcagaacacc cccatcggcg | 3660 |
| acggccccgt gctgctgccc gacaaccact acctgagcac ccagagcgtg ctgagcaagg | 3720 |
| accccaacga gaagagagac cacatggtgc tgctggagga cgtgaccgcc gccggcatca | 3780 |
| cccacggcat ggacgagctg tacaagtgat caacctctgg attacaaaat ttgtgaaaga | 3840 |
| ttgactggta ttcttaacta tgttgctcct tttacgctat gtggatacgc tgctttaatg | 3900 |
| cctttgtatc atgctattgc ttcccgtatg gctttcattt tctcctcctt gtataaatcc | 3960 |
| tggttgctgt ctctttatga ggagttgtgg cccgttgtca ggcaacgtgg cgtggtgtgc | 4020 |
| actgtgtttg ctgacgcaac ccccactggt tggggcattg ccaccacctg tcagctcctt | 4080 |
| tccgggactt tcgctttccc cctccctatt gccacggcgg aactcatcgc cgcctgcctt | 4140 |
| gcccgctgct ggacaggggc tcggctgttg ggcactgaca attccgtggt gttgtcgggg | 4200 |
| aaatcatcgt cctttccttg gctgctcgcc tgtgttgcca cctggattct gcgcgggacg | 4260 |
| tccttctgct acgtcccttc ggccctcaat ccagcggacc ttccttcccg cggcctgctg | 4320 |
| ccggctctgc ggcctcttcc gcgtcttcgc cttcgccctc agacgagtcg gatctccctt | 4380 |
| tgggccgcct ccccgcactg cccgggtggc atccctgtga cccctcccca gtgcctctcc | 4440 |
| tggccctgga agttgccact ccagtgccca ccagccttgt cctaataaaa ttaagttgca | 4500 |
| tcattttgtc tgactaggtg tccttctata atattatggg gtggaggggg gtggtatgga | 4560 |
| gcaaggggcc caagttggga agaaacctgt agggcctgcc ctaaggagga acccctagtg | 4620 |
| atggagttgg ccactccctc tctgcgcgct cgctcgctca ctgaggccgg gcgaccaaag | 4680 |
| gtcgcccgac gcccgggctt tgcccgggcg gcctcagtga gcgagcgagc gcgcagagag | 4740 |
| ggagtggcca a | 4751 |

In a certain embodiment, the vector genome comprises, in 5′ to 3′ order, a 5′ITR; a hTnnT2 promoter; a JPH2 3mutAKAAKS (V155A, R156K, L204A, L205A, R572K, and T573S) transgene; a GFP tag; an WPRE(x) element; a hGH sequence; and a 3′ ITR. The vector genome may comprise, in 5′ to 3′ order, any one of the polynucleotide sequences SEQ ID NO: 94; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length JPH2 3mutAKAAKS (V155A, R156K, L204A, L205A, R572K, and T573S) transgene, i.e. a transgene encoding a JPH2 of at least 600 or at least 630 amino acids.

| SEQ ID NO: 94 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtct cagtccatta ggagccagta | 180 |
| gcctggaaga tgtctttacc cccagcatca gttcaagtgg agcagcacat aactcttgcc | 240 |
| ctctgccttc caagattctg gtgctgagac ttatggagtg tcttggaggt tgccttctgc | 300 |
| cccccaaccc tgctcccagc tggccctccc aggcctgggt tgctggcctc tgctttatca | 360 |
| ggattctcaa gagggacagc tggtttatgt tgcatgactg ttccctgcat atctgctctg | 420 |
| gttttaaata gcttatctga gcagctggag gaccacatgg gcttatatgg cgtggggtac | 480 |
| atgttcctgt agccttgtcc ctggcacctg ccaaaatagc agccaacacc ccccaccccc | 540 |
| accgccatcc ccctgcccca cccgtcccct gtcgcacatt cctccctccg cagggctggc | 600 |
| tcaccaggcc ccagcccaca tgcctgctta aagccctctc catcctctgc ctcacccagt | 660 |
| ccccgctgag actgagcaga cgcctccagg atctgtcggc aggccaccat gagtgggggc | 720 |
| cgcttcgact ttgatgatgg aggggcgtac tgcgggggct gggagggggg aaaggcccat | 780 |
| gggcatggac tgtgcacagg ccccaagggc cagggcgaat actctggctc ctggaacttt | 840 |
| ggctttgagg tggcaggtgt ctacacctgg cccagcggaa acacctttga gggatactgg | 900 |
| agccagggca aacggcatgg gctgggcata gagaccaagg ggcgctggct ctacaagggc | 960 |
| gagtggacac atggcttcaa gggacgctac ggaatccggc agagctcaag cagcggtgcc | 1020 |
| aagtatgagg gcacctggaa caatggcctg caagacggct atggcaccga gacctatgct | 1080 |
| gatggaggga cgtaccaagg ccagttcacc aacggcatgc gccatggcta cggagtacgc | 1140 |
| cagagcgtgc cctacgggat ggccgtggtg gccaagtcgc cgctgcgcac gtcgctgtcg | 1200 |
| tccctgcgca gcgagcacag caacggcacg gtggccccgg actctcccgc ctcgccggcc | 1260 |
| tccgacggcc ccgcgctgcc ctcgcccgcc atcccgcgtg gcggcttcgc gctcagcgcc | 1320 |
| gccgccaatg ccgaggcggc cgcgcgggcg cccaagggcg gcggcctctt ccagcggggc | 1380 |
| gcgctgctgg gcaagctgcg gcgcgcagag tcgcgcacgt ccgtgggtag ccagcgcagc | 1440 |
| cgtgtcagct tccttaagag cgacctcagc tcgggcgcca gcgacgccgc gtccaccgcc | 1500 |
| agcctgggag aggccgccga gggcgccgac gaggccgcac ccttcgaggc cgatatcgac | 1560 |
| gccaccacca ccgagaccta catgggcgag tggaagaacg acaaacgctc gggcttcggc | 1620 |
| gtgagcgaac gctccagtgg cctccgctac gagggcgagt ggctggacaa cctgcgccac | 1680 |
| ggctatggct gcaccacgct gcccgacggc caccgcgagg agggcaagta ccgccacaac | 1740 |
| gtgctggtca aggacaccaa gcgccgcatg ctgcagctca agagcaacaa ggtccgccag | 1800 |
| aaagtggagc acagtgtgga gggtgcccag cgcgccgctg ctatcgcgcg ccagaaggcc | 1860 |
| gagattgccg cctccaggac aagccacgcc aaggccaaag ctgaggcagc ggaacaggcc | 1920 |
| gccctggctg ccaaccagga gtccaacatt gctcgcactt tggccaggga gctggctccg | 1980 |
| gacttctacc agccaggtcc ggaatatcag aagcgccggc tgctgcagga gatcctggag | 2040 |
| aactcggaga gcctgctgga gccccccgac cggggcgccg gcgcagcggg cctcccacag | 2100 |
| ccgccccgcg agagcccgca gctgcacgag cgtgagaccc ctcggcccga gggtggctcc | 2160 |
| ccgtcaccgg ccgggacgcc cccgcagccc aagcggccca ggcccggggt gtccaaggac | 2220 |
| ggcctgctga gcccaggcgc ctggaacggc gagcccagcg gtgagggcag ccggtcagtc | 2280 |
| actccgtccg agggcgcggg ccgccgcagc cccgcgcgtc cagccaccga gcgcatggcc | 2340 |
| atcgaggctc tgcaggcacc gcctgcgccg tcgcgggagc cggaggtggc gctttaccag | 2400 |
| ggctaccaca gctatgctgt gaagagcacg ccgcccgagc ccccaccctt tgaggaccag | 2460 |
| cccgagcccg aggtctccgg gtccgagtcc gcgccctcgt ccccggccac cgccccgctg | 2520 |
| caggccccca cgctccgagg ccccgagcct gcacgcgaga cccccgccaa gctggagccc | 2580 |
| aagcccatca tccccaaagc cgagcccagg gccaaggccc gcaagactga ggctcgaggg | 2640 |
| ctgaccaagg cgggggccaa gaagaaggcg cggaaggagg ccgcactggc ggcagaggcg | 2700 |
| gaggtggagg tggaagaggt ccccaacacc atcctcatct gcatggtgat cctgctgaac | 2760 |
| atcggcctgg ccatcctctt tgttcacctc ctgaccggat ccggcagtgg agagggcaga | 2820 |
| ggaagtctgc taacatgcgg tgacgtcgag gagaatcctg gcccaatgag caagggcgag | 2880 |
| gagctgttca ccggcgtggt gcccatcctg gtggagctgg acggcgacgt gaacggccac | 2940 |
| aagttcagcg tgagaggcga gggcgagggc gacgccacca acggcaagct gaccctgaag | 3000 |
| ttcatctgca ccaccggcaa gctgcccgtg ccctggccca ccctggtgac caccctgacc | 3060 |
| tacggcgtgc tgtgcttcag cagatacccc gaccacatga agagacacga cttcttcaag | 3120 |
| agcgccatgc ccgagggcta cgtgcaggag agaaccatca gcttcaagga cgacggcacc | 3180 |
| tacaagacca gagccgaggt gaagttcgag ggcgacaccc tggtgaacag aatcgagctg | 3240 |
| aagggcatcg acttcaagga ggacggcaac atcctgggcc acaagctgga gtacaacttc | 3300 |
| aacagccaca acgtgtacat caccgccgac aagcagaaga acggcatcaa ggcctacttc | 3360 |
| aagatcagac acaacgtgga ggacggcagc gtgcagctgg ccgaccacta ccagcagaac | 3420 |
| acccccatcg gcgacggccc cgtgctgctg cccgacaacc actacctgag cacccagagc | 3480 |
| gtgctgagca aggaccccaa cgagaagaga gaccacatgg tgctgctgga ggacgtgacc | 3540 |
| gccgccggca tcacccacgg catggacgag ctgtacaagt gatcaacctc tggattacaa | 3600 |
| aatttgtgaa agattgactg gtattcttaa ctatgttgct ccttttacgc tatgtggata | 3660 |
| cgctgcttta atgcctttgt atcatgctat tgcttcccgt atggctttca ttttctcctc | 3720 |
| cttgtataaa tcctggttgc tgtctcttta tgaggagttg tggcccgttg tcaggcaacg | 3780 |
| tggcgtggtg tgcactgtgt ttgctgacgc aacccccact ggttggggca ttgccaccac | 3840 |
| ctgtcagctc ctttccggga ctttcgcttt ccccctccct attgccacgg cggaactcat | 3900 |
| cgccgcctgc cttgcccgct gctggacagg ggctcggctg ttgggcactg acaattccgt | 3960 |
| ggtgttgtcg gggaaatcat cgtcctttcc ttggctgctc gcctgtgttg ccacctggat | 4020 |
| tctgcgcggg acgtccttct gctacgtccc ttcggccctc aatccagcgg accttccttc | 4080 |
| ccgcggcctg ctgccggctc tgcggcctct tccgcgtctt cgccttcgcc ctcagacgag | 4140 |
| tcggatctcc ctttgggccg cctccccgca ctgcccgggt ggcatccctg tgacccctcc | 4200 |
| ccagtgcctc tcctggccct ggaagttgcc actccagtgc ccaccagcct tgtcctaata | 4260 |
| aaattaagtt gcatcatttt gtctgactag gtgtccttct ataatattat ggggtggagg | 4320 |
| ggggtggtat ggagcaaggg gcccaagttg ggaagaaacc tgtagggcct gccctaagga | 4380 |
| ggaaccccta gtgatggagt tggccactcc ctctctgcgc gctcgctcgc tcactgaggc | 4440 |
| cgggcgacca aaggtcgccc gacgcccggg ctttgcccgg gcggcctcag tgagcgagcg | 4500 |
| agcgcgcaga gagggagtgg ccaa | 4524 |

In a certain embodiment, the vector genome comprises, in 5′ to 3′ order, a 5′ITR; a CMV enhancer element; a CMV promoter; a JPH2 3mutAKAAKS (V155A, R156K, L204A, L205A, R572K, and T573S) transgene; a GFP tag; an WPRE(x) element; a hGH sequence; and a 3′ ITR. The vector genome may comprise, in 5′ to 3′ order, any one of the polynucleotide sequences SEQ ID NO: 95; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The JPH2 transgene of this embodiment is a full length 3mutAKAAKS (V155A, R156K, L204A, L205A, R572K, and T573S) transgene, i.e. a transgene encoding a JPH2 of at least 600 or at least 630 amino acids.

| SEQ ID NO: 95 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtta catacgttac ataacttacg | 180 |
| gtaaatggcc cgcctggctg accgcccaac gacccccgcc cattgacgtc aataatgacg | 240 |
| tatgttccca tagtaacgcc aatagggact ttccattgac gtcaatgggt ggagtattta | 300 |
| cggtaaactg cccacttggc agtacatcaa gtgtatcata tgccaagtac gccccctatt | 360 |
| gacgtcaatg acggtaaatg gcccgcctgg cattatgccc agtacatgac cttatgggac | 420 |
| tttcctactt ggcagtacat ctacgtatta gtcatcgcta ttaccatggt gatgcggttt | 480 |
| tggcagtaca tcaatgggcg tggatagcgg tttgactcac ggggatttcc aagtctccac | 540 |
| cccattgacg tcaatgggag tttgttttgg caccaaaatc aacgggactt tccaaaatgt | 600 |
| cgtaacaact ccgccccatt gacgcaaatg ggcggtaggc gtgtacggtg ggaggtctat | 660 |
| ataagcagag ctcgtttagt gaaccgtcag atcgcctgga gacgccatcc acgctgtttt | 720 |
| gacctccata gaagacaccg ggaccgatcc agcctccgcg ggccaccatg agtgggggcc | 780 |
| gcttcgactt tgatgatgga ggggcgtact gcgggggctg ggagggggga aaggcccatg | 840 |
| ggcatggact gtgcacaggc cccaagggcc agggcgaata ctctggctcc tggaactttg | 900 |
| gctttgaggt ggcaggtgtc tacacctggc ccagcggaaa cacctttgag ggatactgga | 960 |
| gccagggcaa acggcatggg ctgggcatag agaccaaggg gcgctggctc tacaagggcg | 1020 |
| agtggacaca tggcttcaag ggacgctacg gaatccggca gagctcaagc agcggtgcca | 1080 |
| agtatgaggg cacctggaac aatggcctgc aagacggcta tggcaccgag acctatgctg | 1140 |
| atggagggac gtaccaaggc cagttcacca acggcatgcg ccatggctac ggagtacgcc | 1200 |
| agagcgtgcc ctacgggatg gccgtggtgg ccaagtcgcc gctgcgcacg tcgctgtcgt | 1260 |
| ccctgcgcag cgagcacagc aacggcacgg tggccccgga ctctcccgcc tcgccggcct | 1320 |
| ccgacggccc cgcgctgccc tcgcccgcca tcccgcgtgg cggcttcgcg ctcagcgccg | 1380 |
| ccgccaatgc cgaggcggcc gcgcgggcgc ccaagggcgg cggcctcttc cagcggggcg | 1440 |
| cgctgctggg caagctgcgg cgcgcagagt cgcgcacgtc cgtgggtagc cagcgcagcc | 1500 |
| gtgtcagctt ccttaagagc gacctcagct cgggcgccag cgacgccgcg tccaccgcca | 1560 |
| gcctgggaga ggccgccgag ggcgccgacg aggccgcacc cttcgaggcc gatatcgacg | 1620 |
| ccaccaccac cgagacctac atgggcgagt ggaagaacga caaacgctcg ggcttcggcg | 1680 |
| tgagcgaacg ctccagtggc ctccgctacg agggcgagtg gctggacaac ctgcgccacg | 1740 |
| gctatggctg caccacgctg cccgacggcc accgcgagga gggcaagtac cgccacaacg | 1800 |
| tgctggtcaa ggacaccaag cgccgcatgc tgcagctcaa gagcaacaag gtccgccaga | 1860 |
| aagtggagca cagtgtggag ggtgcccagc gcgccgctgc tatcgcgcgc cagaaggccg | 1920 |
| agattgccgc ctccaggaca agccacgcca aggccaaagc tgaggcagcg gaacaggccg | 1980 |
| ccctggctgc caaccaggag tccaacattg ctcgcacttt ggccagggag ctggctccgg | 2040 |
| acttctacca gccaggtccg gaatatcaga agcgccggct gctgcaggag atcctggaga | 2100 |
| actcggagag cctgctggag ccccccgacc ggggcgccgg cgcagcgggc ctcccacagc | 2160 |
| cgccccgcga gagcccgcag ctgcacgagc gtgagacccc tcggcccgag ggtggctccc | 2220 |
| cgtcaccggc cgggacgccc ccgcagccca agcggcccag gcccggggtg tccaaggacg | 2280 |
| gcctgctgag cccaggcgcc tggaacggcg agcccagcgg tgagggcagc cggtcagtca | 2340 |
| ctccgtccga gggcgggggc cgccgcagcc ccgcgcgtcc agccaccgag cgcatggcca | 2400 |
| tcgaggctct gcaggcaccg cctgcgccgt cgcgggagcc ggaggtggcg ctttaccagg | 2460 |
| gctaccacag ctatgctgtg aagagcacgc cgcccgagcc cccacccttt gaggaccagc | 2520 |
| ccgagcccga ggtctccggg tccgagtccg cgccctcgtc cccggccacc gccccgctgc | 2580 |
| aggcccccac gctccgaggc cccgagcctg cacgcgagac ccccgccaag ctggagccca | 2640 |
| agcccatcat ccccaaagcc gagcccaggg ccaaggcccg caagactgag gctcgagggc | 2700 |
| tgaccaaggc gggggccaag aagaaggcgc ggaaggaggc cgcactggcg gcagaggcgg | 2760 |
| aggtggaggt ggaagaggtc cccaacacca tcctcatctg catggtgatc ctgctgaaca | 2820 |
| tcggcctggc catcctcttt gttcacctcc tgaccggatc cggcagtgga gagggcagag | 2880 |
| gaagtctgct aacatgcggt gacgtcgagg agaatcctgg cccaatgagc aagggcgagg | 2940 |
| agctgttcac cggcgtggtg cccatcctgg tggagctgga cggcgacgtg aacggccaca | 3000 |
| agttcagcgt gagaggcgag ggcgagggcg acgccaccaa cggcaagctg accctgaagt | 3060 |
| tcatctgcac caccggcaag ctgcccgtgc cctggcccac cctggtgacc accctgacct | 3120 |
| acggcgtgct gtgcttcagc agataccccg accacatgaa gagacacgac ttcttcaaga | 3180 |
| gcgccatgcc cgagggctac gtgcaggaga gaaccatcag cttcaaggac gacggcacct | 3240 |
| acaagaccag agccgaggtg aagttcgagg gcgacaccct ggtgaacaga atcgagctga | 3300 |
| agggcatcga cttcaaggag gacggcaaca tcctgggcca caagctggag tacaacttca | 3360 |
| acagccacaa cgtgtacatc accgccgaca agcagaagaa cggcatcaag gcctacttca | 3420 |
| agatcagaca caacgtggag gacggcagcg tgcagctggc cgaccactac cagcagaaca | 3480 |
| cccccatcgg cgacggcccc gtgctgctgc ccgacaacca ctacctgagc acccagagcg | 3540 |
| tgctgagcaa ggaccccaac gagaagagag accacatggt gctgctggag gacgtgaccg | 3600 |
| ccgccggcat cacccacggc atggacgagc tgtacaagtg atcaacctct ggattacaaa | 3660 |
| atttgtgaaa gattgactgg tattcttaac tatgttgctc cttttacgct atgtggatac | 3720 |
| gctgctttaa tgcctttgta tcatgctatt gcttcccgta tggctttcat tttctcctcc | 3780 |
| ttgtataaat cctggttgct gtctctttat gaggagttgt ggcccgttgt caggcaacgt | 3840 |
| ggcgtggtgt gcactgtgtt tgctgacgca acccccactg gttggggcat tgccaccacc | 3900 |
| tgtcagctcc tttccgggac tttcgctttc cccctcccta ttgccacggc ggaactcatc | 3960 |
| gccgcctgcc ttgcccgctg ctggacaggg gctcggctgt tgggcactga caattccgtg | 4020 |
| gtgttgtcgg ggaaatcatc gtcctttcct tggctgctcg cctgtgttgc cacctggatt | 4080 |
| ctgcgcggga cgtccttctg ctacgtccct tcggccctca atccagcgga ccttccttcc | 4140 |
| cgcggcctgc tgccggctct gcggcctctt ccgcgtcttc gccttcgccc tcagacgagt | 4200 |
| cggatctccc tttgggccgc ctccccgcac tgcccgggtg gcatccctgt gacccctccc | 4260 |
| cagtgcctct cctggccctg gaagttgcca ctccagtgcc caccagcctt gtcctaataa | 4320 |
| aattaagttg catcattttg tctgactagg tgtccttcta taatattatg gggtggaggg | 4380 |
| gggtggtatg gagcaagggg cccaagttgg gaagaaacct gtagggcctg ccctaaggag | 4440 |
| gaacccctag tgatggagtt ggccactccc tctctgcgcg ctcgctcgct cactgaggcc | 4500 |
| gggcgaccaa aggtcgcccg acgcccgggc tttgcccggg cggcctcagt gagcgagcga | 4560 |
| gcgcgcagag agggagtggc caa | 4583 |

In each case, the optional WPRE element may be present or absent.

### Adeno-Associated Virus Vector and Uses Thereof

AAV vectors useful in the practice of the present disclosure can be packaged into AAV virions (viral particles) using various systems including adenovirus-based and helper-free systems. Standard methods in AAV biology include those described in Kwon and Schaffer. Pharm Res. (2008) 25 (3): 489-99; Wu et al. Mol. Ther. (2006) 14 (3): 316-27. Burger et al. Mol. Ther. (2004) 10 (2): 302-17; Grimm et al. Curr Gene Ther. (2003) 3 (4): 281-304; Deyle D R, Russell D W. Curr Opin Mol Ther. (2009) 11 (4): 442-447; McCarty et al. Gene Ther. (2001) 8 (16): 1248-54; and Duan et al. Mol Ther. (2001) 4 (4): 383-91. Helper-free systems included those described in U.S. Pat. Nos. 6,004,797; 7,588,772; and 7,094,604;

AAV DNA in the rAAV genomes may be from any AAV variant or serotype for which a recombinant virus can be derived including, but not limited to, AAV variants or serotypes AAV-1, AAV-2, AAV-3, AAV-4, AAV-5, AAV-6, AAV-7, AAV-8, AAV-9, AAV-10, AAV-11, AAV-12, AAV-13, AAVrh.74, and AAVrh10. Production of pseudotyped rAAV is disclosed in, for example, WO 01/83692. Other types of rAAV variants, for example rAAV with capsid mutations, are also contemplated. See, for example, Marsic et al., Molecular Therapy, 22 (11): 1900-1909 (2014). The nucleotide sequences of the genomes of various AAV serotypes are known in the art.

In some cases, the rAAV comprises a self-complementary genome. As defined herein, an rAAV comprising a “self-complementary” or “double stranded” genome refers to an rAAV which has been engineered such that the coding region of the rAAV is configured to form an intra-molecular double-stranded DNA template, as described in McCarty et al. Self-complementary recombinant adeno-associated virus (scAAV) vectors promoter efficient transduction independently of DNA synthesis. Gene Therapy. 8 (16): 1248-54 (2001). The present disclosure contemplates the use, in some cases, of an rAAV comprising a self-complementary genome because upon infection (such transduction), rather than waiting for cell mediated synthesis of the second strand of the rAAV genome, the two complementary halves of scAAV will associate to form one double stranded DNA (dsDNA) unit that is ready for immediate replication and transcription. It will be understood that instead of the full coding capacity found in rAAV (4.7-6 kb), rAAV comprising a self-complementary genome can only hold about half of that amount (≈2.4 kb).

In other cases, the rAAV vector comprises a single stranded genome. As defined herein, a “single standard” genome refers to a genome that is not self-complementary. In most cases, non-recombinant AAVs have singled stranded DNA genomes. There have been some indications that rAAVs should be scAAVs to achieve efficient transduction of cells. The present disclosure contemplates, however, rAAV vectors that maybe have singled stranded genomes, rather than self-complementary genomes, with the understanding that other genetic modifications of the rAAV vector may be beneficial to obtain optimal gene transcription in target cells.

In some cases, the rAAV vector is of the serotype AAV1, AAV2, AAV4, AAV5, AAV6, AAV7, AAV8, AAV9, AAV10, AAV11, AAV12, AAV13, AAVrh10, or AAVrh74. Production of pseudotyped rAAV is disclosed in, for example, WO 01/83692. Other types of rAAV variants, for example rAAV with capsid mutations, are also contemplated. See, for example, Marsic et al., Molecular Therapy, 22 (11): 1900-1909 (2014). In some cases, the rAAV vector is of the serotype AAV9. In some embodiments, said rAAV vector is of serotype AAV9 and comprises a single stranded genome. In some embodiments, said rAAV vector is of serotype AAV9 and comprises a self-complementary genome. In some embodiments, a rAAV vector comprises the inverted terminal repeat (ITR) sequences of AAV2. In some embodiments, the rAAV vector comprises an AAV2 genome, such that the rAAV vector is an AAV-2/9 vector, an AAV-2/6 vector, or an AAV-2/8 vector.

Full-length sequences and sequences for capsid genes for most known AAVs are provided in U.S. Pat. No. 8,524,446, which is incorporated herein in its entirety.

AAV vectors may comprise wild-type AAV sequence or they may comprise one or more modifications to a wild-type AAV sequence. In certain embodiments, an AAV vector comprises one or more amino acid modifications, optionally substitutions, deletions, or insertions, within a capsid protein, optionally VP1, VP2 and/or VP3. In particular embodiments, the modification provides for reduced immunogenicity when the AAV vector is provided to a subject.

Capsid proteins of a rAAV may be modified so that the rAAV is targeted to a particular target tissue of interest such as cardiomyocytes. In some embodiments, the rAAV is directly injected into the intracerebroventricular space of the subject.

In some embodiments, the rAAV virion is an AAV2 rAAV virion. The capsid many be an AAV2 capsid or functional variant thereof. In some embodiments, the AAV2 capsid shares at least 98%, 99%, or 100% identity to a reference AAV2 capsid, e.g., SEQ ID NO: 96.

In some embodiments, the rAAV virion is an AAV9 rAAV virion. The capsid many be an AAV9 capsid or functional variant thereof. In some embodiments, the AAV9 capsid shares at least 98%, 99%, or 100% identity to a reference AAV9 capsid, e.g., SEQ ID NO: 97.

In some embodiments, the rAAV virion is an AAV6 rAAV virion. The capsid many be an AAV9 capsid or functional variant thereof. In some embodiments, the AAV6 capsid shares at least 98%, 99%, or 100% identity to a reference AAV6 capsid, e.g., SEQ ID NO: 98.

In some embodiments, the rAAV virion is an AAVrh. 10 rAAV virion. The capsid many be an AAV9 capsid or functional variant thereof. In some embodiments, the AAVrh. 10 capsid shares at least 98%, 99%, or 100% identity to a reference AAVrh. 10 capsid, e.g., SEQ ID NO: 99.

In some embodiments, the capsid protein is encoded by a polynucleotide supplied on a plasmid in trans to the transfer plasmid. The polynucleotide sequence of wild-type AAVrh74 cap is provided as SEQ ID NO: 100.

The disclosure further provides protein sequences for AAVrh74 VP1, VP2, and VP3, including SEQ ID NOs: 101-103, and homologs or functional variants thereof.

In certain cases, the AAVrh74 capsid comprises the amino acid sequence set forth in SEQ ID NO: 101. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh74 VP1 which is set forth in SEQ ID NO: 101. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh74 VP2 which is set forth in SEQ ID NO: 102. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh74 VP3 which is set forth in SEQ ID NO: 103.

In some embodiments, the rAAV virion is an AAV-PHP.B rAAV virion or a neutrotrophic variant thereof, such as, without limitation, those disclosed in Int'l Pat. Pub. Nos. WO 2015/038958 A1 and WO 2017/100671 A1. For example, the AAV capsid may comprise at least 4 contiguous amino acids from the sequence TLAVPFK (SEQ ID NO: 105) or KFPVALT (SEQ ID NO: 106), e.g., inserted between a sequence encoding for amino acids 588 and 589 of AAV9.

The capsid many be an AAV-PHP.B capsid or functional variant thereof. In some embodiments, the AAV-PHP.B capsid shares at least 98%, 99%, or 100% identity to a reference AAV-PHP.B capsid, e.g., SEQ ID NO: 104.

Further AAV capsids used in the rAAV virions of the disclosure include those disclosed in Pat. Pub. Nos. WO 2009/012176 A2 and WO 2015/168666 A2.

Without being bound by theory, the present inventors have determined that an AAV9 vector, AAVrh.74, or an AAVrh. 10 vector will confer desirable cardiac tropism on the vector. Without being bound by theory, the present inventors have further determined that an AAV9 vector, AAVrh.74, or an AAVrh. 10 vector may provide desired specificity to cardiac cells.

In an aspect, the disclosure provides pharmaceutical compositions comprising the rAAV virion of the disclosure and one or more pharmaceutically acceptable carriers, diluents, or excipients.

For purposes of administration, optionally by injection, various solutions can be employed, such as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as Poloxamer 188, e.g., at 0.001% or 0.01%. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include but are not limited to sterile aqueous solutions or dispersions and sterile powders for the extemporaneous preparation of sterile injectable solutions or dispersions. In all cases the form is sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In some embodiments, isotonic agents, such as sugars or sodium chloride, may be included. Prolonged absorption of the injectable compositions can be brought about by use of agents delaying absorption, for example, aluminum monostearate and gelatin.

Sterile injectable solutions may be prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the certain methods of preparation are vacuum drying and the freeze-drying technique that yield a powder of the active ingredient plus any additional desired ingredient from the previously sterile-filtered solution thereof.

In another aspect, the disclosure comprises a kit comprising an rAAV virion of the disclosure and instructions for use.

In an aspect, the disclosure provides a method of increasing JPH2 expression and/or activity in a cell, comprising contacting the cell with an rAAV of the disclosure. In another aspect, the disclosure provides a method of increasing JPH2 expression and/or activity in a subject, comprising administering to the subject an rAAV of the disclosure. In some embodiments, the cell and/or subject is deficient in JPH2 messenger RNA or JPH2 protein expression levels and/or activity and/or comprises a loss-of-function mutation in JPH2. In some embodiments, the cell and/or subject is deficient in JPH2 messenger RNA or JPH2 protein expression levels and/or activity and/or comprises a truncated variant of JPH2 having at most 150 or at most 200 amino acids. The cell may be a cardiac cell, e.g. a cardiomyocyte cell. In particular embodiments, the subject is a mammal, e.g., a human.

In some embodiments, the method promotes survival of cardiac cell, e.g. a cardiomyocyte cell, in cell culture and/or in vivo. In some embodiments, the method promotes and/or restores function of the heart.

In another aspect, the disclosure provides a method of treating a disease or disorder in a subject in need thereof, comprising administering to the subject an effective amount of an rAAV virion of the disclosure. In some embodiments, the disease or disorder is a cardiac disease or disorder. Illustrative cardiac disorders include heart failure, dilated cardiomyopathy, hypertrophic cardiomyopathy, atrial fibrillation, arrhythmia, sinus node disease, hypertensive heart disease, cardiac hypertrophy, atrial fibrosis, myocardial infarction, symptomatic sick sinus syndrome, atrial disease, myocardial infarction, and familial hypertrophic cardiomyopathy 17 (CMH17). In certain embodiments, the subject suffers from or is at risk for CMH17. In particular embodiments, the subject is a mammal, e.g., a human, having a loss-of-function mutation in a JPH2 gene. In particular embodiments, the subject is a mammal, e.g., a human, having a stress-induced truncated variant of JPH2. In particular methods, treatment with the rAAV virion results in expression of the JPH2 protein encoded by the rAAV virion in the subject, e.g., in the subject's heart or cardiac tissue. In certain embodiments, treatment with the rAAV virion results in at least two-fold, at least five-fold, at least ten-fold, or more JPH2 protein levels detectable in the subject's heart. In certain embodiments, treatment with the rAAV virion results in at least two-fold, at least five-fold, at least ten-fold, or more JPH2 protein levels detectable in cardiac fibroblasts (CFs) in the subject's heart. In certain embodiments, treatment with the rAAV virion results in at least two-fold, at least five-fold, at least ten-fold, or more JPH2 protein levels detectable in cardiomyocytes in the subject's heart. In certain embodiments, treatment with the rAAV virion results in at least two-fold, at least five-fold, at least ten-fold, or more JPH2 protein levels detectable in smooth muscle cells (SMCs) in the subject's heart. In certain embodiments, treatment with the rAAV virion results in at least two-fold, at least five-fold, at least ten-fold, or more JPH2 protein levels detectable in endothelial cells (ECs) in the subject's heart. In certain embodiments, treatment with the rAAV virion results in at least two-fold, at least five-fold, at least ten-fold, or more JPH2 protein levels detectable in the epicardium in the subject's heart. In certain embodiments, treatment with the rAAV virion results in at least two-fold, at least five-fold, at least ten-fold, or more JPH2 protein levels detectable in the myocardium in the subject's heart. In certain embodiments, treatment with the rAAV virion results in at least two-fold, at least five-fold, at least ten-fold, or more JPH2 protein levels detectable in the endocardium in the subject's heart.

The AAV-mediated delivery of JPH2 protein to the heart may increase life span, prevent or attenuate cardiac cell degeneration, heart failure, scarring, reduced ejection fraction, arrythmia, angina, exercise intolerance, angina (chest pain), sudden cardiac death, exertional myalgias and cramps. The AAV-mediated delivery of JPH2 protein to the heart may show improvement from, or prevent normal disease course detected by use of echocardiography, pathological electrocardiogram, cardiac MRI, heart biopsy, decrease in paroxysmal ventricular arrhythmias, and/or decrease in sudden cardiac death.

The methods disclosed herein may provide efficient biodistribution of JPH2 in the heart. They may result in sustained expression in all, or a substantial fraction of, cardiac cells, e.g., cardiomyocytes. Notably, the methods disclosed herein may provide long-lasting expression of JPH2 protein throughout the life of the subject following AAV vector administration. In some embodiments, JPH2 protein expression in response to treatment lasts at least 1, 2, 3, 4, 5, 6, 7, 8, 9, 10, 15, 20, 25, 30, 35, or 40 years.

Combination therapies are also contemplated by the disclosure. Combinations of methods of the disclosure with standard medical treatments (e.g., corticosteroids or topical pressure reducing medications) are specifically contemplated, as are combinations with novel therapies. In some cases, a subject may be treated with a steroid and/or combination of immune suppressing agents to prevent or to reduce an immune response to administration of a rAAV described herein.

In some embodiments, the AAV vector is administered at a dose of between about 1×10¹² and 5×10¹⁴ vector genomes (vg) or between about 1×10¹² and 6×10¹⁴ vg of the AAV vector per kilogram (vg) of total body mass of the subject (vg/kg). In some embodiments, the AAV vector is administered at a dose of between about 1×10¹³ and 5×10¹⁴ vg/kg. In some embodiments, the AAV vector is administered at a dose of between about 5×10¹³ and 3×10¹⁴ vg/kg. In some embodiments, the AAV vector is administered at a dose of between about 5×10¹³ and 1×10¹⁴ vg/kg. In certain embodiments, the AAV vector is administered at a dose of between about 5×10¹³ and 5×10¹⁴ vg/kg. In certain embodiments, the AAV vector is administered at a dose of between about 1×10¹³ and 1×10¹⁵ vg/kg. In certain embodiments, the AAV vector is administered at a dose of between about 5×10¹³ and 1×10¹⁴ vg/kg. In certain embodiments, the AAV vector is administered at a dose of between about 8×10¹³ and 1×10¹⁴ vg/kg.

In some embodiments, the AAV vector is administered at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, less than about 7×10¹⁵ vg/kg, less than about 1×10¹⁶ vg/kg, less than about 3×10¹⁶ vg/kg, less than about 5×10¹⁶ vg/kg, less than about 7×10¹⁶ vg/kg, less than about 1×10¹⁷ vg/kg, less than about 3×10¹⁷ vg/kg, less than about 5×10¹⁷ vg/kg, less than about 7×10¹⁷ vg/kg, less than about 1×10¹⁸ vg/kg, less than about 3×10¹⁸ vg/kg, less than about 5×10¹⁸ vg/kg, or less than about 7×10¹⁸ vg/kg. In certain embodiments, the AAV vector delivered at any of these doses is an AAV9 vector or an AAV rh74 vector. In some cases, it may be advantageous to use a higher dose for an AAV rh74 vector than for an AAV9 vector.

In some embodiments, the AAV vector is administered at a dose of about 1×10¹² vg/kg, about 3×10¹² vg/kg, about 5×10¹² vg/kg, about 7×10¹² vg/kg, about 1×10¹³ vg/kg, about 3×10¹³ vg/kg, about 5×10¹³ vg/kg, about 6×10¹³ vg/kg, about 7×10¹³ vg/kg, about 8×10¹³ vg/kg, about 9×10¹³ vg/kg, about 1×10¹⁴ vg/kg, about 3×10¹⁴ vg/kg, about 5×10¹⁴ vg/kg, about 7×10¹⁴ vg/kg, about 1×10¹⁵ vg/kg, about 3×10¹⁵ vg/kg, about 5×10¹⁵ vg/kg, about 7×10¹⁵ vg/kg, about 1×10¹⁶ vg/kg, about 3×10¹⁶ vg/kg, about 5×10¹⁶ vg/kg, about 7×10¹⁶ vg/kg, about 1×10¹⁷ vg/kg, about 3×10¹⁷ vg/kg, about 5×10¹⁷ vg/kg, about 7×10¹⁷ vg/kg, about 1×10¹⁸ vg/kg, about 3×10¹⁸ vg/kg, about 5×10¹⁸ vg/kg, or about 7×10¹⁸ vg/kg. In certain embodiments, the AAV vector delivered at any of these doses is an AAV9 vector or an AAV rh74 vector.

In some embodiments, the AAV vector is administered at a dose of 1×10¹² vg/kg, 3×10¹² vg/kg, 5×10¹² vg/kg, 7×10¹² vg/kg, 1×10¹³ vg/kg, 3×10¹³ vg/kg, 5×10¹³ vg/kg, 6×10¹³ vg/kg, 7×10¹³ vg/kg, 8×10¹³ vg/kg, 9×10¹³ vg/kg, 1×10¹⁴ vg/kg, 3×10¹⁴ vg/kg, 5×10¹⁴ vg/kg, 7×10¹⁴ vg/kg, 1×10¹⁵ vg/kg, 3×10¹⁵ vg/kg, 5×10¹⁵ vg/kg, or 7×10¹⁵ vg/kg, 1×10¹⁶ vg/kg, 3×10¹⁶ vg/kg, 5×10¹⁶ vg/kg, 7×10¹⁶ vg/kg, 1×10¹⁷ vg/kg, 3×10¹⁷ vg/kg, 5×10¹⁷ vg/kg, 7×10¹⁷ vg/kg, 1×10¹⁸ vg/kg, 3×10¹⁸ vg/kg, 5×10¹⁸ vg/kg, 7×10¹⁸ vg/kg, or a range between any of these values. In certain embodiments, the AAV vector delivered at any of these doses is an AAV9 vector or an AAV rh74 vector.

In some embodiments, the AAV vector is administered systemically at a dose of between about 1×10¹² and 5×10¹⁴ vector genomes (vg) of the AAV vector per kilogram (vg) of total body mass of the subject (vg/kg). In some embodiments, the AAV vector is administered systemically at a dose of between about 1×10¹³ and 5×10¹⁴ vg/kg. In some embodiments, the AAV vector is administered systemically at a dose of between about 5×10¹³ and 3×10¹⁴ vg/kg. In some embodiments, the AAV vector is administered systemically at a dose of between about 5×10¹³ and 1×10¹⁴ vg/kg. In some embodiments, the AAV vector is administered systemically at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, less than about 7×10¹⁵ vg/kg, less than about 1×10¹⁶ vg/kg, less than about 3×10¹⁶ vg/kg, less than about 5×10¹⁶ vg/kg, less than about 7×10¹⁶ vg/kg, less than about 1×10¹⁷ vg/kg, less than about 3×10¹⁷ vg/kg, less than about 5×10¹⁷ vg/kg, less than about 7×10¹⁷ vg/kg, less than about 1×10¹⁸ vg/kg, less than about 3×10¹⁸ vg/kg, less than about 5×10¹⁸ vg/kg, or less than about 7×10¹⁸ vg/kg. In certain embodiments, the AAV vector delivered at any of these doses is an AAV9 vector or an AAV rh74 vector.

In some embodiments, the AAV vector is administered systemically at a dose of about 1×10¹² vg/kg, about 3×10¹² vg/kg, about 5×10¹² vg/kg, about 7×10¹² vg/kg, about 1×10¹³ vg/kg, about 3×10¹³ vg/kg, about 5×10¹³ vg/kg, about 6×10¹³ vg/kg, about 7×10¹³ vg/kg, about 8×10¹³ vg/kg, about 9×10¹³ vg/kg, about 1×10¹⁴ vg/kg, about 3×10¹⁴ vg/kg, about 5×10¹⁴ vg/kg, about 7×10¹⁴ vg/kg, about 1×10¹⁵ vg/kg, about 3×10¹⁵ vg/kg, about 5×10¹⁵ vg/kg, about 7×10¹⁵ vg/kg, about 1×10¹⁶ vg/kg, about 3×10¹⁶ vg/kg, about 5×10¹⁶ vg/kg, about 7×10¹⁶ vg/kg, about 1×10¹⁷ vg/kg, about 3×10¹⁷ vg/kg, about 5×10¹⁷ vg/kg, about 7×10¹⁷ vg/kg, about 1×10¹⁸ vg/kg, about 3×10¹⁸ vg/kg, about 5×10¹⁸ vg/kg, or about 7×10¹⁸ vg/kg. In certain embodiments, the AAV vector delivered at any of these doses is an AAV9 vector or an AAV rh74 vector.

In some embodiments, the AAV vector is administered systemically at a dose of 1×10¹² vg/kg, 3×10¹² vg/kg, 5×10¹² vg/kg, 7×10¹² vg/kg, 1×10¹³ vg/kg, 3×10¹³ vg/kg, 5×10¹³ vg/kg, 6×10¹³ vg/kg, 7×10¹³ vg/kg, 8×10¹³ vg/kg, 9x 10¹³ vg/kg, 1×10¹⁴ vg/kg, 3×10¹⁴ vg/kg, 5×10¹⁴ vg/kg, 7×10¹⁴ vg/kg, 1×10¹⁵ vg/kg, 3×10¹⁵ vg/kg, 5×10¹⁵ vg/kg, 7×10¹⁵ vg/kg, 1×10¹⁶ vg/kg, 3×10¹⁶ vg/kg, 5×10¹⁶ vg/kg, 7×10¹⁶ vg/kg, 1×10¹⁷ vg/kg, 3×10¹⁷ vg/kg, 5×10¹⁷ vg/kg, 7×10¹⁷ vg/kg, 1×10¹⁸ vg/kg, 3×10¹⁸ vg/kg, 5×10¹⁸ vg/kg, 7×10¹⁸ vg/kg. In certain embodiments, the AAV vector delivered at any of these doses is an AAV9 vector or an AAV rh74 vector.

In some embodiments, the AAV vector is administered intravenously at a dose of between about 1×10¹² and 5×10¹⁴ vector genomes (vg) of the AAV vector per kilogram (vg) of total body mass of the subject (vg/kg). In some embodiments, the AAV vector is administered intravenously at a dose of between about 1×10¹³ and 5×10¹⁴ vg/kg. In some embodiments, the AAV vector is administered intravenously at a dose of between about 5×10¹³ and 3×10¹⁴ vg/kg. In some embodiments, the AAV vector is administered intravenously at a dose of between about 5×10¹³ and 1×10¹⁴ vg/kg. In some embodiments, the AAV vector is administered intravenously at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, less than about 7×10¹⁵ vg/kg, less than about 1×10¹⁶ vg/kg, less than about 3×10¹⁶ vg/kg, less than about 5×10¹⁶ vg/kg, less than about 7×10¹⁶ vg/kg, less than about 1×10¹⁷ vg/kg, less than about 3×10¹⁷ vg/kg, less than about 5×10¹⁷ vg/kg, less than about 7×10¹⁷ vg/kg, less than about 1×10¹⁸ vg/kg, less than about 3×10¹⁸ vg/kg, less than about 5×10¹⁸ vg/kg, or less than about 7×10¹⁸ vg/kg. In certain embodiments, the AAV vector delivered at any of these doses is an AAV9 vector or an AAV rh74 vector.

In some embodiments, the AAV vector is administered intravenously at a dose of about 1×10¹² vg/kg, about 3×10¹² vg/kg, about 5×10¹² vg/kg, about 7×10¹² vg/kg, about 1×10¹³ vg/kg, about 3×10¹³ vg/kg, about 5×10¹³ vg/kg, about 6×10¹³ vg/kg, about 7×10¹³ vg/kg, about 8×10¹³ vg/kg, about 9×10¹³ vg/kg, about 1×10¹⁴ vg/kg, about 3×10¹⁴ vg/kg, about 5×10¹⁴ vg/kg, about 7×10¹⁴ vg/kg, about 1×10¹⁵ vg/kg, about 3×10¹⁵ vg/kg, about 5×10¹⁵ vg/kg, about 7×10¹⁵ vg/kg, about 1×10¹⁶ vg/kg, about 3×10¹⁶ vg/kg, about 5×10¹⁶ vg/kg, about 7×10¹⁶ vg/kg, about 1×10¹⁷ vg/kg, about 3×10¹⁷ vg/kg, about 5×10¹⁷ vg/kg, about 7×10¹⁷ vg/kg, about 1×10¹⁸ vg/kg, about 3×10¹⁸ vg/kg, about 5×10¹⁸ vg/kg, or about 7×10¹⁸ vg/kg.

In some embodiments, the AAV vector is administered intravenously at a dose of 1×10¹² vg/kg, 3×10¹² vg/kg, 5×10¹² vg/kg, 7×10¹² vg/kg, 1×10¹³ vg/kg, 3×10¹³ vg/kg, 5×10¹³ vg/kg, 6×10¹³ vg/kg, 7×10¹³ vg/kg, 8×10¹³ vg/kg, 9×10¹³ vg/kg, 1×10¹⁴ vg/kg, 3×10¹⁴ vg/kg, 5×10¹⁴ vg/kg, 7×10¹⁴ vg/kg, 1×10¹⁵ vg/kg, 3×10¹⁵ vg/kg, 5×10¹⁵ vg/kg, 7×10¹⁵ vg/kg, 1×10¹⁶ vg/kg, 3×10¹⁶ vg/kg, 5×10¹⁶ vg/kg, 7×10¹⁶ vg/kg, 1×10¹⁷ vg/kg, 3×10¹⁷ vg/kg, 5×10¹⁷ vg/kg, 7×10¹⁷ vg/kg, 1×10¹⁸ vg/kg, 3×10¹⁸ vg/kg, 5×10¹⁸ vg/kg, 7×10¹⁸ vg/kg. In certain embodiments, the AAV vector delivered at any of these doses is an AAV9 vector or an AAV rh74 vector.

Evidence of functional improvement, clinical benefit or efficacy in patients may be revealed by improvements in New York Heart Association functional classification (NYHA Class), echocardiography (stabilized or improved left ventricle ejection fraction, fractional shortening, left ventricular outflow tract obstruction, left ventricular wall thickness, left or right ventricular volumes, right ventricular area and/or velocity time integral), electrocardiogra (stabilized or improved ST-segment alterations, T-wave inversion, Q waves, atrial fibrillation, and/or supraventricular tachycardia), cardiac MRI, heart biopsy, decrease in paroxysmal ventricular arrhythmias, decrease in sudden cardiac death, and/or decrease in or lack of further development of fibro-fatty deposits.

Administration of an effective dose of the compositions may be by routes standard in the art including, but not limited to, systemic, local, direct injection, intravenous, intracardiac administration. In some cases, administration comprises systemic, local, direct injection, intravenous, intracardiac injection. Administration may be performed by cardiac catheterization.

In some embodiments, the disclosure provides for local administration and systemic administration of an effective dose of rAAV and compositions of the disclosure. For example, systemic administration may be administration into the circulatory system so that the entire body is affected. Systemic administration includes parental administration through injection, infusion or implantation. Routes of administration for the compositions disclosed herein include intravenous (“IV”) administration, intraperitoneal (“IP”) administration, intramuscular (“IM”) administration, intralesional administration, or subcutaneous (“SC”) administration, or the implantation of a slow-release device, e.g., a mini-osmotic pump, a depot formulation, etc. In some embodiments, the methods of the disclosure comprise administering an AAV vector of the disclosure, or pharmaceutical composition thereof by intravenous, intramuscular, intraarterial, intrarenal, intraurethral, intracardiac, intracoronary, intramyocardial, intradermal, epidural, subcutaneous, intraperitoneal, intraventricular, or ionophoretic administration.

In particular, administration of rAAV of the present disclosure may be accomplished by using any physical method that will transport the rAAV recombinant vector into the target tissue of an animal. Administration includes, but is not limited to, injection into the heart.

In some embodiments, the methods of the disclosure comprise intracardiac delivery. Infusion may be performed using specialized cannula, catheter, syringe/needle using an infusion pump. Administration may comprise delivery of an effective amount of the rAAV virion, or a pharmaceutical composition comprising the rAAV virion, to the heart. These may be achieved, e.g., via intravenous, intramuscular, intraarterial, intrarenal, intraurethral, intracardiac, intracoronary, intramyocardial, intradermal, epidural, subcutaneous, intraperitoneal, intraventricular, or ionophoretic administration. The compositions of the disclosure may further be administered intravenously.

Effects of rAAV Administration

In some embodiments, administration of rAAV of the present disclosure may have beneficial effects for the subject. For example, administration of rAAV of the present disclosure may increase survivability of the subject compared to a subject that is not administered the rAAV of the present disclosure.

In some embodiments, administration of rAAV of the present disclosure increases survivability by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, or at least about 500% compared to a subject that is not administered the rAAV of the present disclosure.

In some embodiments, administration of rAAV of the present disclosure increases survivability by between 1% and 90%, between 20% and 80%, between 30% and 80%, between 40% and 80%, between 50% and 80%, between 1% to 2%, between 2% to 3%, between 3% to 4%, between 4% to 5%, between 5% to 6%, between 6% to 7%, between 7% to 8%, between 8% to 9%, between 9% to 10%, between 10% to 15%, between 15% to 20%, between 20% to 35%, between 25% to 30%, between 30% to 35%, between 35% to 40%, between 40% to 45%, between 45% to 50%, between 50% to 55%, between 55% to 60%, between 60% to 65%, between 65% to 70%, between 70% to 75%, between 75% to 80%, between 80% to 85%, between 85% to 90%, between 90% to 95%, between 95% to 100%, between 100% to 200%, between 200% to 300%, between 300% to 400%, or between 400% to 500% compared to a subject that is not administered the rAAV of the present disclosure.

In some embodiments, administration of rAAV of the present disclosure prevents a decrease in the ejection fraction in a subject compared to a subject that is not administered the rAAV of the present disclosure. In some embodiments, administration of rAAV of the present disclosure prevents a decrease in the ejection fraction by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure.

In some embodiments, administration of rAAV of the present disclosure prevents a decrease in the ejection fraction by between 1% and 90%, between 20% and 80%, between 30% and 80%, between 40% and 80%, between 50% and 80%, between 1% to 2%, between 2% to 3%, between 3% to 4%, between 4% to 5%, between 5% to 6%, between 6% to 7%, between 7% to 8%, between 8% to 9%, between 9% to 10%, between 10% to 15%, between 15% to 20%, between 20% to 35%, between 25% to 30%, between 30% to 35%, between 35% to 40%, between 40% to 45%, between 45% to 50%, between 50% to 55%, between 55% to 60%, between 60% to 65%, between 65% to 70%, between 70% to 75%, between 75% to 80%, between 80% to 85%, between 85% to 90%, between 90% to 95%, or between 95% to 100% compared to a subject that is not administered the rAAV of the present disclosure.

In some embodiments, administration of rAAV of the present disclosure prevents an increase in end-diastolic diameter (EDD) in a subject compared to a subject that is not administered the rAAV of the present disclosure. In some embodiments, administration of rAAV of the present disclosure prevents an increase in end-diastolic diameter (EDD) in a subject by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, or at least about 500% compared to a subject that is not administered the rAAV of the present disclosure.

In some embodiments, administration of rAAV of the present disclosure prevents an increase in EDD in a subject by between 1% and 90%, between 20% and 80%, between 30% and 80%, between 40% and 80%, between 50% and 80%, between 1% to 2%, between 2% to 3%, between 3% to 4%, between 4% to 5%, between 5% to 6%, between 6% to 7%, between 7% to 8%, between 8% to 9%, between 9% to 10%, between 10% to 15%, between 15% to 20%, between 20% to 35%, between 25% to 30%, between 30% to 35%, between 35% to 40%, between 40% to 45%, between 45% to 50%, between 50% to 55%, between 55% to 60%, between 60% to 65%, between 65% to 70%, between 70% to 75%, between 75% to 80%, between 80% to 85%, between 85% to 90%, between 90% to 95%, between 95% to 100%, between 100% to 200%, between 200% to 300%, between 300% to 400%, or between 400% to 500% compared to a subject that is not administered the rAAV of the present disclosure.

In some embodiments, administration of rAAV of the present disclosure prevents an increase in systolic left ventricular posterior wall thickness (LVPW) in a subject compared to a subject that is not administered the rAAV of the present disclosure. In some embodiments, administration of rAAV of the present disclosure prevents an increase in LVPW in a subject by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, or at least about 500% compared to a subject that is not administered the rAAV of the present disclosure.

In some embodiments, administration of rAAV of the present disclosure prevents an increase in LVPW in a subject by between 1% and 90%, between 20% and 80%, between 30% and 80%, between 40% and 80%, between 50% and 80%, between 1% to 2%, between 2% to 3%, between 3% to 4%, between 4% to 5%, between 5% to 6%, between 6% to 7%, between 7% to 8%, between 8% to 9%, between 9% to 10%, between 10% to 15%, between 15% to 20%, between 20% to 35%, between 25% to 30%, between 30% to 35%, between 35% to 40%, between 40% to 45%, between 45% to 50%, between 50% to 55%, between 55% to 60%, between 60% to 65%, between 65% to 70%, between 70% to 75%, between 75% to 80%, between 80% to 85%, between 85% to 90%, between 90% to 95%, between 95% to 100%, between 100% to 200%, between 200% to 300%, between 300% to 400%, or between 400% to 500% compared to a subject that is not administered the rAAV of the present disclosure.

### EXAMPLES

### Example 1: Pre-Clinical Bioactivity and Efficacy In Vitro

Vectors illustrated in FIGS. 1-25 are tested. AAV vectors or respective expression cassettes are tested in vitro using cultured cardiomyocytes (e.g., induced pluripotent stem cell cardiomyocytes (iPSC-CMs) from patients or primary cardiomyocytes collected from animal models) or other cells amenable to transfection or transduction with these constructs. Expression of JPH2 is assessed by immunofluorescence and Western blot. Cell-based studies employing mutated cardiomyocytes will reveal benefit of overexpression of JPH2 transgene (either following AAV vector transduction and/or transfection with vector plasmids) by a) normalization of calcium handling and consequent normalization of contraction pace and/or b) restoration of normal T-tubule structure and/or attenuation of remodeling.

### Example 2: Rescue of Heart Failure In Vivo after Transverse Aortic Constriction (TAC)

AAV-JHP2 gene therapy with select AAV vectors described above is performed essentially as described in Reynolds et al. (Int J Cardiol. 2016 Dec. 15; 225:371-380). AAV expression cassettes are packaged and delivered in vivo using different capsid serotypes such as AAV9 and/or AAV rh.74.

Mouse TAC Model: Transaortic constriction (TAC) in the mouse is an experimentally induced cardiac hypertrophy due to pressure overload with subsequent heart failure. Compared to other experimental mouse models of heart failure, the TAC model results in more reproducible cardiac hypertrophy and a gradual time course of development of heart failure. Following TAC in the mouse, a progressive decrease in ejection fraction and other measures of heart function are paralleled by a progressive decrease of cardiac JPH2 levels. Male C57BI/6J mice (approximately 4 months of age) are anesthetized and the aortic arch is visualized by performing an anterior thoracotomy to the level of the third intercostal space. Constriction is performed by tying a silk suture against a 28-gauge needle between the first and second trunk of the aortic arch. For consistency, constriction levels are quantified by measuring alterations in Doppler velocities of the right and left carotid arteries 7 days post-surgery. Right-to-left carotid peak velocity ratios may range from 5.0 to 6.5 and 2-week post TAC ejection fractions may range from 40%-50%.

Functional Evidence of Efficacy by Echocardiography: Evidence of bioactivity and efficacy for cardiac benefit in the TAC model is evaluated using transthoracic echocardiography at predefined timepoints including baseline and various intervals after TAC. To screen animals with sufficient heart failure suitable for this mouse model, Doppler ratios of right carotid to left carotid peak velocity (RC/LC) are determined 1-week post-TAC and those that do not meet criteria (RC/LC of 5.0-6.5) are excluded from study. Additionally, echocardiography at 2 weeks post-TAC is performed and animals outside the range of 40-50% ejection fraction (EF) are also excluded. Mice with appropriate Doppler RC/EV and EF by echocardiogram are then injected (either intra-venously or intra-retro-orbitally) at week 3 post-Tac with AAV constructs overexpressing JPH2 protein or with formulation buffer (FB; vehicle control). Efficacy is evident in AAV-JPH2 treated animals by significantly increased EF compared to the FB control group across time. Echocardiography will reveal that FB injected mice will be found to have an EF that declines progressively across time, and end-diastolic diameter (EDD) that increases with time and a systolic left ventricular posterior wall thickness (LVPW) that also increases with time. In contrast, AAV-JPH2 injected animals will be found to have an EF, and EDD that remains stable or improves slightly with time, and an LVPW that is greater than FB controls across time following AAV-JPH2 treatment.

Morphological Evidence of Efficacy by Attenuating Transverse Tubule Remodeling: To study the effects of AAV9-JPH2 on T-tubule structure, isolated myocytes are evaluated for potential remodeling as a consequence of TAC. As a consequence of TAC, FB control injected mice will display the typical cardiac remodeling evident by significantly lower T-tubule area and T-tubule power, a measure of the integrity of the T-tubule structure in myocytes. Evidence of efficacy by overexpression of JPH2 will be observed by mitigation of the cardiac remodeling and attenuation of the changes in T-tubule area and T-tubule power in AAV-JPH2 injected animals compared to FB injected controls.

Functional Evidence of Efficacy by Improved Calcium Handling: As a consequence of TAC, sarcoplasmic reticulum (SR) Ca²⁺ handling in isolated ventricular myocytes is significantly impaired as measured by lower Ca²⁺ transient amplitudes, and a significantly lower Ca²⁺ SR load using a caffeine dump protocol, with alterations in the normal Na²⁺/Ca²⁺-exchanger, are observed. Evidence of benefit or efficacy of AAV-mediated overexpression of JPH2 in the TAC model will be evident by normalization of the Ca²⁺ transient amplitude, improvement of the SR Ca²⁺ load and normalization of the Na²⁺/Ca²⁺-exchanger in cardiomyocytes.

Transgene Protein Expression and Evidence of Efficacy by Ameliorating Downstream Hypertrophic Responses: Expression levels of JPH2 protein as a consequence of AAV administration are assessed in heart lysates by Western blot. It is expected that while JPH2 protein levels will be reduced in FB injected animals as a consequence of TAC, AAV-mediated overexpression of JPH2 will result in sustained levels of protein up to 9 weeks after TAC. Furthermore, quantitative polymerase chain reaction (qPCR) will reveal an increase in mRNA levels of several pro-hypertrophic markers in FB control injected TAC mice compared to normal, sham operated controls. Increases in pro-hypertrophic markers will include, but may not be limited to, ‘regulator of calcineurin 1 isoform 4’ (Rcan1.4), a marker of ‘nuclear factor of activated T cells’ (NFAT), myosin heavy chain 7 (Myh7), natriuretic peptide type A (Nppa), and natriuretic peptide type B (Nppb). The beneficial effects of JHP2 delivery by AAV gene therapy will be evident as attenuating or significantly lowering mRNA levels of one or several of these pro-hypertrophic markers in heart lysates from AAV-JPH2 injected animals compared to FB injected TAC controls.

### Example 3: Pre-Clinical Efficacy In Vivo in the JPH2-A399S Knock-In Mouse

The JPH2-A399S knock-in mouse is a genetic model that captures elements of human disease, corresponds to hypertrophic cardiomyopathy (HCM) variants, and results in left ventricular hypertrophy and fibrosis by 6 months of age in the mutant mouse. As a mouse model of the human disease, it further permits the evaluation of the potential bioactivity and efficacy of AAV overexpression of JPH2.

Morphological Evidence of Efficacy by Attenuating Transverse Tubule Remodeling: To study the effects of AAV9-JPH2 on T-tubule structure in the JPH2-A399S mouse model, isolated myocytes are evaluated for potential remodeling as a consequence of the A399S mutation. As a consequence of the A399S mutation, FB control injected A399S mice may display the typical cardiac remodeling evident by significantly lower T-tubule area and T-tubule power, a measure of the integrity of the T-tubule structure in myocytes. Evidence of efficacy by overexpression of JPH2 would be observed by mitigation of the cardiac remodeling and attenuation of the changes in T-tubule area and T-tubule power in AAV-JPH2 injected animals compared to FB injected A399S knock-in controls.

Functional Evidence of Efficacy in A399S Mutant Mouse by Improved Calcium Handling: In the JPH2 HCM genetic mouse model, A399S, mice express a mutation analogous to that found in humans (A405S) which leads to cardiomyocyte hypertrophy and significant fibrosis over a time course of many weeks to months. It has been revealed that A399S mice exhibit various features associated with HC including hypertrophic interventricular septum, increased LV mass, asymmetric LV hypertrophy, reduced diastolic filling and myofiber disarray. Evidence of therapeutic benefit as a consequence of overexpression of JPH2 in the A399S mouse model will be revealed by mitigation of the above abnormal consequences on heart morphology and function. In addition, is it possible that as a consequence of the A399S knock-in mutation, sarcoplasmic reticulum (SR) Ca²⁺ handling in isolated ventricular myocytes may be significantly impaired as measured by lower Ca²⁺ transient amplitudes, and a significantly lower Ca²⁺ SR load, with alterations in the normal Na²⁺/Ca²⁺-exchanger. Evidence of benefit or efficacy of AAV-mediated overexpression of JPH2 in the A399S model may be evident by normalization of the Ca²⁺ transient amplitude, improvement of the SR Ca²⁺ load, and/or normalization of the Na²⁺/Ca²⁺-exchanger in cardiomyocytes.

### Example 4: Pre-Clinical Transgene Expression

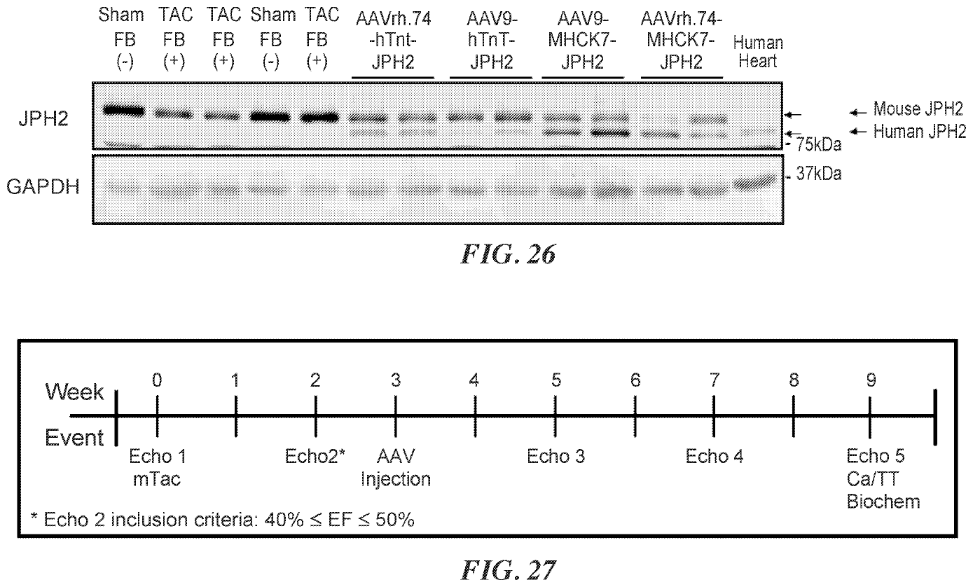

Expression cassettes illustrated in FIG. 1 and FIG. 2 were tested following packaging into AAV.rh74 or AAV9 vectors. The resulting AAV vectors (both AAVrh.74 and AAV9) were tested in vivo using C57BL/6J mice, and the expression levels of JPH2 were assessed by Western Blot (WB) of heart tissue proteins (FIG. 26). The MHCK7 promoter produced the highest expression levels of JPH2 by WB in the mouse heart, following delivery of AAV9-MHCK7-JPH2 and AAVrh.74-MHCK7-JPH2 respectively. The hTnnT2 promoter (“hTnT”) was found to drive lower levels of JPH2 protein expression, with AAVrh.74 yielding higher levels of expression than the AAV9. Based on these results, it can be concluded that AAVrh.74 and AAV9 vectors can effectively be used to express JPH2 in the heart.

### Example 5: Rescue of Heart Failure In Vivo after Transverse Aortic Constriction (TAC)

AAV-JHP2 gene therapy with select AAV vectors described above was performed essentially as described in Reynolds et al. (Int J Cardiol. 2016 Dec. 15; 225:371-380). AAV expression cassettes were packaged and delivered in vivo using different capsid serotypes, AAVrh.74 and AAV9.

Mouse TAC Model: Transaortic constriction (TAC) in the mouse is an experimentally induced cardiac hypertrophy due to pressure overload with subsequent heart failure. Compared to other experimental mouse models of heart failure, the TAC model results in more reproducible cardiac hypertrophy and a gradual time course of development of heart failure. Following TAC in the mouse, a progressive decrease in ejection fraction (EF) and other measures of heart function are paralleled by a progressive decrease of cardiac JPH2 levels. To examine the extent to which AAV-mediated overexpression of JPH2 could confer benefit in this mouse model of cardiac hypertrophy, male C57BL/6J mice (approximately 4 months of age) were anesthetized and the aortic arch was visualized by performing an anterior thoracotomy to the level of the third intercostal space. Constriction was performed by tying a silk suture against a 28-gauge needle between the first and second trunk of the aortic arch. For consistency, constriction levels were quantified by measuring alterations by echocardiography and mice with ejection fractions that ranged from 40%-50% 2-weeks post-TAC were selected for this study.

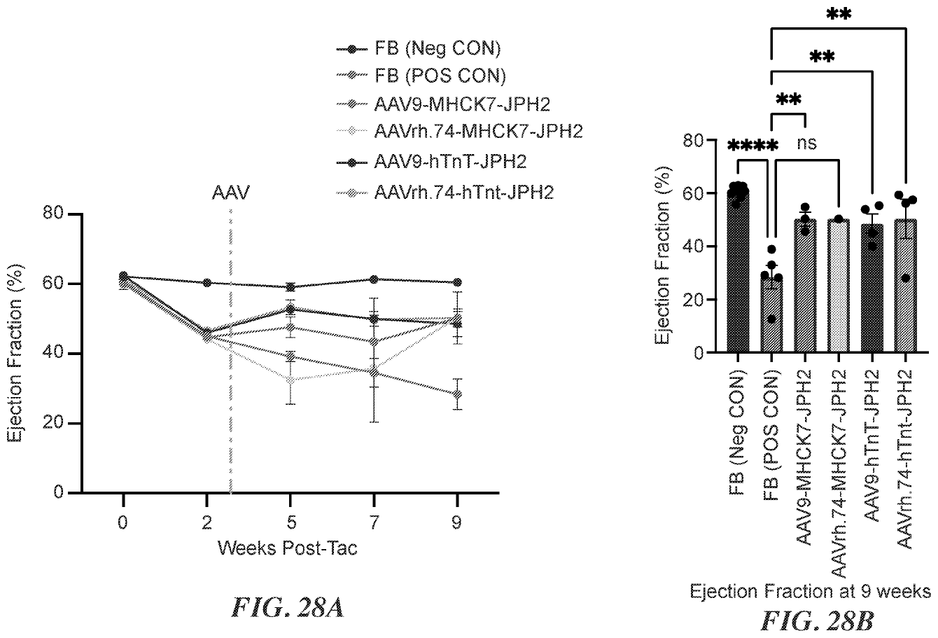

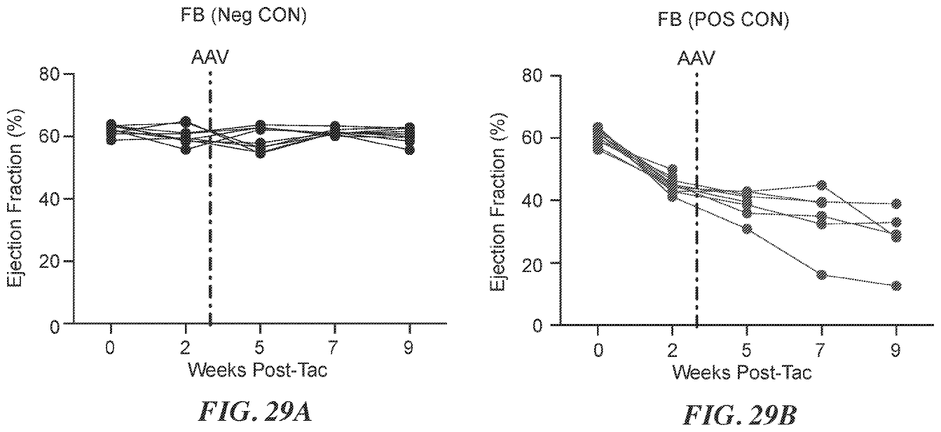

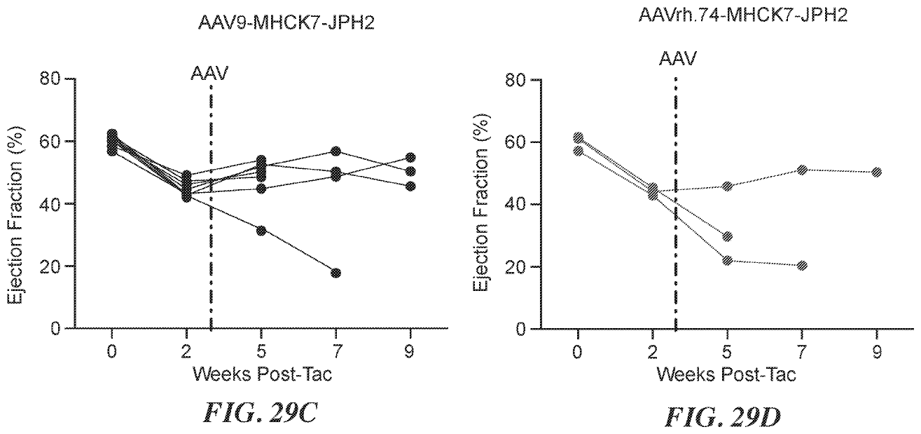

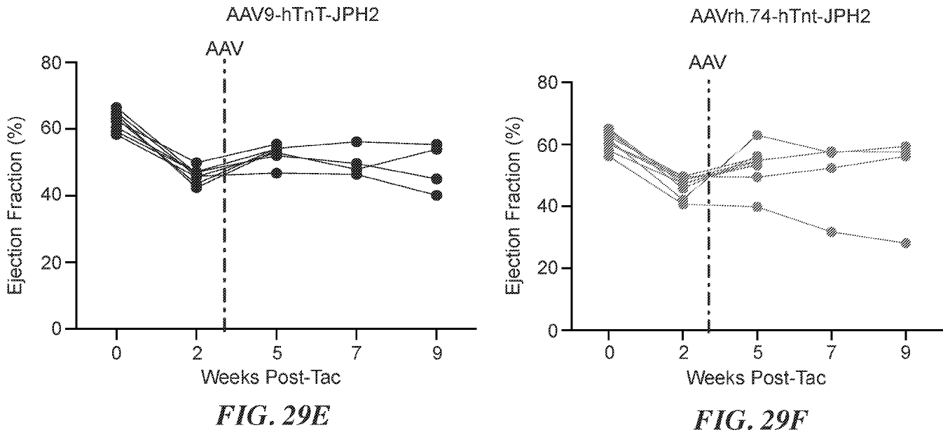

Functional Evidence of Efficacy by Echocardiography: Evidence of bioactivity and efficacy for cardiac benefit in the TAC model was evaluated using transthoracic echocardiography at predefined timepoints including baseline and various intervals after TAC (FIG. 27). To screen animals with sufficient heart failure suitable for this mouse model, echocardiography at 2 weeks post-TAC was performed and animals outside the range of 40-50% ejection fraction (EF) were excluded. Mice with appropriate EF by echocardiogram then received retro-orbital injections at week 3 post-Tac with AAV constructs overexpressing JPH2 protein a dose of 3×10¹³ vg/kg or with formulation buffer (FB; vehicle control). The results are compared to either sham surgery, FB (POS CON), or TAC surgery, FB (Neg CON), mice. Efficacy was evident in AAV-JPH2 treated animals by significantly increased EF compared to the FB control group across time (FIGS. 28A-28B and FIGS. 29A-29F).

Echocardiography revealed that FB (POS CON) injected mice have an EF that declined progressively across time (FIG. 28A). In contrast, AAV-JPH2 injected animals demonstrated a clear halting of progression of EF loss after the TAC surgery, evidenced by the EF data at 9 weeks following AAV-JPH2 treatment (FIG. 28B).

Evidence for mitigation of the disease phenotype was observed following both AAVrh.74- and AAV9-mediated JPH2 expression, to varying degrees (FIGS. 29A-29F). These results demonstrate both AAVrh.74 and AAV9 may confer benefit in cardiac indications with JPH2-deficiency, for which the TAC mouse is considered an appropriate model. Additionally, vectors with either hTnT promoter or MHCK7 promoter have been demonstrated to be effective in treating JPH2-related deficiency in this mouse model. Nevertheless, given the time-course data (FIG. 28A and FIGS. 29A-29F), more robust and consistent preservation of EF was observed with vector constructs employing the hTnT promoter. Additionally, no animals injected with AAV constructs with the hTnT promoter died prior to the planned 9-week sacrifice time point. In contrast, 4 of the 8 FB (POS CON) animals died early, 2 of 8 animals in the AAV9-MHCK7 group died early, and 2 of 3 animals in the AAVrh. 74-MHCK7 died early.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A polynucleotide, comprising an expression cassette and optionally flanking adeno-associated virus (AAV) inverted terminal repeats (ITRs), wherein the polynucleotide comprises a polynucleotide sequence encoding a Junctophilin-2 (JPH2), or a functional variant thereof, operatively linked to a promoter.

2. The polynucleotide of claim 1, wherein the promoter is a cardiac-specific promoter.

3. The polynucleotide of claim 1 or claim 2, wherein the promoter is a muscle-specific promoter.

4. The polynucleotide of any one of claims 1 to 3, wherein the promoter is a cardiomyocyte-specific promoter.

5. The polynucleotide of any one of claims 1 to 4, wherein the promoter is a Myosin Heavy-chain Creatine Kinase 7 (MHCK7) promoter.

6. The polynucleotide of claim 5, wherein the MHCK7 promoter shares at least 75%, 80%, 85%, 90%, 95%, 96%, 97%, 98%, 99% or 100% identity with SEQ ID NO: 31.

7. The polynucleotide of any one of claims 1 to 4, wherein the promoter is a cardiac troponin T (hTNNT2) promoter.

8. The polynucleotide of claim 7, wherein the hTNNT2 promoter shares at least 75%, 80%, 85%, 90%, 95%, 96%, 97%, 98%, 99% or 100% identity with SEQ ID NO: 33.

9. The polynucleotide of any one of claims 1 to 8, wherein the expression cassette comprises exon 1 of the cardiac troponin T (hTNNT2) gene, wherein optionally the hTNNT2 promoter and exon 1 together share at least 75%, 80%, 85%, 90%, 95%, 96%, 97%, 98%, 99% or 100% identity with SEQ ID NO: 32.

10. The polynucleotide of any one of claims 1 to 4, wherein the promoter is a ubiquitous promoter, optionally a CMV promoter or a CAG promoter.

11. The polynucleotide of any one of claims 1 to 10, wherein the expression cassette comprises a polyA signal.

12. The polynucleotide of claim 11, wherein the polyA signal is a human growth hormone (hGH) polyA.

13. The polynucleotide of any one of claims 1 to 12, wherein the expression cassette comprises a Woodchuck Hepatitis Virus Posttranscriptional Regulatory Element (WPRE), optionally a WPRE(x).

14. The polynucleotide of any one of claims 1 to 12, wherein the expression cassette comprises a Green Fluorescence Protein (GFP).

15. The polynucleotide of any one of claims 1 to 14, wherein the Junctophilin-2 (JPH2) or functional variant thereof is a JPH2.

16. The polynucleotide of claim 14 or claim 15, wherein the JPH2 is a human JPH2.

17. The polynucleotide of any one of claims 1 to 16, wherein the polynucleotide sequence encoding JPH2 shares at least 75%, 80%, 85%, 90%, 95%, 96%, 97%, 98%, 99% or 100% identity with SEQ ID NO: 2.

18. The polynucleotide of any one of claims 1 to 16, wherein the polynucleotide sequence encoding JPH2 shares at least 75%, 80%, 85%, 90%, 95%, 96%, 97%, 98%, 99% or 100% identity with SEQ ID NO: 4.

19. The polynucleotide of any one of claims 1 to 16, wherein the polynucleotide sequence encoding JPH2 shares at least 75%, 80%, 85%, 90%, 95%, 96%, 97%, 98%, 99% or 100% identity with SEQ ID NO: 6.

20. The polynucleotide of any one of claims 1 to 16, wherein the polynucleotide sequence encoding JPH2 shares at least 75%, 80%, 85%, 90%, 95%, 96%, 97%, 98%, 99% or 100% identity with SEQ ID NO: 8.

21. The polynucleotide of any one of claims 1 to 16, wherein the polynucleotide sequence encoding JPH2 shares at least 75%, 80%, 85%, 90%, 95%, 96%, 97%, 98%, 99% or 100% identity with SEQ ID NO: 10.

22. The polynucleotide of any one of claims 1 to 21, wherein the polynucleotide sequence encoding JPH2 is a human JPH2 polynucleotide.

23. The polynucleotide of any one of claims 1 to 22, wherein the polynucleotide comprises at least about 3.0 kb, at least about 3.2 kb, at least about 3.4 kb, at least about 3.5 kb, at least about 3.7 kb, at least about 4.0 kb, at least about 4.1 kb, at least about 4.2 kb, at least about 4.3 kb, at least about 4.4 kb, at least about 4.5 kb, at least about 4.6 kb, at least about 4.7 kb, at least about 4.8 kb, or at least about 5.0 kb.

24. The polynucleotide of any one of claims 1 to 23, wherein the polynucleotide comprises at most about 3.1 kb, at most about 3.3 kb, at most about 3.5 kb, at most about 3.7 kb, at most about 3.9 kb, at most about 4.1 kb, at most about 4.2 kb, at most about 4.3 kb, at most about 4.4 kb, at most about 4.5 kb, at most about 4.6 kb, at most about 4.7 kb, at most about 4.8 kb, at most about 4.9 kb, or at most about 5.0 kb.

25. The polynucleotide of any one of claims 1 to 24, wherein the polynucleotide comprises 4.4 kb to 5.0 kb, 4.4 kb to 4.9 kb, or 4.4 kb to 4.8 kb, wherein the polynucleotide comprises 4.0 kb to 4.6 kb, 4.0 kb to 4.5 kb, or 4.0 kb to 4.4 kb, wherein the polynucleotide comprises 4.0 kb to 4.3 kb, 4.0 kb to 4.2 kb, or 4.0 kb to 4.1 kb, or wherein the polynucleotide comprises 3.0 kb to 3.9 kb, 3.0 kb to 3.8 kb, or 3.0 kb to 3.7 kb.

26. The polynucleotide of any one of claims 1 to 24, wherein the JPH2 or functional variant thereof comprises at least 600 or at least 630 amino acids.

27. The polynucleotide of any one of claims 1 to 26, wherein the expression cassette is flanked by 5′ and 3′ inverted terminal repeats (ITRs).

28. The polynucleotide of claim 27, wherein the ITRs are AAV2 ITRs and/or the ITRs share at least 75%, 80%, 85%, 90%, 95%, 96%, 97%, 98%, 99% or 100% identity with any one of SEQ ID NO: 15-21.

29. A gene therapy vector, comprising the polynucleotide of any one of claims 1 to 28.

30. The vector of claim 29, wherein the gene therapy vector is a recombinant adeno-associated virus (rAAV) vector.

31. The vector of claim 30, wherein the rAAV vector is an AAV9 or a functional variant thereof.

32. The vector of claim 31, wherein the rAAV vector comprises a capsid protein that shares 90%, 91%, 92%, 93%, 94%, 95%, 96%, 97%, 98%, 99% or 100% identity to any one of SEQ ID NO: 97.

33. The vector of claim 30, wherein the rAAV vector is an AAVrh10 or a functional variant thereof.

34. The vector of claim 33, wherein the rAAV vector comprises a capsid protein that shares 90%, 91%, 92%, 93%, 94%, 95%, 96%, 97%, 98%, 99% or 100% identity to any one of SEQ ID NO: 99.

35. The vector of claim 30, wherein the rAAV vector is an AAV6 or a functional variant thereof.

36. The vector of claim 35, wherein the rAAV vector comprises a capsid protein that shares 90%, 91%, 92%, 93%, 94%, 95%, 96%, 97%, 98%, 99% or 100% identity to any one of SEQ ID NO: 98.

37. The vector of claim 36, wherein the rAAV vector is an AAVrh74 or a functional variant thereof.

38. The vector of claim 37, wherein the rAAV vector comprises a capsid protein that shares 90%, 91%, 92%, 93%, 94%, 95%, 96%, 97%, 98%, 99% or 100% identity to any one of SEQ ID NO: 100.

39. A method of treating and/or preventing a disease or disorder in a subject in need thereof, comprising administering the vector of any one of claims 29 to 38 to the subject.

40. The method of claim 39, wherein the disease or disorder is a cardiac disorder.

41. The method of claim 40, wherein the cardiac disorder is a cardiomyopathy, optionally familial hypertrophic cardiomyopathy 17.

42. The method of claim 41, wherein the cardiomyopathy is a hypertrophic cardiomyopathy (HCM) (hypertrophic).

43. The method of claim 41, wherein the cardiomyopathy is a dilated cardiomyopathy (DCM).

44. The method of claim 39 or 40, wherein the disease or disorder is arrhythmia, optionally atrial fibrillation or sinus node disease.

45. The method of claim 39 or 40, wherein the disease or disorder is heart failure.

46. The method of any one of claims 39 to 45, wherein the subject is a mammal.

47. The method of claim 46, wherein the subject is a primate.

48. The method of claim 47, wherein the subject is a human.

49. The method of any one of claims 39 to 48, wherein the subject has a mutation in a JPH2 gene.

50. The method of any one of claims 39 to 48, wherein the subject has a truncated variant of JPH2.

51. The method of any one of claims 39 to 50, wherein the vector is administered by intravenous injection, intracardiac injection, intracardiac infusion, and/or cardiac catheterization.

52. The method of any one of claims 39 to 51, wherein the administration increases JPH2 expression by at least about 5%.

53. The method of any one of claims 39 to 51, wherein the administration increases JPH2 expression by at least about 30%.

54. The method of any one of claims 39 to 51, wherein the administration increases JPH2 expression by at least about 70%.

55. The method of any one of claims 39 to 51, wherein the administration increases JPH2 expression by about 5% to about 10%.

56. The method of any one of claims 39 to 51, wherein the administration increases JPH2 expression by about 30% to about 50%.

57. The method of any one of claims 39 to 51, wherein the administration increases JPH2 expression by about 50% to about 70%.

58. The method of any one of claims 39 to 51, wherein the administration increases JPH2 expression by about 70% to about 100%.

59. The method of any one of claims 39 to 58, wherein the method treats and/or prevents the disease or disorder.

60. The method of any one of claims 39 to 59, wherein the method comprises administering an effective amount of the vector.

61. The method of any one of claims 39 to 60, wherein the disease or disorder is related to or caused by truncation of JPH2 in the subject.

62. The method of any one of claims 39 to 61, wherein the method comprises administering a pharmaceutical composition comprising an effective amount of the vector.

63. The method of any one of claims 39 to 62, wherein the method comprises administering between about 1×10¹¹ vector genomes and about 1×10¹³ vector genomes of the vector to the subject, administering between about 1×10¹² vector genomes and about 1×10¹⁴ vector genomes of the vector to the subject, or administering between about 1×10¹³ vector genomes and about 1×10¹⁵ vector genomes of the vector to the subject.

64. A pharmaceutical composition comprising the vector of any one of claims 29 to 38.

65. A kit comprising the vector of any one of claims 29 to 38 or the pharmaceutical composition of claim 64 and optionally instructions for use.

66. Use of the vector of any one of claims 29 to 38 in treating a disease or disorder, optionally according to the method of any one of claims 39 to 63.

67. A vector according to any one of claims 29 to 38 for use in treating a disease or disorder, optionally according to the method of any one of claims 39 to 63.

68. A polynucleotide, comprising a polynucleotide sequence that shares at least 90%, 91%, 92%, 93%, 94%, 95%, 96%, 97%, 98%, 99%, or 100% identity to any one of SEQ ID NOs: 26-30 or to any one of SEQ ID NOs: 76-95.

69. The polynucleotide of claim 68, comprising a MHCK7 promoter.

70. The polynucleotide of claim 69, wherein the MHCK7 promoter shares at least 75%, 80%, 85%, 90%, 95%, 96%, 97%, 98%, 99% or 100% identity with SEQ ID NO: 31.

71. The polynucleotide of claim 68, comprising a sequence encoding a human JPH2.

72. A gene therapy vector, comprising the polynucleotide of any one of claims 68 to 71.

73. The vector of claim 72, wherein the gene therapy vector is a recombinant adeno-associated virus (rAAV) vector.

74. The vector of claim 73, wherein the rAAV vector is an AAV9 vector.

75. The vector of claim 73, wherein the rAAV vector is an AAVrh74 vector.

76. A method of treating and/or preventing a cardiac disorder in a subject identified as having a truncation in JPH2, comprising administering the vector of any one of claims 72 to 75 to the subject.

77. The method of claim 76, wherein the cardiac disorder is a cardiomyopathy, optionally familial hypertrophic cardiomyopathy 17.

78. The method of claim 77, wherein the cardiomyopathy is a hypertrophic cardiomyopathy (HCM) (hypertrophic).

79. The method of claim 77, wherein the cardiomyopathy is a dilated cardiomyopathy (DCM).

80. The method of claim 76, wherein the cardiac disorder is an arrhythmia, optionally atrial fibrillation or sinus node disease, or familial hypertrophic cardiomyopathy 17.

81. The method of claim 76, wherein the cardiac disorder is heart failure.

82. The method of any one of claims 76 to 81, wherein the subject is a mammal.

83. The method of any one of claims 76 to 82, wherein the vector is administered by intravenous injection, intracardiac injection, intracardiac infusion, and/or cardiac catheterization.


SEQUENCE LISTING (108 sequences)

SEQ ID NO: 1 - protein, 696 aa, Homo sapiens
MSGGRFDFDDGGAYCGGWEGGKAHGHGLCTGPKGQGEYSGSWNFGFEVAGVYTWPSGNTFEGYWSQGKRHGLGIETKGRWLYKGEWTHGFKGRYGIRQSSSSGAKYEGTWNNGLQDGYGTETYADGGTYQGQFTNGMRHGYGVRQSVPYGMAVVVRSPLRTSLSSLRSEHSNGTVAPDSPASPASDGPALPSPAIPRGGFALSLLANAEAAARAPKGGGLFQRGALLGKLRRAESRTSVGSQRSRVSFLKSDLSSGASDAASTASLGEAAEGADEAAPFEADIDATTTETYMGEWKNDKRSGFGVSERSSGLRYEGEWLDNLRHGYGCTTLPDGHREEGKYRHNVLVKDTKRRMLQLKSNKVRQKVEHSVEGAQRAAAIARQKAEIAASRTSHAKAKAEAAEQAALAANQESNIARTLARELAPDFYQPGPEYQKRRLLQEILENSESLLEPPDRGAGAAGLPQPPRESPQLHERETPRPEGGSPSPAGTPPQPKRPRPGVSKDGLLSPGAWNGEPSGEGSRSVTPSEGAGRRSPARPATERMAIEALQAPPAPSREPEVALYQGYHSYAVRTTPPEPPPFEDQPEPEVSGSESAPSSPATAPLQAPTLRGPEPARETPAKLEPKPIIPKAEPRAKARKTEARGLTKAGAKKKARKEAALAAEAEVEVEEVPNTILICMVILLNIGLAILFVHLLT

SEQ ID NO: 2 - DNA, 2091 bp, Homo sapiens
  source 1..2091: mol_type = genomic DNA
atgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgcgcaccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctga

SEQ ID NO: 3 - protein, 696 aa, synthetic construct
  REGION 1..696: note = Mutated human JPH2 sequence
MSGGRFDFDDGGAYCGGWEGGKAHGHGLCTGPKGQGEYSGSWNFGFEVAGVYTWPSGNTFEGYWSQGKRHGLGIETKGRWLYKGEWTHGFKGRYGIRQSSSSGAKYEGTWNNGLQDGYGTETYADGGTYQGQFTNGMRHGYGVRQSVPYGMAVVVRSPLRTSLSSLRSEHSNGTVAPDSPASPASDGPALPSPAIPRGGFALSLLANAEAAARAPKGGGLFQRGALLGKLRRAESRTSVGSQRSRVSFLKSDLSSGASDAASTASLGEAAEGADEAAPFEADIDATTTETYMGEWKNDKRSGFGVSERSSGLRYEGEWLDNLRHGYGCTTLPDGHREEGKYRHNVLVKDTKRRMLQLKSNKVRQKVEHSVEGAQRAAAIARQKAEIAASRTSHAKAKAEAAEQAALAANQESNIARTLARELAPDFYQPGPEYQKRRLLQEILENSESLLEPPDRGAGAAGLPQPPRESPQLHERETPRPEGGSPSPAGTPPQPKRPRPGVSKDGLLSPGAWNGEPSGEGSRSVTPSEGAGRRSPARPATERMAIEALQAPPAPSREPEVALYQGYHSYAVAATPPEPPPFEDQPEPEVSGSESAPSSPATAPLQAPTLRGPEPARETPAKLEPKPIIPKAEPRAKARKTEARGLTKAGAKKKARKEAALAAEAEVEVEEVPNTILICMVILLNIGLAILFVHLLT

SEQ ID NO: 4 - DNA, 2091 bp, synthetic construct
  misc_feature 1..2091: note = Mutated human JPH2 sequence
  source 1..2091: mol_type = other DNA
atgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtggccgccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctga

SEQ ID NO: 5 - protein, 696 aa, synthetic construct
  REGION 1..696: note = Mutated human JPH2 sequence
MSGGRFDFDDGGAYCGGWEGGKAHGHGLCTGPKGQGEYSGSWNFGFEVAGVYTWPSGNTFEGYWSQGKRHGLGIETKGRWLYKGEWTHGFKGRYGIRQSSSSGAKYEGTWNNGLQDGYGTETYADGGTYQGQFTNGMRHGYGVRQSVPYGMAVVVRSPLRTSLSSLRSEHSNGTVAPDSPASPASDGPALPSPAIPRGGFALSLLANAEAAARAPKGGGLFQRGALLGKLRRAESRTSVGSQRSRVSFLKSDLSSGASDAASTASLGEAAEGADEAAPFEADIDATTTETYMGEWKNDKRSGFGVSERSSGLRYEGEWLDNLRHGYGCTTLPDGHREEGKYRHNVLVKDTKRRMLQLKSNKVRQKVEHSVEGAQRAAAIARQKAEIAASRTSHAKAKAEAAEQAALAANQESNIARTLARELAPDFYQPGPEYQKRRLLQEILENSESLLEPPDRGAGAAGLPQPPRESPQLHERETPRPEGGSPSPAGTPPQPKRPRPGVSKDGLLSPGAWNGEPSGEGSRSVTPSEGAGRRSPARPATERMAIEALQAPPAPSREPEVALYQGYHSYAVKSTPPEPPPFEDQPEPEVSGSESAPSSPATAPLQAPTLRGPEPARETPAKLEPKPIIPKAEPRAKARKTEARGLTKAGAKKKARKEAALAAEAEVEVEEVPNTILICMVILLNIGLAILFVHLLT

SEQ ID NO: 6 - DNA, 2091 bp, synthetic construct
  misc_feature 1..2091: note = Mutated human JPH2 sequence
  source 1..2091: mol_type = other DNA
atgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgaagagcacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctga

SEQ ID NO: 7 - protein, 696 aa, synthetic construct
  REGION 1..696: note = Mutated human JPH2 sequence
MSGGRFDFDDGGAYCGGWEGGKAHGHGLCTGPKGQGEYSGSWNFGFEVAGVYTWPSGNTFEGYWSQGKRHGLGIETKGRWLYKGEWTHGFKGRYGIRQSSSSGAKYEGTWNNGLQDGYGTETYADGGTYQGQFTNGMRHGYGVRQSVPYGMAVVAASPLRTSLSSLRSEHSNGTVAPDSPASPASDGPALPSPAIPRGGFALSAAANAEAAARAPKGGGLFQRGALLGKLRRAESRTSVGSQRSRVSFLKSDLSSGASDAASTASLGEAAEGADEAAPFEADIDATTTETYMGEWKNDKRSGFGVSERSSGLRYEGEWLDNLRHGYGCTTLPDGHREEGKYRHNVLVKDTKRRMLQLKSNKVRQKVEHSVEGAQRAAAIARQKAEIAASRTSHAKAKAEAAEQAALAANQESNIARTLARELAPDFYQPGPEYQKRRLLQEILENSESLLEPPDRGAGAAGLPQPPRESPQLHERETPRPEGGSPSPAGTPPQPKRPRPGVSKDGLLSPGAWNGEPSGEGSRSVTPSEGAGRRSPARPATERMAIEALQAPPAPSREPEVALYQGYHSYAVAATPPEPPPFEDQPEPEVSGSESAPSSPATAPLQAPTLRGPEPARETPAKLEPKPIIPKAEPRAKARKTEARGLTKAGAKKKARKEAALAAEAEVEVEEVPNTILICMVILLNIGLAILFVHLLT

SEQ ID NO: 8 - DNA, 2091 bp, synthetic construct
  misc_feature 1..2091: note = Mutated human JPH2 sequence
  source 1..2091: mol_type = other DNA
atgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggccgcctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcgccgccgccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtggccgccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctga

SEQ ID NO: 9 - protein, 696 aa, synthetic construct
  REGION 1..696: note = Mutated human JPH2 sequence
MSGGRFDFDDGGAYCGGWEGGKAHGHGLCTGPKGQGEYSGSWNFGFEVAGVYTWPSGNTFEGYWSQGKRHGLGIETKGRWLYKGEWTHGFKGRYGIRQSSSSGAKYEGTWNNGLQDGYGTETYADGGTYQGQFTNGMRHGYGVRQSVPYGMAVVAKSPLRTSLSSLRSEHSNGTVAPDSPASPASDGPALPSPAIPRGGFALSAAANAEAAARAPKGGGLFQRGALLGKLRRAESRTSVGSQRSRVSFLKSDLSSGASDAASTASLGEAAEGADEAAPFEADIDATTTETYMGEWKNDKRSGFGVSERSSGLRYEGEWLDNLRHGYGCTTLPDGHREEGKYRHNVLVKDTKRRMLQLKSNKVRQKVEHSVEGAQRAAAIARQKAEIAASRTSHAKAKAEAAEQAALAANQESNIARTLARELAPDFYQPGPEYQKRRLLQEILENSESLLEPPDRGAGAAGLPQPPRESPQLHERETPRPEGGSPSPAGTPPQPKRPRPGVSKDGLLSPGAWNGEPSGEGSRSVTPSEGAGRRSPARPATERMAIEALQAPPAPSREPEVALYQGYHSYAVKSTPPEPPPFEDQPEPEVSGSESAPSSPATAPLQAPTLRGPEPARETPAKLEPKPIIPKAEPRAKARKTEARGLTKAGAKKKARKEAALAAEAEVEVEEVPNTILICMVILLNIGLAILFVHLLT

SEQ ID NO: 10 - DNA, 2091 bp, synthetic construct
  misc_feature 1..2091: note = Mutated human JPH2 sequence
  source 1..2091: mol_type = other DNA
atgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggccaagtcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcgccgccgccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgaagagcacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctga

SEQ ID NO: 11 - DNA, 10 bp, synthetic construct
  misc_feature 1..10: note = Koozak sequence motif
  source 1..10: mol_type = other DNA
gccaccatgg

SEQ ID NO: 12 - DNA, 2094 bp, synthetic construct
  misc_feature 1..2094: note = kozak sequence and JPH2 wt
  source 1..2094: mol_type = other DNA
gccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgcgcaccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacc

SEQ ID NO: 13 - DNA, 13 bp, synthetic construct
  misc_feature 1..13: note = Kozak sequence motif
  source 1..13: mol_type = other DNA
gccgccrccatgg

SEQ ID NO: 14 - DNA, 10 bp, synthetic construct
  misc_feature 1..10: note = Kozak sequence motif
  source 1..10: mol_type = other DNA
gacaccatgg

SEQ ID NO: 15 - DNA, 141 bp, unidentified
  source 1..141: mol_type = genomic DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 16 - DNA, 168 bp, unidentified
  source 1..168: mol_type = genomic DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 17 - DNA, 170 bp, unidentified
  source 1..170: mol_type = genomic DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 18 - DNA, 145 bp, unidentified
  source 1..145: mol_type = genomic DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 19 - DNA, 141 bp, unidentified
  source 1..141: mol_type = genomic DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 20 - DNA, 168 bp, unidentified
  source 1..168: mol_type = genomic DNA
tacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 21 - DNA, 133 bp, unidentified
  source 1..133: mol_type = genomic DNA
aggaacccctagtgatggagactccctctctgcgcgctcgctcgctcactgaggccgggcgaccaaaggtcgcccgacgcccgggctttgcccgggcggcctcagtgagcgagcgagcgcgcagagagggagt

SEQ ID NO: 22 - DNA, 124 bp, synthetic construct
  misc_feature 1..124: note = Made in Lab - vector filler sequence
  source 1..124: mol_type = other DNA
gcggcaattcagtcgataactataacggtcctaaggtagcgatttaaatacgcgctctcttaaggtagccccgggacgcgtcaattgactacaaaccgagtatctgcagagggccctgcgtatg

SEQ ID NO: 23 - DNA, 84 bp, synthetic construct
  misc_feature 1..84: note = Made in Lab - vector filler sequence
  source 1..84: mol_type = other DNA
cttctgaggcggaaagaaccagatcctctcttaaggtagcatcgagatttaaattagggataacagggtaatggcgcgggccgc

SEQ ID NO: 24 - DNA, 63 bp, synthetic construct
  misc_feature 1..63: note = Made in Lab - vector filler sequence
  source 1..63: mol_type = other DNA
gttacccaggctggagtgcagtggcacatttctgctcactgcaacctcctcctccctgggttc

SEQ ID NO: 25 - DNA, 573 bp, synthetic construct
  misc_feature 1..573: note = Made in lab - CAG promoter in part Human betaherpesvirus 5
  source 1..573: mol_type = other DNA
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 26 - DNA, 4117 bp, synthetic construct
  misc_feature 1..4117: note = Made in Lab - vector genome polynucleotide sequence
  source 1..4117: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgcgcaccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 27 - DNA, 3741 bp, synthetic construct
  misc_feature 1..3741: note = Made in Lab - vector genome polynucleotide sequence
  source 1..3741: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgcgcaccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 28 - DNA, 4751 bp, synthetic construct
  misc_feature 1..4751: note = Made in Lab - vector genome polynucleotide sequence
  source 1..4751: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgcgcaccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgaccggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 29 - DNA, 4524 bp, synthetic construct
  misc_feature 1..4524: note = Made in Lab - vector genome polynucleotide sequence
  source 1..4524: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgcgcaccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgaccggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 30 - DNA, 4583 bp, synthetic construct
  misc_feature 1..4583: note = Made in Lab - vector genome polynucleotide sequence
  source 1..4583: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgttacatacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgcgcaccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgaccggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 31 - DNA, 771 bp, synthetic construct
  misc_feature 1..771: note = Made in Lab - MHCK7 promoter
  source 1..771: mol_type = other DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccag

SEQ ID NO: 32 - DNA, 544 bp, Homo sapiens
  source 1..544: mol_type = genomic DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcag

SEQ ID NO: 33 - DNA, 502 bp, Homo sapiens
  source 1..502: mol_type = genomic DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagt

SEQ ID NO: 34 - DNA, 5464 bp, Mus musculus
  source 1..5464: mol_type = genomic DNA
ggtaccggatcctgcaaggtcacacaagggtctccacccaccaggtgccctagtctcaatttcagtttccatgccttgttctcacaatgctggcctccccagagctaatttggactttgtttttatttcaaaagggcctgaatgaggagtagatcttgtgctacccagctctaagggtgcccgtgaagccctcagacctggagcctttgcaacagccctttaggtggaagcagaataaagcaattttccttaaagccaaaatcctgcctctagactcttcttctctgacctcggtccctgggctctagggtggggaggtggggcttggaagaagaaggtggggaagtggcaaaagccgatccctagggccctgtgaagttcggagccttccctgtacagcactggctcatagatcctcctccagccaaacatagcaagaagtgatacctcctttgtgacttccccaggcccagtacctgtcaggttgaaacaggatttagagaagcctctgaactcacctgaactctgaagctcatccaccaagcaagcacctaggtgccactgctagttagtatcctacgctgataatatgcagagctgggccacagaagtcctggggtgtaggaactgaccagtgacttttcagtcggcaaaggtatgaccccctcagcagatgtagtaatgtccccttagatcccatcccaggcaggtctctaagaggacatgggatgagagatgtagtcatgtggcattccaaacacagctatccacagtgtcccttgccccttccacttagccaggaggacagtaaccttagcctatctttcttcctccccatcctcccaggacacaccccctggtctgcagtattcatttcttccttcacgtcccctctgtgacttccatttgcaaggcttttgacctctgcagctgctggaagatagagtttggccctaggtgtggcaagccatctcaagagaaagcagacaacagggggaccagattttggaaggatcaggaactaaatcactggcgggcctgggggtagaaaaaagagtgagtgagtccgctccagctaagccaagctagtccccgagatactctgccacagctgggctgctcggggtagctttaggaatgtgggtctgaaagacaatgggattggaagacatctctttgagtctcccctcaaccccacctacagacacactcgtgtgtggccagactcctgttcaacagccctctgtgttctgaccactgagctaggcaaccagagcatgggccctgtgctgaggatgaagagttggttaccaatagcaaaaacagcaggggagggagaacagagaacgaaataaggaaggaagaaggaaaggccagtcaatcagatgcagtcagaagagatgggaagccaacacacagcttgagcagaggaaacagaaaagggagagattctgggcataaggaggccacagaaagaagagcccaggccccccaagtctcctctttataccctcatcccgtctcccaattaagcccactcttcttcctagatcagacctgagctgcagcgaagagacccgtagggaggatcacactggatgaaggagatgtgtggagaagtccagggaacctaagagccagagcctaaaagagcaagagataaaggtgcttcaaaggtggccaggctgtgcacacagagggtcgaggactggtggtagagcctcaagataaggatgatgctcagaatgggcggggggggggattctgggggggggagagagaaggtgagaaggagcctggaacagagaatctggaagcgctggaaacgataccataaagggaagaacccaggctacctttagatgtaaatcatgaaagacagggagaagggaagctggagagagtagaaggaccccggggcaagacattgaagcaaggacaagccaggttgagcgctccgtgaaatcagcctgctgaaggcagagccctggtatgagcaccagaacagcagaggctagggttaatgtcgagacagggaacagaaggtagacacaggaacagacagagacgggggagccaggtaacaaaggaatggtccttctcacctgtggccagagcgtccatctgtgtccacatactctagaatgttcatcagactgcagggctggcttgggaggcagctggaaagagtatgtgagagccaggggagacaagggggcctaggaaaggaagaagagggcaaaccaggccacacaagagggcagagcccagaactgagttaactccttccttgttgcatcttccataggaggcagtgggaactctgtgaccaccatcccccatgagcccccactacccataccaagtttggcctgagtggcattctaggttccctgaggacagagcctggcctttgtctcttggacctgacccaagctgacccaatgttctcagtaccttatcatgccctcaagagcttgagaaccaggcagtgacatattaggccatgggctaaccctggagcttgcacacaggagcctcaagtgacctccagggacacagctgcagacaggtggcctttatccccaaagagcaaccatttggcataggtggctgcaaatgggaatgcaaggttgaatcaggtcccttcaagaatactgcatgcaagacctaagacccctggagagaggggtatgctcctgcccccacccaccataaggggagtgaactatcctagggggctggcgaccttggggagacaccacattactgagagtgctgagcccagaaaaactgaccgccctgtgtcctgcccacctccacactctagagctatattgagaggtgacagtagatagggtgggagctggtagcagggagagtgttcctgggtgtgagggtgtaggggaaagccagagcaggggagtctggctttgtctcctgaacacaatgtctacttagttataacaggcatgacctgctaaagacccaacatctacgacctctgaaaagacagcagccctggaggacaggggttgtctctgagccttgggtgcttgatggtgccacaaaggagggcatgagtgtgagtataaggccccaggagcgttagagaagggcacttgggaaggggtcagtctgcagagcccctatccatggaatctggagcctggggccaactggtgtaaatctctgggcctgccaggcattcaaagcagcacctgcatcctctggcagcctggggaggcggaagggagcaaccccccacttataccctttctccctcagccccaggattaacacctctggccttcccccttcccacctcccatcaggagtggagggttgcagagggagggtaaaaacctacatgtccaaacatcatggtgcacgatatatggatcagtatgtgtagaggcaagaaaggaaatctgcaggcttaactgggttaatgtgtaaagtctgtgtgcatgtgtgtgtgtctgactgaaaacgggcatggctgtgcagctgttcagttctgtgcgtgaggttaccagactgcaggtttgtgtgtaaattgcccaaggcaaagtgggtgaatcccttccatggtttaaagagattggatgatggcctgcatctcaaggaccatggaaaatagaatggacactctatatgtgtctctaagctaaggtagcaaggtctttggaggacacctgtctagagatgtgggcaacagagactacagacagtatctgtacagagtaaggagagagaggagggggtgtagaattctcttactatcaaagggaaactgagtcgtgcacctgcaaagtggatgctctccctagacatcatgactttgtctctggggagccagcactgtggaacttcaggtctgagagagtaggaggctcccctcagcctgaagctatgcagatagccagggttgaaagggggaagggagagcctgggatgggagcttgtgtgttggaggcaggggacagatattaagcctggaagagaaggtgacccttacccagttgttcaactcacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtcctcctgtcacctccagagccaagggatcaaaggaggaggagccaggacaggagggaagtgggagggagggtcccagcagaggactccaaatttaggcagcaggcatatgggatgggatataaaggggctggagcactgagagctgtcagagatttctccaacccaggtaagagggagtttcgggtgggggctcttcacccacaccagacctctccccacctagaaggaaactgcctttcctggaagtggggttcaggccggtcagagatctgacagggtggccttccaccagcctgggaagttctcagtggcaggaggtttccacaagaaacactggatgccccttcccttacgctgtcttctccatcttcctcctggggatgctcctccccgtcttggtttatcttggctcttcgtcttcagcaagatttgccctgtgctgtccactccatctttctctactgtctccgtgccttgccttgccttcttgcgtgtccttcctttccacccatttctcacttcaccttttctccccttctcatttgtattcatccttccttccttccttccttccttccttccttccttccttccttcctttctcccttccttccttccttccttccttccttccttccttccttcctgtgtcagagtgctgagaatcacacctggggttcccacccttatgtaaacaatcttccagtgagccacagcttcagtgctgctgggtgctctcttaccttcctcaccccctggcttgtcctgttccatcctggtcaggatctctagattggtctcccagcctctgctactcctcttcctgcctgttcctctctctgtccagctgcgccactgtggtgcctcgttccagctgtggtccacattcttcaggattctctgaaaagttaaccaggtgagaatgtttcccctgtagacagcagatcacgattctcccggaagtcaggcttccagccctctctttctctgcccagctgcccggcactcttagcaaacctcaggcacccttaccccacatagacctctgacagagaagcaggcactttacatggagtcctggtgggagagccataggctacggtgtaaaagaggcagggaagtggtggtgtaggaaagtcaggacttcacatagaagcctagcccacaccagaaatgacagacagatccctcctatctcccccataagagtttgagtcgacccgcggccccgaattg

SEQ ID NO: 35 - DNA, 413 bp, Gallus gallus
  source 1..413: mol_type = genomic DNA
gggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtggga

SEQ ID NO: 36 - DNA, 1090 bp, Homo sapiens
  source 1..1090: mol_type = genomic DNA
ctctcagccctggaagtccttgctcacagccgaggcgccgagagcgcttgctctgcccagatctgcgcgagtctggcgcccgcgctctgaacggcgtcgctgcccagcccccttccccgggaggtgggagcggccacccagggccccgtggctgcccttgtaaggaggcgaggcccgaggacacccgagacgcccggttataattaaccaggacacgtggcgaacccccctccaacacctgcccccgaacccccccatacccagcgcctcgggtctcggcctttgcggcagaggagacagcaaagcgccctctaaaaataactcctttcccggcgaccgagaccctccctgtcccccgcacagcggaaatctcccagtggcaccgagggggcgagggttaagtgggggggagggtgaccaccgcctcccacccttgccctgagtttgaatctctccaactcagccagcctcagtttcccctccactcagtccctaggaggaaggggcgcccaagcgcgggtttctggggttagactgccctccattgcaattggtccttctcccggcctctgcttcctccagctcacagggtatctgctcctcctggagccacaccttggttccccgaggtgccgctgggactcgggtaggggtgagggcccagggggcacagggggagccgagggccacaggaagggctggtggctgaaggagactcaggggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttcctttgggtcagtgtcacctccaggatacagaca

SEQ ID NO: 37 - DNA, 253 bp, Homo sapiens
  source 1..253: mol_type = genomic DNA
gcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagtc

SEQ ID NO: 38 - DNA, 281 bp, Gallus gallus
  source 1..281: mol_type = genomic DNA
ggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggga

SEQ ID NO: 39 - DNA, 220 bp, Human betaherpesvirus 5
  source 1..220: mol_type = genomic DNA
tggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccg

SEQ ID NO: 40 - DNA, 583 bp, Human betaherpesvirus 5
  source 1..583: mol_type = genomic DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgt

SEQ ID NO: 41 - DNA, 508 bp, Human betaherpesvirus 5
  source 1..508: mol_type = genomic DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 42 - DNA, 573 bp, synthetic construct; identical to SEQ ID NO: 25
  misc_feature 1..573: note = Made in lab - CAG promoter in part Human betaherpesvirus 5
  source 1..573: mol_type = other DNA

SEQ ID NO: 43 - DNA, 580 bp, synthetic construct
  misc_feature 1..580: note = Made in lab - CAG promoter in part Human betaherpesvirus 5
  source 1..580: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 44 - DNA, 455 bp, Homo sapiens
  source 1..455: mol_type = genomic DNA
caacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaatagcatgcgtgaggctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgtt

SEQ ID NO: 45-47 - skipped (3 entries)

SEQ ID NO: 48 - DNA, 281 bp, Homo sapiens
  source 1..281: mol_type = genomic DNA
acttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgc

SEQ ID NO: 49 - skipped

SEQ ID NO: 50 - DNA, 293 bp, Human betaherpesvirus 5
  source 1..293: mol_type = genomic DNA
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattacca

SEQ ID NO: 51 - DNA, 953 bp, Homo sapiens
  source 1..953: mol_type = genomic DNA
cgcgtccgcccgcgagcacagagcctcgcctttgccgatccgccgcccgtccacacccgccgccaggtaagcccggccagccgaccggggcatgcggccgcggcccttcgcccgtgcagagccgccgtctgggccgcagcggggggcgcatggggcggaaccggaccgccgtggggggcgcgggagaagcccctgggcctccggagatgggggacaccccacgccagttcgcaggcgcgaggccgcgctcgggcgggcgcgctccgggggtgccgctctcggggcgggggcaaccggcggggtctttgtctgagccgggctcttgccaatggggatcgcacggtgggcgcggcgtagcccccgtcaggcccggtgggggctggggcgccatgcgcgtgcgcgctggtcctttgggcgctaactgcgtgcgcgctgggaattggcgctaattgcgcgtgcgcgctgggactcaatggcgctaatcgcgcgtgcgttctggggcccgggcgcttgcgccacttcctgcccgagccgctggcgcccgagggtgtggccgctgcgtgcgcgcgcgcgacccggtcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcggcagcggctgctcttggcggccccgaggtgactatagccttcttttgtgtcttgatagttcgccagcctctgctaaccatgttcatgccttcttctttttcctacagctcctgggcaacgtgctggttattgtgctgtctcatcattttggcaaagaattc

SEQ ID NO: 52 - DNA, 1068 bp, synthetic construct
  misc_feature 1..1068: note = Made in Lab - Chicken beta-actin exon/intron plus rabbit globin intron
  source 1..1068: mol_type = other DNA
gtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 53 - DNA, 149 bp, synthetic construct
  misc_feature 1..149: note = Made in Lab - chimeric intron sequence
  source 1..149: mol_type = other DNA
ggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgc

SEQ ID NO: 54 - DNA, 126 bp, Homo sapiens
  source 1..126: mol_type = genomic DNA
agtctgcggtgggcagcggaggagtcgtgtcgtgcctgagagcgcagctgtgctcctgggcaccgcgcagtccgcccccgcggctcctggccagaccacccctaggaccccctgccccaagtcgca

SEQ ID NO: 55 - DNA, 121 bp, Human betaherpesvirus 5
  source 1..121: mol_type = unassigned DNA
tcagatcgcctggagaggccatccacgctgttttgacctccatagtggacaccgggaccgatccagcctccgcggccgggaacggtgcattggaacgcggattccccgtgccaagagtgac

SEQ ID NO: 56 - DNA, 512 bp, synthetic construct
  misc_feature 1..512: note = Made in Lab - adenovirus derived enhancer element
  source 1..512: mol_type = other DNA
ctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccag

SEQ ID NO: 57 - DNA, 956 bp, Homo sapiens
  source 1..956: mol_type = genomic DNA
ctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacgtgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcag

SEQ ID NO: 58 - DNA, 939 bp, Homo sapiens
  source 1..939: mol_type = genomic DNA
gtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 59 - DNA, 83 bp, Homo sapiens
  source 1..83: mol_type = genomic DNA
tcagaagccccgggctcgtcagtcaaaccggttctctgtttgcactcggcagcacgggcaggcaagtggtccctaggttcggg

SEQ ID NO: 60 - DNA, 476 bp, Homo sapiens
  source 1..476: mol_type = genomic DNA
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 61 - DNA, 196 bp, unidentified
  source 1..196: mol_type = genomic DNA
tctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgc

SEQ ID NO: 62 - DNA, 589 bp, synthetic construct
  misc_feature 1..589: note = Made in Lab - mutated woodchuck hepatitis regulatory element
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 63 - DNA, 588 bp, synthetic construct
  misc_feature 1..588: note = Made in Lab - mutated woodchuck hepatitis regulatory element
  source 1..588: mol_type = other DNA
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgca

SEQ ID NO: 64 - DNA, 755 bp, synthetic construct
  misc_feature 1..755: note = Made in Lab - mutated woodchuck hepatitis regulatory element
  source 1..755: mol_type = other DNA
ttcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 65 - DNA, 12 bp, synthetic construct
  misc_feature 1..12: note = CAAX motif
  source 1..12: mol_type = other DNA
tgtgtgataatg

SEQ ID NO: 66 - DNA, 810 bp, Homo sapiens
  source 1..810: mol_type = genomic DNA
ctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagtta

SEQ ID NO: 67 - DNA, 726 bp, Hepatitis B virus
  source 1..726: mol_type = genomic DNA
ataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatc

SEQ ID NO: 68 - DNA, 755 bp, synthetic construct
  misc_feature 1..755: note = Made in Lab - HepB derived enhancer element
  source 1..755: mol_type = other DNA
ttcctgtaaacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagctggactggagcaaacctcatcgggaccgacaattctgtcgtactctcccgcaagcactcaccgtttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 69 - DNA, 94 bp, Homo sapiens
  source 1..94: mol_type = genomic DNA
gctggagcctcggtagccgttcctcctgcccgctgggcctcccaacgggccctcctcccctccttgcaccggcccttcctggtctttgaataaa

SEQ ID NO: 70 - DNA, 596 bp, Woodchuck hepatitis virus
  source 1..596: mol_type = genomic DNA
attcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcc

SEQ ID NO: 71 - DNA, 387 bp, Oryctolagus cuniculus
  source 1..387: mol_type = genomic DNA
tggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 72 - DNA, 251 bp, Bos taurus
  source 1..251: mol_type = genomic DNA
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaatacaatagcaggcatgctggggatgcggtgggctctatgggtacccaggtgctgaagaattgacccggttcctcctggg

SEQ ID NO: 73 - DNA, 251 bp, Bos taurus
  source 1..251: mol_type = genomic DNA
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctggg

SEQ ID NO: 74 - DNA, 225 bp, Bos taurus
  source 1..225: mol_type = genomic DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 75 - DNA, 202 bp, Homo sapiens
  source 1..202: mol_type = genomic DNA
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 76 - DNA, 4117 bp, synthetic construct
  misc_feature 1..4117: note = Made in Lab - vector genome polynucleotide sequence
  source 1..4117: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtggccgccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 77 - DNA, 3741 bp, synthetic construct
  misc_feature 1..3741: note = Made in Lab - vector genome polynucleotide sequence
  source 1..3741: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtggccgccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 78 - DNA, 4751 bp, synthetic construct
  misc_feature 1..4751: note = Made in Lab - vector genome polynucleotide sequence
  source 1..4751: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtggccgccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgaccggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 79 - DNA, 4524 bp, synthetic construct
  misc_feature 1..4524: note = Made in Lab - vector genome polynucleotide sequence
  source 1..4524: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtggccgccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgaccggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 80 - DNA, 4583 bp, synthetic construct
  misc_feature 1..4583: note = Made in Lab - vector genome polynucleotide sequence
  source 1..4583: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgttacatacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtggccgccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgaccggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 81 - DNA, 4117 bp, synthetic construct
  misc_feature 1..4117: note = Made in Lab - vector genome polynucleotide sequence
  source 1..4117: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgaagagcacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 82 - DNA, 3741 bp, synthetic construct
  misc_feature 1..3741: note = Made in Lab - vector genome polynucleotide sequence
  source 1..3741: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgaagagcacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 83 - DNA, 4751 bp, synthetic construct
  misc_feature 1..4751: note = Made in Lab - vector genome polynucleotide sequence
  source 1..4751: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgaagagcacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgaccggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 84 - DNA, 4524 bp, synthetic construct
  misc_feature 1..4524: note = Made in Lab - vector genome polynucleotide sequence
  source 1..4524: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgaagagcacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgaccggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 85 - DNA, 4583 bp, synthetic construct
  misc_feature 1..4583: note = Made in Lab - vector genome polynucleotide sequence
  source 1..4583: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgttacatacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgaagagcacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgaccggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 86 - DNA, 4117 bp, synthetic construct
  misc_feature 1..4117: note = Made in Lab - vector genome polynucleotide sequence
  source 1..4117: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggccgcctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcgccgccgccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtggccgccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 87 - DNA, 3741 bp, synthetic construct
  misc_feature 1..3741: note = Made in Lab - vector genome polynucleotide sequence
  source 1..3741: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggccgcctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcgccgccgccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtggccgccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 88 - DNA, 4751 bp, synthetic construct
  misc_feature 1..4751: note = Made in Lab - vector genome polynucleotide sequence
  source 1..4751: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggccgcctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcgccgccgccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtggccgccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgaccggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 89 - DNA, 4524 bp, synthetic construct
  misc_feature 1..4524: note = Made in Lab - vector genome polynucleotide sequence
  source 1..4524: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggccgcctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcgccgccgccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtggccgccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgaccggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 90 - DNA, 4583 bp, synthetic construct
  misc_feature 1..4583: note = Made in Lab - vector genome polynucleotide sequence
  source 1..4583: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgttacatacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggccgcctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcgccgccgccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtggccgccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgaccggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 91 - DNA, 4117 bp, synthetic construct
  misc_feature 1..4117: note = Made in Lab - vector genome polynucleotide sequence
  source 1..4117: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggccaagtcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcgccgccgccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgaagagcacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 92 - DNA, 3741 bp, synthetic construct
  misc_feature 1..3741: note = Made in Lab - vector genome polynucleotide sequence
  source 1..3741: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggccaagtcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcgccgccgccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgaagagcacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 93 - DNA, 4751 bp, synthetic construct
  misc_feature 1..4751: note = Made in Lab - vector genome polynucleotide sequence
  source 1..4751: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggccaagtcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcgccgccgccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgaagagcacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgaccggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 94 - DNA, 4524 bp, synthetic construct
  misc_feature 1..4524: note = Made in Lab - vector genome polynucleotide sequence
  source 1..4524: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggccaagtcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcgccgccgccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgaagagcacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgaccggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 95 - DNA, 4583 bp, synthetic construct
  misc_feature 1..4583: note = Made in Lab - vector genome polynucleotide sequence
  source 1..4583: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgttacatacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggccaccatgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggccaagtcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcgccgccgccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgaagagcacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgaccggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 96 - protein, 735 aa, unidentified [sequence omitted]

SEQ ID NO: 97 - protein, 736 aa, unidentified
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 98 - protein, 736 aa, unidentified
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 99 - protein, 738 aa, unidentified
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 100 - DNA, 2217 bp, unidentified
  source 1..2217: mol_type = genomic DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 101 - protein, 535 aa, unidentified
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 102 - protein, 398 aa, unidentified
STIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 103 - protein, 332 aa, unidentified
RTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 104 - protein, 743 aa, synthetic construct
  REGION 1..743: note = Synthetic construct - AAV variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 105 - protein, 7 aa, synthetic construct
  REGION 1..7: note = Peptide insert
TLAVPFK

SEQ ID NO: 106 - protein, 7 aa, synthetic construct
  REGION 1..7: note = Peptide insert
KFPVALT

SEQ ID NO: 107 - DNA, 390 bp, Homo sapiens
  source 1..390: mol_type = other DNA
atgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggatgtgttaa

SEQ ID NO: 108 - protein, 129 aa, Homo sapiens
MSGGRFDFDDGGAYCGGWEGGKAHGHGLCTGPKGQGEYSGSWNFGFEVAGVYTWPSGNTFEGYWSQGKRHGLGIETKGRWLYKGEWTHGFKGRYGIRQSSSSGAKYEGTWNNGLQDGYGTETYADGGMC